(19) United States
(12) Patent Application Publication
(10) Pub. No.: US 2005/0203151 A1
(43) Pub. Date: Sep. 15, 2005

(54) Novel compounds, compositions and uses thereof for treatment of metabolic disorders and related conditions

(75) Inventors: James William Malecha, San Diego, CA (US); Cunxiang Zhao, San Diego, CA (US); Carmen Ka Man Robinson, San Diego, CA (US); Sergio Gonzalez Duron, San Diego, CA (US); Michael Sertic, Del Mar, CA (US); Stewart Alwyn Noble, San Diego, CA (US)

Correspondence Address: PILLSBURY WINTHROP SHAW PITTMAN LLP, ATTENTION: DOCKETING DEPARTMENT, 11682 EL CAMINO REAL, SUITE 200, SAN DIEGO, CA, 92130, US

(73) Assignee: Kalypsys, Inc., San Diego, CA (US)

(21) Appl. No.: 11/019,146

(22) Filed: Dec. 20, 2004

Related U.S. Application Data
(60) Provisional application No. 60/531,497, filed on Dec. 19, 2003.

(51) Int. Cl. A61K 31/427; C07D 417/02

(52) U.S. Cl. 514/365; 548/181

(57) ABSTRACT

Described herein are novel mono- and bicyclic compounds compounds, including compounds capable of modulating the activity of human peroxisome proliferator activated receptor of the subtype delta (hPPAR-delta), and methods for utilizing such modulation to treat a disease or condition mediated or impacted by hPPAR-delta activity such as Type 2 diabetes, syndrome X, dyslipidemia, and atherosclerotic diseases including vascular disease, coronary heart disease, cerebrovascular disease, and peripheral vessel disease. Also described are compounds that mediate and/or inhibit the activity of hPPAR-delta, and pharmaceutical compositions containing such compounds or pharmaceutically acceptable prodrugs, solvates, salts, esters, thioesters, or amides or pharmaceutically active metabolites thereof. Further described are methods for making and producing such compounds. Also described are the therapeutic or prophylactic use of such compounds or compositions, and methods of treating metabolic disorders and conditions, by administering effective amounts of such compounds.

### CROSS REFERENCE TO RELATED APPLICATION

This application claims priority of U.S. provisional patent application serial No. 60/531,497, filed Dec. 19, 2003.

### FIELD OF THE INVENTION

Described herein are novel compounds and compositions and methods for using them to treat metabolic disorders or related conditions, such as Type 2 diabetes, syndrome X, dyslipidemia, and atherosclerotic diseases including vascular disease, coronary heart disease, cerebrovascular disease, and peripheral vessel disease. In particular, an aspect of the present invention relates to compounds that mediate the delta subtype of the human peroxisome proliferator activated receptor (“hPPAR-delta”). An aspect of the present invention also relates to methods for preparing and using the novel compounds and to methods for modulating hPPAR-delta.

### BACKGROUND OF THE INVENTION

Peroxisome proliferators are a structurally diverse group of compounds which, when administered to mammals, elicit dramatic increases in the size and number of hepatic and renal peroxisomes, as well as concomitant increases in the capacity of peroxisomes to metabolize fatty acids via increased expression of the enzymes required for the β-oxidation cycle (Lazarow and Fujiki, Ann. Rev. Cell Biol. 1:489-530 (1985); Vamecq and Draye, Essays Biochem. 24:1115-225 (1989); and Nelali et al., Cancer Res. 48:5316-5324 (1988)). Compounds that activate or otherwise interact with one or more of the PPARs have been implicated in the regulation of triglyceride and cholesterol levels in animal models. Compounds included in this group are the fibrate class of hypolipidermic drugs, herbicides, and phthalate plasticizers (Reddy and Lalwani, Crit. Rev. Toxicol. 12:1-58 (1983)). Peroxisome proliferation can also be elicited by dietary or physiological factors such as a high-fat diet and cold acclimatization.

Biological processes modulated by PPAR are those modulated by receptors, or receptor combinations, which are responsive to the PPAR receptor ligands. These processes include, for example, plasma lipid transport and fatty acid catabolism, regulation of insulin sensitivity and blood glucose levels, which are involved in hypoglycemia/hyperinsulinemia (resulting from, for example, abnormal pancreatic beta cell function, insulin secreting tumors and/or autoimmune hypoglycemia due to autoantibodies to insulin, the insulin receptor, or autoantibodies that are stimulatory to pancreatic beta cells), macrophage differentiation which lead to the formation of atherosclerotic plaques, inflammatory response, carcinogenesis, hyperplasia, and adipocyte differentiation.

Subtypes of PPAR include PPAR-alpha, PPAR-delta (also known as NUC1, PPAR-beta, and FAAR) and two isoforms of PPAR-gamma. These PPARs can regulate expression of target genes by binding to DNA sequence elements, termed PPAR response elements (PPRE). To date, PPRE's have been identified in the enhancers of a number of genes encoding proteins that regulate lipid metabolism suggesting that PPARs play a pivotal role in the adipogenic signaling cascade and lipid homeostasis (H. Keller and W. Wahli, Trends Endoodn. Met. 291-296, 4 (1993)).

Insight into the mechanism whereby peroxisome proliferators exert their pleiotropic effects was provided by the identification of a member of the nuclear hormone receptor superfamily activated by these chemicals (Isseman and Green, Nature 347-645-650 (1990)). The receptor, termed PPAR-alpha (or alternatively, PPARα), was subsequently shown to be activated by a variety of medium and long-chain fatty acids and to stimulate expression of the genes encoding rat acyl-CoA oxidase and hydratase-dehydrogenase (enzymes required for peroxisomal β-oxidation), as well as rabbit cytochrome P450 4A6, a fatty acid ω-hydroxylase (Gottlicher et al., Proc. Natl. Acad. Sci. USA 89:4653-4657 (1992); Tugwood et al., EMBO J 11:433-439 (1992); Bardot et al., Biochem. Biophys. Res. Comm. 192:37-45 (1993); Muerhoff et al., J Biol. Chem. 267:19051-19053 (1992); and Marcus et al., Proc. Natl. Acad Sci. USA 90(12):5723-5727 (1993).

Activators of the nuclear receptor PPAR-gamma (or alternatively, PPARγ), for example troglitazone, have been clinically shown to enhance insulin-action, to reduce serum glucose and to have small but significant effects on reducing serum triglyceride levels in patients with Type 2 diabetes. See, for example, D. E. Kelly et al., Curr. Opin. Endocrinol. Diabetes, 90-96, 5 (2), (1998); M. D. Johnson et al., Ann. Pharmacother., 337-348, 32 (3), (1997); and M. Leutenegger et al., Curr. Ther. Res., 403-416, 58 (7), (1997).

PPAR-delta (or alternatively, PPARδ) is broadly expressed in the body and has been shown to be a valuable molecular target for treatment of dyslipedimia and other diseases. For example, in a recent study in insulin-resistant obese rhesus monkeys, a potent and selective PPAR-delta compound was shown to decrease VLDL and increase HDL in a dose response manner (Oliver et al., Proc. Natl. Acad. Sci. U. S. A.98: 5305, 2001).

Because there are three isoforms of PPAR and all of them have been shown to play important roles in energy homeostasis and other important biological processes in human body and have been shown to be important molecular targets for treatment of metabolic and other diseases (see Willson, et al. J. Med. Chem. 43: 527-550 (2000)), it is desired in the art to identify compounds which are capable of selectively interacting with only one of the PPAR isoforms or compounds which are capable of interacting with multiple PPAR isoforms. Such compounds would find a wide variety of uses, such as, for example, in the treatment or prevention of obesity, for the treatment or prevention of diabetes, dyslipidemia, metabolic syndrome X and other uses.

### SUMMARY OF THE INVENTION

Described herein are novel compounds, including compounds capable of modulating the activity of human peroxisome proliferator activated receptor of the subtype delta (hPPAR-delta), and methods for utilizing such modulation to treat a disease or condition mediated or impacted by hPPAR-delta activity. Also described are compounds that mediate and/or inhibit the activity of hPPAR-delta, and pharmaceutical compositions containing such compounds. Further described are methods for making and producing such compounds. Also described are the therapeutic or prophylactic use of such compounds or compositions, and methods of treating metabolic disorders and conditions, by administering effective amounts of such compounds.

In one aspect of the present invention are novel mono- and bicyclic compounds, including pharmaceutically acceptable prodrugs, pharmaceutically active metabolites, pharmaceutically acceptable solvates, and pharmaceutically acceptable salts thereof. In another aspect of the present invention is the synthesis of such novel mono- and bicyclic compounds, and pharmaceutically acceptable prodrugs, pharmaceutically active metabolites, pharmaceutically acceptable solvates or pharmaceutically acceptable salts thereof. In yet another aspect of the present invention are pharmaceutical compositions of such mono- and bicyclic compounds, including pharmaceutically acceptable prodrugs, pharmaceutically active metabolites, pharmaceutically acceptable solvates or pharmaceutically acceptable salts thereof. In another aspect of the present invention are mono- and bicyclic compounds that can modulate the activity of hPPAR-delta in vitro and/or in vivo. In yet another aspect of the present invention are mono- and bicyclic compounds that can selectively modulate the activity of hPPAR-delta. In yet another aspect are methods for modulating hPPAR-delta comprising contacting the hPPAR-delta-modulating compounds, or pharmaceutically acceptable prodrugs, pharmaceutically active metabolites, pharmaceutically acceptable solvates or pharmaceutically acceptable salts thereof, described herein, with the hPPAR-delta or with cells comprising hPPAR-delta. In yet another aspect are methods for treating a disease or condition in a patient comprising administering a therapeutically effective amount of a hPPAR-delta-modulating compound, or a pharmaceutically acceptable prodrug, pharmaceutically active metabolite, pharmaceutically acceptable solvate or pharmaceutically acceptable salt thereof. In yet another aspect are methods for preventing a condition or disease in a patient comprising administering a prophylactically effective amount of a hPPAR-delta-modulating compound, or a pharmaceutically acceptable prodrug, pharmaceutically active metabolite, pharmaceutically acceptable solvate or pharmaceutically acceptable salt thereof.

One embodiment of the invention are compounds having the structure of Formula (I) and pharmaceutically acceptable salts and solvates thereof
[A]-[B]-[C] (I)
wherein
(a) [A] is [H]-[L];
   wherein [H] represents a COOH (or a hydrolyzable ester thereof) or tetrazole group
   [L] is: 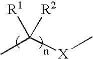
   wherein:
   each R¹ and each R² are independently H or C₁₋₃ alkyl, or R¹ and R² which are bonded to the same carbon atom may together with the carbon atom to which they are bonded, form a 3-6 membered cycloalkyl ring
   n =0, 1 or 2
   X=O, S or null
(b) [B] is a ring system selected from the group consisting of: 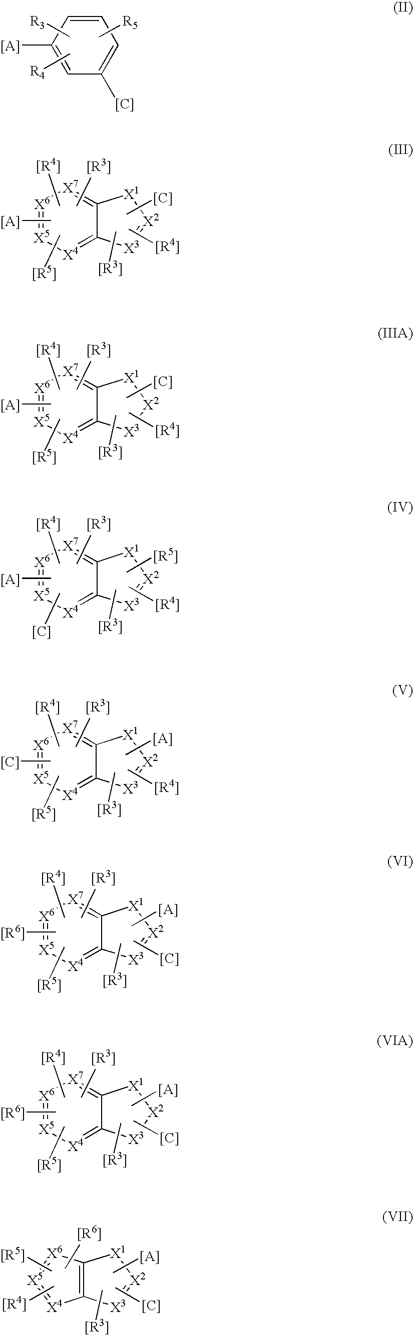 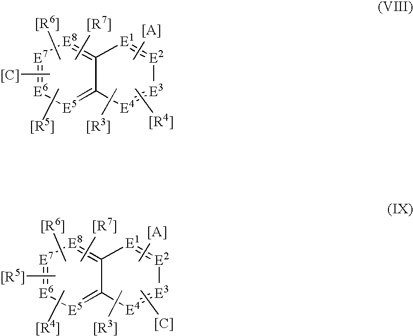
   wherein X¹ is NH, O, or S; except that when any of [C], [A], or R³—R⁵ is attached to X¹, X¹ is N;
   X²-X⁷ are each independently CH, N, or C when [C], [A], R³, R⁴, R⁵, R⁶, or R⁷ is attached; or, alternatively, when [B] is IIIA or VIA, X₂ and X₃ are each independently CH₂ or, when [C], [A], R³, or R⁴ is attached, CH or C;

Each R³, each R⁴, each R⁵, each R⁵, each R⁶, and each R⁷ are each independently hydrogen, perhaloaryloxy, alkanoylalkyl, alkanoylalkoxy, alkanoyloxy, N-aryl-N-alkylamino, heterocyclylalkoxy, heterocyclylthio, hydroxyalkoxy, carboxamidoalkoxy, alkoxycarbonylalkoxy, alkoxycarbonylalkenyloxy, aralkanoylalkoxy, aralkenoyl, N-alkylcarboxamido, N-haloalkylcarboxami do, N-cycloalkylcarbox amido, N-arylcarboxamidoalkoxy, cycloalkylcarbonyl, cyanoalkoxy, heterocyclylcarbonyl, carboxy, heteroaralkylthio, heteroaralkoxy, cycloalkylamino, acylalkyl, acylalkoxy, aroylalkoxy, heterocyclyloxy, aralkylaryl, aralkyl, aralkenyl, aralkynyl, heterocyclyl, haloalkylthio, alkanoyloxy, alkoxy, alkoxyalkyl, cycloalkoxy, cycloalkylalkoxy, hydroxy, amino, thio, nitro, alkylamino, alkylthio, arylamino, aralkylamino, arylthio, arylthioalkyl, alkylsulfonyl, alkylsulfonamido, monoarylamidosulfonyl, arylsulfonyl, heteroarylthio, heteroarylsulfonyl, heterocyclylsulfonyl, heterocyclylthio, alkanoyl, alkenoyl, aroyl, heteroaroyl, aralkanoyl, heteroaralkanoyl, haloalkanoyl, alkyl, alkenyl, alkynyl, alkenyloxy, alkylenedioxy, haloalkylenedioxy, cycloalkyl, cycloalkylalkanoyl, halo, haloalkyl, haloalkoxy, hydroxyhaloalkyl, hydroxyhaloalkoxy, hydroxyalkyl, aryl, aryloxy, aralkoxy, saturated heterocyclyl, heteroaryl, heteroaryloxy, heteroaryloxyalkyl, heteroaralkyl, arylalkenyl, carboalkoxy, alkoxycarboxamido, alkylamidocarbonylamido, arylamidocarbonylamido, carboalkoxyalkyl, carboalkoxyalkenyl, carboxamido, carboxamidoalkyl, and cyanocycloalkylalkyl, cycloalkenyl, alkoxycarbonyl, aralkylthio, alkylthio, alkylsulfinyl, arylsulfinyl, dialkylamino, aminoalkyl, dialkylaminoalkyl, aminoaryl, alkylaminoaryl, acylamino; aminocarbonylalkoxy, aminocarbonylamino, aminocarbonylaminoalkyl, aminothiocarbonylamino, aminothiocarbonylaminoalkyl and may be attached to any X¹-X⁷or E¹-E⁸;
E¹-E⁸ are each independently CH, N, or C when [C], [A], R³, R⁴, R⁵, R⁶, or R⁷ is attached;
c) [C] is 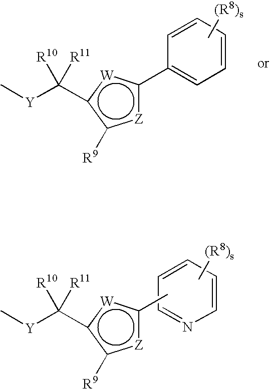
   wherein Y is O, S, or (CR¹²R¹³)ᵣ where r is 0-2;
   each R¹² and each R¹³ are each independently H, fluorine or C₁₋₆ alkyl;
   one of W and Z is N, the other is S or O;
   R¹⁰ and R¹¹ are independently H, phenyl, benzyl, fluorine, C₁₋₆ alkyl, or allyl;
   R⁹ is H, CH₃, or CF₃;
   Each R⁸ is independently CF₃, C₁₋₆ alkyl, OCH₃ or halogen;
   s is 0, 1, 2, 3, 4 or 5;
   further wherein the optional pyridyl ring in the substructure [C] may be replaced with another monocyclic heteroaryl ring.

### DETAILED DESCRIPTION OF THE INVENTION

The present invention discloses that substituted bicyclic heterocyclic moieties linked to an acid moiety can be combined with thiazole and oxazole moieties in such a manner as to confer selective activation of hPPAR-delta. Novel monocyclic aryls which bear electronic and structural resemblance to the bicyclic compounds of the invention are also active and selective hPPAR modulators.

In another aspect, the present invention relates to a method of modulating at least one peroxisome proliferator-activated receptor (PPAR) function comprising the step of contacting the PPAR with a compound of Formula I, as described herein. The change in cell phenotype, cell proliferation, activity of the PPAR, or binding of the PPAR with a natural binding partner may be monitored. Such methods may be modes of treatment of disease, biological assays, cellular assays, biochemical assays, or the like. In certain embodiments, the PPAR may be selected from the group consisting of PPARα, PPARδ, and PPARγ.

The present invention describes methods of treating a disease comprising identifying a patient in need thereof, and administering a therapeutically effective amount of a compound of Formula I, as described herein, to a patient.

Thus, in certain embodiments, the disease to be treated by the methods of the present invention is selected from the group consisting of obesity, diabetes, hyperinsulinemia, metabolic syndrome X, polycystic ovary syndrome, climacteric disorders associated with oxidative stress, inflammatory response to tissue injury, pathogenesis of emphysema, ischemia-associated organ injury, doxorubicin-induced cardiac injury, drug-induced hepatotoxicity, atherosclerosis, and hypertoxic lung injury.

Compounds described herein may be activating both PPAR-delta and PPAR-gamma or PPAR-alpha and PPAR-delta, or all three PPAR subtypes, or preferably selectively activating hPPAR-delta, and therefore may be used in the treatment of dyslipidemia associated with atherosclerosis, non-insulin dependent diabetes mellitus, Syndrome X, (Staels, B. et al., Curr. Pharm. Des., 3 (1),1-14 (1997>> and familial combined hyperlipidemia (FCH). Syndrome X is the syndrome characterized by an initial insulin resistant state, generating hyperinsulinaemia, dyslipidaemia and impaired glucose tolerance, which can progress to non-insulin dependent diabetes mellitus (Type 2 diabetes), characterized by hyperglycemia. FCH is characterized by hypercholesterolemia and hypertriglyceridemia within the same patient and family.

Other embodiments of the invention are compounds having the structure of Formula (I) are compounds wherein [B] has the structure of Formula (II):

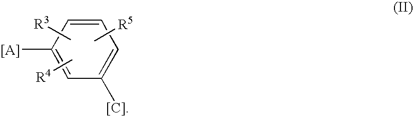

Other embodiments of the invention are compounds wherein [B] is selected from the group consisting of:

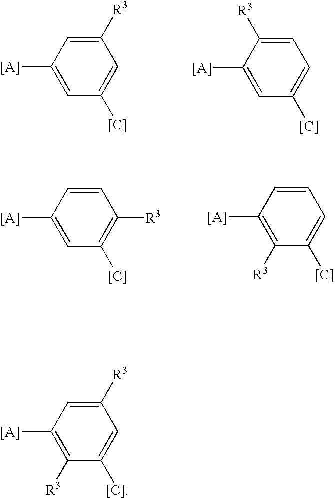

Other embodiments of the invention are compounds wherein [B] has the structure of Formula (III):

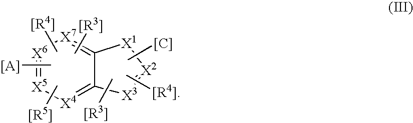

Other embodiments of the invention are compounds wherein [B] has the structure of Formula (IIIA):

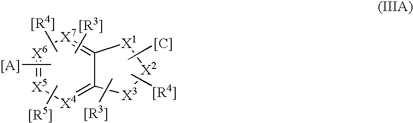

Other embodiments of the invention are compounds wherein [B] has the structure of Formula (IV):

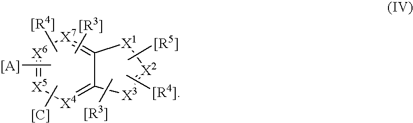

Other embodiments of the invention are compounds wherein [B] has the structure of Formula (V):

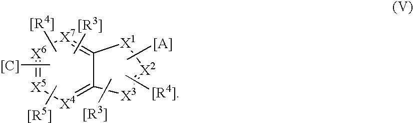

Other embodiments of the invention are compounds wherein [B] has the structure of Formula (VI).

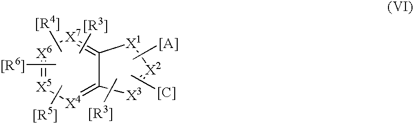

Other embodiments of the invention are compounds wherein [B] has the structure of Formula (VIA).

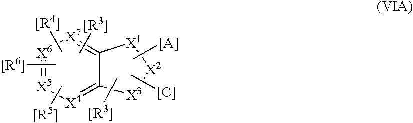

One embodiment of the invention is a group of compounds wherein [B] is an optionally substituted indole, benzimidazole, indazole, Benzothiophene, or benzofuran moiety.

Another embodiment of the invention is a group of compounds wherein [B] is an optionally substituted benzoxazole, benzthiazole, benziridazole, indazole, Benzothiophene, or benzofuran moiety.

Another embodiment are compounds wherein [B] is an optionally substituted pyrrolothiophene, imidazolothiazole, as depicted below:

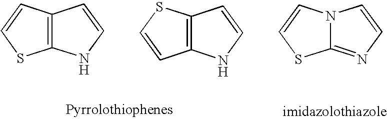

Another embodiment are compounds wherein [B] is an optionally substituted naphthalene or quinoline moiety.

An aspect of the invention are compounds wherein the independent substituent on the ring moieties, R³ is H, C₁₋₃ alkyl, OCH₃, CF₃, or halogen, or preferably H or CH₃.

Another aspect of the invention are compounds wherein R¹ and R² are both H.

Another aspect of the invention are compounds wherein one or both of R¹ and R are CH₃.

Another aspect of the invention are compounds wherein both R¹ and R² are CH₃.

Another aspect of the invention are compounds wherein n is 1 or 2.

Another aspect of the invention are compounds wherein X is O or null.

Another aspect of the invention are compounds wherein s is 0, 1 or 2.

Another aspect of the invention are compounds wherein the R⁸ substitution pattern is selected from the group consisting of: 4-perhaloalkyl; 4-halogen; 3,4, dihalo; 3-halo, 4-perfluoroalkyl.

Another aspect of the invention are compounds wherein said halo or halogen is fluorine or chlorine.

Another aspect of the invention are compounds wherein R¹⁰ and R¹¹ are H.

Another aspect of the invention are compounds wherein one or both of R¹⁰ and R¹¹ is methyl.

Another aspect of the invention are compounds wherein R⁹ is H, C₁₋₃alkyl, or perhaloalkyl.

Another aspect of the invention are compounds wherein R⁹ is methyl.

Another aspect of the invention are compounds wherein Z is N and W is O, or S.

Another aspect of the invention are compounds wherein Y is O or S.

Another aspect of the invention are compounds wherein Y is (CR¹²R¹³)ᵣ.

Another aspect of the invention are compounds wherein r is 0 or 1

Another aspect of the invention are compounds wherein R¹² and R¹³ are H.

Another aspect of the invention are compounds wherein one or both of said R¹² and R¹³ are methyl.

Another aspect of the invention are compounds where [C] has the substructure described above with an optionally substituted terminal phenyl ring.

Another aspect of the invention are compounds where [C] has the substructure described above with an optionally substituted terminal pyridyl ring.

Another aspect of the invention are compounds where [C] has the substructure described above wherein the optionally substituted terminal pyridyl ring is replaced with an optionally substituted monocyclic heteroaryl ring. A further aspect of the invention are such compounds wherein the optionally substituted monocyclic heteroaryl ring is selected from the group consisting of optionally substituted thienyl, furanyl, pyrrolyl, pyrimidyl, imidazolyl, pyrazinyl, oxazolyl, isoxazolyl, thiazolyl, and isothiazolyl, quinolinyl, isoquinolinyl, and quinazolinyl.

Another embodiment of the invention, are compounds of the invention which are hPPAR-delta modulators, or preferably, selective hPPAR-delta modulators.

Another embodiment of the invention are pharmaceutical compositions comprising the hPPAR-delta modulators of the invention.

Another aspect are pharmaceutical compositions of the invention further comprising a pharmaceutical acceptable diluent or carrier. In another aspect, the present invention relates to a method of treating. a disease comprising identifying a patient in need thereof, and administering a therapeutically effective amount of a compound of Formula I, as described herein, to the patient.

The third subtype of PPARs, PPARδ (PPARβ, NUC1), is broadly expressed in the body and has been shown to be a valuable molecular target for treatment of dyslipedimia and other diseases. For example, in a recent study in insulin-resistant obese rhesus monkeys, a potent and selective PPARδ compound was shown to decrease VLDL and increase HDL in a dose response manner (Oliver et al., Proc. Natl. Acad. Sci. U.S.A. 98: 5305, 2001).

The compounds of the invention are useful in the treatment of a disease or condition ameliorated by the modulation of an hPPAR-delta. Specific diseases and conditions modulated by PPAR-delta and for which the compounds and compositions are useful include but are not limited to dyslipidemia, syndrome X, heart failure, hypercholesteremia, cardiovascular disease, type II diabetes mellitus, type 1 diabetes, insulin resistance hyperlipidemia, obesity, anorexia bulimia, inflammation and anorexia nervosa.

The compounds of the invention may also be used (a) for raising HDL in a subject; (b) for treating Type 2 diabetes, decreasing insulin resistance or lowering blood pressure in a subject; (c) for decreasing LDLc in a subject; (d) for shifting LDL particle size from small dense to normal dense LDL in a subject; (e) for treating atherosclerotic diseases including vascular disease, coronary heart disease, cerebrovascular disease and peripheral vessel disease in a subject; and (f) for treating inflammatory diseases, including rheumatoid arthritis, asthma, osteoarthritis and autoimmune disease in a subject.

The compounds of the invention may also be used for treating, ameliorating, or preventing a disease or condition selected from the group consisting of obesity, diabetes, hyperinsulinemia, metabolic syndrome X, polycystic ovary syndrome, climacteric disorders associated with oxidative stress, inflammatory response to tissue injury, pathogenesis of emphysema, ischemia-associated organ injury, doxorubicin-induced cardiac injury, drug-induced hepatotoxicity, atherosclerosis, and hypertoxic lung injury.

Another aspect of the compounds and compositions of invention is their use in the manufacture of a medicament for the prevention or treatment of a hPPAR-delta-mediated disease or condition.

Another aspect of the compounds, pharmaceutically acceptable prodrug, pharmaceutically active metabolite, or pharmaceutically acceptable salt comprising a compound having an EC50 value less than 1 μM as measured by a functional cell assay.

Another aspect of the invention are methods for raising HDL in a subject comprising the administration of a therapeutic amount of a hPPAR-delta modulators disclosed herein.

Another aspect of the invention is the use of a hPPAR-delta modulators disclosed herein for the manufacture of a medicament for the raising of HDL in a patient in need thereof.

Another aspect of the invention are methods for treating Type 2 diabetes, decreasing insulin resistance or lowering blood pressure in a subject comprising the administration of a therapeutic amount of a hPPAR-delta modulators disclosed herein.

Another aspect of the invention is the use of a hPPAR-delta modulator disclosed herein for the manufacture of a medicament for the treatment of Type 2 diabetes, for decreasing insulin resistance or for lowering blood pressure in a patient in need thereof.

Another aspect of the invention is the use and administration of hPPAR-delta selective modulators.

Another aspect of the invention are methods for decreasing LDLc in a subject comprising the administration of a therapeutic amount of a hPPAR delta modulator disclosed herein.

Another aspect of the invention is the use of a hPPAR-delta modulators disclosed herein for the manufacture of a medicament for decreasing LDLc in a patient in need thereof.

Another aspect of the invention are methods for shifting LDL particle size from small dense to normal dense LDL in a subject comprising the administration of a therapeutic amount of a hPPAR-delta modulators as disclosed herein.

Another aspect of the invention is the use of a hPPAR-delta modulator as disclosed herein for the manufacture of a medicament for shifting LDL particle size from small dense to normal LDL in a patient in need thereof.

Another aspect of the invention is the use of a hPPAR-delta modulator as disclosed herein for treating atherosclerotic diseases including vascular disease, coronary heart disease, cerebrovascular disease and peripheral vessel disease in a subject comprising the administration of a therapeutic amount of a hPPAR-delta modulator as disclosed herein.

Another aspect of the invention is the use of a hPPAR-delta modulator disclosed herein for the manufacture of a medicament for the treatment of atherosclerotic diseases including vascular disease, coronary heart disease, cerebrovascular disease and peripheral vessel disease in a patient in need thereof.

Another aspect of the invention are methods for treating inflammatory diseases, including rheumatoid arthritis, asthma, osteoarthritis and autoimmune disease in a subject comprising the administration of a therapeutic amount of a hPPAR-delta modulator as disclosed herein.

Another aspect of the invention is the use of a hPPAR-delta modulator as disclosed herein for the manufacture of a medicament for the treatment of inflammatory diseases, including rheumatoid arthritis, asthma, osteoarthritis and autoimmune disease in a patient in need thereof, including those hPPAR-delta modulators which are hPPAR-delta selective modulator.

Another aspect of the invention are methods of treatment of a hPPAR-delta mediated disease or condition comprising administering a therapeutically effective amount of a compound disclosed herein or a pharmaceutically acceptable salt, ester, amide, or prodrug thereof.

Another aspect of the invention are methods of modulating a peroxisome proliferator-activated receptor (PPAR) function comprising contacting said PPAR with a compound disclosed herein and monitoring a change in cell phenotype, cell proliferation, activity of said PPAR, or binding of said PPAR with a natural binding partner.

Another aspect of the invention are methods of treating a disease or condition, comprising identifying a patient in need thereof, and administering a therapeutically effective amount of a compound disclosed herein to said patient, wherein said disease is selected from the group consisting of obesity, diabetes, hyperinsulinemia, metabolic syndrome X, polycystic ovary syndrome, climacteric disorders associated with oxidative stress, inflammatory response to tissue injury, pathogenesis of emphysema, ischemia-associated organ injury, doxorubicin-induced cardiac injury, drug-induced hepatotoxicity, atherosclerosis, and hypertoxic lung injury.

Another embodiment of the invention are compounds wherein [B] is selected from the group consisting of III, IIIA, VI, and VIA. Further embodiments of the invention are characterized by X¹ being N or NH. In additional embodiments of the invention, one of X²-X⁷ is N or NH. In further embodiments of the invention, the compounds of the invention are characterized by [B] having a structure selected from the group consisting of: 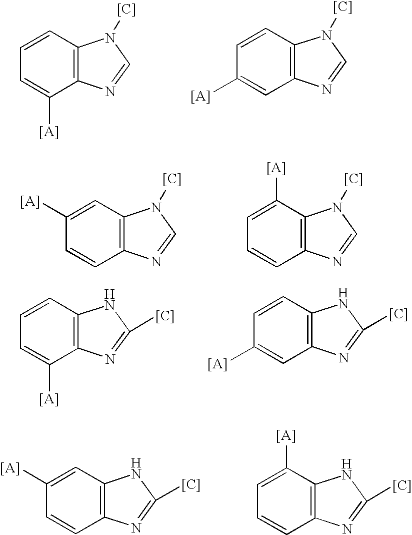
wherein [B] is optionally singly or doubly substituted with R³. In further embodiments of the invention, the compounds of the invention are characterized by [B] having a structure selected from the group consisting of: 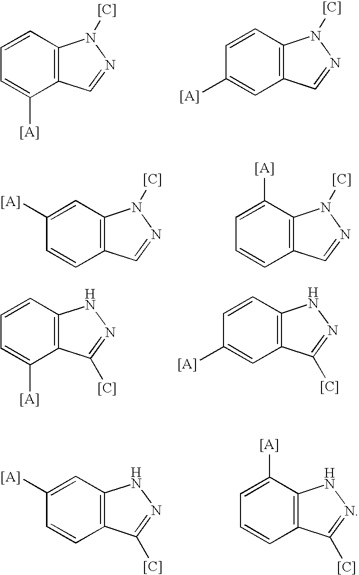
wherein [B] is optionally singly or doubly substituted with R³.

Another embodiment of the invention are compounds wherein [B] is selected from the group consisting of III and IIIA, X¹ is N or NH and wherein none of X²-X⁷ are heteroatoms. In further embodiments [B] is an optionally substituted indole moiety. In further embodiments, [B] is an optionally substituted dihydro indole moiety.

Another embodiment of the invention are compounds having structural formula I and wherein [B] is selected from the group consisting of the following: 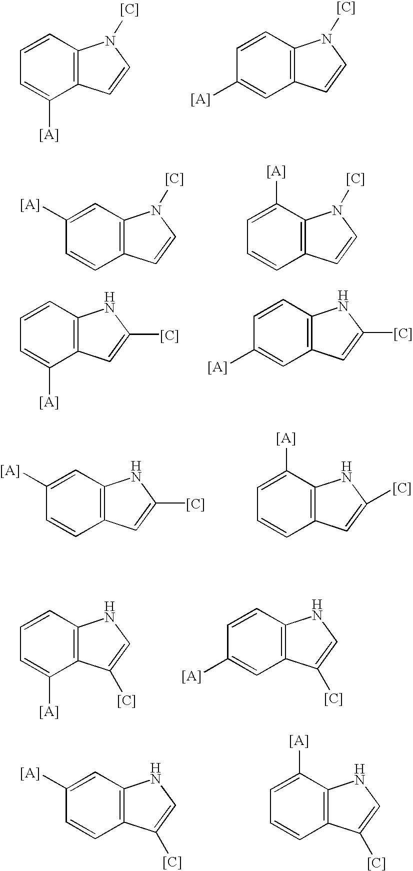
wherein [B] is optionally singly or doubly substituted with R³. A further embodiment of the invention is characterized additionally by X¹ being N and [C] being attached to X¹.

In another embodiment of the invention, X=O. In a further embodiment of the invention, n=1. In a further embodiment of the invention, R¹═R²═H. In an alternate embodiment of the invention, R¹═=R²=methyl.

In another embodiment of the invention, the compounds of the invention are additionally characterized by Y═C R¹²R¹³ and r=1 or 2. In further embodiments of the invention, W═S and Z=N. In further embodiments of the invention, R⁹=methyl. In further embodiments of the invention, the R⁸ substitution pattern is selected from the group consisting of: 4-perhaloalkyl; 4-halogen; 3,4, dihalo; 3-halo, 4-perfluoroalkyl.

In another aspect of the invention, compounds having structural formula I, wherein [B] is III or IIIA are further characterized in that [A] is attached to X⁵ or X⁶.

Other embodiments of the invention include compounds of Examples 1-25 or salts, esters, thioesters, amides, or prodrugs thereof.

Other embodiments of the invention include pharmaceutically acceptable salts, esters, thioesters, amides, or prodrugs of any compound of the invention or any set of compounds in an embodiment of the invention.

Other embodiments of the invention include compounds having the structure of Formula I wherein each R³, each R⁴, each R⁵, each R⁵, each R⁶, and each R⁷ are each independently H, C₁₋₃alkyl, OCH₃, CF₃, or halogen and may be attached to any X¹-X⁷ or E¹-E⁸;

Other embodiments of the invention include compounds having the structure of Formula I wherein [B] has the structure of Formula (II):

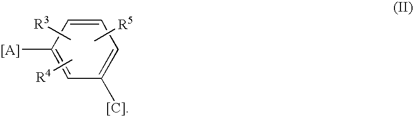

Other embodiments of the invention include compounds having the structure of Formula I wherein [B] has the structure selected from the group consisting of:

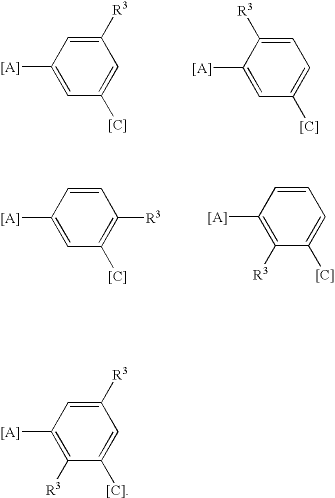

Other embodiments of the invention include compounds having the structure of Formula I wherein [B] has the structure selected from the group consisting of:

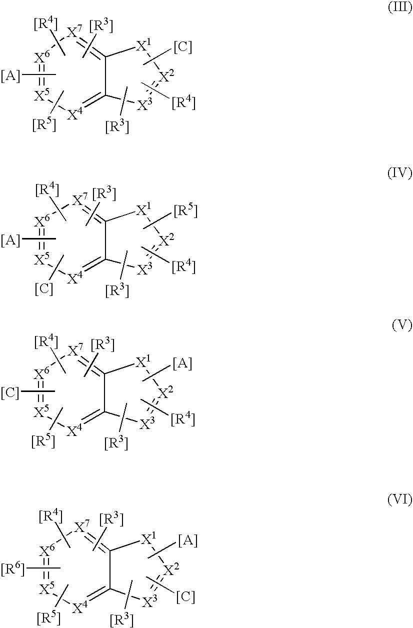

Other embodiments of the invention include compounds having the structure of Formula I wherein [B] has the structure as follows:

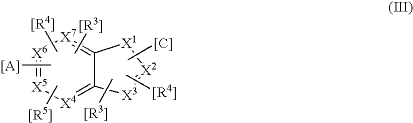

Other embodiments of the invention include compounds having the structure of Formula I wherein [B] has the structure selected from the group consisting of:

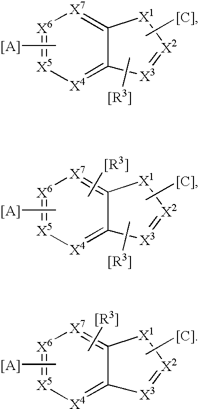

Other embodiments of the invention include compounds having the structure of Formula I wherein [B] has the structure as follows:

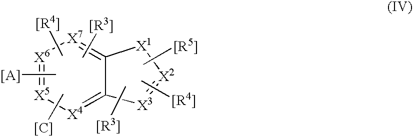

Other embodiments of the invention include compounds having the structure of Formula I wherein [B] has the structure selected from the group consisting of:

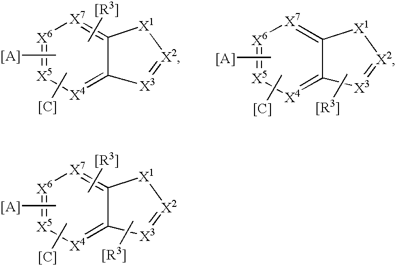

Other embodiments of the invention include compounds having the structure of Formula I wherein [B] has the structure as follows:

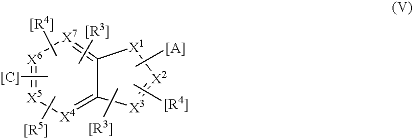

Other embodiments of the invention include compounds having the structure of Formula I wherein [B] has the structure selected from the group consisting of:

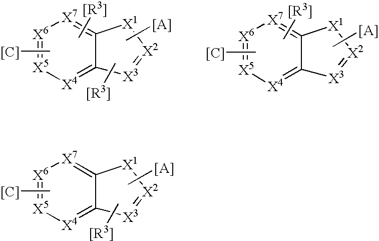

Other embodiments of the invention include compounds having the structure of Formula I wherein [B] has the structure as follows:

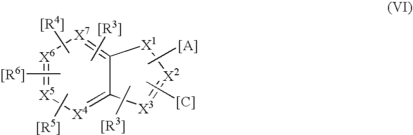

Other embodiments of the invention include compounds having the structure of Formula I wherein [B] has the structure selected from the group consisting of:

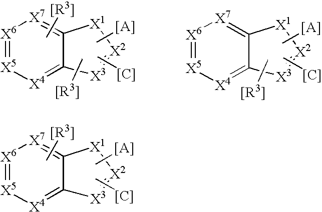

Other embodiments of the invention include compounds having the structure of Formula I wherein [B] has the structure selected from the group consisting of: 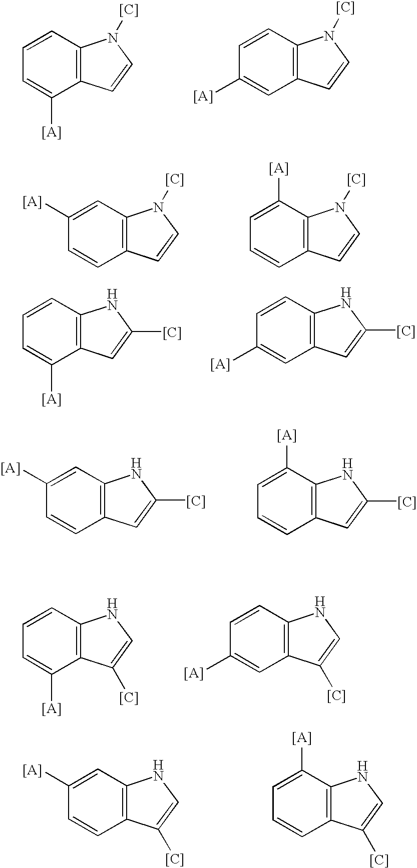
wherein [B] is optionally singly or doubly substituted with R³.

Other embodiments of the invention include compounds having the structure of Formula I wherein [B] has the structure selected from the group consisting of: 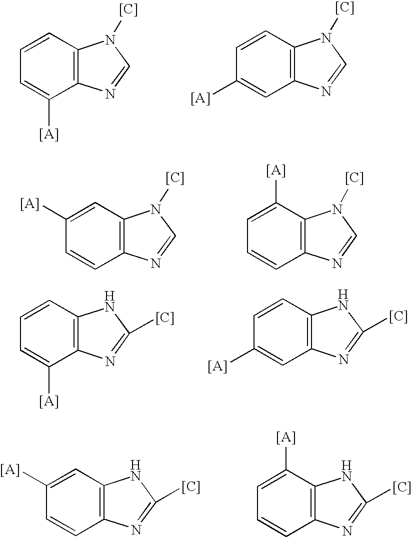
wherein [B] is optionally singly or doubly substituted with R³.

Other embodiments of the invention include compounds having the structure of Formula I wherein [B] has the structure selected from the group consisting of: 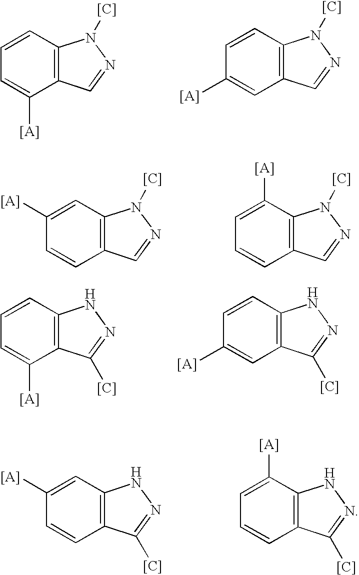
wherein [B] is optionally singly or doubly substituted with R³.

Other embodiments of the invention include compounds having the structure of Formula I wherein [B] has the structure selected from the group consisting of: 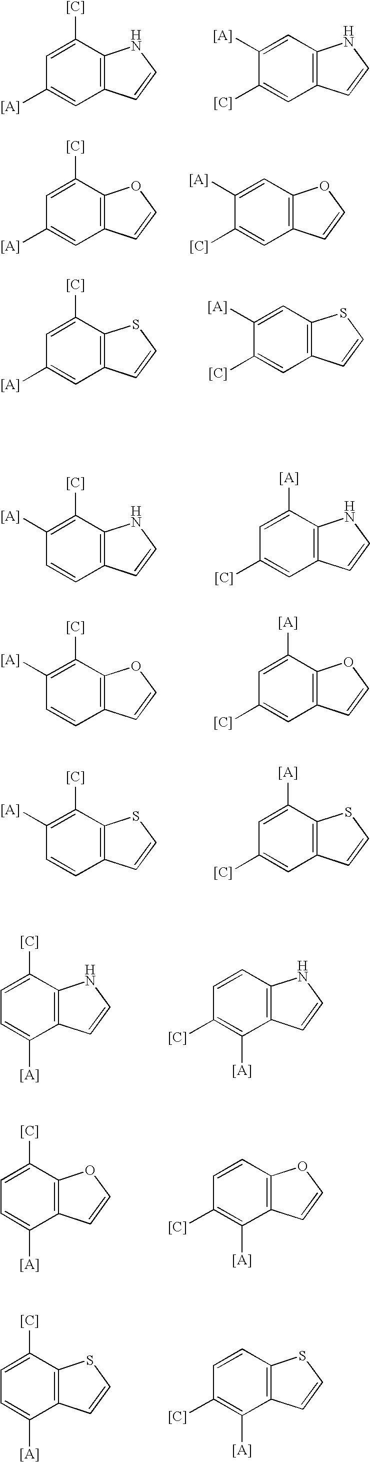 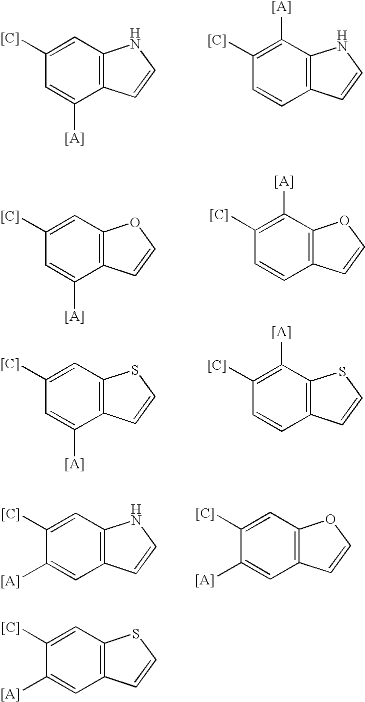
wherein [B] is optionally singly or doubly substituted with R³.

Other embodiments of the invention include compounds having the structure of Formula I wherein [B] has the structure selected from the group consisting of: 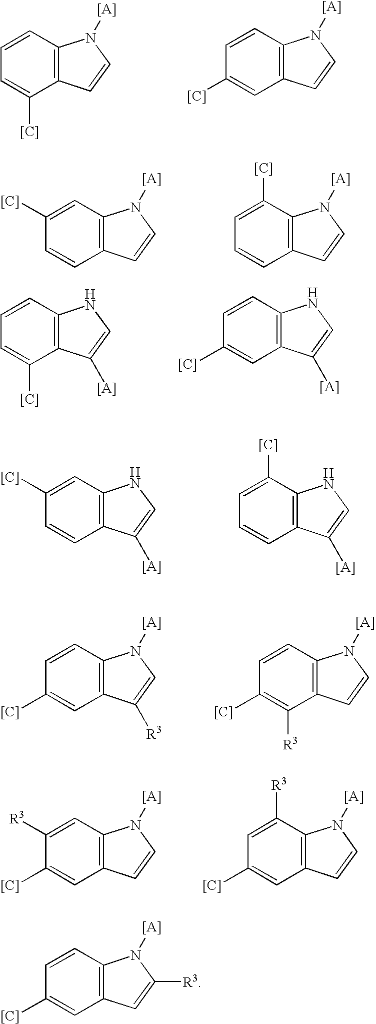
wherein [B] is optionally singly or doubly substituted with R³.

Other embodiments of the invention include compounds having the structure of Formula I wherein [B] has the structure selected from the group consisting of: 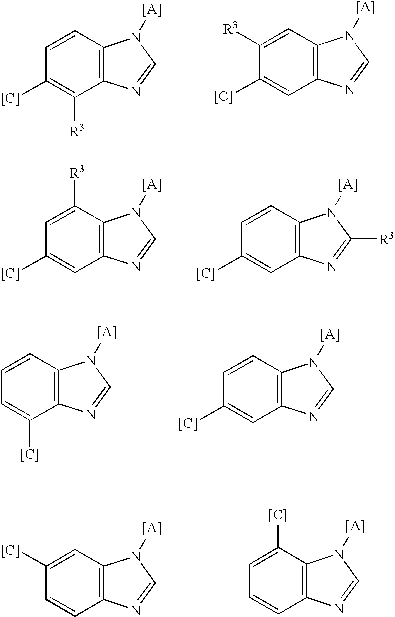
wherein [B] is optionally singly or doubly substituted with R³.

Other embodiments of the invention include compounds having the structure of Formula I wherein [B] has the structure selected from the group consisting of: 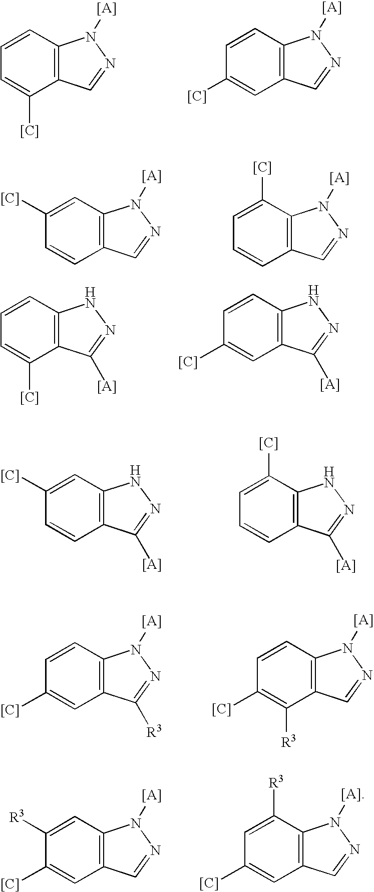
wherein [B] is optionally singly or doubly substituted with R³.

Other embodiments of the invention include compounds having the structure of Formula I wherein [B] has the structure selected from the group consisting of:

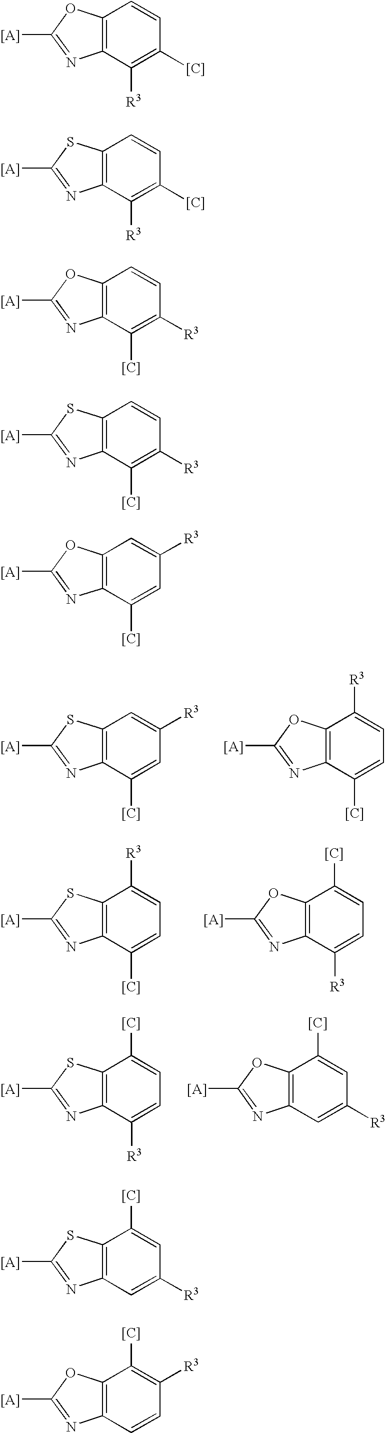

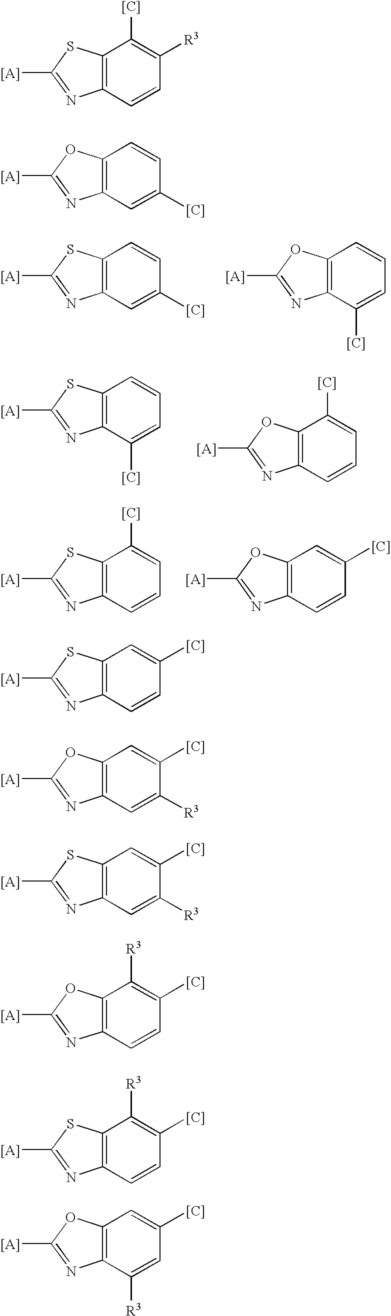

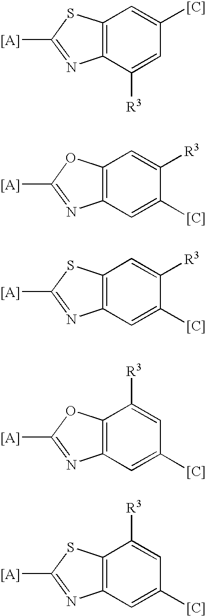

Other embodiments of the invention include compounds having the structure of Formula I wherein [B] has the structure selected from the group consisting of:

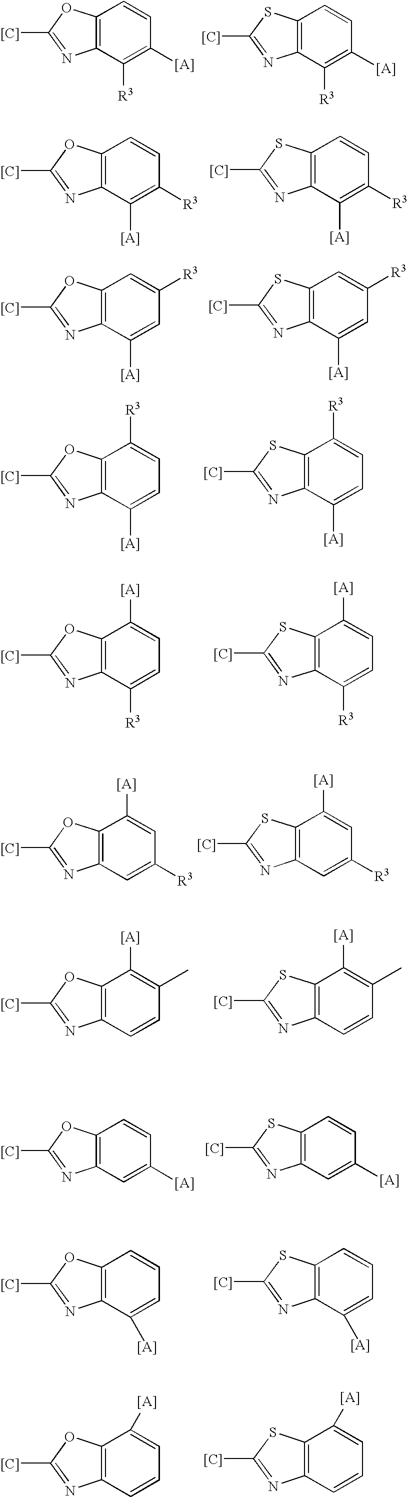

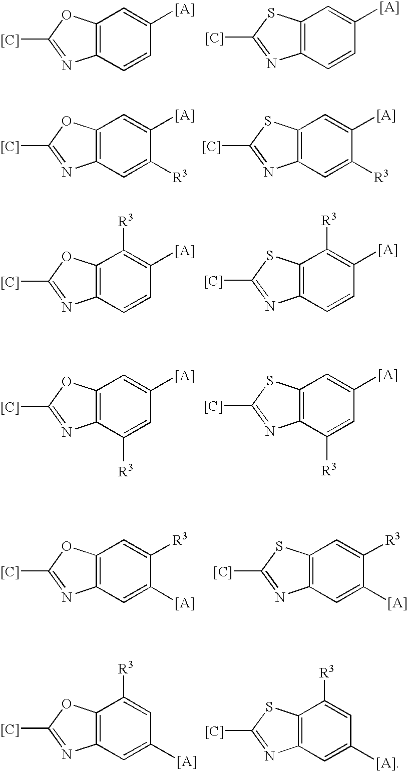

Other embodiments of the invention include compounds having the structure of Formula I wherein [B] has the structure selected from the group consisting of:

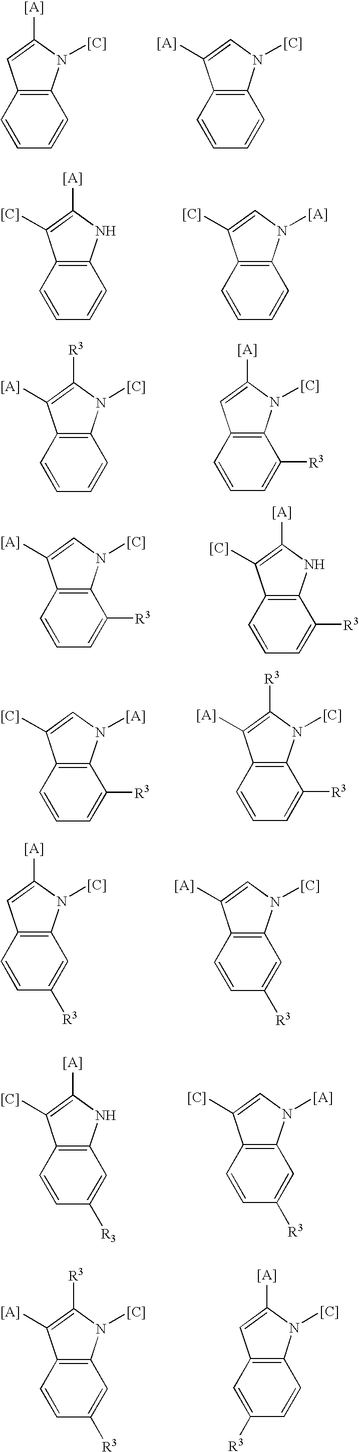

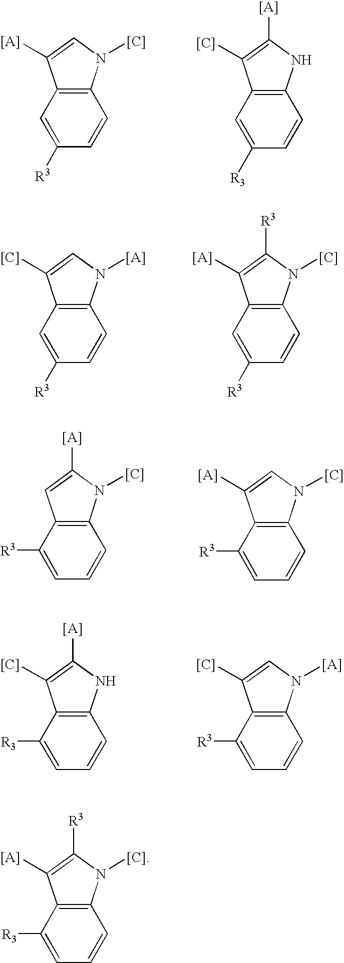

Other embodiments of the invention include compounds having the structure of Formula I wherein [B] has the structure selected from the group consisting of: 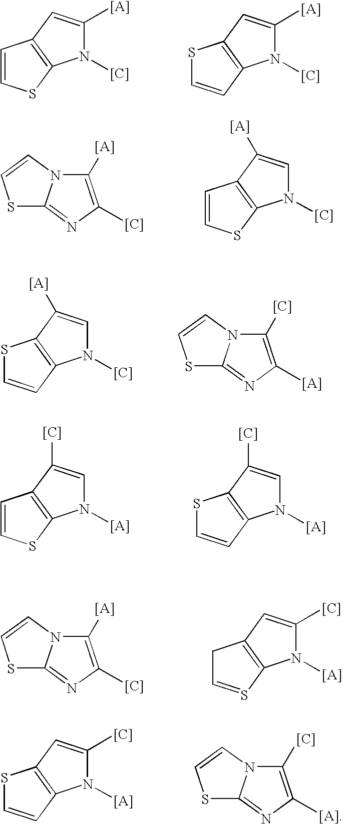
wherein [B] is optionally singly or doubly substituted with R³.

Other embodiments of the invention include compounds having the structure of Formula I wherein [B] has the structure selected from the group consisting of: 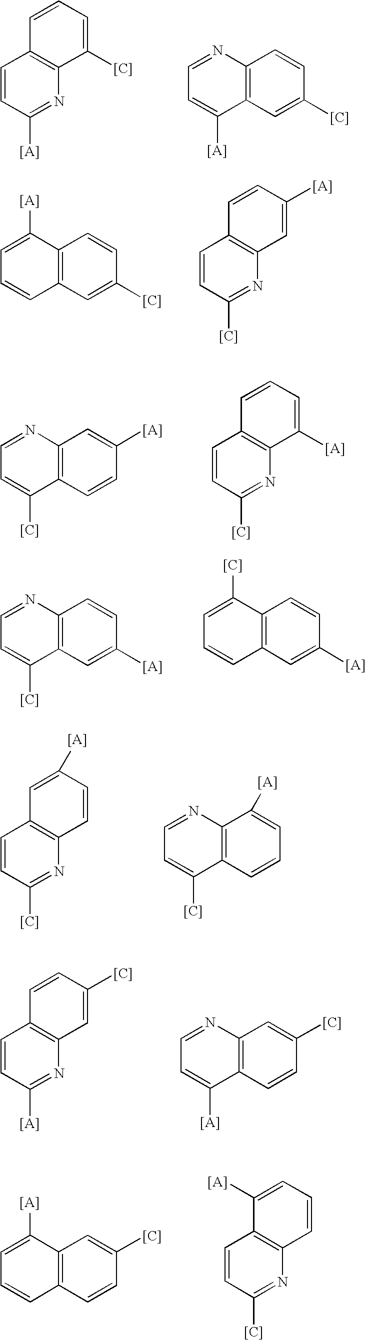 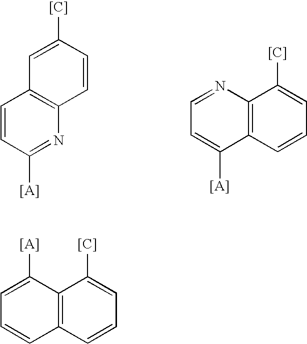
wherein [B] is optionally singly or doubly substituted with R³.

Other embodiments of the invention include compounds having the structure of Formula I wherein [B] has the structure selected from the group consisting of: 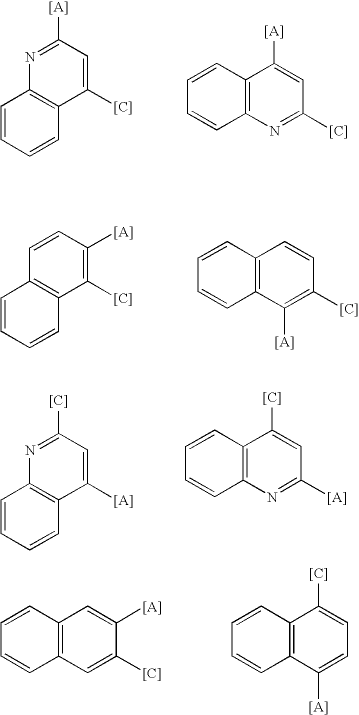
wherein [B] is optionally singly or doubly substituted with R³.

Other embodiments of the invention include compounds having the structure of Formula I according to any of the embodiments herein and additionally wherein R³ is H or methyl.

Other embodiments of the invention include compounds having the structure of Formula I according to any of the embodiments herein and additionally wherein R¹ and R² are both H.

Other embodiments of the invention include compounds having the structure of Formula I according to any of the embodiments herein and additionally wherein one or both of R¹ and R² are CH₃.

Other embodiments of the invention include compounds having the structure of Formula I according to any of the embodiments herein and additionally wherein both R¹ and R²are CH₃.

Other embodiments of the invention include compounds having the structure of Formula I according to any of the embodiments herein and additionally wherein n is 1 or 2.

Other embodiments of the invention include compounds having the structure of Formula I according to any of the embodiments herein and additionally wherein X is O or null.

Other embodiments of the invention include compounds having the structure of Formula I according to any of the embodiments herein and additionally wherein s is 0, 1 or 2.

Other embodiments of the invention include compounds having the structure of Formula I according to any of the embodiments herein and additionally wherein the R⁸ substitution pattern is selected from the group consisting of: 4-perhaloalkyl; 4-halogen; 3,4, dihalo; 3-halo, 4-perfluoroalkyl.

Other embodiments of the invention include compounds having the structure of Formula I according to any of the embodiments herein and additionally wherein said halo or halogen is fluorine or chlorine.

Other embodiments of the invention include compounds having the structure of Formula I according to any of the embodiments herein and additionally wherein R¹⁰and R¹¹ are H.

Other embodiments of the invention include compounds having the structure of Formula I according to any of the embodiments herein and additionally wherein one or both of R¹⁰ and R¹¹ is methyl.

Other embodiments of the invention include compounds having the structure of Formula I according to any of the embodiments herein and additionally wherein R⁹ is H, C₁₋₃alkyl, or perhaloalkyl.

Other embodiments of the invention include compounds having the structure of Formula I according to any of the embodiments herein and additionally wherein R⁹ is methyl.

Other embodiments of the invention include compounds having the structure of Formula I according to any of the embodiments herein and additionally wherein Z is N and W is O, or S.

Other embodiments of the invention include compounds having the structure of Formula I according to any of the embodiments herein and additionally wherein Y is O or S.

Other embodiments of the invention include compounds having the structure of Formula I according to any of the embodiments herein and additionally wherein Y is (CR¹²R¹³)ᵣ.

Other embodiments of the invention include compounds having the structure of Formula I according to any of the embodiments herein and additionally wherein r is 0 or 1

Other embodiments of the invention include compounds having the structure of Formula I according to any of the embodiments herein and additionally wherein R¹² and R¹³ are H.

Other embodiments of the invention include compounds having the structure of Formula I according to any of the embodiments herein and additionally wherein one or both of said R¹² and R¹³ are methyl.

Other embodiments of the invention include any of the following compounds or pharmaceutically acceptable salts, amides, esters, thioesters, or pro-drugs thereof:

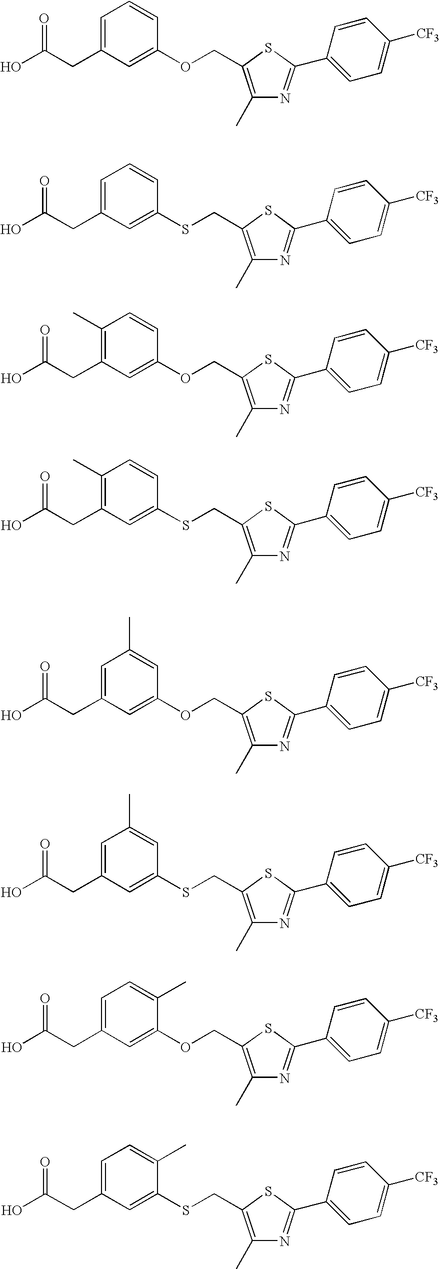

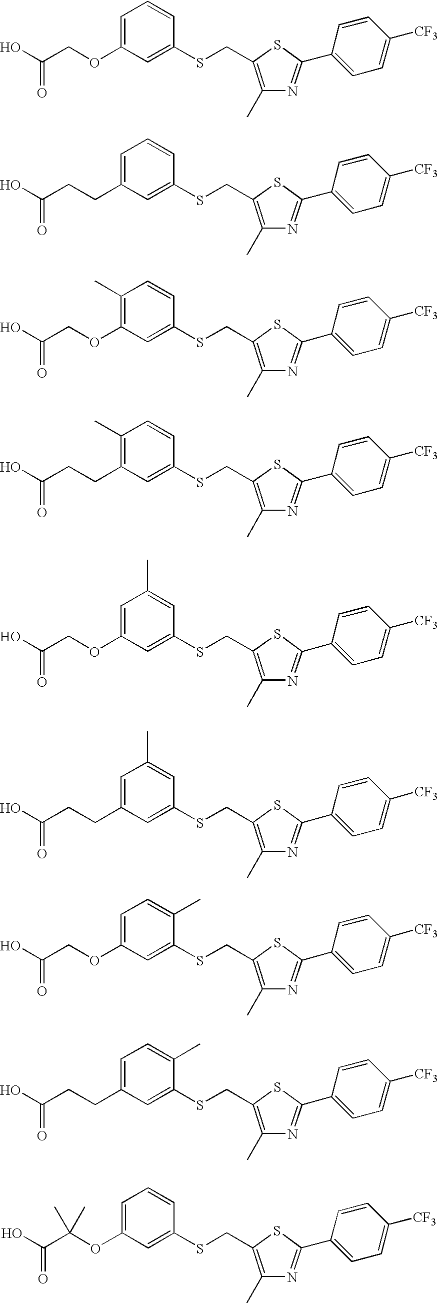

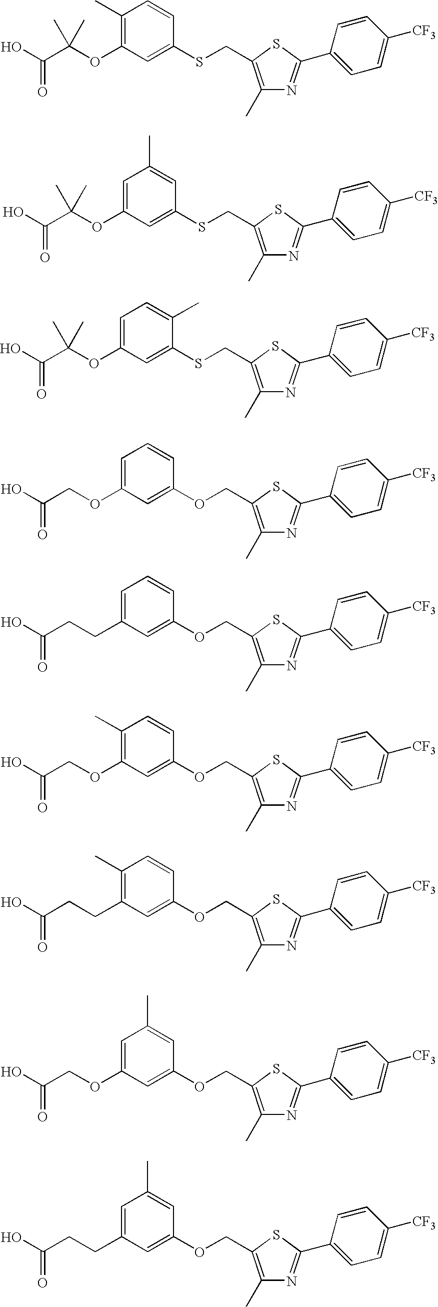

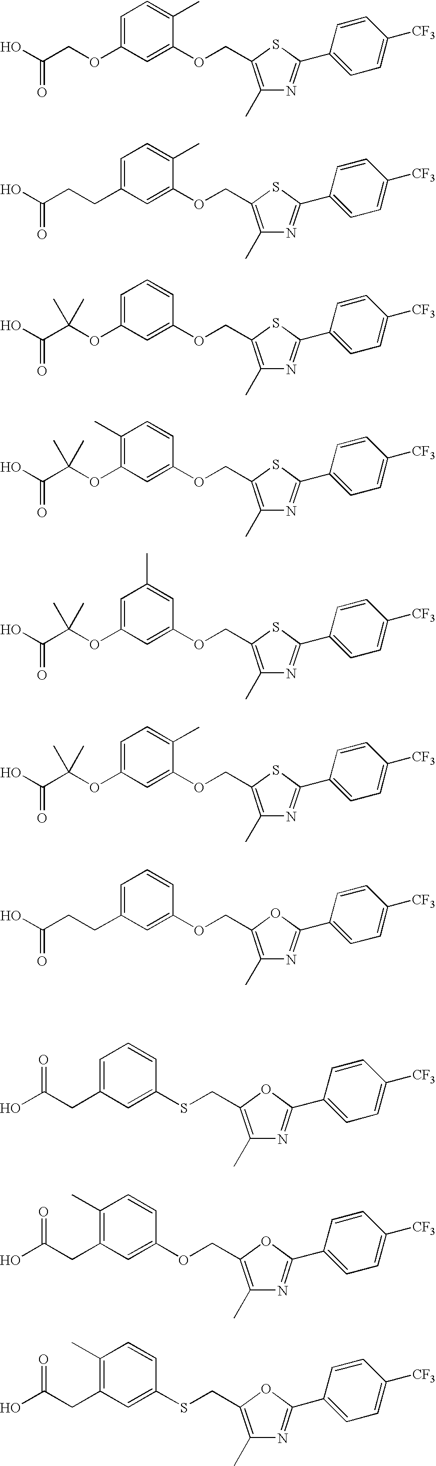

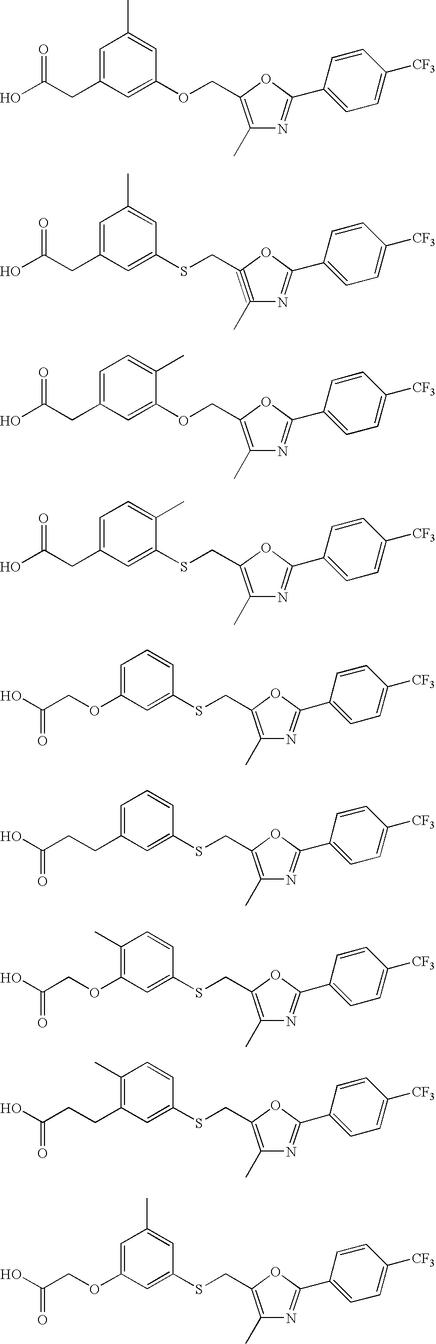

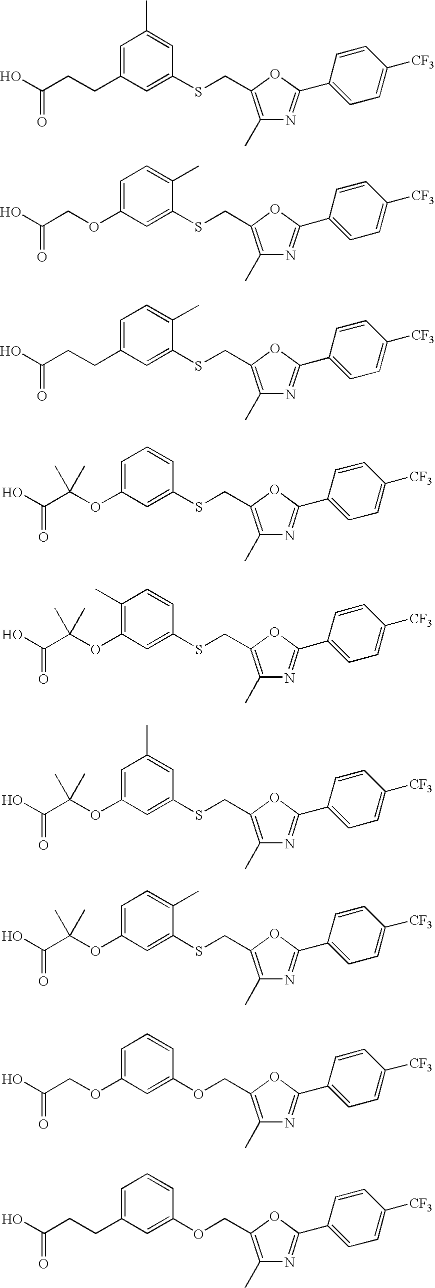

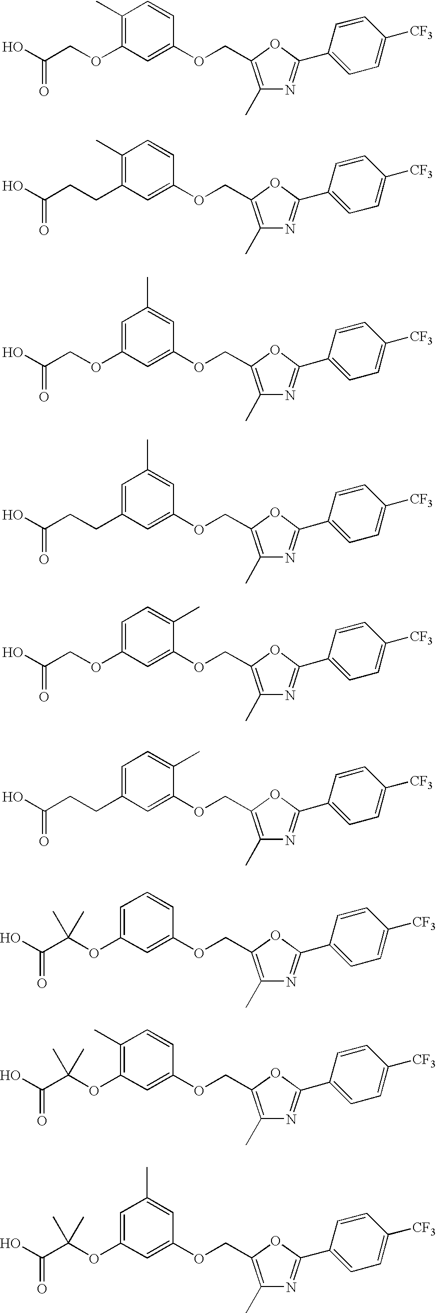

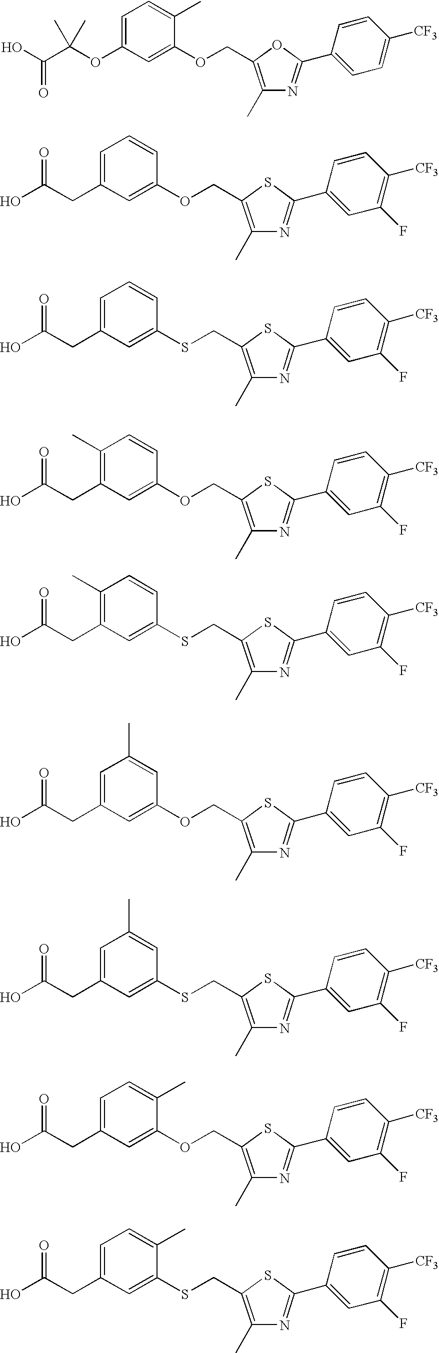

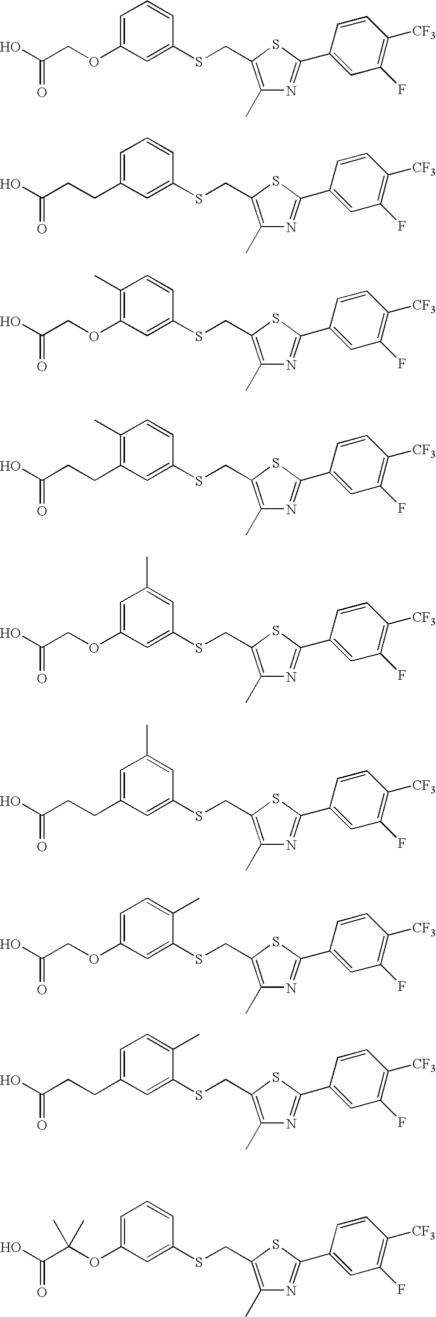

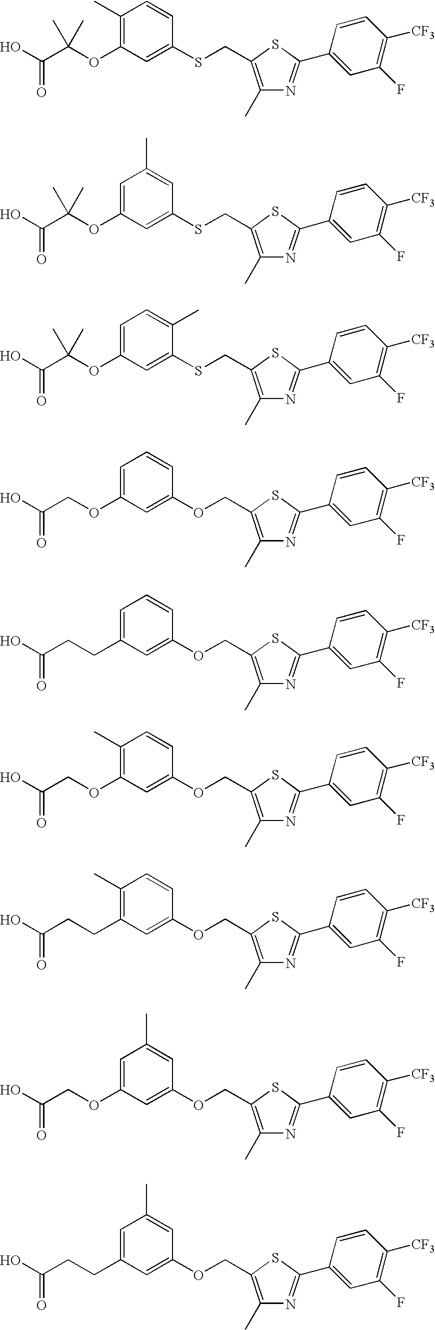

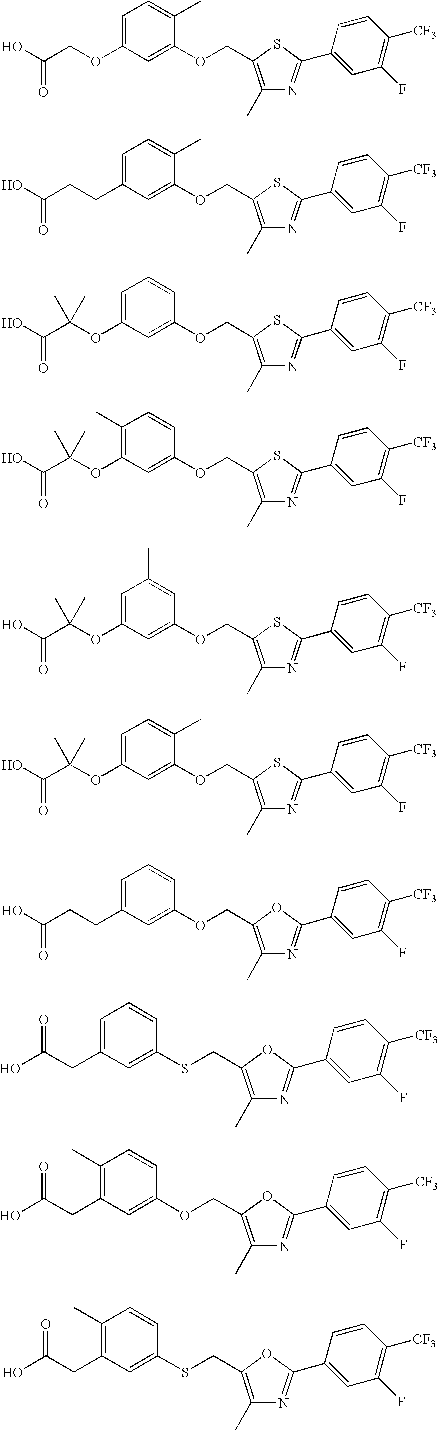

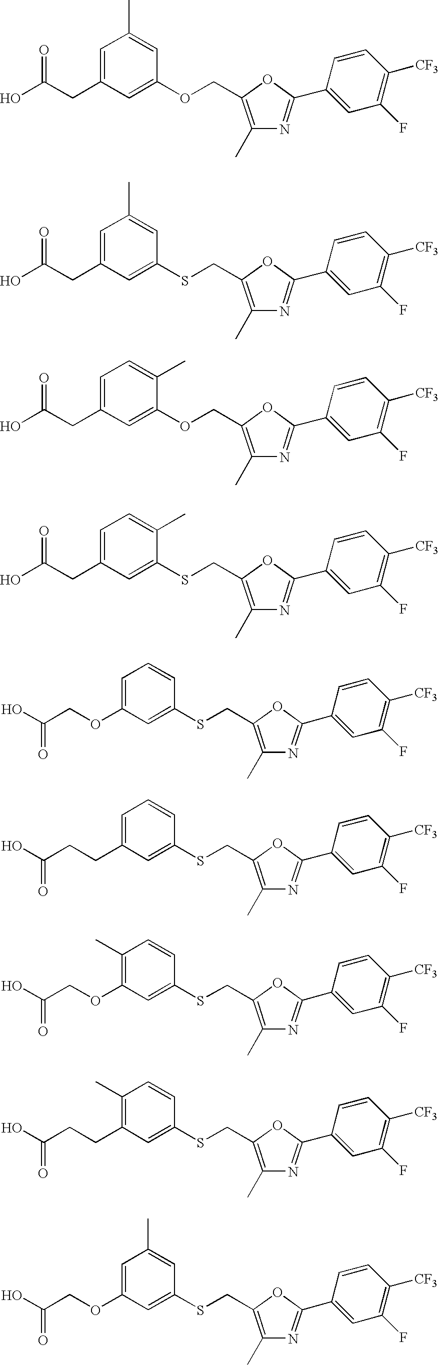

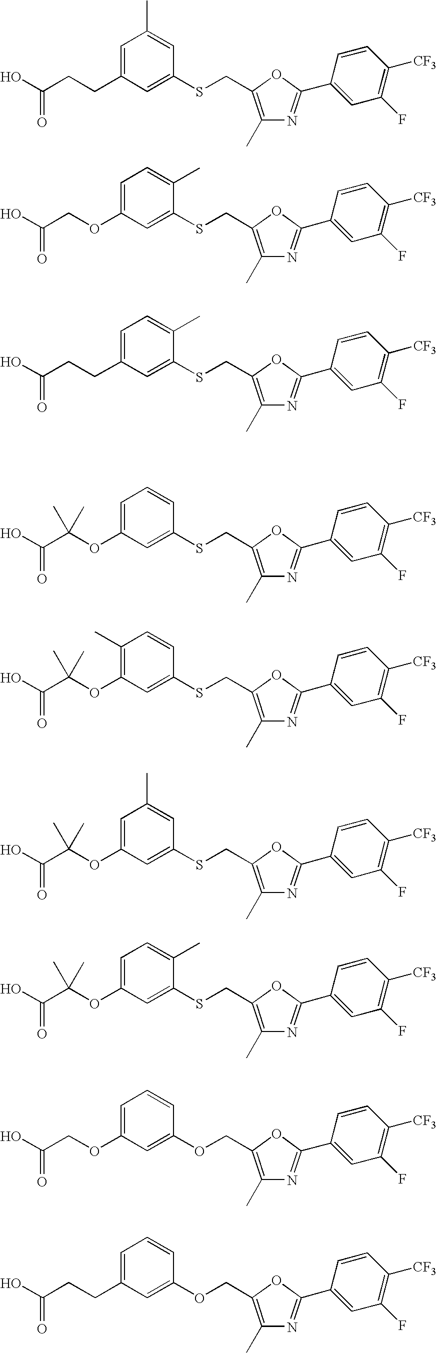

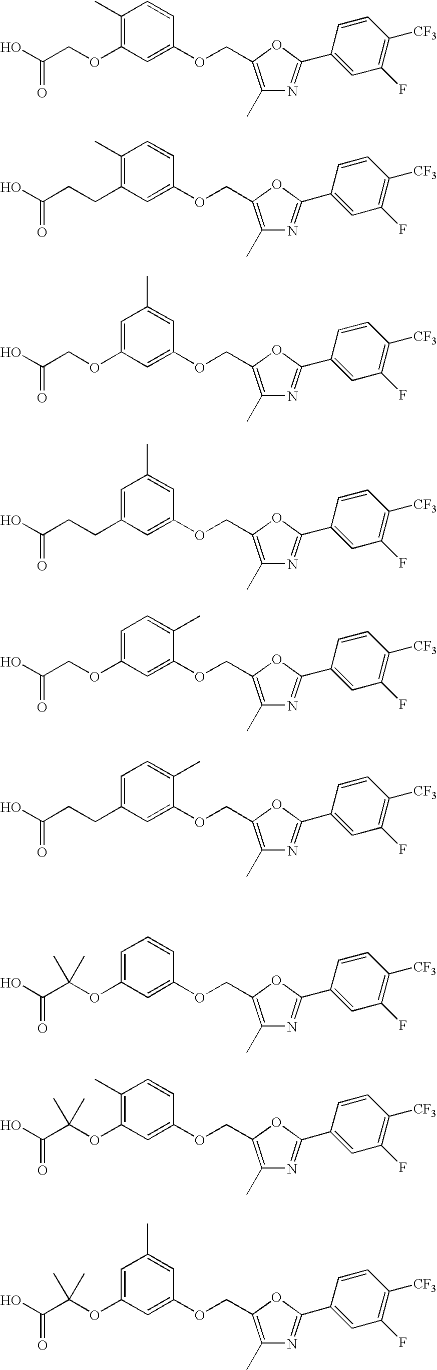

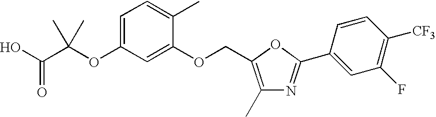

Other embodiments of the invention include any of the following compounds or pharmaceutically acceptable salts, amides, esters, thioesters, or pro-drugs thereof:

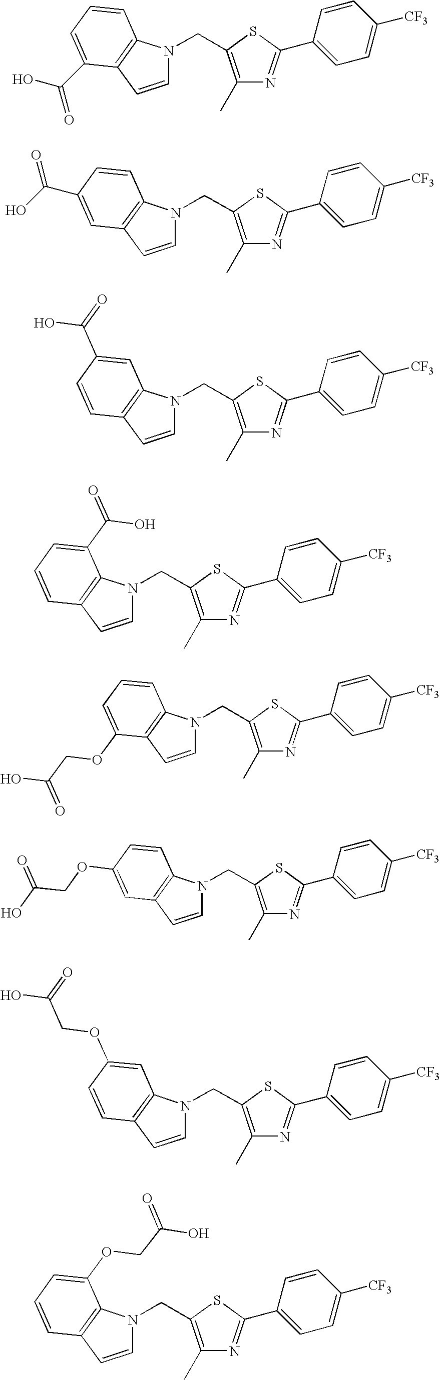

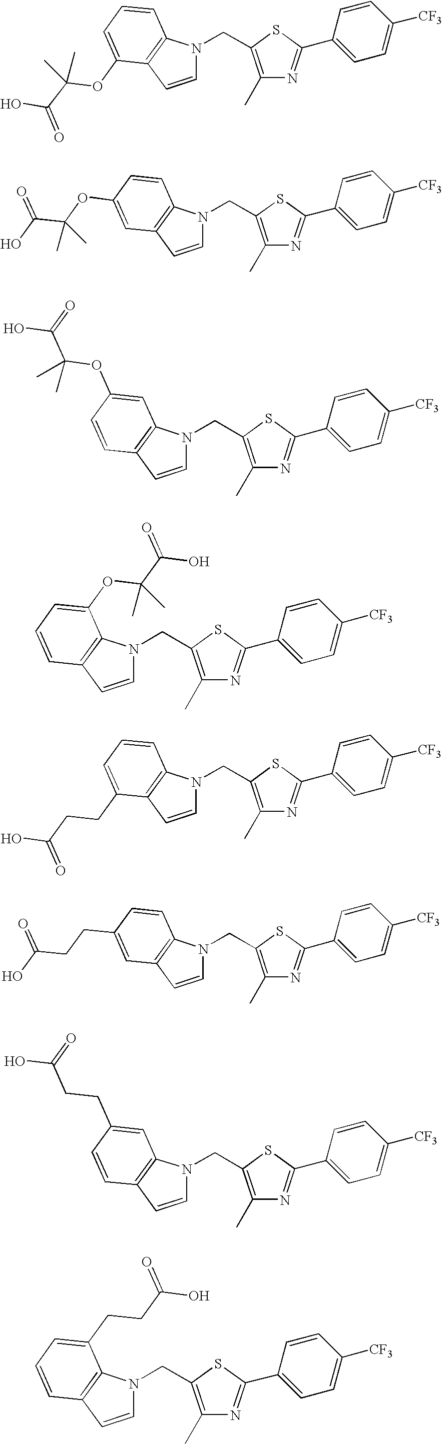

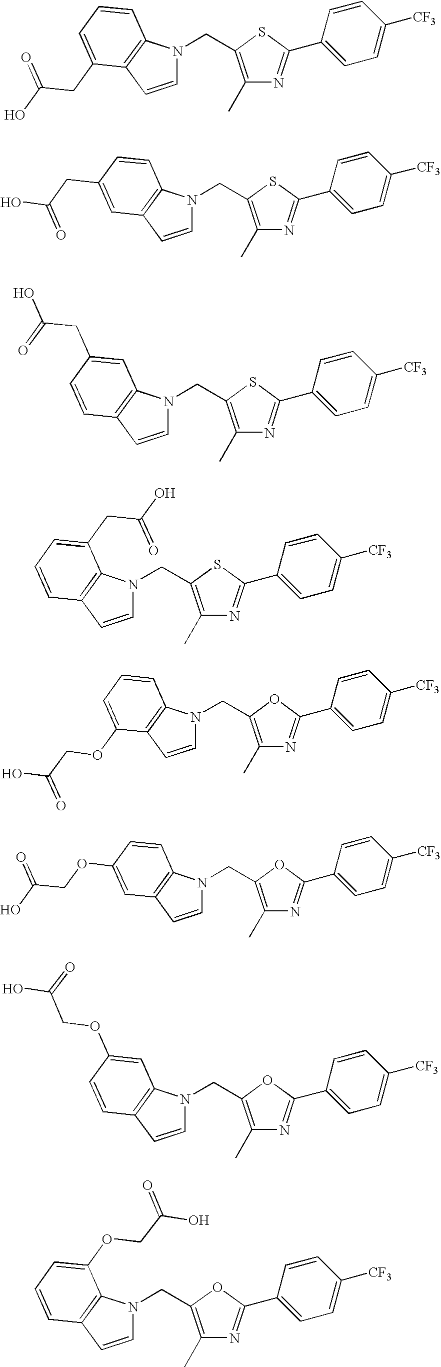

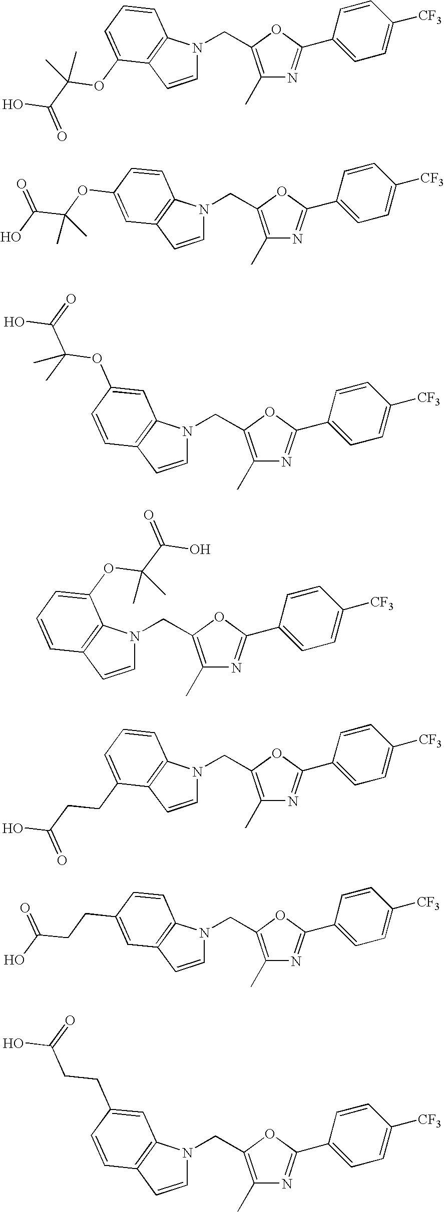

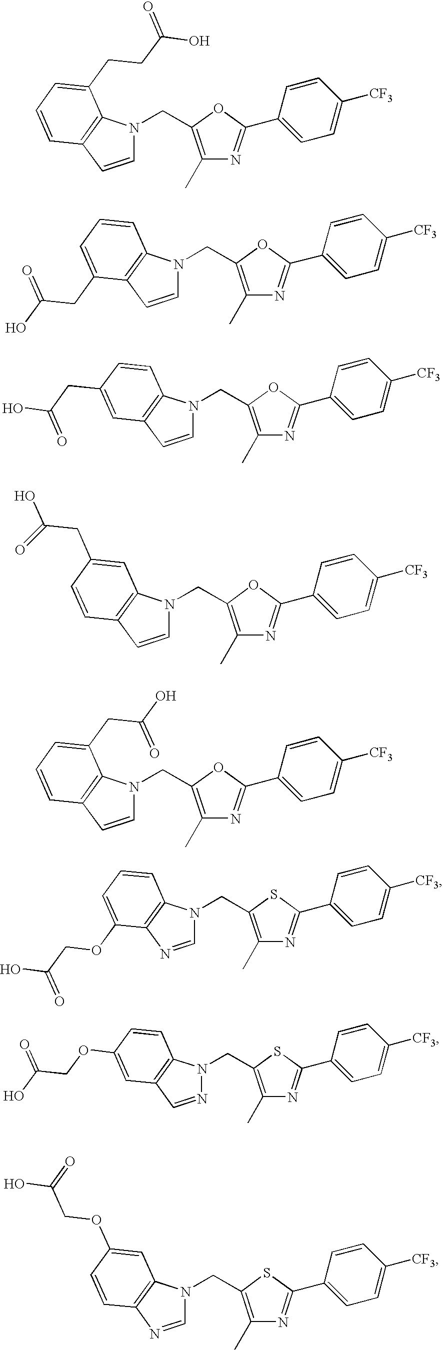

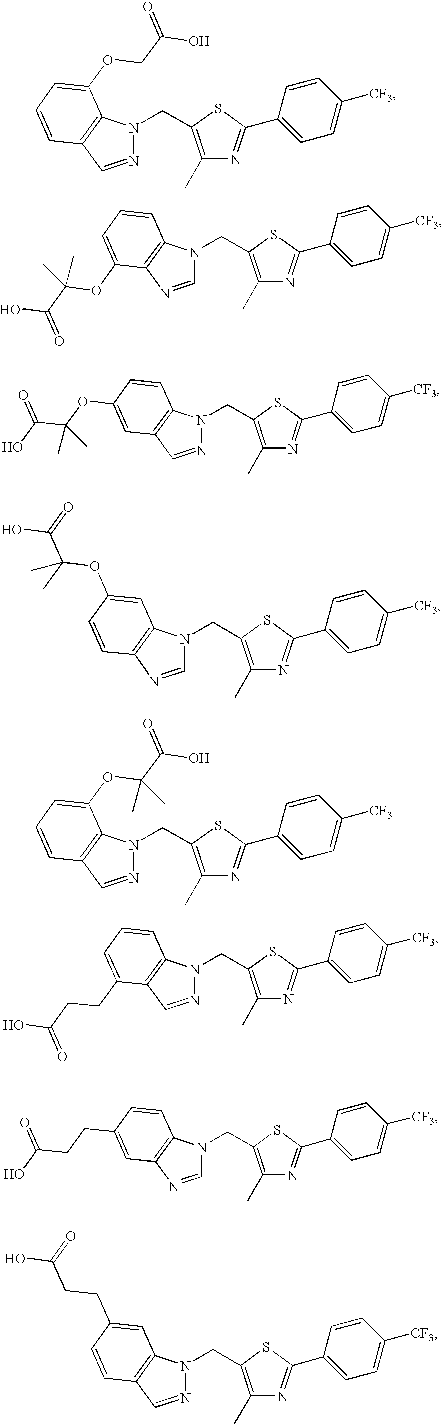

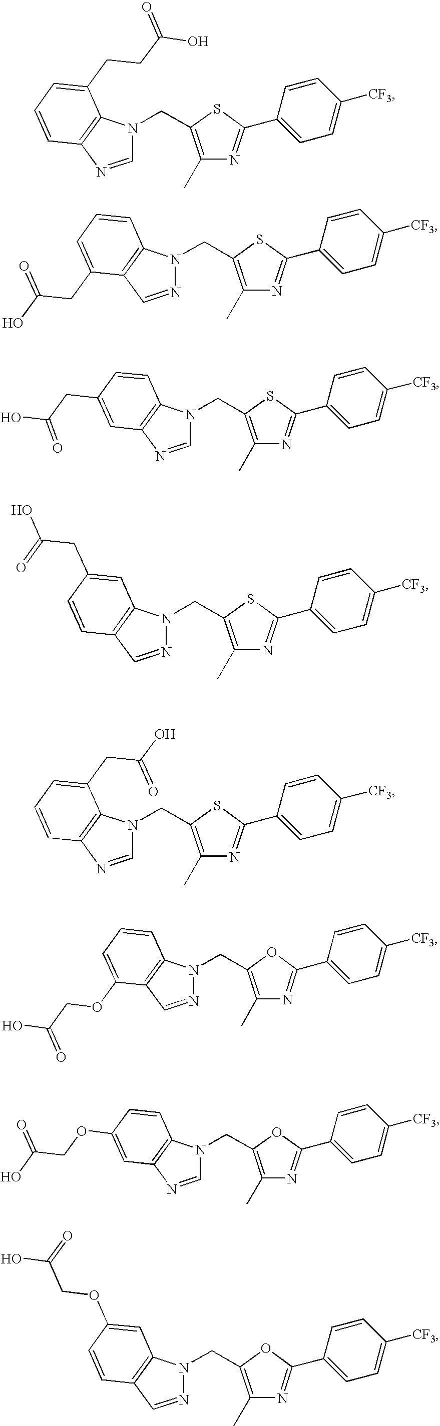

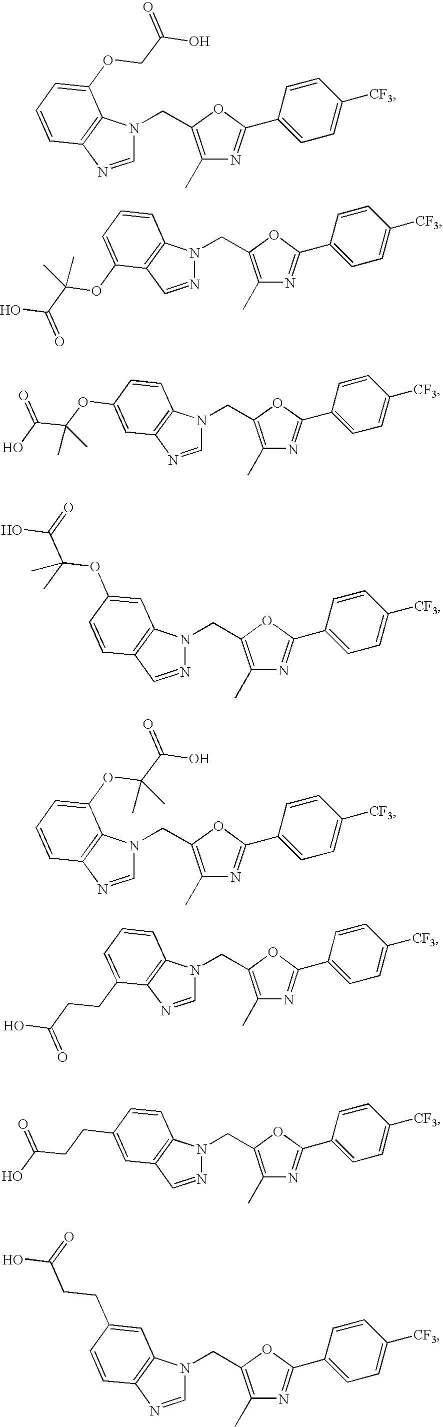

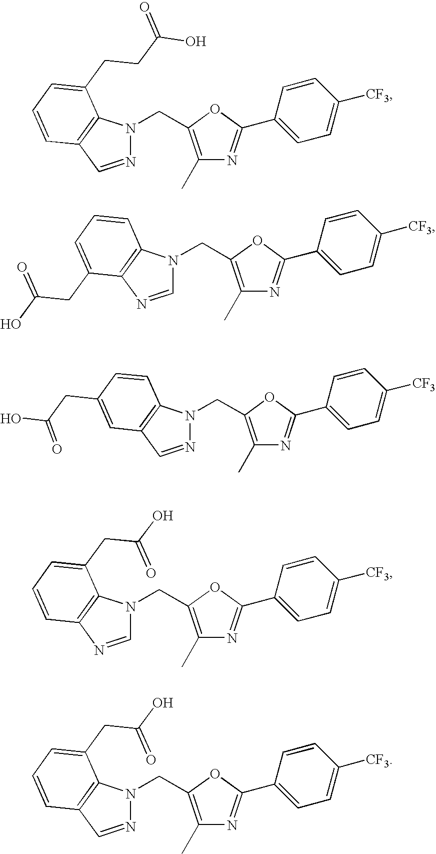

Other embodiments of the invention include any of the following compounds or pharmaceutically acceptable salts, amides, esters, thioesters, or pro-drugs thereof:

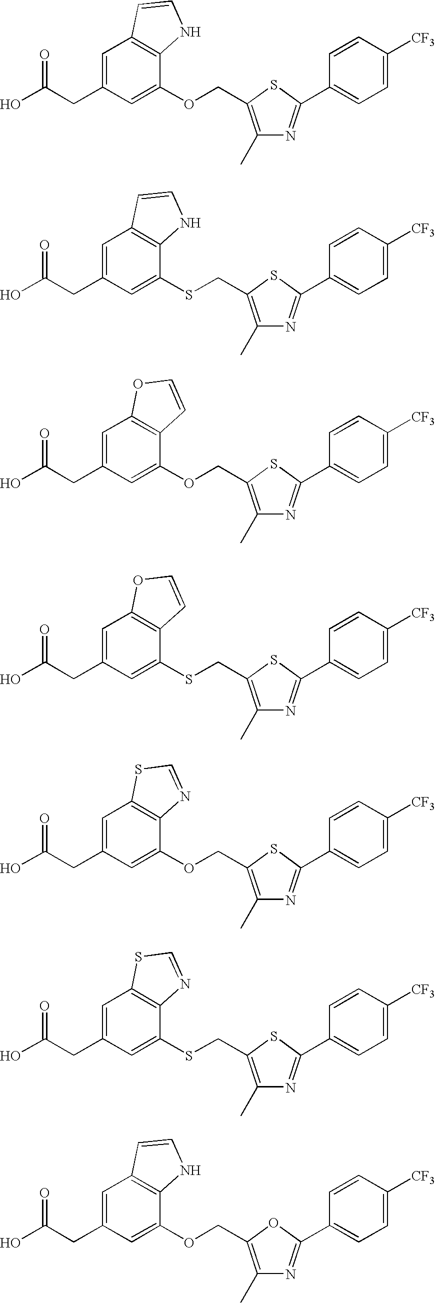

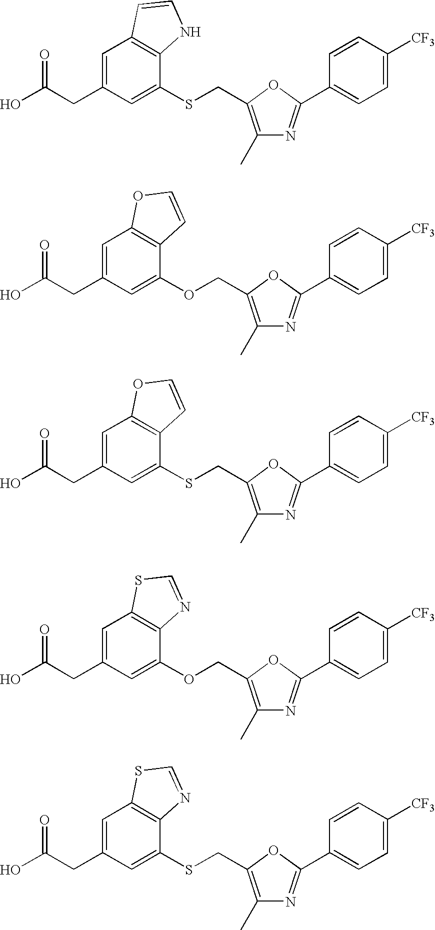

Other embodiments of the invention include any of the following compounds or pharmaceutically acceptable salts, amides, esters, thioesters, or pro-drugs thereof:

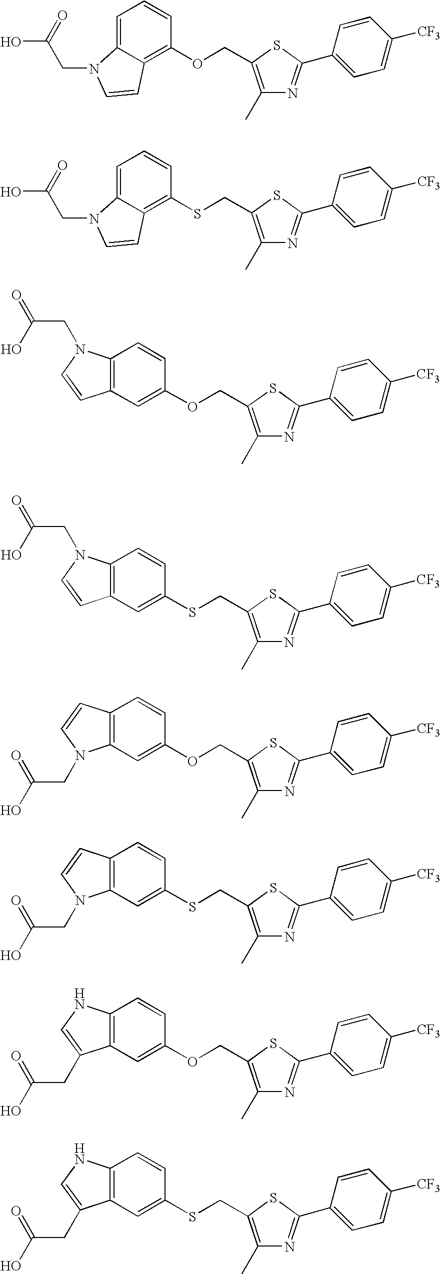

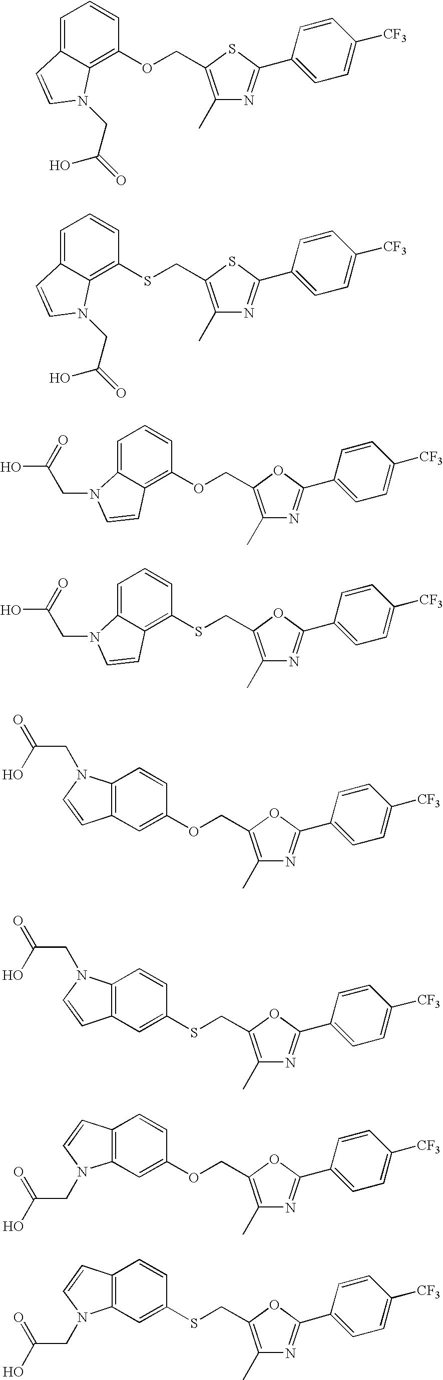

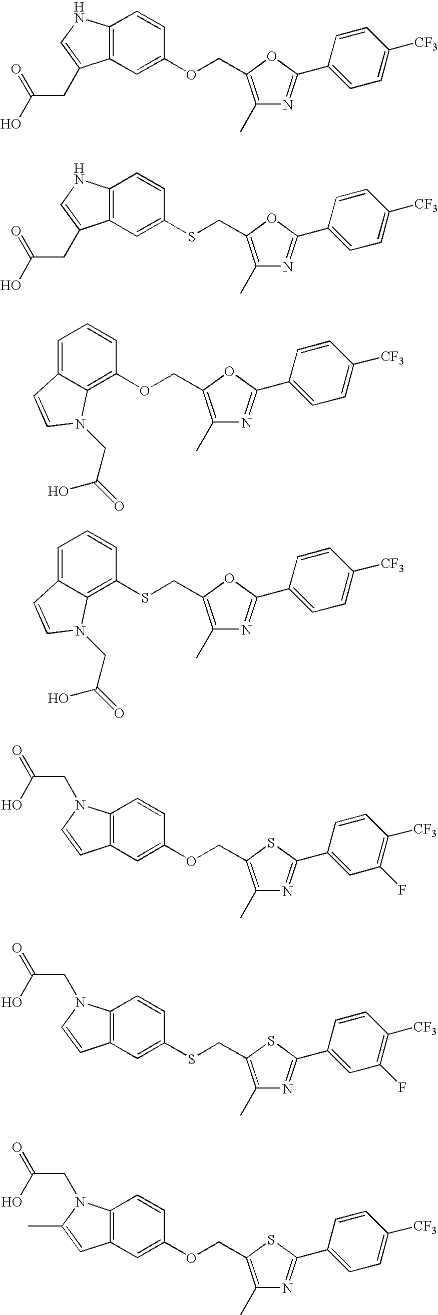

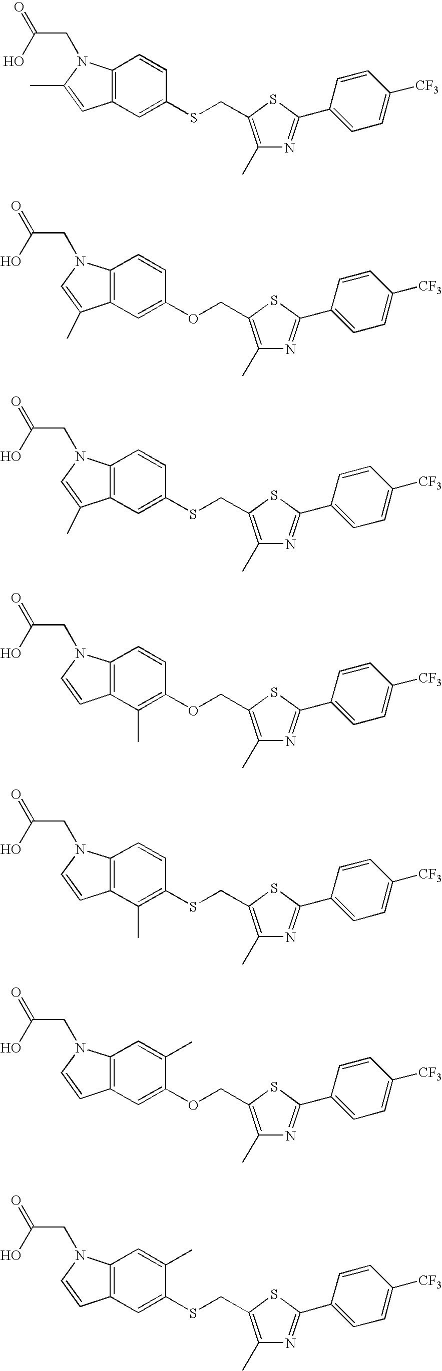

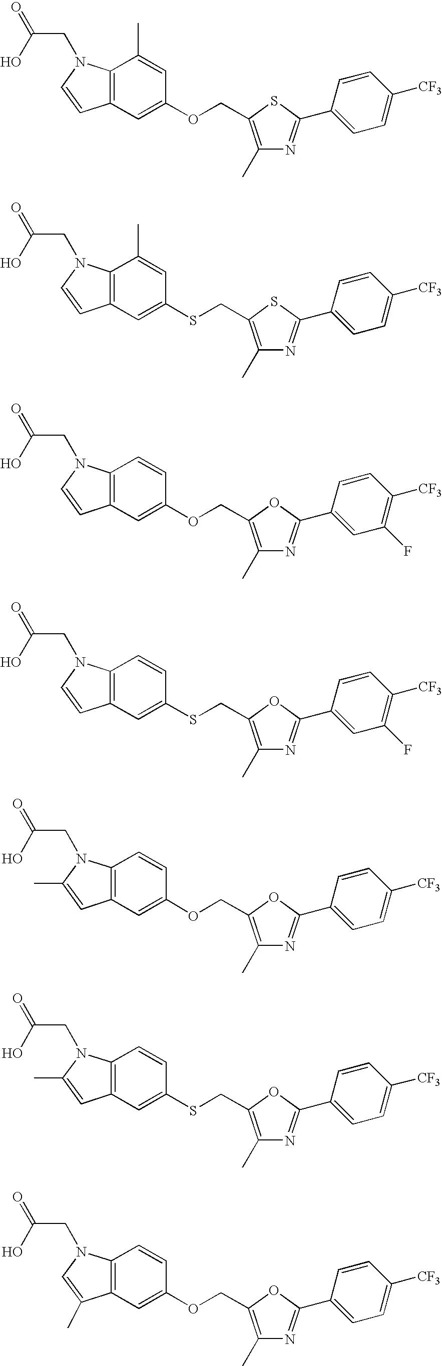

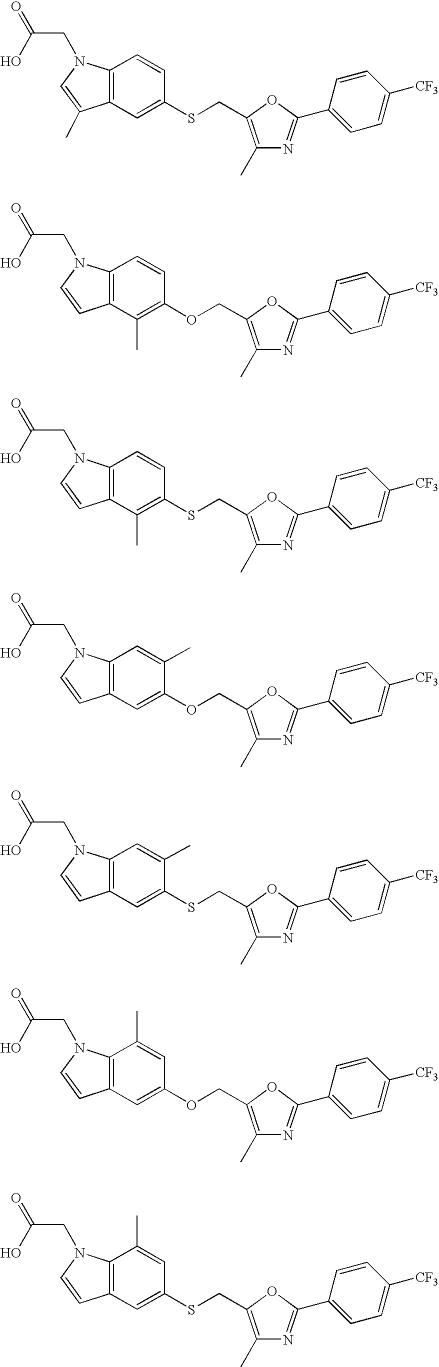

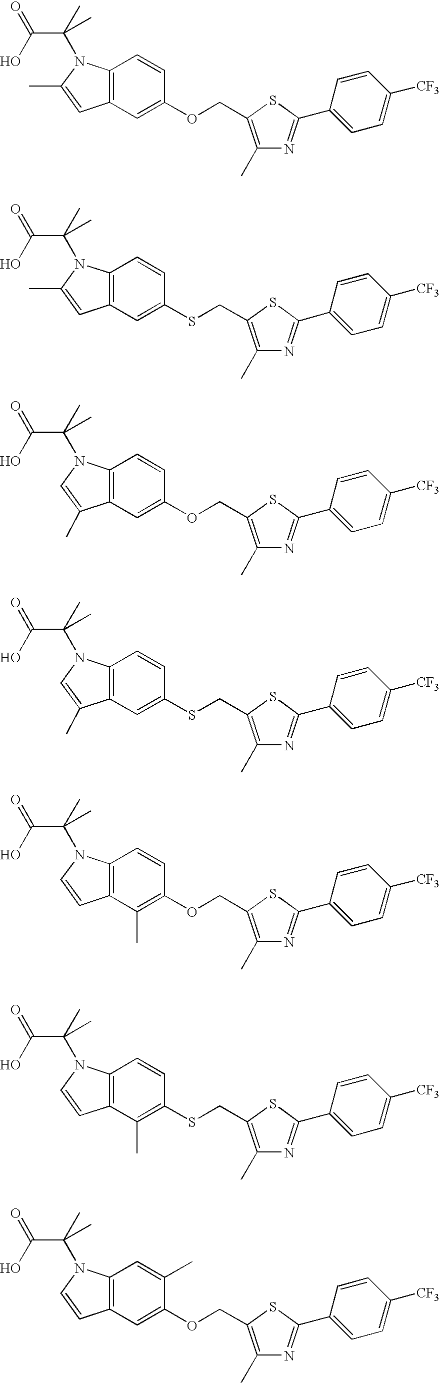

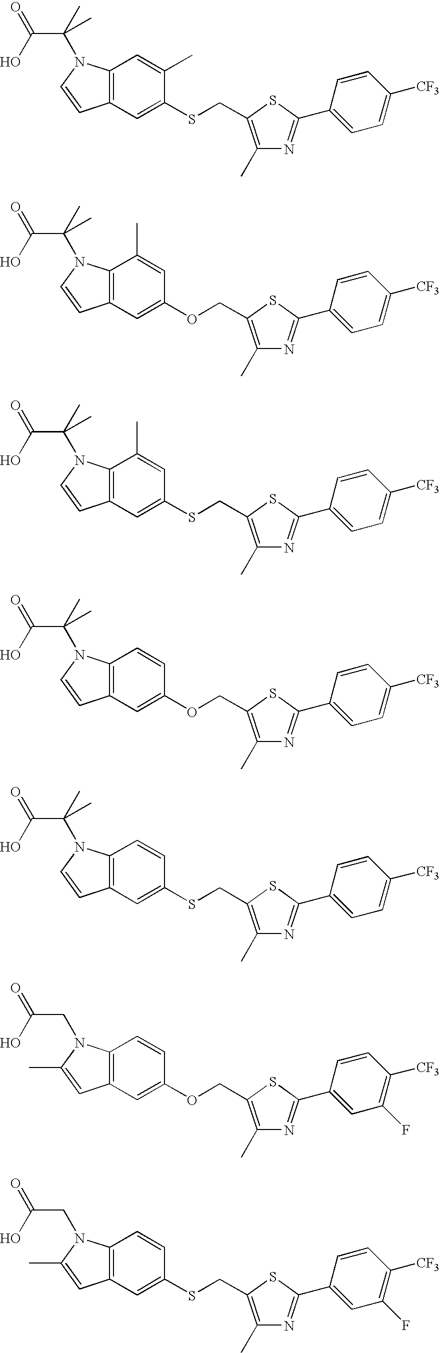

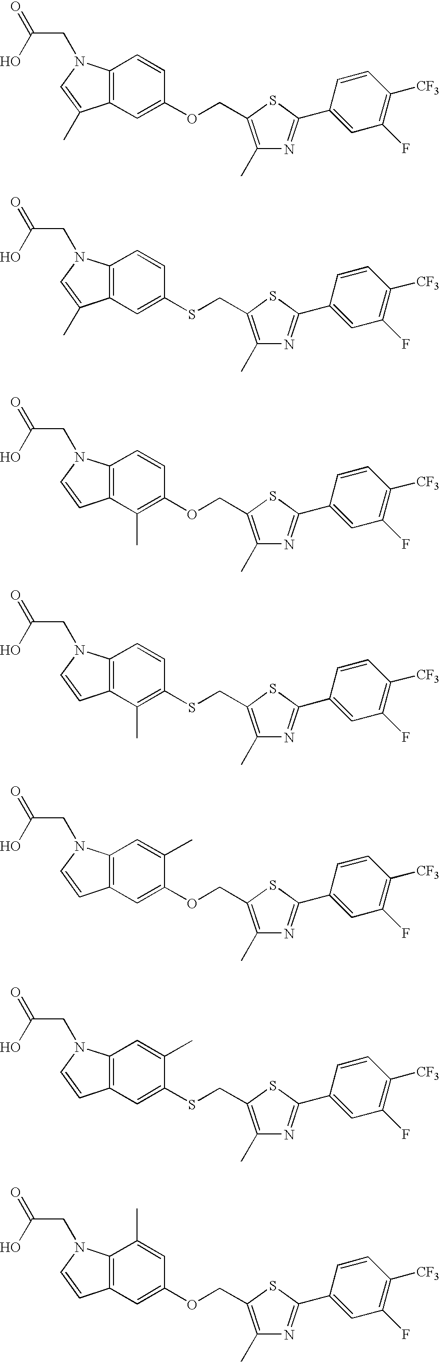

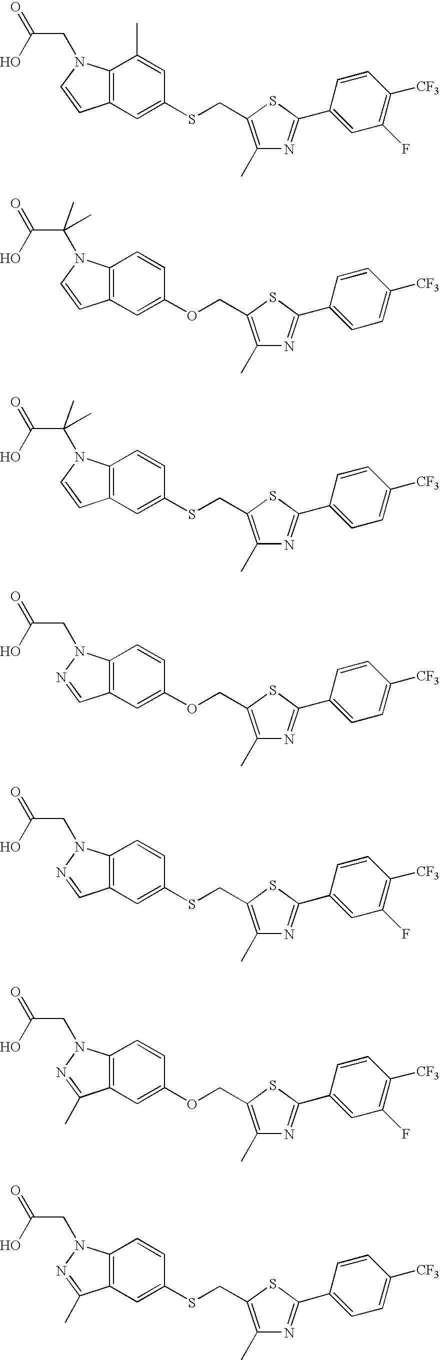

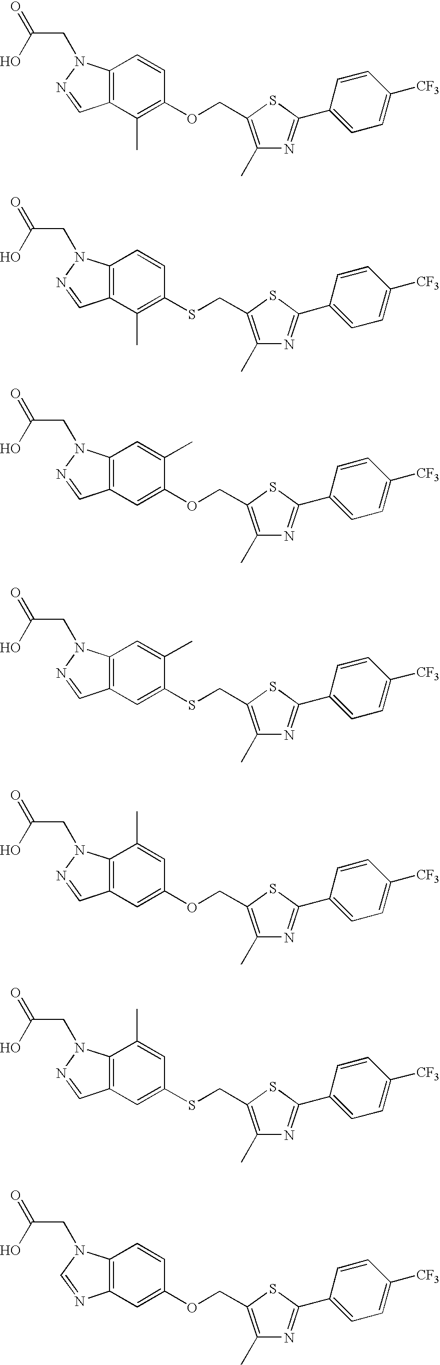

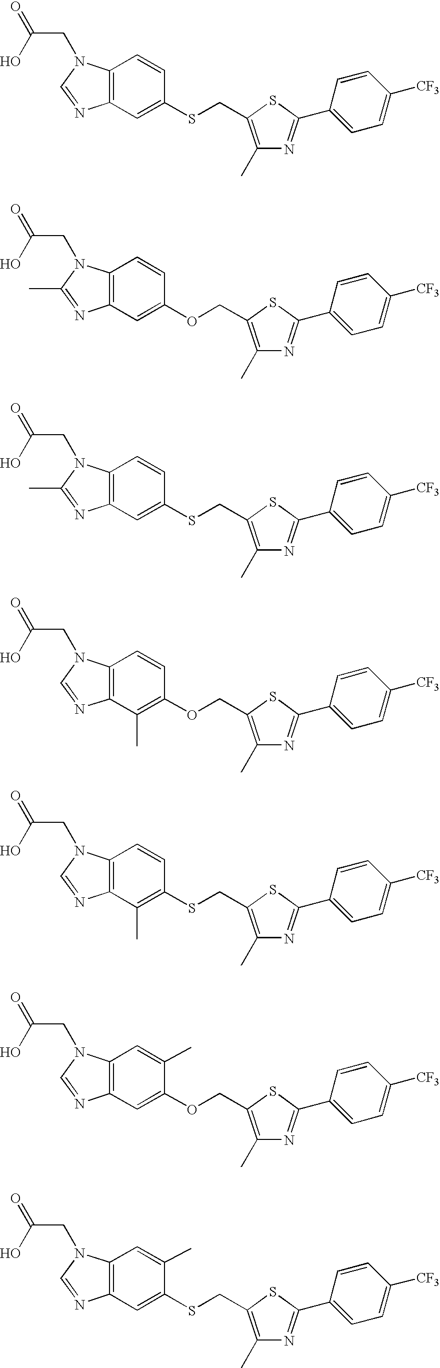

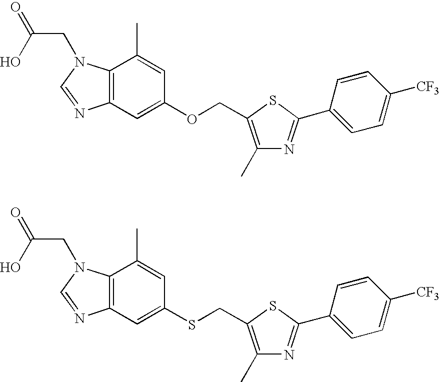

Other embodiments of the invention include any of the following compounds or pharmaceutically acceptable salts, amides, esters, thioesters, or pro-drugs thereof:

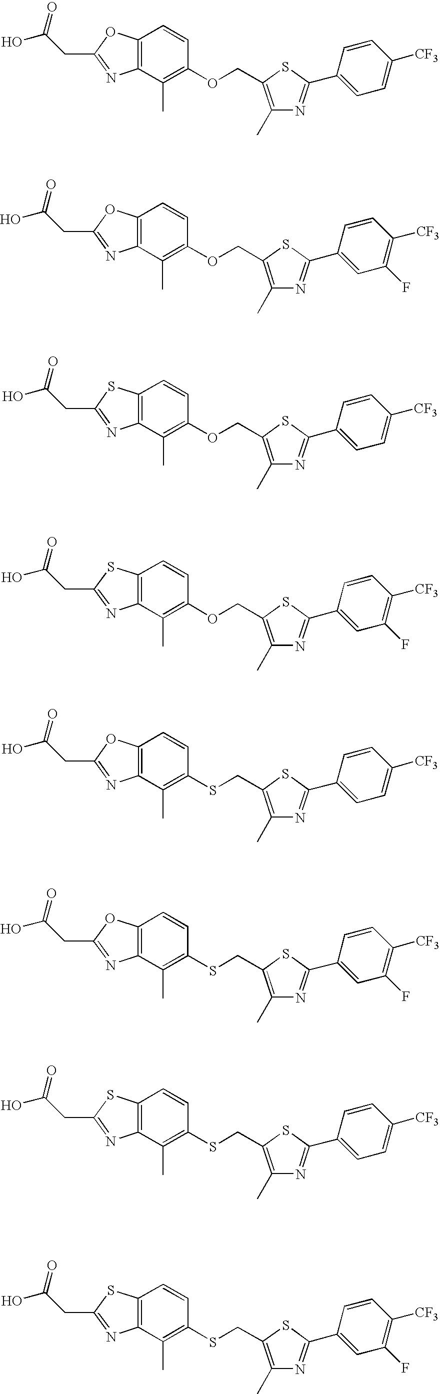

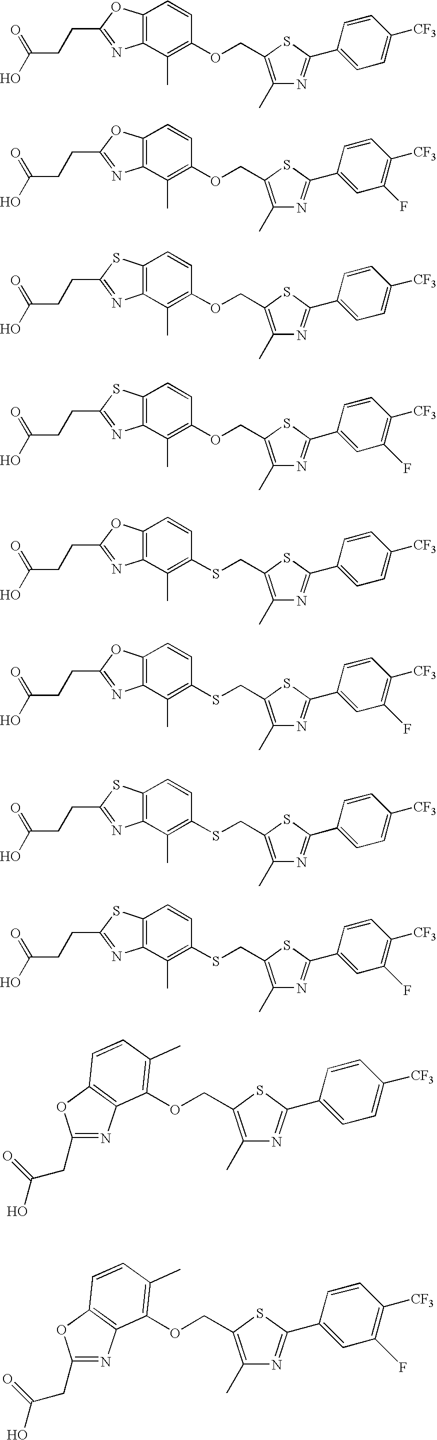

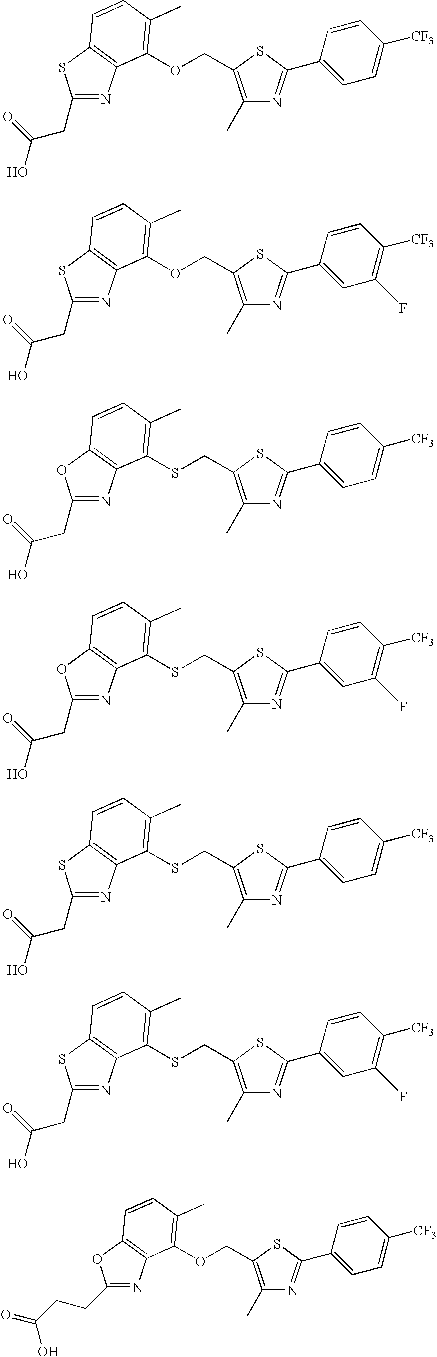

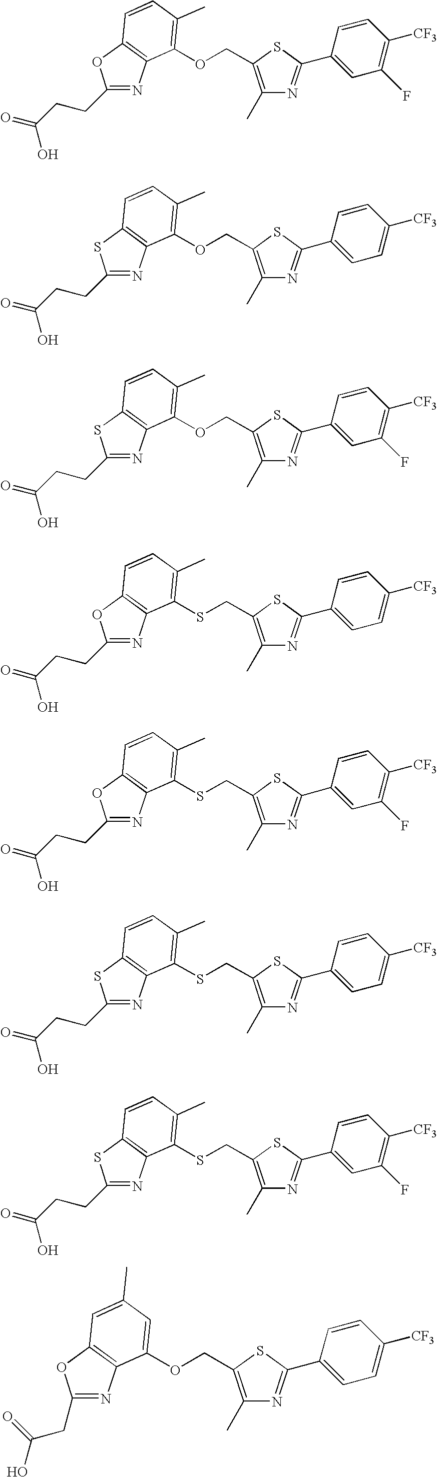

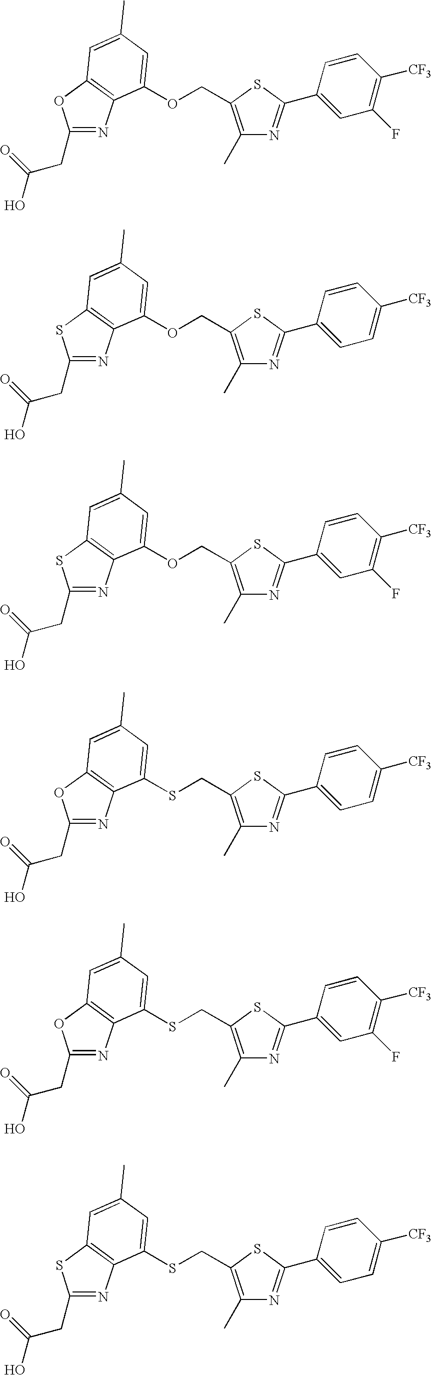

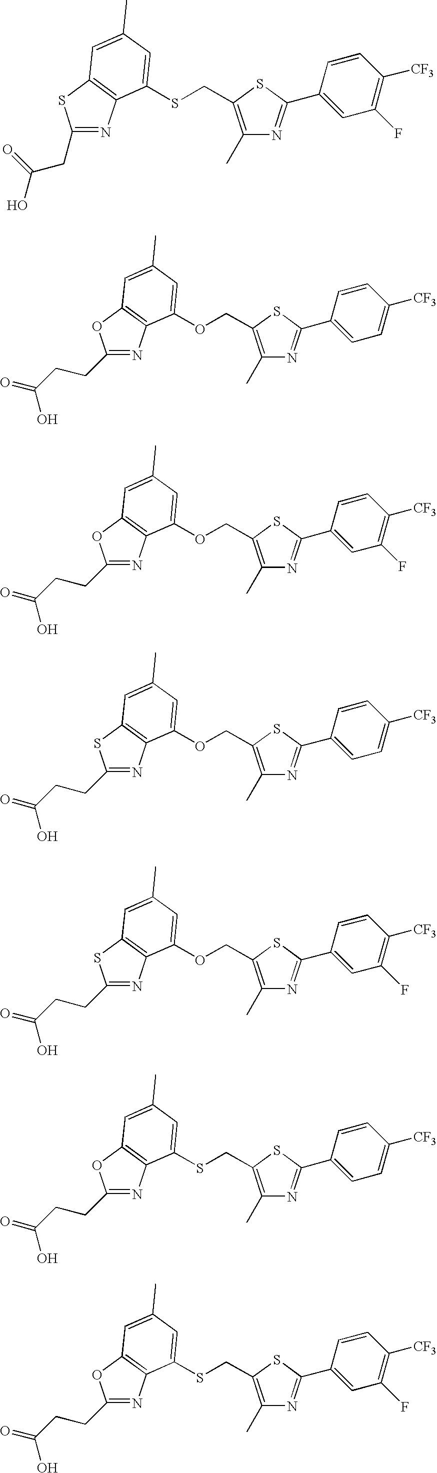

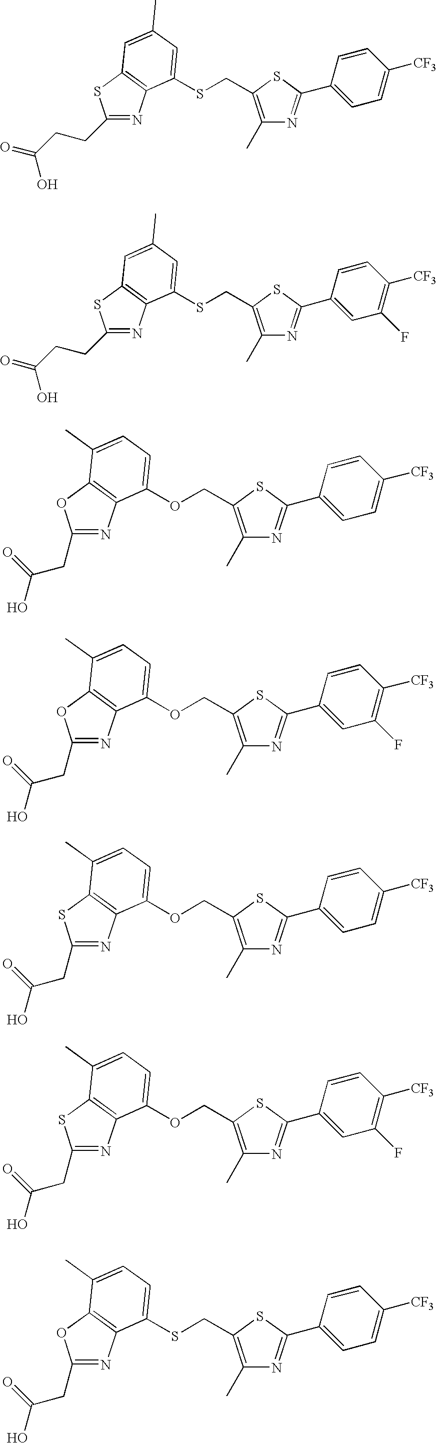

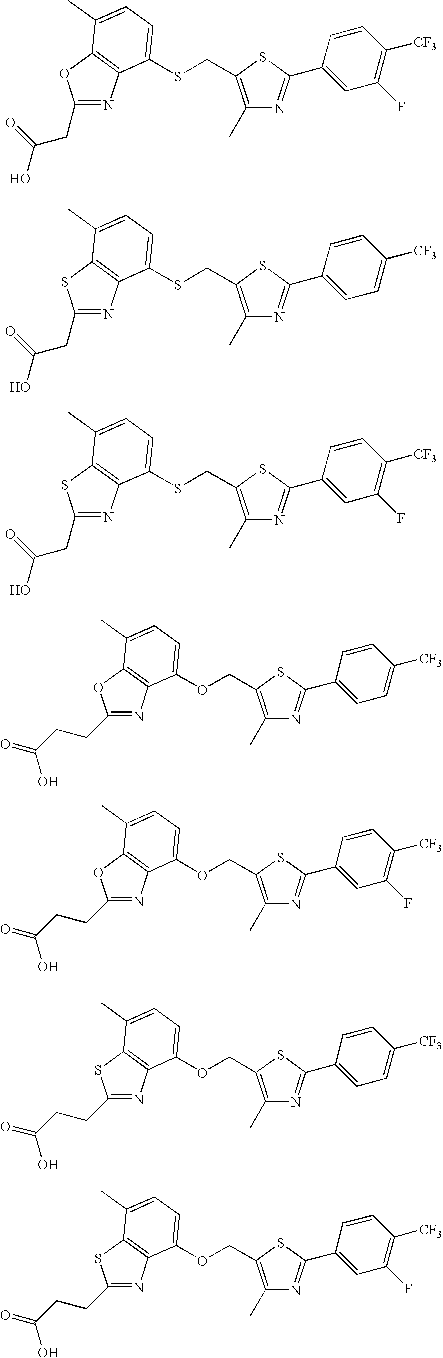

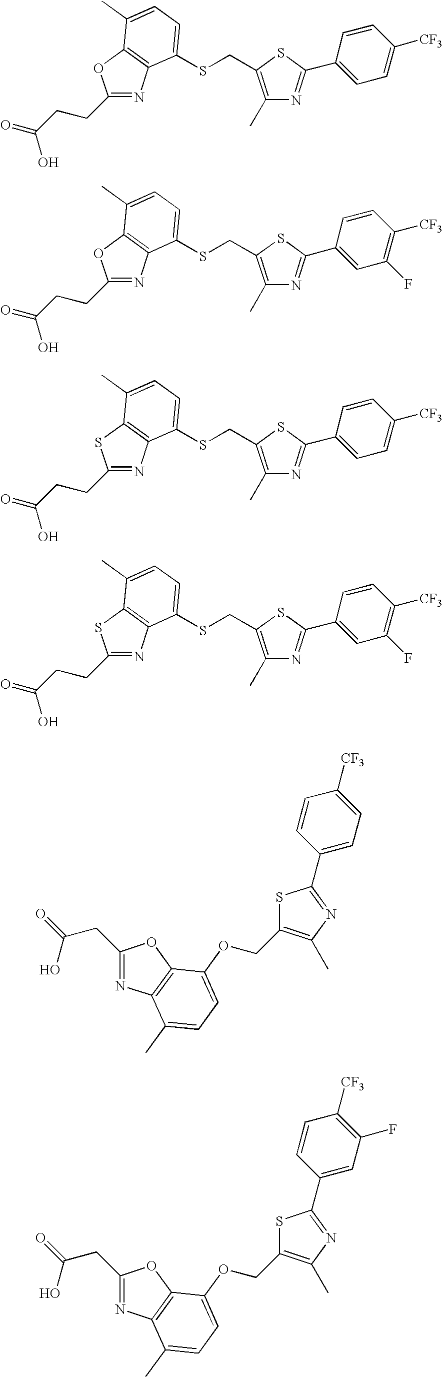

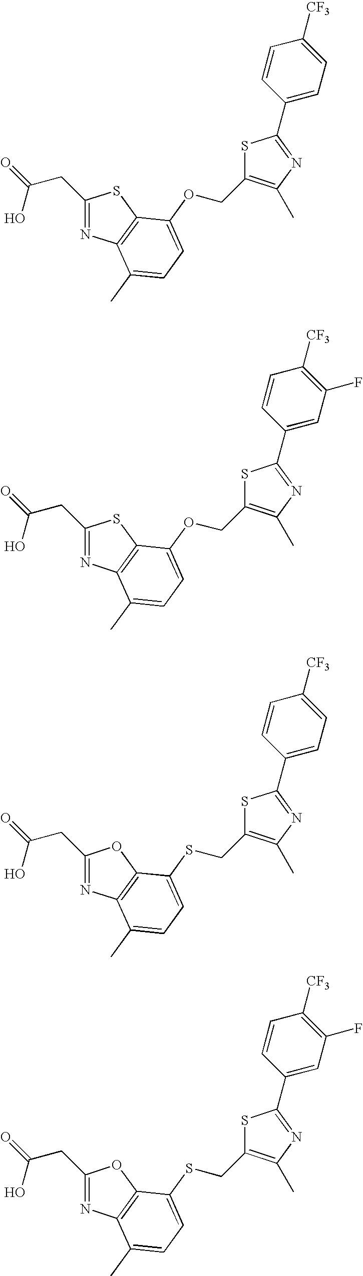

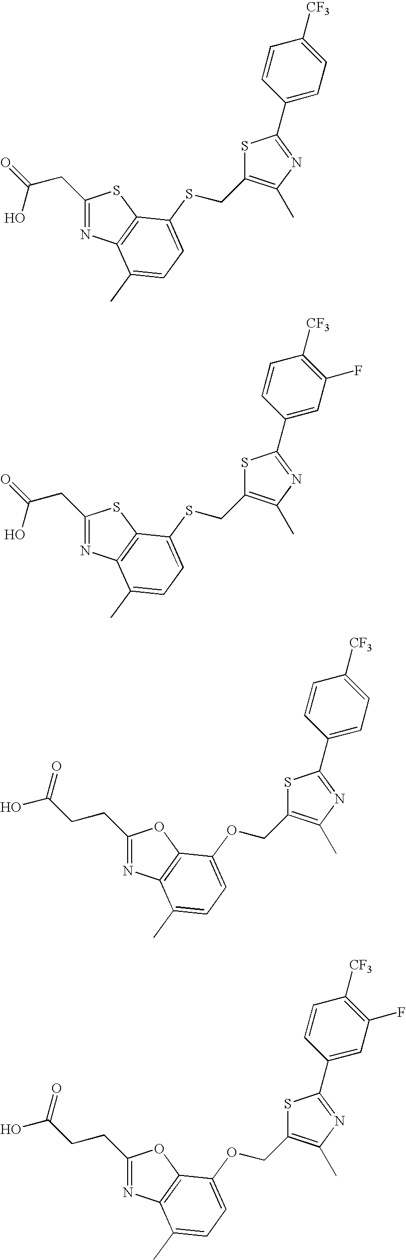

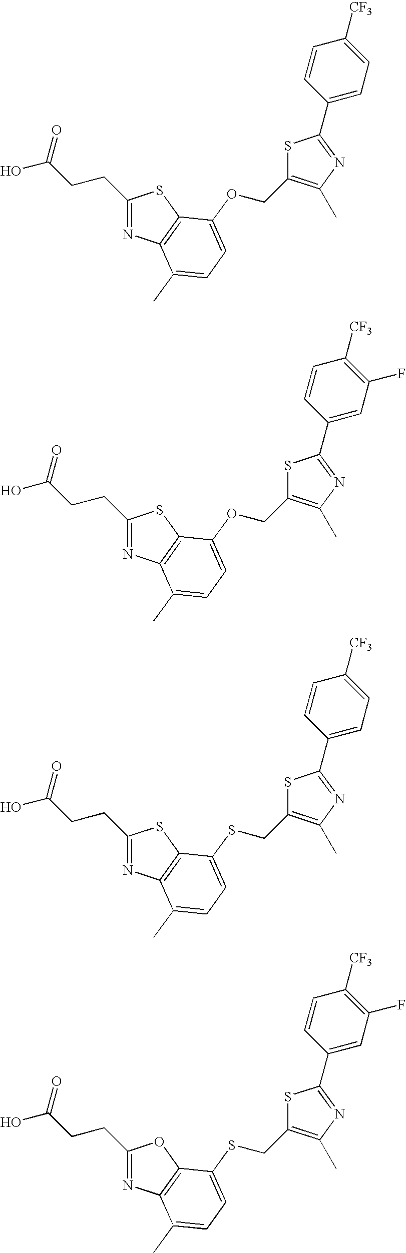

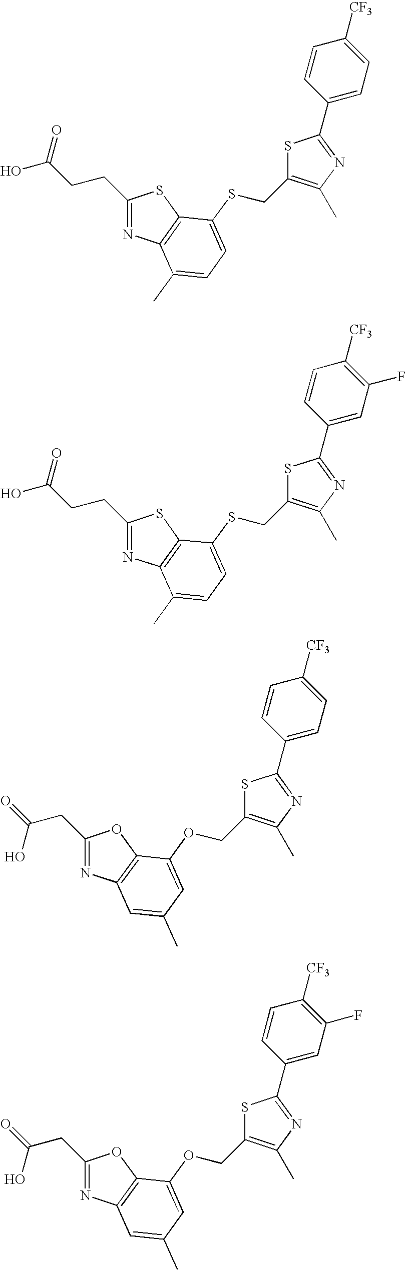

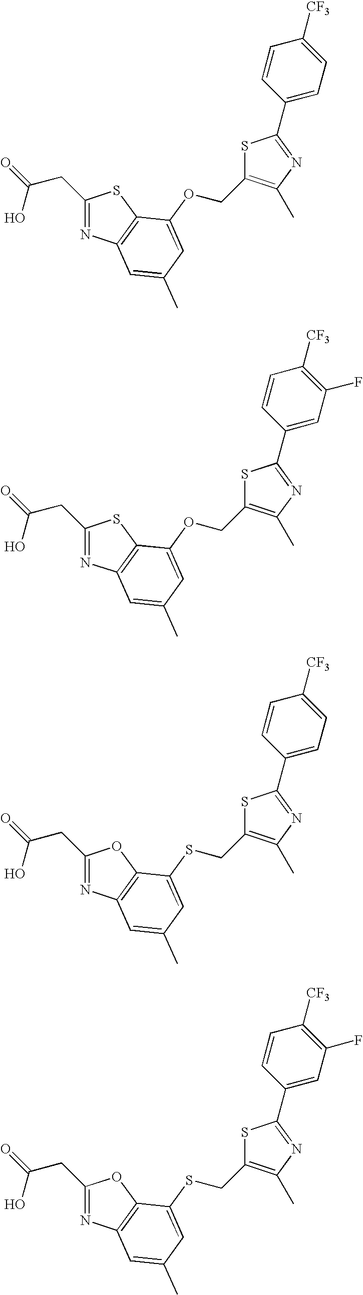

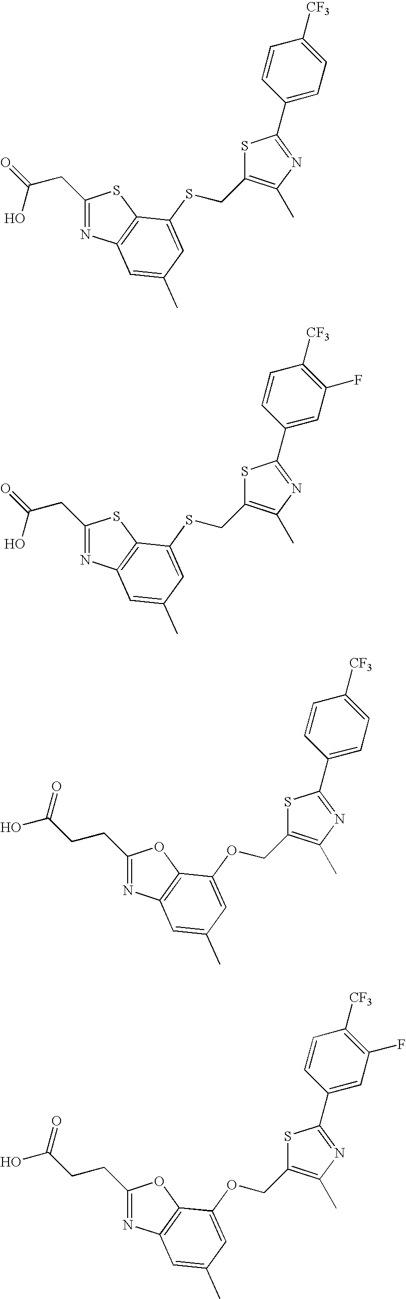

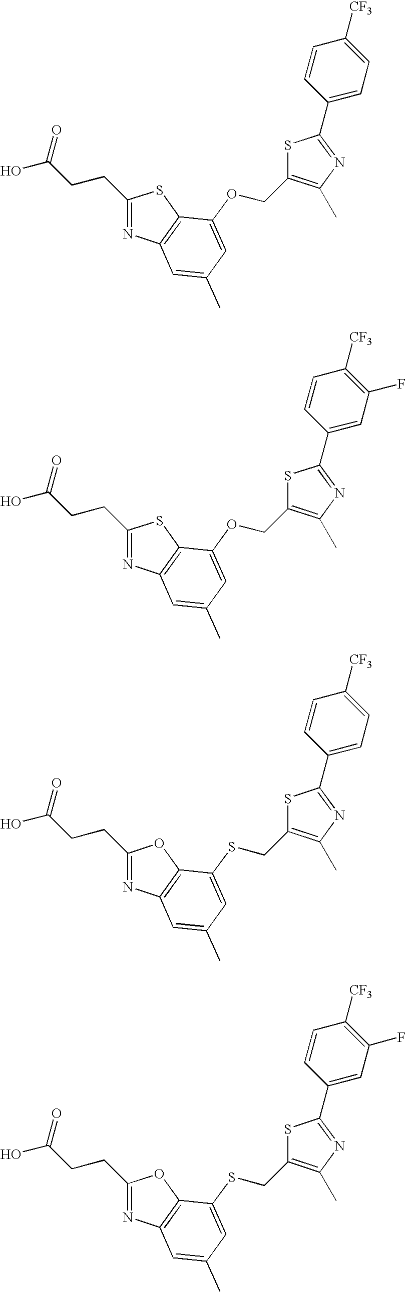

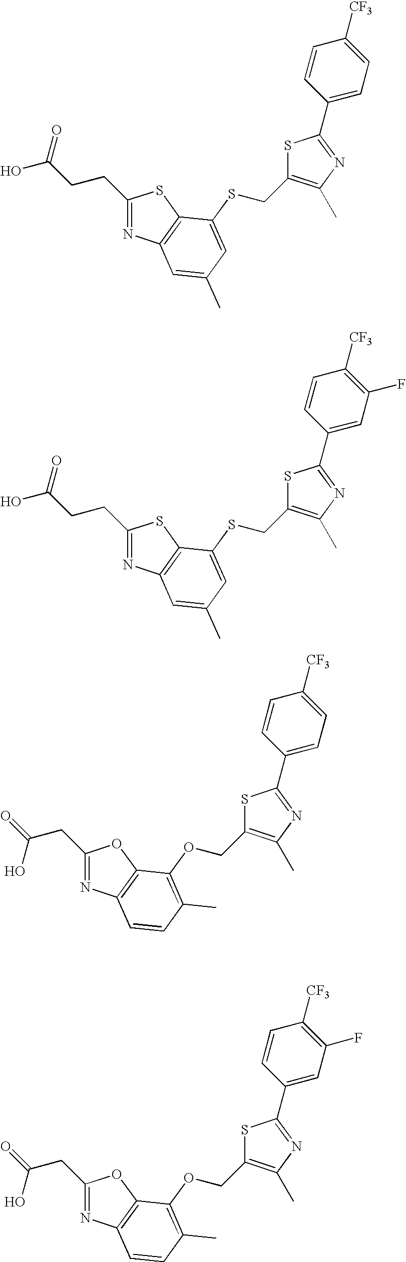

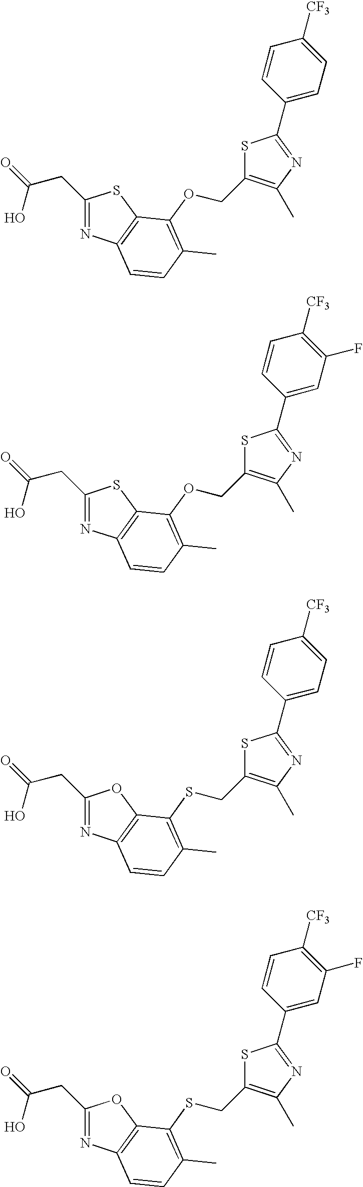

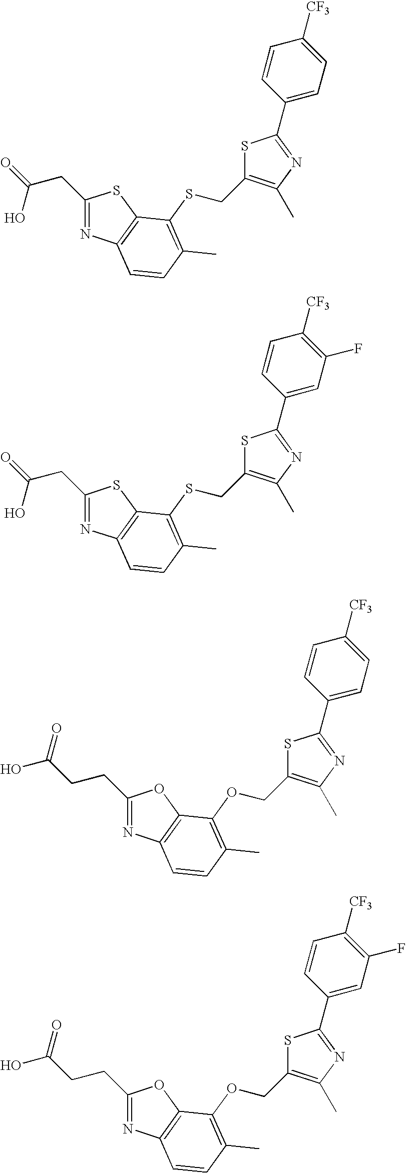

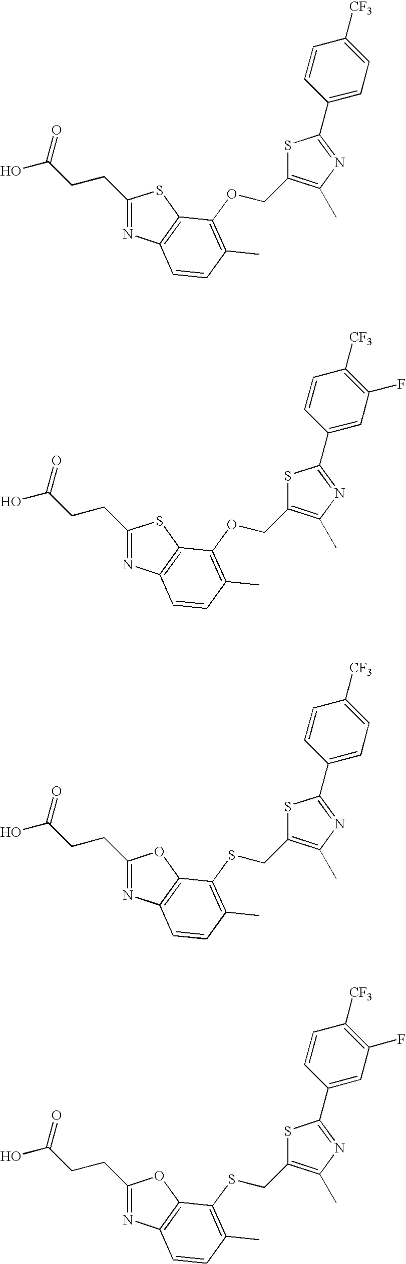

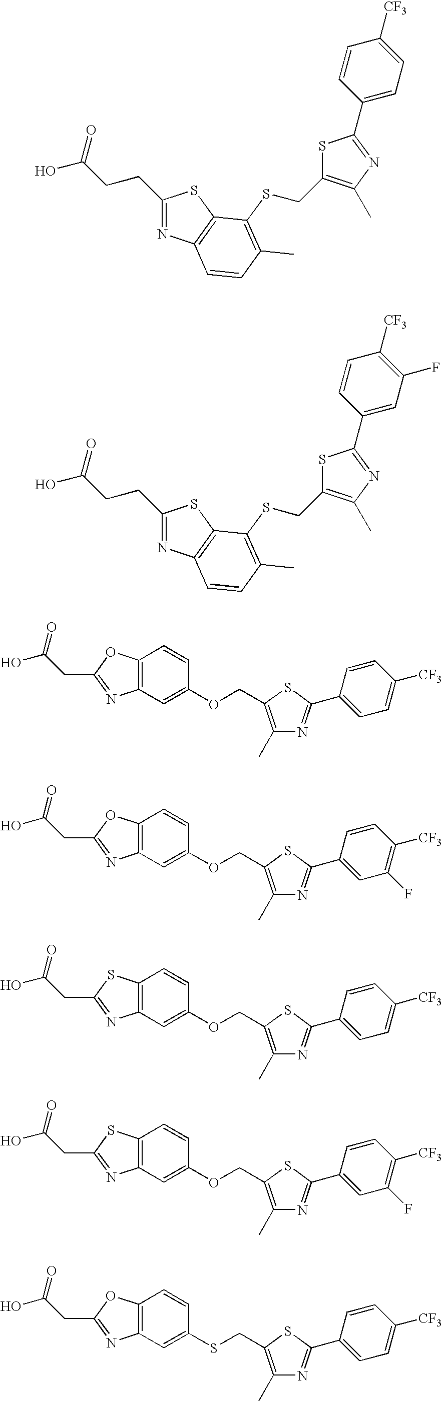

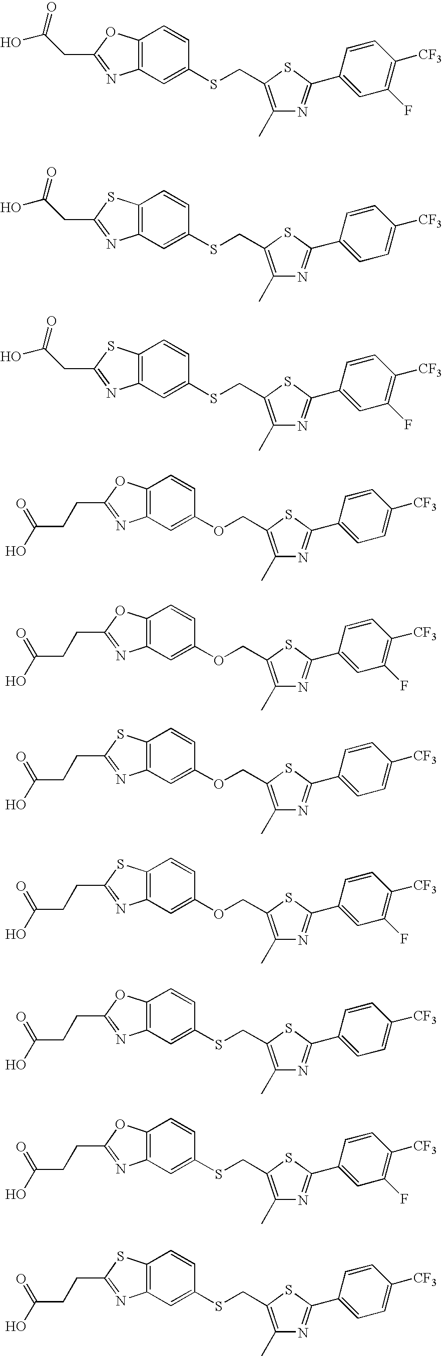

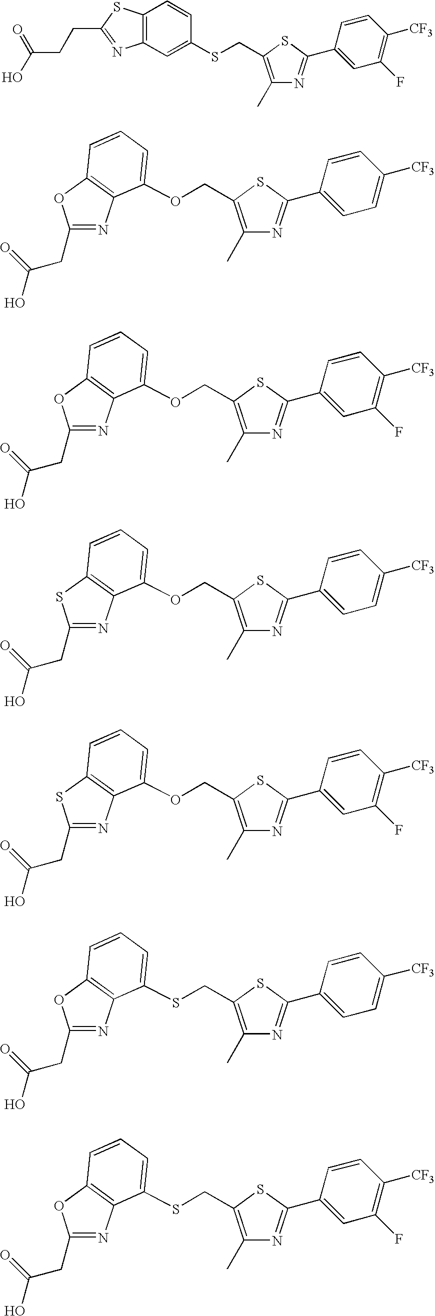

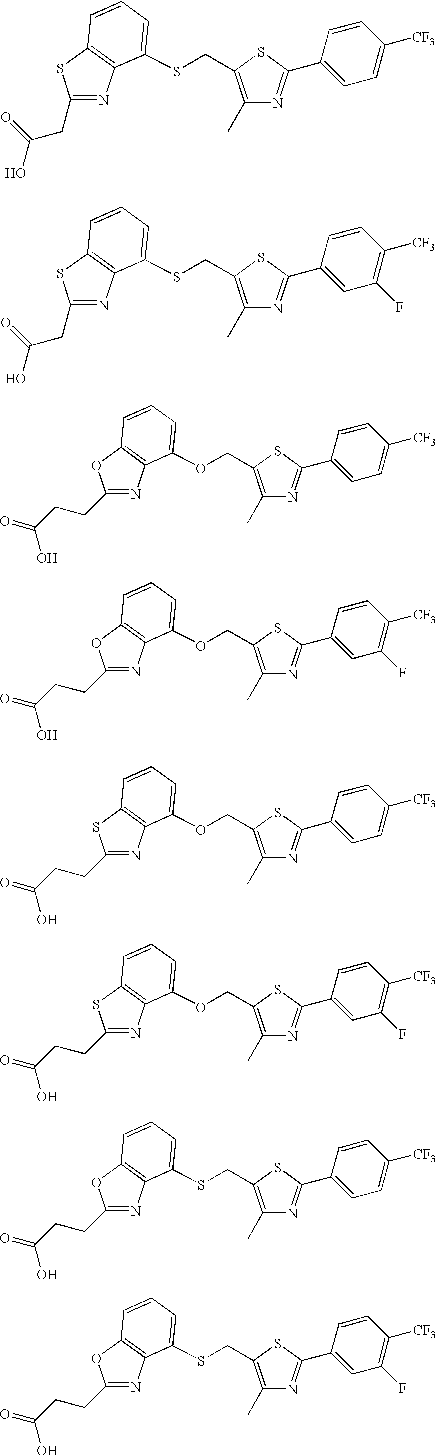

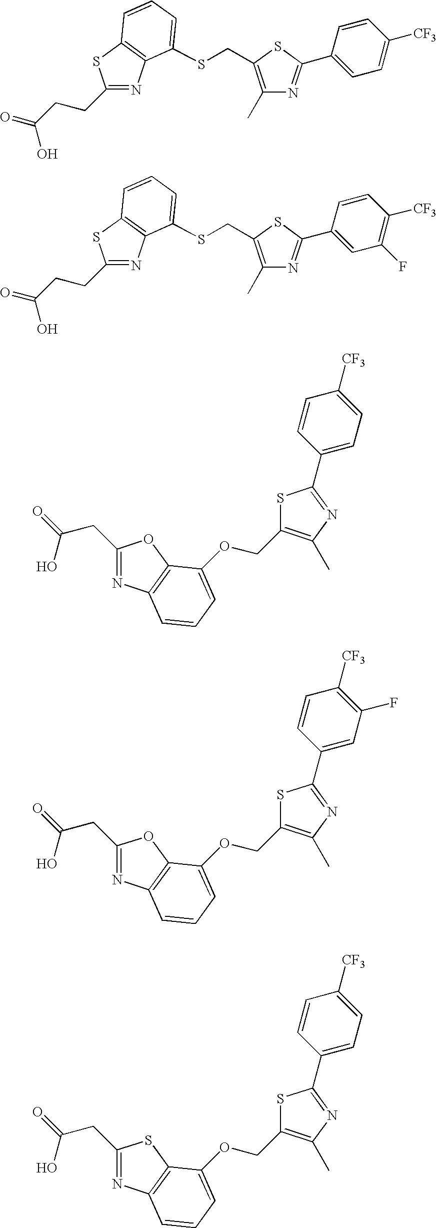

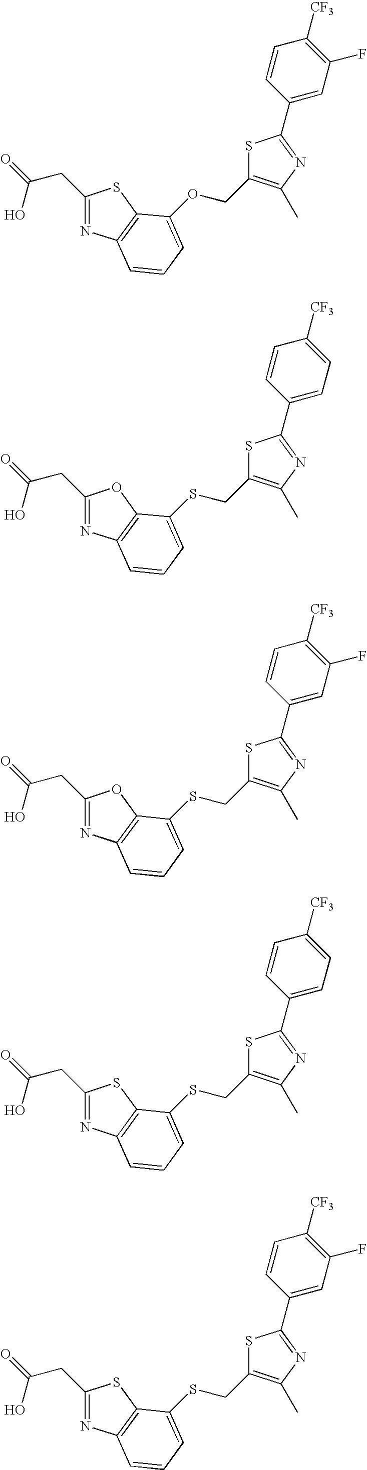

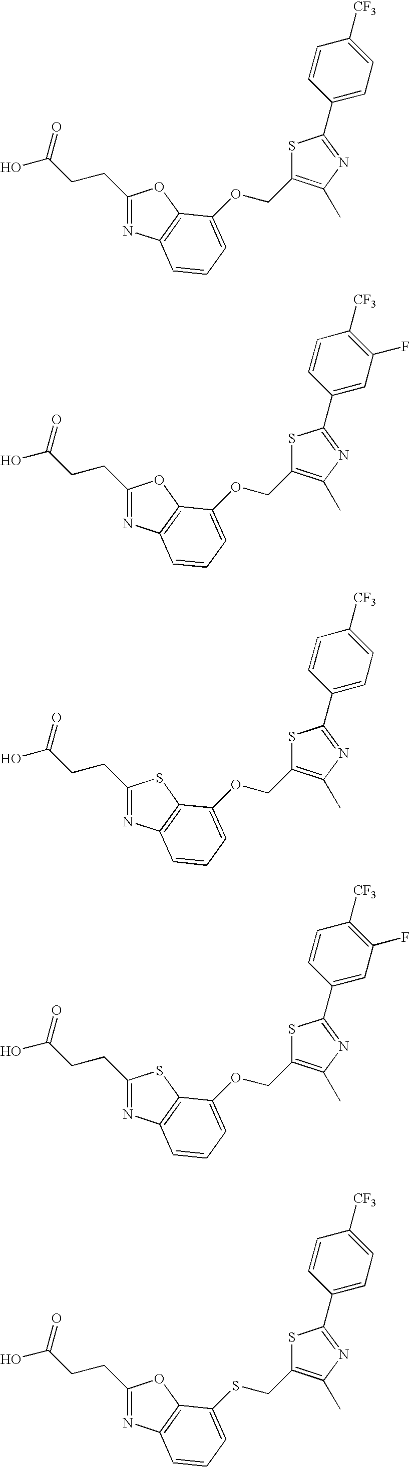

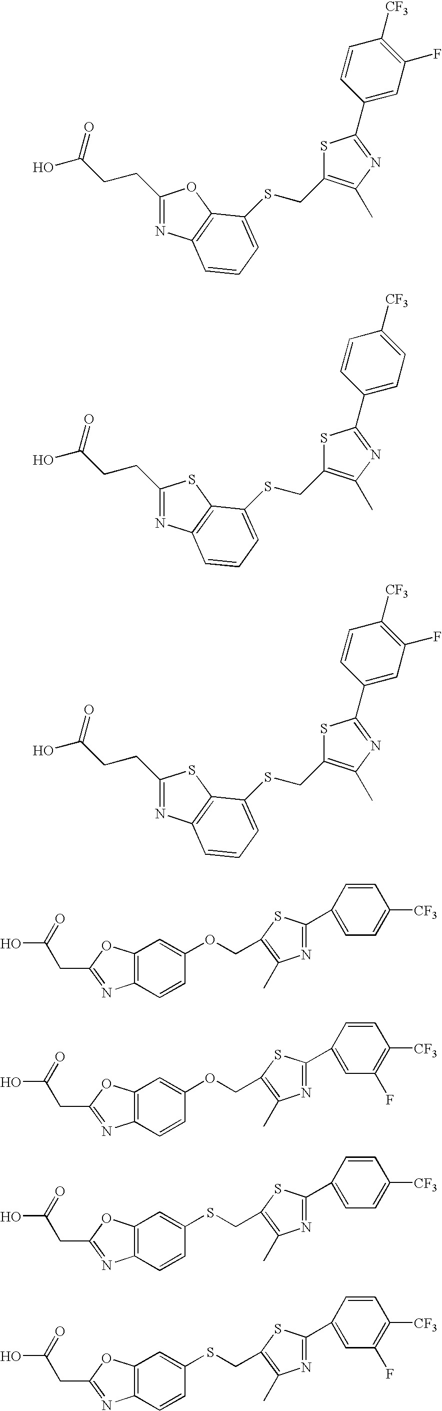

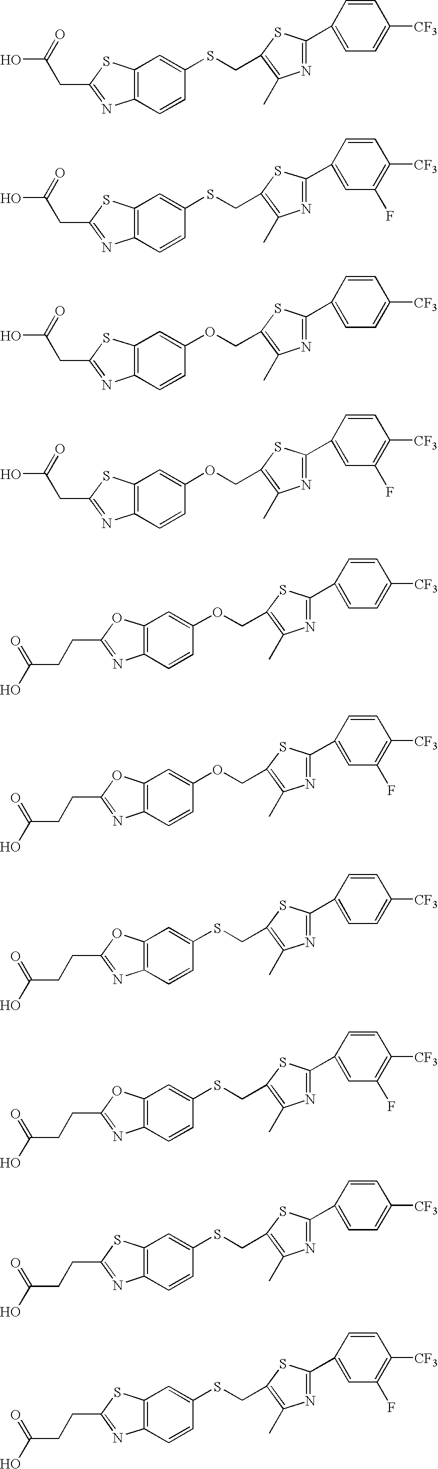

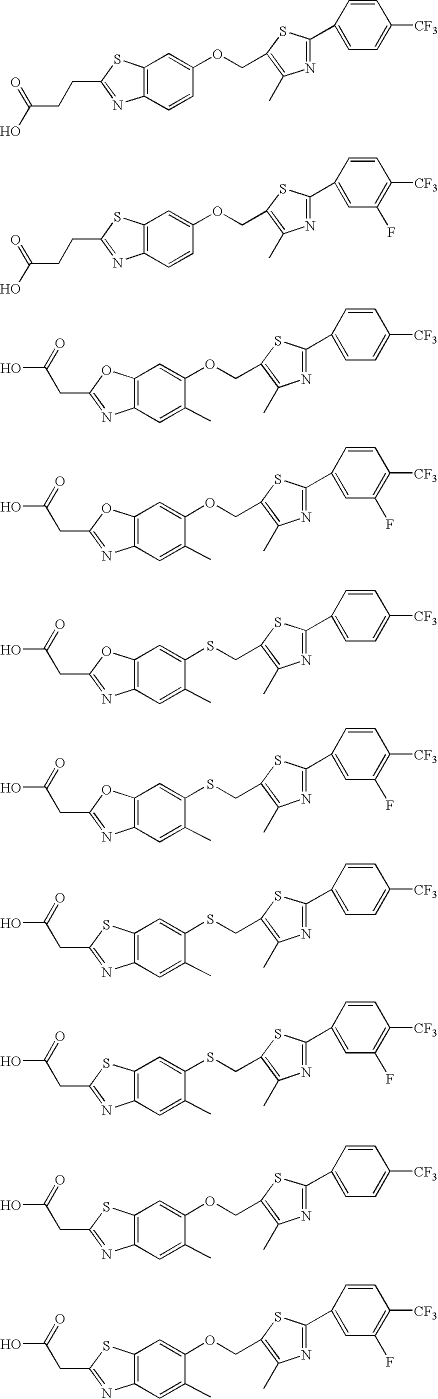

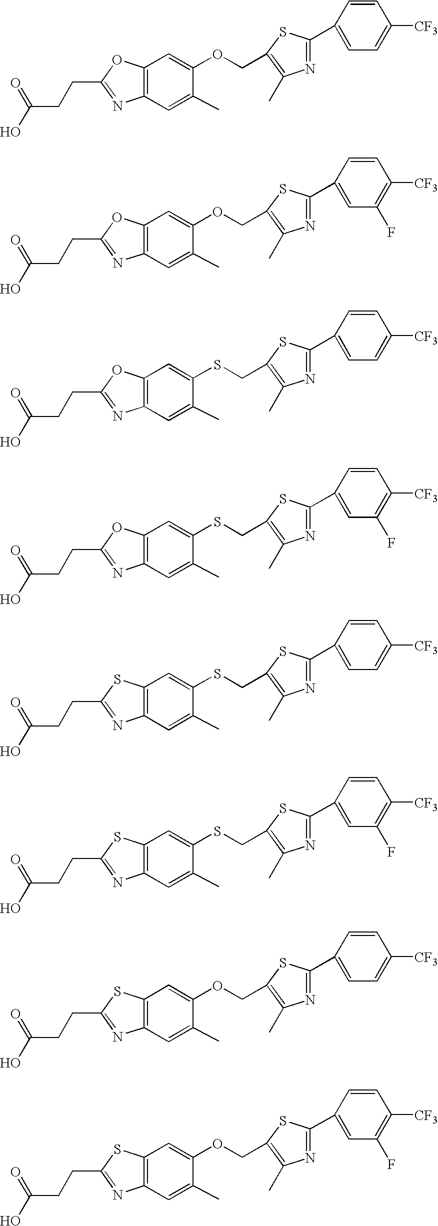

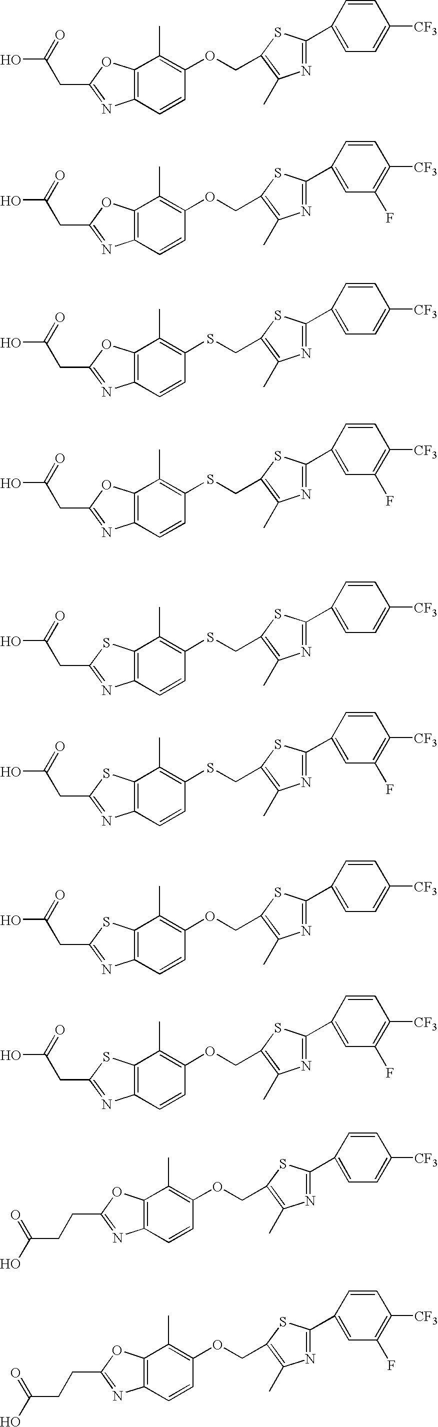

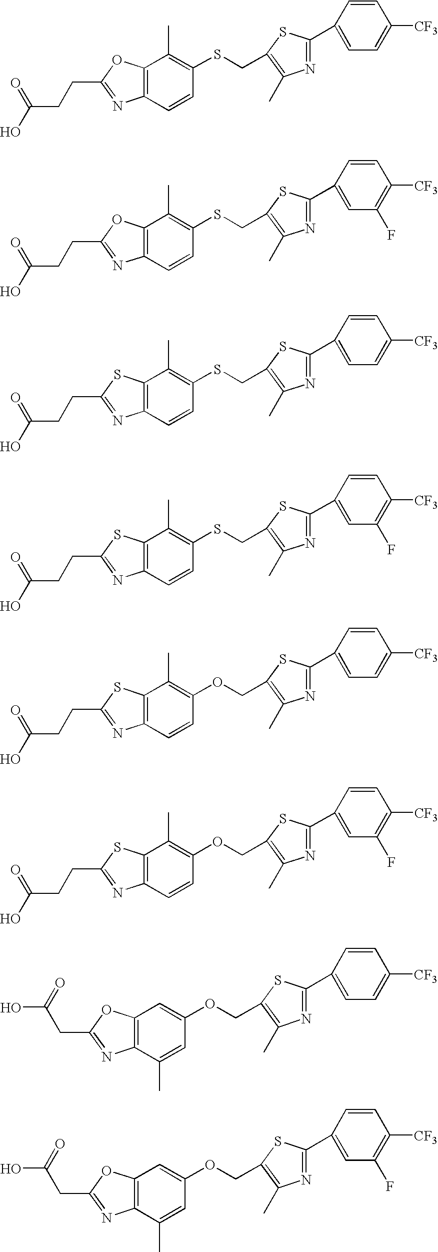

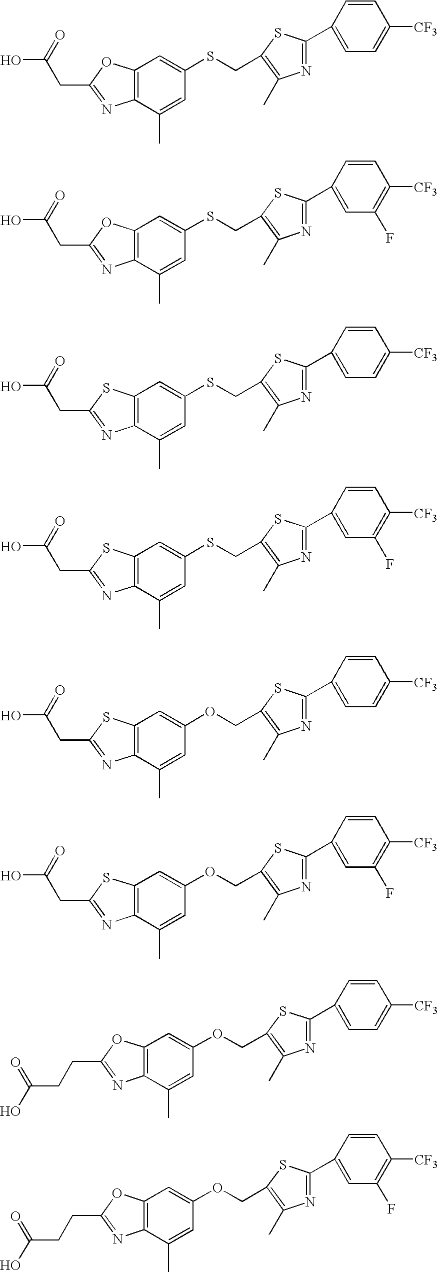

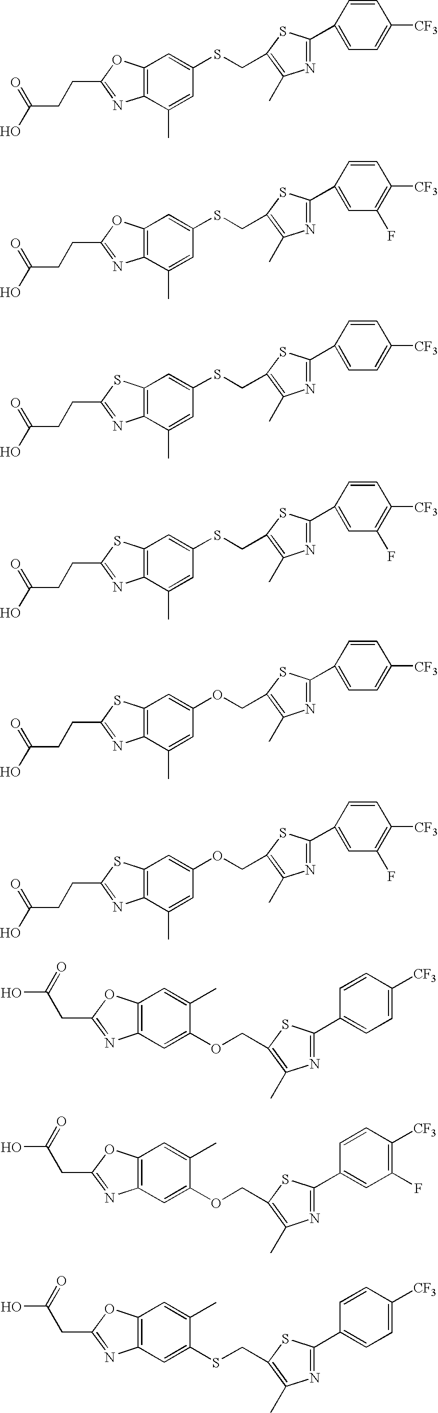

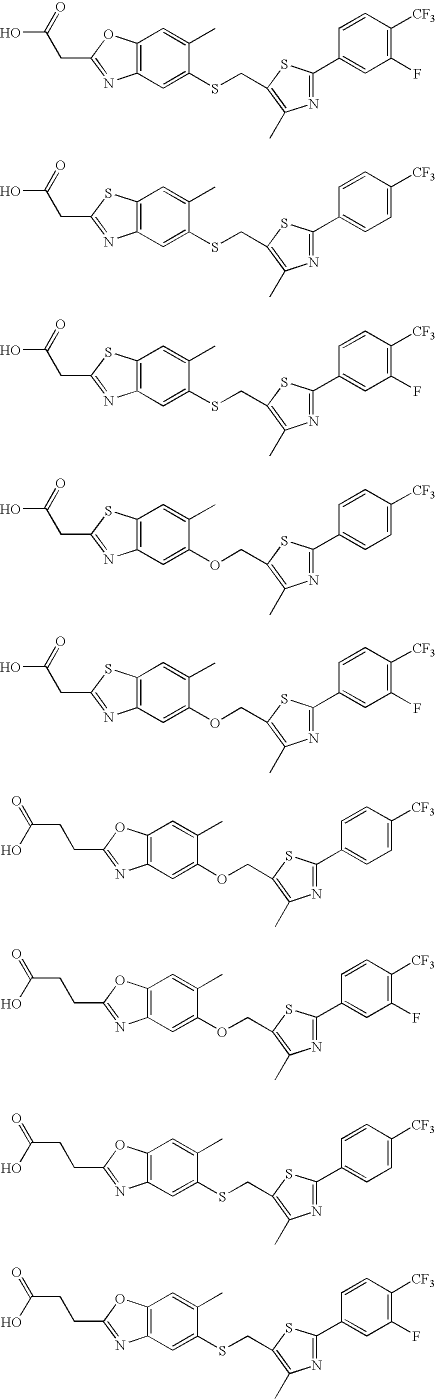

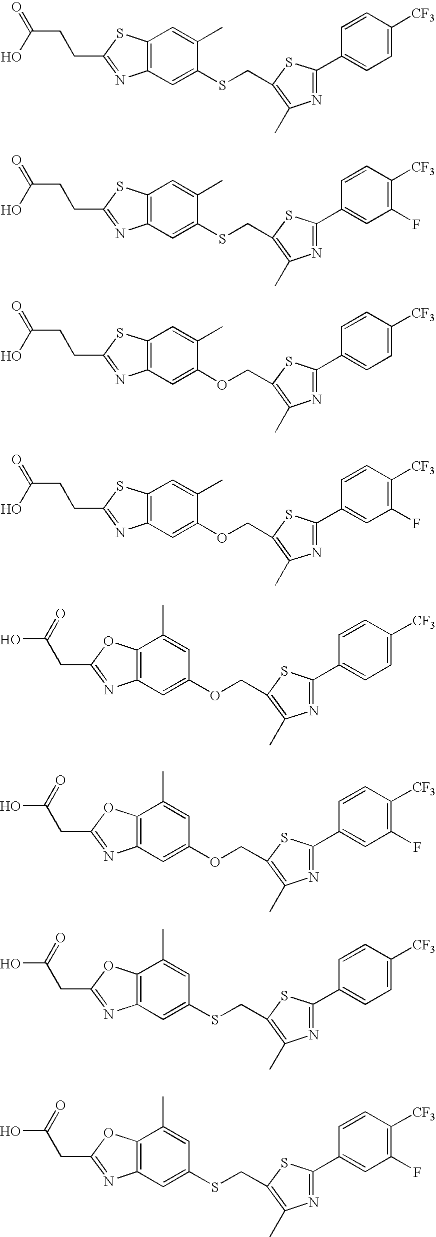

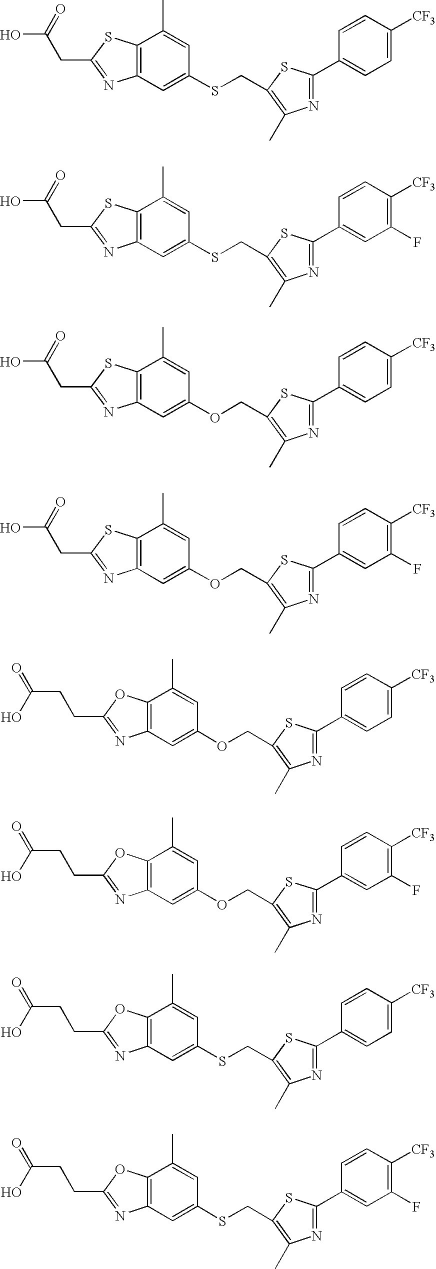

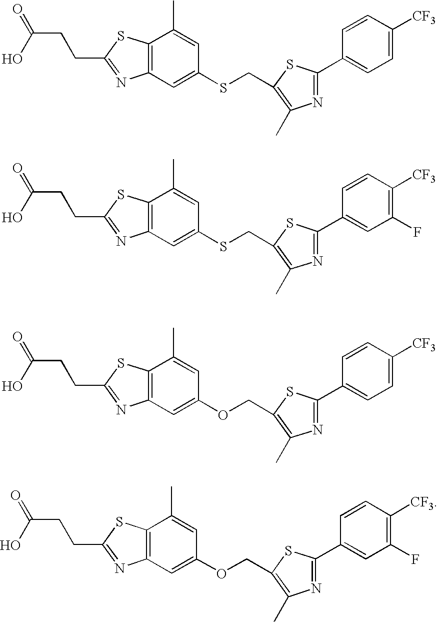

Other embodiments of the invention include any of the following compounds or pharmaceutically acceptable salts, amides, esters, thioesters, or pro-drugs thereof:

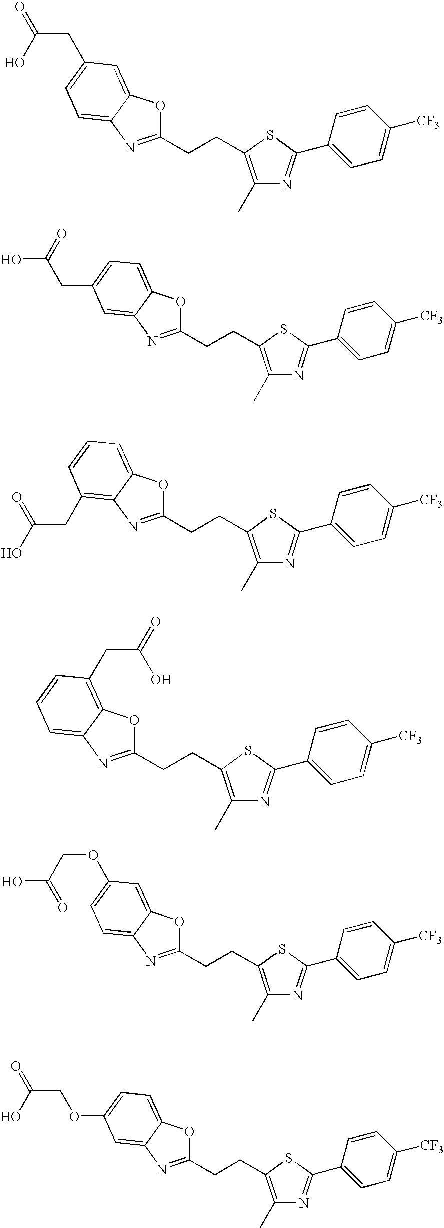

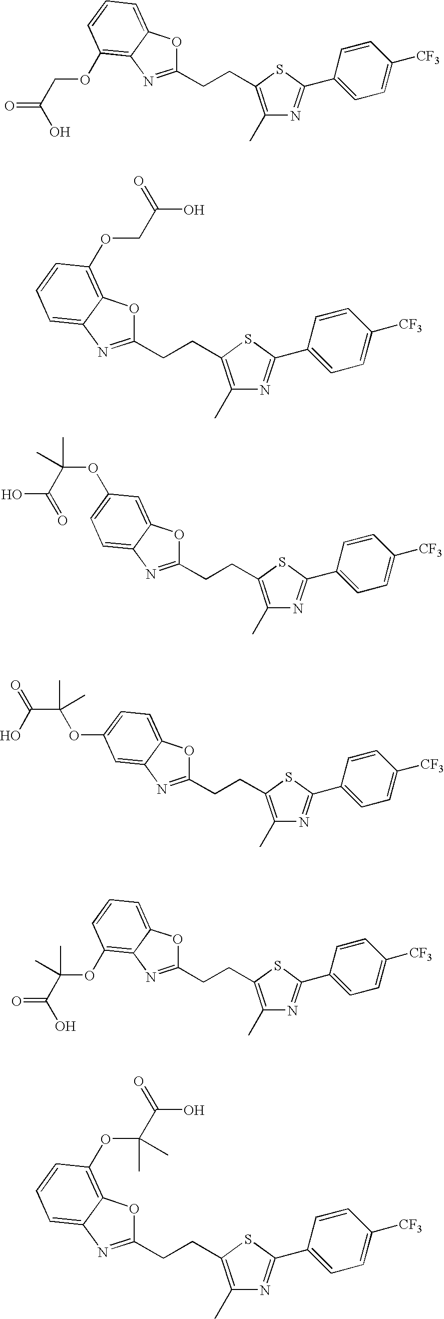

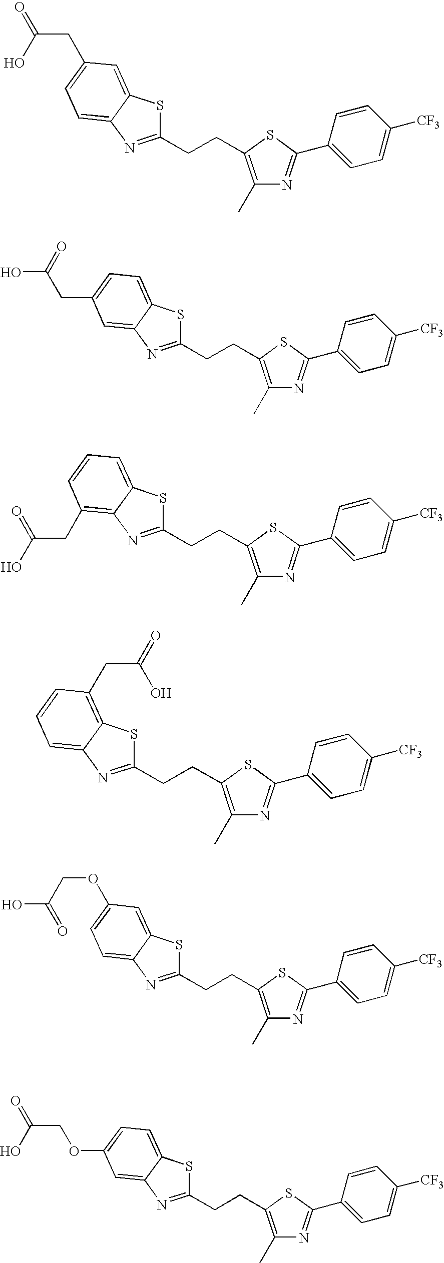

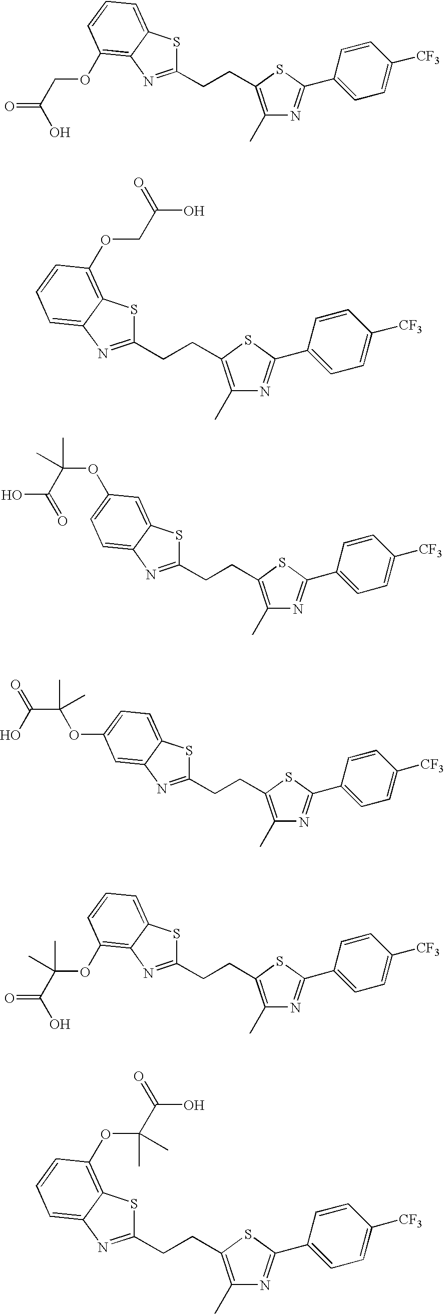

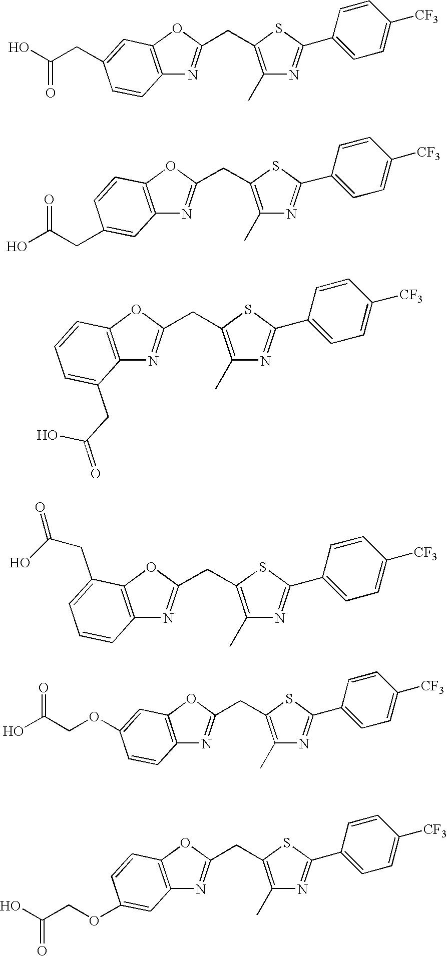

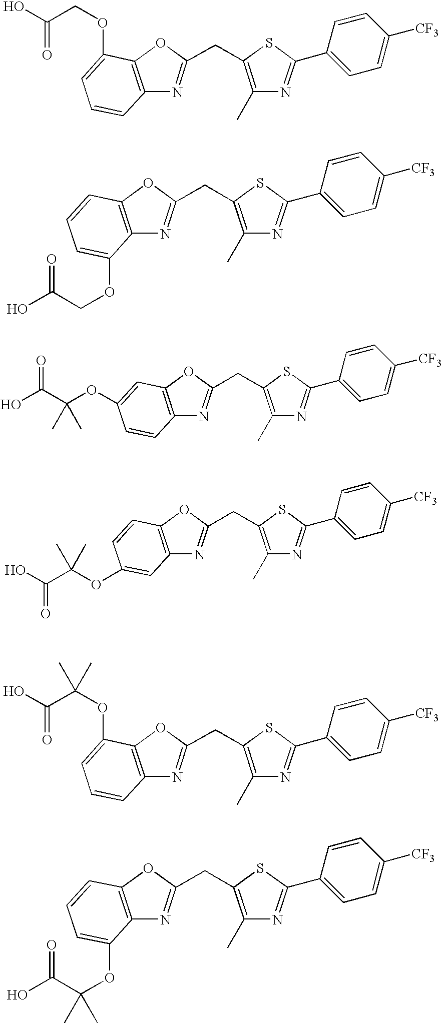

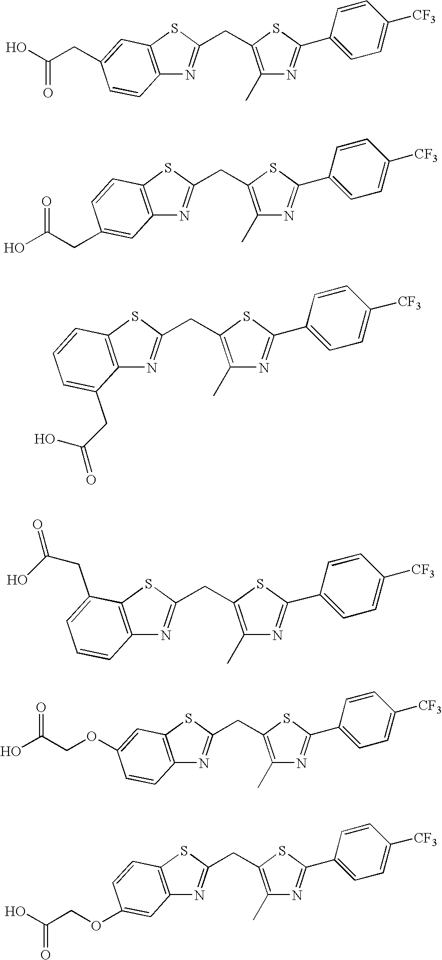

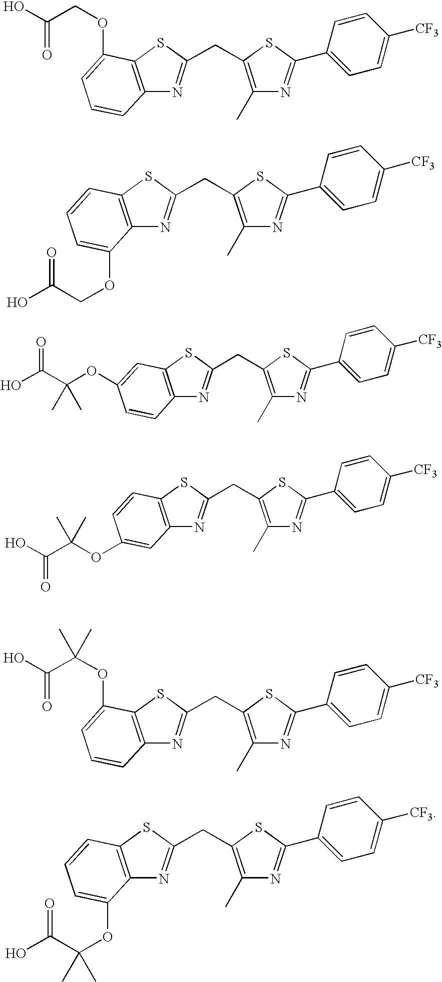

Other embodiments of the invention include any of the following compounds or pharmaceutically acceptable. salts, amides, esters, thioesters, or pro-drugs thereof:

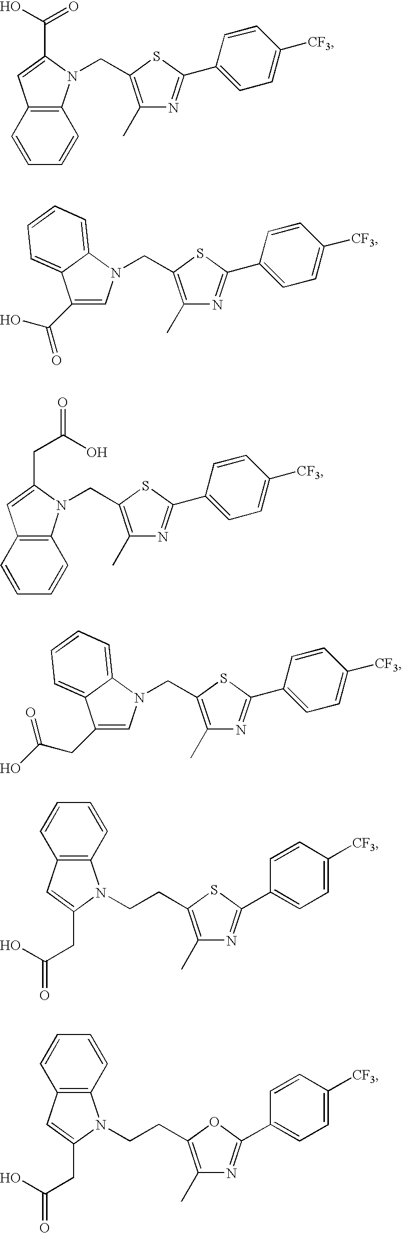

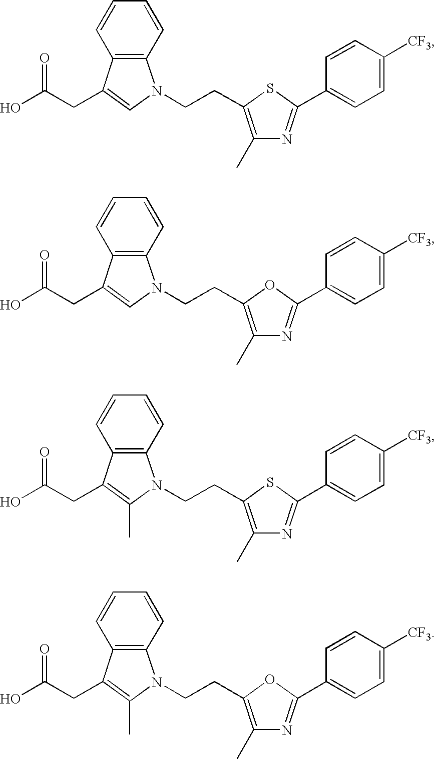

Other embodiments of the invention include any of the following compounds or pharmaceutically acceptable salts, amides, esters, thioesters, or pro-drugs thereof:

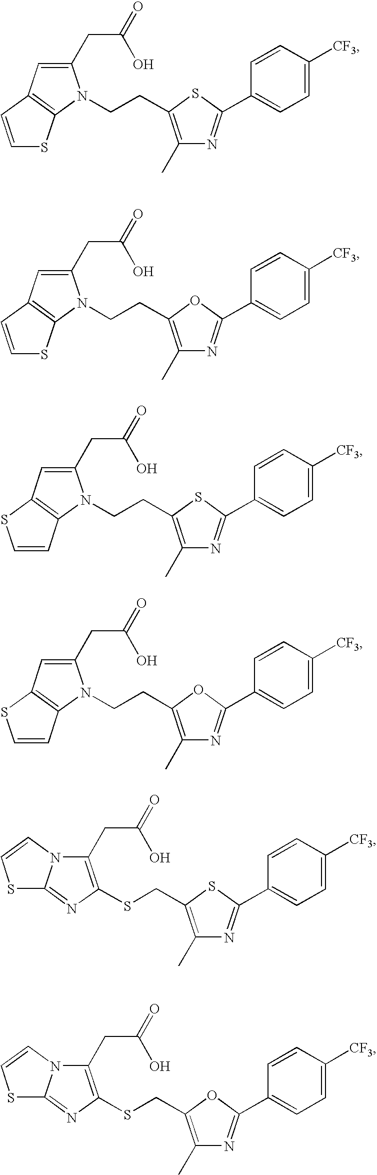

Other embodiments of the invention include any of the following compounds or pharmaceutically acceptable salts, amides, esters, thioesters, or pro-drugs thereof:

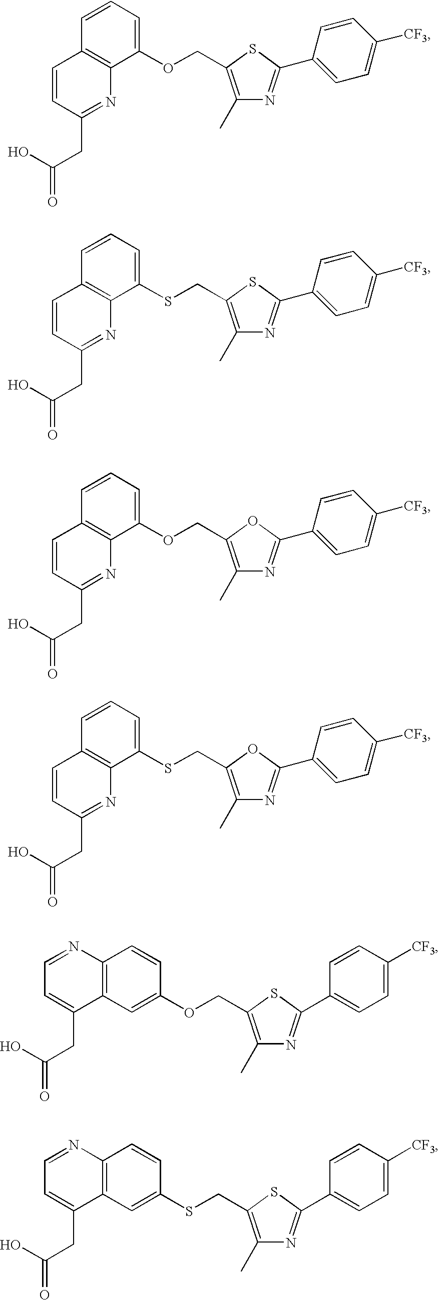

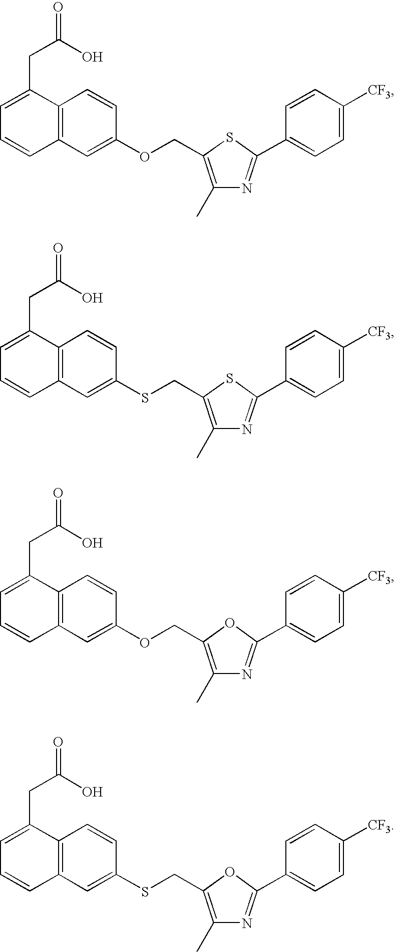

Other embodiments of the invention include any of the following compounds or pharmaceutically acceptable salts, amids, esters, thioesters, or pro-drugs thereof:

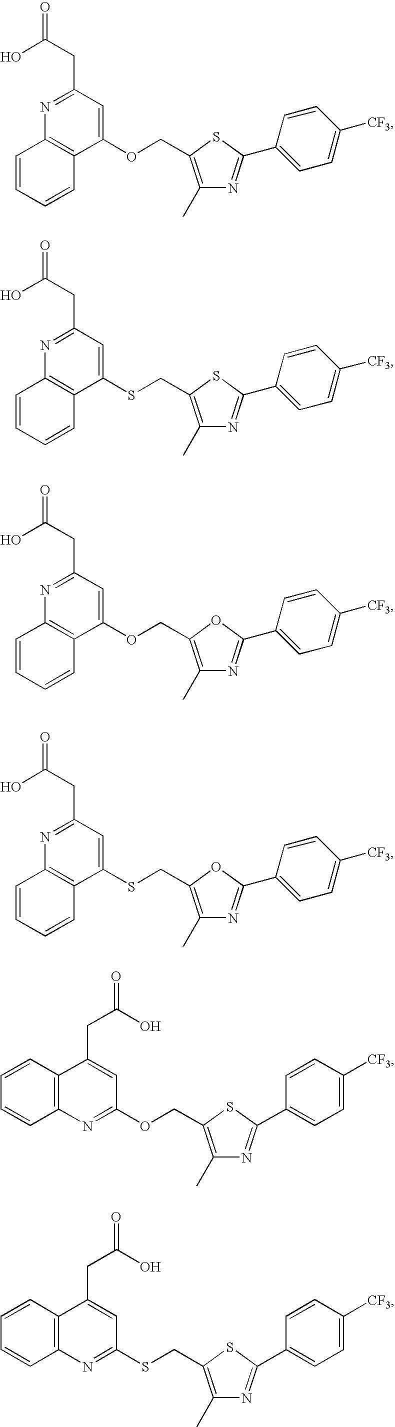

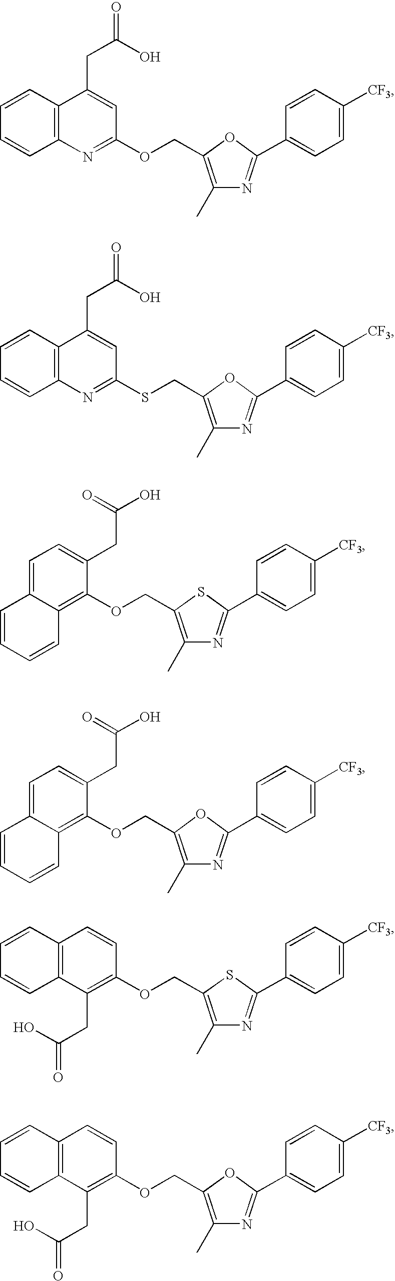

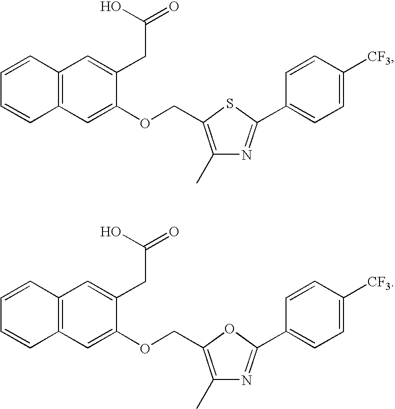

### GLOSSARY

Understanding the present invention as described herein is aided by the following glossary, intended as a guide to meaning of terms certain embodiments.

The term “activate” refers to increasing the cellular function of a PPAR. The term “inhibit” refers to decreasing the cellular function of a PPAR. The PPAR function may be the interaction with a natural binding partner or catalytic activity.

The term “alkenyl” means a straight or branched unsaturated hydrocarbon radical having from 2 to 12 carbon atoms and includes, for example, ethenyl, 1-propenyl, 2-propenyl, 1-butenyl, 2-butenyl, 1-pentenyl, 2-pentenyl, 3-methyl-3-butenyl, 1-hexenyl, 2-hexenyl, 3-hexenyl, 3-heptenyl, 1-octenyl, 1-nonenyl, 1-decenyl, 1-undecenyl, 1-dodecenyl, and the like.

The term “alkynyl” means a straight or branched hydrocarbon radical having from 2 to 12 carbon atoms having at least one triple bond and includes, for example, 1-propynyl, 1-butynyl, 3-butynyl, 1-pentynyl, 3-pentynyl, 3-methyl-3-butynyl, 1-hexynyl, 3-hexynyl, 3-heptynyl, 1-octynyl, 1-nonynyl, 1-decynyl, 1-undecynyl, 1-dodecynyl, and the like.

The term “alkylene” as used herein refers to a divalent group derived from a straight or branched chain saturated hydrocarbon having from 1 to 10 carbon atoms by the removal of two hydrogen atoms, for example methylene, 1,2-ethylene, 1,1-ethylene, 1,3-propylene, 2,2-dimethylpropylene, and the like. The alkylene groups of this invention can be optionally substituted. The alkylene group can also be substituted with one or more of the substituents selected from lower alkyl, lower alkoxy, lower thioalkoxy, halogen, nitro, cyano, .═O, ═S, —OH, —SH, —CF₃, —CO₂H, —CO₂C1-C6 alkyl, —NH₂, —NHC1-C6 alkyl, -CONR′R″, or —N(C1-C6alkyl)₂ where R′ and R″ are independently alkyl, alkenyl, alkynyl, aryl, or joined together to form a 4 to 7 member ring. Useful alkylene groups have from 1 to 6 carbon atoms (C1-C6 alkylene).

The term “aryl” as used herein refers to an aromatic ring which is unsubstituted or optionally substituted by 1 to 4 substituents selected from lower alkyl, lower alkoxy, lower thioalkoxy, halogen, nitro, cyano —OH, —SH, —CF₃, —CO₂H, —CO₂C1-C6 alkyl, —(CH₂)₀₋₂CF₃, —NH₂, —NHC1-C6 alkyl, —SO₂alkyl, —SO₂NH₂, —CONR′R″, or —N(C1-C6alkyl)₂ where R′ and R″ are independently alkyl, akenyl, alkynyl, aryl, or joined together to form a 4 to 7 member ring. Examples include, but are not limited to, phenyl, 2-chlorophenyl, 3-chlorophenyl, 4-chlorophenyl, 2-methylphenyl, 3-methylphenyl, 4-methylphenyl, 2-methoxyphenyl, 3-methoxyphenyl, 4-methoxyphenyl, 2-chloro-3-methylphenyl, 2-chloro-4-methylphenyl, 2-chloro-5-methylphenyl-, 3-chloro-2-methylphenyl, 3-chloro-4-methylphenyl, 4-chloro-2-methylphenyl, 4-chloro-3-methylphenyl, 5-chloro-2-methylphenyl-, 2,3-dichlorophenyl, 2,5-dichlorophenyl, 3,4-dichlorophenyl, 2,3-dimethylphenyl, 3,4-dimethylphenyl, and the like.

The term “cycloalkylene” as used herein refers to a divalent group derived from a cyclic saturated hydrocarbon having from 3 to 8 carbon atoms by the removal of two hydrogen atoms. The cycloalkylene groups of this invention can be optionally substituted. The cycloalkylene group can also be substituted with one or more of the substituents selected from lower alkyl, lower alkoxy, lower thioalkoxy, —O(CH₂), halogen, nitro, cyano, ═O, ═S, —OH, —SH, —OCF₃, —CO₂H, —CO₂C1-C6 alkyl, —NH₂, —NHC1-C6 alkyl, —CONR′R″, or —N(C1-C6alkyl)₂ where R′ and R″ are independently alkyl, akenyl, alkynyl, aryl, or joined together to form a 4 to 7 member ring. Useful cycloalkylene groups have from 3 to 6 carbon atoms (C₃-C₆ alkyl).

As used herein, the term “alkyl” refers to an aliphatic hydrocarbon group. The alkyl moiety may be a “saturated alkyl” group, which means that it does not contain any alkene or alkyne moieties. The alkyl moiety may also be an “unsaturated alkyl” moiety, which means that it contains at least one alkene or alkyne moiety. An “alkene” moiety refers to a group consisting of at least two carbon atoms and at least one carbon-carbon double bond, and an “alkyne” moiety refers to a group consisting of at least two carbon atoms and at least one carbon-carbon triple bond. The alkyl moiety, whether saturated or unsaturated, may be branched, straight chain, or cyclic.

The “alkyl” moiety may have 1 to 40 carbon atoms (whenever it appears herein, a numerical range such as “1 to 40” refers to each integer in the given range; e.g., “1 to 40 carbon atoms” means that the alkyl group may consist of 1 carbon atom, 2 carbon atoms, 3 carbon atoms, etc., up to and including 40 carbon atoms, although the present definition also covers the occurrence of the term “alkyl” where no numerical range is designated). The alkyl group may also be a “medium alkyl” having 1 to 20 carbon atoms. The alkyl group could also be a “lower alkyl” having 1 to 5 carbon atoms. The alkyl group of the compounds of the invention may be designated as “C₁₋₃alkyl” or similar designations. By way of example only, “C₁₋₃ alkyl” indicates that there are one to three carbon atoms in the alkyl chain, i.e., the alkyl chain is selected from the group consisting of methyl, ethyl, propyl, iso-propyl, n-butyl, iso-butyl, sec-butyl, and t-butyl.

The alkyl group may be substituted or unsubstituted. When substituted, the substituent group(s) is(are) one or more group(s) individually and independently selected from cycloalkyl, aryl, heteroaryl, heteroalicyclic, hydroxy, alkoxy, aryloxy, mercapto, alkylthio, arylthio, cyano, halo, carbonyl, thiocarbonyl, O-carbamyl, N carbamyl, O thiocarbamyl, N thiocarbamyl, C amido, N amido, S-sulfonamido, N sulfonamido, C carboxy, O carboxy, isocyanato, thiocyanato, isothiocyanato, nitro, silyl, trihalomethanesulfonyl, and amino, including mono and di substituted amino groups, and the protected derivatives thereof. Typical alkyl groups include, but are in no way limited to, methyl, ethyl, propyl, isopropyl, butyl, isobutyl, tertiary butyl, pentyl, hexyl, ethenyl, propenyl, butenyl, cyclopropyl, cyclobutyl, cyclopentyl, cyclohexyl, and the like. Wherever a substituent is described as being “optionally substituted” that substituent may be substituted with one of the above substituents.

The term “alkylene” refers to an alkyl group that is substituted at two ends (i.e., a diradical). Thus, methylene (—CH₂—) ethylene (—CH₂CH₂—), and propylene (—CH₂CH₂CH₂—) are examples of alkylene groups. Similarly, “alkenylene” and “alkynylene” groups refer to diradical alkene and alkyne moieties, respectively.

An “amide” is a chemical moiety with formula C(O)NHR or NHC(O)R, where R is are optionally substituted and is selected from the group consisting of alkyl, cycloalkyl, aryl, heteroaryl (bonded through a ring carbon) and heteroalicyclic (bonded through a ring carbon). An amide may be an amino acid or a peptide molecule attached to a molecule of the present invention, thereby forming a prodrug. Any amine, hydroxy, or carboxyl side chain on the compounds of the present invention can be esterified or amidified. The procedures and specific groups to be used to achieve this end is known to those of skill in the art and can readily be found in reference sources such as Greene and Wuts, Protective Groups in Organic Synthesis, 3rd Ed., John Wiley & Sons, New York, N.Y., 1999, which is incorporated herein by reference in its entirety.

The term “aromatic” or “aryl” refers to an aromatic group which has at least one ring having a conjugated pi electron system and includes both carbocyclic aryl (e.g., phenyl) and heterocyclic aryl (or “heteroaryl”) groups (e.g., pyridine). The term includes monocyclic or fused-ring polycyclic (i.e., rings which share adjacent pairs of carbon atoms) groups. The term “carbocyclic” refers to a compound which contains one or more covalently closed ring structures, and that the atoms forming the backbone of the ring are all carbon atoms. The term thus distinguishes carbocyclic from heterocyclic rings in which the ring backbone contains at least one atom which is different from carbon. The term “heteroaromatic” or “heteroaryl” refers to an aromatic group which contains at least one heterocyclic ring.

The term “cell phenotype” refers to the outward appearance of a cell or tissue or the function of the cell or tissue. Examples of cell or tissue phenotype are cell size (reduction or enlargement), cell proliferation (increased or decreased numbers of cells), cell differentiation (a change or absence of a change in cell shape), cell survival, apoptosis (cell death), or the utilization of a metabolic nutrient (e.g., glucose uptake). Changes or the absence of changes in cell phenotype are readily measured by techniques known in the art.

The term “cell proliferation” refers to the rate at which a group of cells divides. The number of cells growing in a vessel can be quantified by a person skilled in the art when that person visually counts the number of cells in a defined area using a common light microscope. Alternatively, cell proliferation rates can be quantified by laboratory apparatus that optically measure the density of cells in an appropriate medium.

The term “contacting” as used herein refers to bringing a compound of this invention and a target PPAR together in such a manner that the compound can affect the activity of the PPAR, either directly; i.e., by interacting with the PPAR itself, or indirectly; i.e., by interacting with another molecule on which the activity of the PPAR is dependent. Such “contacting” can be accomplished in a test tube, a petri dish, a test organism (e.g., murine, hamster or primate), or the like. In a test tube, contacting may involve only a compound and a PPAR of interest or it may involve whole cells. Cells may also be maintained or grown in cell culture dishes and contacted with a compound in that environment. In this context, the ability of a particular compound to affect a PPAR related disorder; i.e., the IC₅₀ of the compound can be determined before use of the compounds in vivo with more complex living organisms is attempted. For cells outside the organism, multiple methods exist, and are well-known to those skilled in the art, to get the PPARs in contact with the compounds including, but not limited to, direct cell microinjection and numerous transmembrane carrier techniques.

The terms “enhance” or “enhancing” means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system. When used in a patient, amounts effective for this use will depend on the severity and course of the disease, disorder or condition (including, but not limited to, metabolic disorders), previous therapy, the patient's health status and response to the drugs, and the judgment of the treating physician. It is considered well within the skill of the art for one to determine such enhancing-effective amounts by routine experimentation.

The term “ester” refers to a chemical moiety with formula COOR, where R is optionally substituted and is selected from the group consisting of alkyl, cycloalkyl, aryl, heteroaryl (bonded through a ring carbon) and heteroalicyclic (bonded through a ring carbon).

The term “halogen” includes chlorine, fluorine, bromine, and iodine.

The term “haloalkyl” as used herein refers to a lower alkyl radical, as defined above, bearing at least one halogen substituent, for example, chloromethyl, fluoroethyl, trifluoromethyl, or 1,1,1-trifluoroethyl and the like. Haloalkyl can also include perfluoroalkyl wherein all hydrogens of a lower alkyl group are replaced with fluorine atoms.

The term “heteroaryl” means an aromatic ring containing one or more heteroatoms. The heteroaryl is optionally substituted with one or more groups enumerated for aryl. Examples of heteroaryl include, but are not limited to, thienyl, furanyl, pyrrolyl, pyridyl, pyrimidyl, imidazolyl, pyrazinyl, oxazolyl, thiazolyl, benzothienyl, benzofuranyl, indolyl, quinolinyl, isoquinolinyl, and quinazolinyl, and the like.

The term “heteroatom” as used herein represents oxygen, nitrogen, or sulfur (O, N, or S) as well as sulfoxyl or sulfonyl (SO or SO₂) unless otherwise indicated.

The term “heterocycle” means a saturated or unsaturated mono- or polycyclic (i.e. bicyclic) ring incorporating one or more (i.e. 1-4) heteroatoms selected from N, O, and S. It is understood that a heterocycle is optionally substituted with —OH, ——O(alkyl), SH, S(alkyl), amine, halogen, acid, ester, amide, amidine, alkyl ketone, aldehyde, nitrile, fluoroalkyl, nitro, sulphone, sulfoxide or C₁₋₆ alkyl. Examples of suitable monocyclic heterocycles include, but are not limited to substituted or unsubstituted thienyl, furanyl, pyrrolyl, imidazolyl, pyrazolyl, thiazolyl, isothiazolyl, oxazolyl, isoxazolyl, triazolyl, tetrazolyl, pyridinyl, pyrazinyl, pyrimidinyl, piperidinyl, pyrrolidinyl, piperazinyl, azetidinyl, aziridinyl, morpholinyl, thietanyl, oxetaryl. Examples of monocyclic diheterocycles include, but are not limited to, 1-, 2-, 4-, or 5-imidazolyl, 1-, 3-, 4-, or 5-pyrazolyl, 2-, 4-, or 5-thiazolyl, 3-, 4-, or 5-isothiazolyl, 2-, 4-, or 5-oxazolyl, 3-, 4-, or 5-isoxazolyl, 1,3-, or 5-triazolyl, 1-, 2-, or 3-tetrazolyl, 2-pyrazinyl, 2-, 4-, or 5-pyrimidinyl, 1 - or 2-piperazinyl, 2-, 3-, or 4-morpholinyl. Examples of suitable bicyclic heterocycles include, but are not limited to indolizinyl, isoindolyl, benzofuranyl, benzothienyl, benzoxazolyl, benzimidazolyl, quinolinyl, isoquinolinyl, quinazolinyl, 1-, 2-, 3-, 4-, 5-, 6-, or 7-indolyl, 1-, 2-, 3-, 5-, 6-, 7-, or 8-indolizinyl, 1-, 2-, 3-, 4-, 5-, 6-, or 7-isoindolyl, 2-, 3-, 4-, 5-, 6-, or 7-benzothienyl, 2-, 4-, 5-, 6-, or 7-benzoxazolyl, 1-, 2-, 4-, 5-, 6-, or 7-benzimidazolyl, 2-, 3-, 4-, 5-, 6-, 7-, or 8-quinolinyl, and 1-, 3-, 4-, 5-, 6-, 7-, 8-isoquinolinyl. The following table correlates structure and name as used herein for several heterocyclic aspects of the invention.

TABLE 1
Nomenclature for Fused 5 and 6 Membered Heterocyclic Ring Systems

| | | | |
|---|---|---|---|
| indole | benzimidazole | pyrrolopyridine | pyrrolopyridine |
| 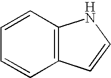 | 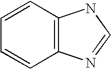 | 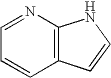 | 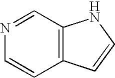 |
| pyrrolopyridine | Pyrrolopyridine | pyrrolopyrimidine | pyrrolopyrimidine |
| 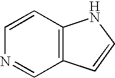 | 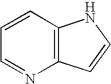 | 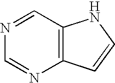 | 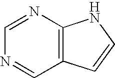 |
| pyrrolopyrazine | pyrrolopyridazine | pyrrolopyridazine | pyrrolopyridazine |
| 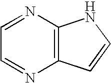 | 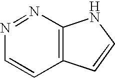 | 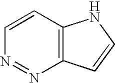 | 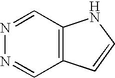 |
| benzofuran | Benzothiophene | benzoxazole | benzthiazole |
| 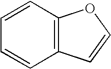 | 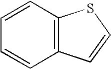 | 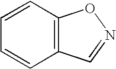 | 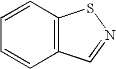 |
| indazole | Pyrrolothiophene | Pyrrolothiophene | Imidazolothiazole |
| 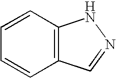 | 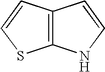 | 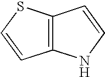 | 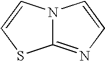 |

The term “inhibit” refers to decreasing the cellular function of a PPAR. The cellular function of a PPAR may be the interaction with a natural binding partner or catalytic activity.

The term “membered ring” can embrace any cyclic structure. The term “membered” is meant to denote the number of skeletal atoms that constitute the ring. Thus, for example, cyclohexyl, pyridine, pyran and thiopyran are 6-membered rings and cyclopentyl, pyrrole, furan, and thiophene are 5-membered rings.

The term “modulate” refers to the ability of a compound of the invention to alter the function of a PPAR. A modulator may activate the activity of a PPAR, may activate or inhibit the activity of a PPAR depending on the concentration of the compound exposed to the PPAR, or may inhibit the activity of a PPAR. The term “modulate” also refers to altering the function of a PPAR by increasing or decreasing the probability that a complex forms between a PPAR and a natural binding partner. A modulator may increase the probability that such a complex forms between the PPAR and the natural binding partner, may increase or decrease the probability that a complex forms between the PPAR and the natural binding partner depending on the concentration of the compound exposed to the PPAR, and or may decrease the probability that a complex forms between the PPAR and the natural binding partner.

The term “monitoring” refers to observing the effect of adding the compound of the invention to the cells of the method. The effect can be manifested in a change in cell phenotype, cell proliferation, PPAR activity, or in the interaction between a PPAR and a natural binding partner. Of course, the term “monitoring” includes detecting whether a change has in fact occurred or not.

The term “optionally substituted,” means that the substituent is a group that may be, but need not be, substituted with one or more group(s) individually and independently selected from moieties such as alkyl, cycloalkyl, aryl, heteroaryl, heteroalicyclic, hydroxy, alkoxy, aryloxy, mercapto, alkylthio, arylthio, cyano, halo, carbonyl, thiocarbonyl, O-carbamyl, N carbamyl, O thiocarbamyl, N thiocarbamyl, C amido, N amido, S-sulfonamido, N sulfonamido, C carboxy, O carboxy, isocyanato, thiocyanato, isothiocyanato, nitro, silyl, trihalomethanesulfonyl, perhalo, alkyl, and amino, including mono and di substituted amino groups, and the protected derivatives thereof. The protecting groups that may form the protective derivatives of the above substituents are known to those of skill in the art and may be found in references such as Greene and Wuts, above.

The term “patient” means all mammals including humans. Examples of patients include humans, cows, dogs, cats, goats, sheep, pigs, and rabbits.

The term “perhaloalkyl” refers to an alkyl group where all of the hydrogen atoms are replaced by halogen atoms.

The term “pharmaceutically acceptable” refers to a formulation of a compound that does not cause significant irritation to an organism to which it is administered and does not abrogate the biological activity and properties of the compound. Pharmaceutically acceptable salts may be obtained by reacting a compound of the invention with inorganic acids such as hydrochloric acid, hydrobromic acid, sulfuric acid, nitric acid, phosphoric acid, methanesulfonic acid, ethanesulfonic acid, p-toluenesulfonic acid, salicylic acid and the like. Pharmaceutically acceptable salts may also be obtained by reacting a compound of the invention with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods known in the art. Similarly pharmaceutically acceptable esters or amides can form pro-drugs for compounds bearing a carboxylic acid moiety wherein hydrolysis of the amide or ester yields pharmaceutically acceptable hydrolysis products in addition to the active drug compound.

A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a compound of the present invention which is administered as an ester (the “prodrug”) to facilitate transmittal across a cell membrane where water solubility is detrimental to mobility but which then is metabolically hydrolyzed to the carboxylic acid, the active entity, once inside the cell where water solubility is beneficial. A further example of a prodrug might be a short peptide (polyaminoacid) bonded to an acid group where the peptide is metabolized to reveal the active moiety.

As used herein, a “selective hPPAR-delta modulator” is a hPPAR-delta modulator whose EC₅₀ for PPAR-delta is about 10 fold lower than its EC50 for either PPARγ or PPAR-alpha. EC50 is defined in the transfection assay described below and is the concentration at which a compound achieves 50% of its maximum activity. Some compounds may have substantially greater than 10-fold selectivity for hPPAR-delta.

The PPAR-delta selective compounds of this invention may elevate HDL-c in db/db mice and primate models and may lower fibrinogen in primate models. These PPAR-delta selective modulators may lower triglycerides and insulin levels in the primate.

The substituent “R” or “R′” appearing by itself and without a number designation refers to an optionally substituted substituent selected from the group consisting of alkyl, cycloalkyl, aryl, heteroaryl (bonded through a ring carbon) and heteroalicyclic (bonded through a ring carbon).

A “sulfinyl” group refers to a S(═O) R group, with R as defined herein.

A “S sulfonamido” group refers to a S(═O)₂NR, group, with R as defined herein.

The term “therapeutically effective amount” as used herein refers to that amount of the compound being administered which will relieve to some extent one or more of the symptoms of the disease, condition or disorder being treated. In reference to the treatment of diabetes or dyslipidemia a therapeutically effective amount refers to that amount which has the effect of (1) reducing the blood glucose levels; (2) normalizing lipids, e.g. triglycerides, low-density lipoprotein; and/or (3) relieving to some extent (or, preferably, eliminating) one or more symptoms associated with the disease, condition or disorder to be treated.

A “therapeutically effective amount” is an amount of a compound of the present invention that when administered to a patient ameliorates a symptom of dyslipidemia, non-insulin dependent diabetes mellitus, obesity, hyperglycemia, hypercholesteremia, hyperlipidemia, atherosclerosis, hypertriglyceridemia, or hyperinsulinemia.

A “thiocyanato” group refers to a CNS group.

As used herein, reference to “treatment” of a patient is intended to include prophylaxis.

A “trihalomethanesulfonyl” group refers to a X₃CS(═O)₂ group where X is a halogen.

A “trihalomethanesulfonarnido” group refers to a X₃CS(═O)₂NR group with X and R as defined herein.

Preferably, the compounds of formula (I) are hPPAR-delta modulators. As used herein, by “modulator”, or “activating compound”, or “activator”, or the like, is meant those compounds which have a pKi of at least 6.0, preferably at least 7.0, to the relevant PPAR, for example hPPAR-delta, in the binding assay described below, and which achieve at least 50% activation of the relevant PPAR relative to the appropriate indicated positive control in the transfection assay described below at concentrations of 10⁻⁵ M or less. Preferably, the modulator of this invention achieve 50% activation of human PPAR-delta in the transfection assay at concentrations of 10⁻⁷ M or less, more preferably 10⁻⁹ M or less.

It will also be appreciated by those skilled in the art that the compounds of the present invention may also be utilized in the form of a pharmaceutically acceptable salt or solvate thereof. The physiologically acceptable salts of the compounds of formula (I) include conventional salts formed from pharmaceutically acceptable inorganic or organic acids or bases as well as quaternary ammonium acid addition salts. More specific examples of suitable acid salts include hydrochloric. hydrobromic, sulfuric, phosphoric, nitric, perchloric, fumaric, acetic, propionic, succinic, glycolic, formic, lactic, maleic, tartaric, citric, palmoic, malonic, hydroxymaleic, phenylacetic, glutamic, benzoic, salicylic, fumaric, toluenesulfonic, methanesulfonic, naphthalene-2-sulfonic, benzenesulfonic hydroxynaphthoic, hydroiodic, malic, steroic, tannic and the like. Other acids such as oxalic, while not in themselves pharmaceutically acceptable, may be useful in the preparation of salts useful as intermediates in obtaining the compounds of the invention and their pharmaceutically acceptable salts. More specific examples of suitable basic salts include sodium, lithium, potassium, magnesium, aluminium, calcium, zinc, N,N′-dibenzylethylenediamine, chloroprocaine, choline, diethanolamine, ethylenediamine, N-methylglucamine and procaine salts. Those skilled in the art of organic chemistry will appreciate that many organic compounds can form complexes with solvents in which they are reacted or from which they are precipitated or crystallized. These complexes are known as “solvates”, For example, a complex with water is known as a “hydrate”. Solvates of the compound of formula (I) are within the scope of the invention. References hereinafter to a compound according to the invention include both compounds of formula (I) and their pharmaceutically acceptable salts and solvates.

It will be appreciated by those skilled in the art that reference herein to treatment extends to prophylaxis as well as the treatment of established diseases or symptoms. Moreover, it will be appreciated that the amount of a compound of the invention required for use in treatment will vary with the nature of the condition being treated and the age and the condition of the patient and will be ultimately at the discretion of the attendant physician or veterinarian. In general, however, doses employed for adult human treatment will typically be in the range of 0.02-5000 mg per day, preferably 1-1500 mg per day. The desired dose may conveniently be presented in a single dose or as divided doses administered at appropriate intervals, for example as two, three, four or more sub-doses per day.

While it is possible that compounds of the present invention may be therapeutically administered in their isolated, pure form, it is preferable to present the active ingredient as a pharmaceutical formulation. Accordingly, the present invention further provides for a pharmaceutical formulation comprising a compound of formula (I) or a pharmaceutically acceptable salt or solvate thereof together with one or more pharmaceutically acceptable carriers therefor and, optionally, other therapeutic and/or prophylactic ingredients.

Formulations of the present invention include those especially formulated for oral, buccal, parenteral, transdermal, inhalation, intranasal, transmucosal, implant, or rectal administration, however, oral administration is preferred. For buccal administration, the formulation may take the form of tablets or lozenges formulated in conventional manner. Tablets and capsules for oral administration may contain conventional excipients such as binding agents, (for example, syrup, acacia, gelatin, sorbitol, tragacanth, mucilage of starch or polyvinylpyrrolidone), fillers (for example, lactose, sugar, microcrystalline cellulose, maize-starch, calcium phosphate or sorbitol), lubricants (for example, magnesium stearate, stearic acid, talc, polyethylene glycol or silica), disintegrants (for example, potato starch or sodium starch glycollate) or wetting agents, such as sodium lauryl sulfate. The tablets may be coated according to methods well-known in the art.

Alternatively, the compounds of the present invention may be incorporated into oral liquid preparations such as aqueous or oily suspensions, solutions, emulsions, syrups or elixirs, for example. Moreover, formulations containing these compounds may be presented as a dry product for constitution with water or other suitable vehicle before use. Such liquid preparations may contain conventional additives such as suspending agents such as sorbitol syrup, methyl cellulose, glucose/sugar syrup, gelatin, hydroxyethylcellulose, carboxymethyl cellulose, aluminum stearate gel or hydrogenated edible fats; emulsifying agents such as lecithin, sorbitan mono-oleate or acacia; nonaqueous vehicles (which may include edible oils) such as almond oil, fractionated coconut oil, oily esters, propylene glycol or ethyl alcohol; and preservatives such as methyl or propyl p-hydroxybenzoates or sorbic acid. Such preparations may also be formulated as suppositories, e.g., containing conventional suppository bases such as cocoa butter or other glycerides.

Additionally, formulations of the present invention may be formulated for parenteral administration by injection or continuous infusion. Formulations for injection may take such forms as suspensions, solutions, or emulsions in oily or aqueous vehicles, and may contain formulatory agents such as suspending, stabilizing and/or dispersing agents. Alternatively, the active ingredient may be in powder form for constitution with a suitable vehicle (e.g., sterile, pyrogen-free water) before use.

The formulations according to the invention may also be formulated as a depot preparation. Such long acting formulations may be administered by implantation (for example, subcutaneously or intramuscularly) or by intramuscular injection. Accordingly, the compounds of the invention may be formulated with suitable polymeric or hydrophobic materials (as an emulsion in an acceptable oil, for example), ion exchange resins or as sparingly soluble derivatives as a sparingly soluble salt, for example.

The formulations according to the invention may contain between 0.199% of the active ingredient, conveniently from 30-95% for tablets and capsules and 3-50% for liquid preparations.

The compound of formula (I) for use in the instant invention may be used in combination with other therapeutic agents for example, stating and/or other lipid lowering drugs for example MTP inhibitors and LDLR upregulators. The compounds of the invention may also be used in combination with antidiabetic agents, e.g. metformin, sulfonylureas, or PPAR-gamma, PPAR-alpha and PPAR-alpha/gamma modulators (for example thiazolidinediones such as e.g. Pioglitazone and Rosiglitazone). The compounds may also be used in combination with antihypertensive agents such as angiotensin antagonists, e.g ., telmisartan, calcium channel antagonists e.g. lacidipine and ACE inhibitors, e.g., enalapril. The invention thus provides in a further aspect the use of a combination comprising a compound of formula (I) with a further therapeutic agent in the treatment of a hPPAR-delta mediated disease.

When the compounds of formula (I) are used in combination with other therapeutic agents, the compounds may be administered either sequentially or simultaneously by any convenient route.

The combinations referred to above may conveniently be presented for use in the form of a pharmaceutical formulation and thus pharmaceutical formulations comprising a combination as defined above optimally together with a pharmaceutically acceptable carrier or excipient comprise a further aspect of the invention. The individual components of such combinations may be administered either sequentially or simultaneously in separate or combined pharmaceutical formulations.

When combined in the same formulation it will be appreciated that the two compounds must be stable and compatible with each other and the other components of the formulation and may be formulated for administration. When formulated separately they may be provided in any convenient formulation, conveniently in such a manner as are known for such compounds in the art.

When a compound of formula (I) is used in combination with a second therapeutic agent active against the same disease, the dose of each compound may differ from that when the compound is used alone. Appropriate doses will be readily appreciated by those skilled in the art.

### RELATED EMBODIMENTS OF THE INVENTION

Molecular embodiments of the present invention may possess one or more chiral centers and each center may exist in the R or S configuration. The present invention includes all diastereomeric, enantiomeric, and epimeric forms as well as the appropriate mixtures thereof. Stereoisomers may be obtained, if desired, by methods known in the art as, for example, the separation of stereoisomers by chiral chromatographic columns. Additionally, the compounds of the present invention may exist as geometric isomers. The present invention includes all cis, trans, syn, anti, entgegen (E), and zusammen (Z) isomers as well as the appropriate mixtures thereof.

In some situations, compounds may exist as tautomers. All tautomers are included within Formula I and are provided by this invention.

In addition, the compounds of the present invention can exist in unsolvated as well as solvated forms with pharmaceutically acceptable solvents such as water, ethanol, and the like. In general, the solvated forms are considered equivalent to the unsolvated forms for the purposes of the present invention.

### BIOLOGICAL ASSAYS

The compounds were evaluated in a cell-based assay to determine their human PPAR activity. The plasmids for human PPAR-GAL4 chimeras were prepared by fusing amplified cDNAs encoding the LBDs of PPARs to the C-terminal end of the yeast GAL4 DNA binding domain. CV-1 cells were grown and transiently transected with PerFectin (GTS, San Diego, Calif.) according to the manufacturer's protocol along with a luciferase reporter. Eight hours after transfection, 50 μl of cells were replated into 384 well plates (1×10⁵ cells/well). Sixteen hours after replating, cells were treated with either compounds or 1% DMSO for 24 hours. Luciferase activity was then assayed with Britelite (Perkin Elmer) following the manufacturer's protocol and measured with either the Perkin Elmer Viewlux or Molecular Devices Acquest.

### ADDITIONAL ASSAYS

Compounds may be tested for their ability to bind to hPPAR-gamma, hPPAR-alpha, or PPAR-delta using a Scintillation Proximity Assay (SPA). The PPAR ligand binding domain (LBO) may be expressed in E. coli as polyHis tagged fusion proteins and purified. The LBO is then labeled with biotin and immobilized on streptavidin modified scintillation proximity beads. The beads are then incubated with a constant amount of the appropriate radioligand eH-BRL 49653 for PPARγ, 2-(4(2-(2,3-Ditritio-1-heptyl-3-(2,4-difluorophenyl)ureido )ethyl)phenoxy)-2 methyl butanoic acid (described in WO1008002) for hPPAR-alpha and GW 2433 (see Brown, P. J et al. Chem. Biol. 1997, 4, 909-918. For the structure and synthesis of this ligand) for PPAR-delta) and variable concentrations of test compound, and after equilibration the radioactivity bound to the beads is measured by a scintillation counter. The amount of nonspecific binding, as assessed by control wells containing 50 μM of the corresponding unlabelled ligand, is subtracted from each data point. For each compound tested, plots of ligand concentration vs. CPM of radioligand bound are constructed and apparent K, values are estimated from nonlinear least squares fit of the data assuming simple competitive binding. The details of this assay have been reported elsewhere (see, Blanchard, S. G. et. al., “Development of a Scintillation Proximity Assay for Peroxisome Proliferator-Activated Receptor gamma Ligand Binding Domain” Anal. Biochem. 1998, 257, 112-119).

### TRANSFECTION ASSAYS

Compounds may be screened for functional potency in transient transfection assays in CV-1 cells for their ability to activate the PPAR subtypes (transactivation assay). A previously established chimeric receptor system was utilized to allow comparison of the relative transcriptional activity of the receptor subtypes on the same synthetic response element and to prevent endogenous receptor activation from complicating the interpretation of results. See, for example, Lehmann, J. M.; Moore, L. B.; Smith-Oliver, T. A; Wilkinson, W. O.; Willson, T. M.; Kliewer, S. A., An antidiabetic thiazolidinedione is a high affinity ligand for peroxisome proliferator-activated receptory γ (PPARγ), J. Biol. Chem., 1995, 270, 12953-6. The ligand binding domains for murine and human PPAR-alpha, PPAR-gamma, and PPAR-delta are each fused to the yeast transcription factor GAL4 DNA binding domain. CV-1 cells were transiently transfected with expression vectors for the respective PPAR chimera along with a reporter construct containing four or five copies of the GAL4 DNA binding site driving expression of luciferase. After 8-16 h, the cells are replated into multi-well assay plates and the media is exchanged to phenol-red free DME medium supplemented with 5% delipidated calf serum. 4 hours after replating, cells were treated with either compounds or 1% DMSO for 20-24 hours. Luciferase activity was then assayed with Britelite (Perkin Elmer) following the manufacturer's protocol and measured with either the Perkin Elmer Viewlux or Molecular Devices Acquest (see, for example, Kliewer, S. A., et. al. Cell 1995, 83, 813-819). Rosiglitazone is used as a positive control in the hPPAR-γ assay. Wy-14643 and GW7647 is used as a positive control in the hPPAR-α assay. GW501516 is used as the positive control in the hPPAR-δ assay.

### ADDITIONAL ASSAYS

Compounds may be tested for their ability to bind to hPPAR-gamma, hPPAR-alpha, or PPAR-delta using a Scintillation Proximity Assay (SPA). The PPAR ligand binding domain (LBO) may be expressed in E. coli as polyHis tagged fusion proteins and purified. The LBO is then labeled with biotin and immobilized on streptavidin modified scintillation proximity beads. The beads are then incubated with a constant amount of the appropriate radioligand eH-BRL 49653 for PPARγ, 2-(4(2-(2,3-Ditritio-1-heptyl-3-(2,4-difluorophenyl)ureido )ethyl)phenoxy)-2 methyl butanoic acid (described in WO1008002) for hPPAR-alpha and GW 2433 (see Brown, P. J et al . Chem. Biol. 1997, 4, 909-918. For the structure and synthesis of this ligand) for PPAR-delta) and variable concentrations of test compound, and after equilibration the radioactivity bound to the beads is measured by a scintillation counter. The amount of nonspecific binding, as assessed by control wells containing 50 μM of the corresponding unlabelled ligand, is subtracted from each data point. For each compound tested, plots of ligand concentration vs. CPM of radioligand bound are constructed and apparent K, values are estimated from nonlinear least squares fit of the data assuming simple competitive binding. The details of this assay have been reported elsewhere (see, Blanchard, S. G. et. al., “Development of a Scintillation Proximity Assay for Peroxisome Proliferator-Activated Receptor gamma Ligand Binding Domain” Anal. Biochem. 1998, 257, 112-119).

### PHARMACEUTICAL COMPOSITIONS

The present invention includes all pharmaceutically acceptable, non-toxic esters of the compounds of Formula I. Such esters include C1-C6 alkyl esters wherein the alkyl group is a straight or branched chain. Acceptable esters also include C5-C7 cycloalkyl esters as well as arylalkyl esters such as, but not limited to benzyl. C1-C4 alkyl esters are preferred. Esters of the compounds of the present invention may be prepared according to conventional methods.

The compounds of the present invention are suitable to be administered to a patient for the treatment, control, or prevention of non-insulin dependent diabetes mellitus, hypercholesteremia, hyperlipidemia, obesity, hyperglycemia, hyperlipidemia, atherosclerosis, hypertriglyceridemia, and hyperinsulinemia. Accordingly, the compounds may be administered to a patient alone or as part of a composition that contains other components such as excipients, diluents, and carriers, all of which are well-known in the art. The compositions can be administered to humans and/or animals either orally, rectally, parenterally (intravenously, intramuscularly, or subcutaneously), intracisternally, intravaginally, intraperitoneally, intravesically, locally (powders, ointments, or drops), or as a buccal or nasal spray.

Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil), and injectable organic esters such as ethyl oleate. Proper fluidity can be maintained, for example, by the use of a coating such as lecithin, by the maintenance of the required particle size in the case of dispersions and by the use of surfactants.

These compositions may also contain adjuvants such as preserving, wetting, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monostearate and gelatin.

The compositions containing the compound(s) described herein can be administered for prophylactic and/or therapeutic treatments. In therapeutic applications, the compositions are administered to a patient already suffering from a disease, condition or disorder mediated, modulated or involving the PPARs, including but not limited to metabolic diseases, conditions, or disorders, as described above, in an amount sufficient to cure or at least partially arrest the symptoms of the disease, disorder or condition. Amounts effective for this use will depend on the severity and course of the disease, disorder or condition, previous therapy, the patient's health status and response to the drugs, and the judgment of the treating physician. It is considered well within the skill of the art for one to determine such therapeutically effective amounts by routine experimentation (e.g., a dose escalation clinical trial).

In prophylactic applications, compositions containing the compounds described herein are administered to a patient susceptible to or otherwise at risk of a particular disease, disorder or condition mediated, modulated or involving the PPARs, including but not limited to metabolic diseases, conditions, or disorders, as described above. Such an amount is defined to be a “prophylactically effective amount or dose.” In this use, the precise amounts also depend on the patient's state of health, weight, and the like. It is considered well within the skill of the art for one to determine such prophylactically effective amounts by routine experimentation (e.g., a dose escalation clinical trial).

### DOSAGE

Solid dosage forms for oral administration include capsules, tablets, pills, powders, and granules. In such solid dosage forms, the active compound is admixed with at least one inert customary excipient (or carrier) such as sodium citrate or dicalcium phosphate or (a) fillers or extenders, as for example, starches, lactose, sucrose, glucose, mannitol, and silicic acid; (b) binders, as for example, carboxymethylcellulose, alignates, gelatin, polyvinylpyrrolidone, sucrose, and acacia; (c) humectants, as for example, glycerol; (d) disintegrating agents, as for example, agar, calcium carbonate, potato or tapioca starch, alginic acid, certain complex silicates, and sodium carbonate; (e) solution retarders, as for example paraffin; (f) absorption accelerators, as for example, quaternary ammonium compounds; (g) wetting agents, as for example, cetyl alcohol and glycerol monostearate; (h) adsorbents, as for example, kaolin and bentonite; and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft and hard-filled gelatin capsules using such excipients as lactose or milk sugar, as well as high molecular weight polyethyleneglycols, and the like.

Solid dosage forms such as tablets, dragees, capsules, pills, and granules can be prepared with coatings and shells, such as enteric coatings and others well-known in the art. They may contain opacifying agents, and can also be of such composition that they release the active compound or compounds in a certain part of the intestinal tract in a delayed manner. Examples of embedding compositions which can be used are polymeric substances and waxes. The active compounds can also be in micro-encapsulated form, if appropriate, with one or more of the above-mentioned excipients.

Liquid dosage forms for oral administration include pharmaceutically acceptable emulsions, solutions, suspensions, syrups, and elixirs. In addition to the active compounds, the liquid dosage forms may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan or mixtures of these substances, and the like. Besides such inert diluents, the composition can also include adjuvants, such as wetting agents, emulsifying and suspending agents, sweetening, flavoring, and perfuming agents.

Suspensions, in addition to the active compounds, may contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Compositions for rectal administrations are preferably suppositories which can be prepared by mixing the compounds of the present invention with suitable non-irritating excipients or carriers such as cocoa butter, polyethyleneglycol, or a suppository wax, which are solid at ordinary temperatures but liquid at body temperature and therefore, melt in the rectum or vaginal cavity and release the active component.

Dosage forms for topical administration of a compound of this invention include ointments, powders, sprays, and inhalants. The active component is admixed under sterile conditions with a physiologically acceptable carrier and any preservatives, buffers, or propellants as may be required. Ophthalmic formulations, eye ointments, powders, and solutions are also contemplated as being within the scope of this invention.

The compounds of the present invention can be administered to a patient at dosage levels in the range of about 0.1 to about 2,000 mg per day. For a normal human adult having a body weight of about 70 kilograms, a dosage in the range of about 0.01 to about 10 mg per kilogram of body weight per day is preferable. However, the specific dosage used can vary. For example, the dosage can depend on a numbers of factors including the requirements of the patient, the severity of the condition being treated, and the pharmacological activity of the compound being used. The determination of optimum dosages for a particular patient is well-known to those skilled in the art. Once improvement of the patient's conditions has occurred, a maintenance dose is administered if necessary. Subsequently, the dosage or the frequency of administration, or both, can be reduced, as a function of the symptoms, to a level at which the improved disease, disorder or condition is retained. When the symptoms have been alleviated to the desired level, treatment can cease. Patients can, however, require intermittent treatment on a long-term basis upon any recurrence of symptoms.

The amount of a given agent that will correspond to such an amount will vary depending upon factors such as the particular compound, disease condition and its severity, the identity (e.g., weight) of the subject or host in need of treatment, but can nevertheless be routinely determined in a manner known in the art according to the particular circumstances surrounding the case, including, e.g., the specific agent being administered, the route of administration, the condition being treated, and the subject or host being treated. In general, however, doses employed for adult human treatment will typically be in the range of 0.02-5000 mg per day, preferably 1-1500 mg per day. The desired dose may conveniently be presented in a single dose or as divided doses administered at appropriate intervals, for example as two, three, four or more sub-doses per day.

In certain instances, it may be appropriate to administer at least one of the compounds described herein (or a pharmaceutically acceptable salt, ester, amide, prodrug, or solvate) in combination with another therapeutic agent. By way of example only, if one of the side effects experienced by a patient upon receiving one of the compounds herein is hypertension, then it may be appropriate to administer an anti-hypertensive agent in combination with the initial therapeutic agent. Or, by way.of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one of the compounds described herein with another therapeutic agent (which also includes a therapeutic regimen) that also has therapeutic benefit. By way of example only, in a treatment for diabetes involving administration of one of the compounds described herein, increased therapeutic benefit may result by also providing the patient with another therapeutic agent for diabetes. In any case, regardless of the disease, disorder or condition being treated, the overall benefit experienced by the patient may simply be additive of the two therapeutic agents or the patient may experience a synergistic benefit.

Specific, non-limiting examples of possible combination therapies include use of the compound of formula (I) with: (a) stating and/or other lipid lowering drugs for example MTP inhibitors and LDLR upregulators; (b) antidiabetic agents, e.g. metformin, sulfonylureas, or PPAR-gamma, PPAR-alpha and PPAR-alpha/gamma modulators (for example thiazolidinediones such as e.g. Pioglitazone and Rosiglitazone); and (c) antihypertensive agents such as angiotensin antagonists, e.g., telmisartan, calcium channel antagonists, e.g. lacidipine and ACE inhibitors, e.g., enalapril.

In any case, the multiple therapeutic agents (one of which is one of the compounds described herein) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may vary from more than zero weeks to less than four weeks.

### GENERAL SYNTHETIC METHODS FOR PREPARING COMPOUNDS

Numerous compounds which embody the present invention can be prepared by the general process in Scheme 1:

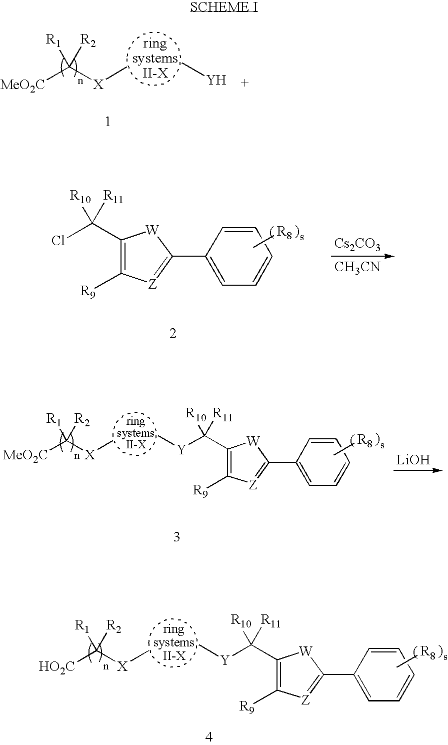

Scheme I depicts the convergent synthesis of a generic embodiment 4, from components 1 and 2 using standard nucleophilic displacement chemistry. Generic intermediates like 3 may be deprotected to form several embodiments of the present invention.

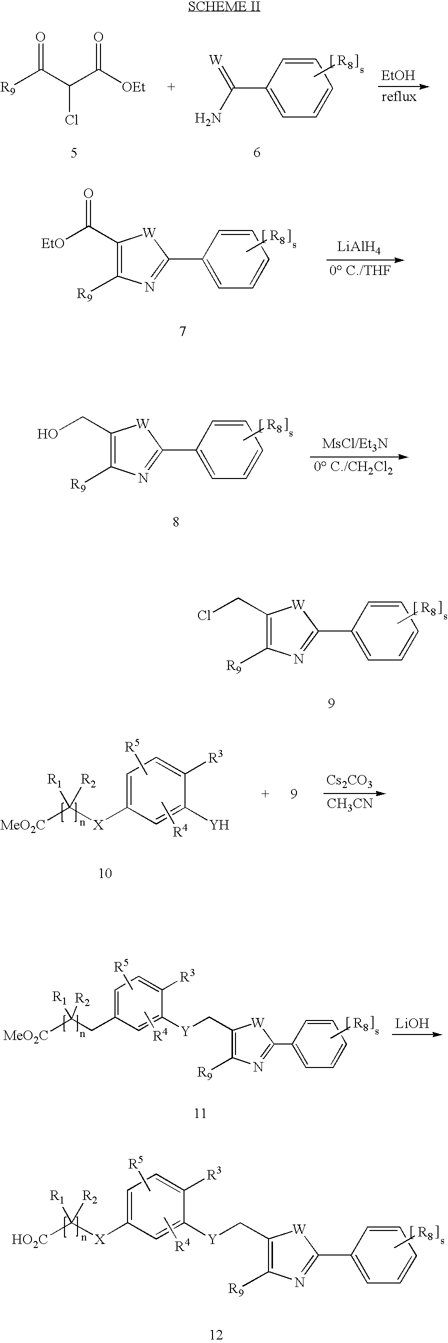

Scheme II depicts the synthesis of intermediates used in the convergent syntheses of numerous embodiments of the present invention. For example, when the [B] ring system has Formula (II), the preparation of oxazole and thiazole derivatives (Z=N, W═O or S), with reference to Scheme II. Benzamide or thiobenzamide (6) is added to 5 to form oxazole or thiazole (7). The ester is reduced to give (8) which is then converted to alkyl chloride (9). Coupling of (9) and (10) with cesium carbonate in acetonitrile followed by hydrolysis affords (12), a generic embodiment of the invention wherein [B] has the structure corresponding to Formula (II).

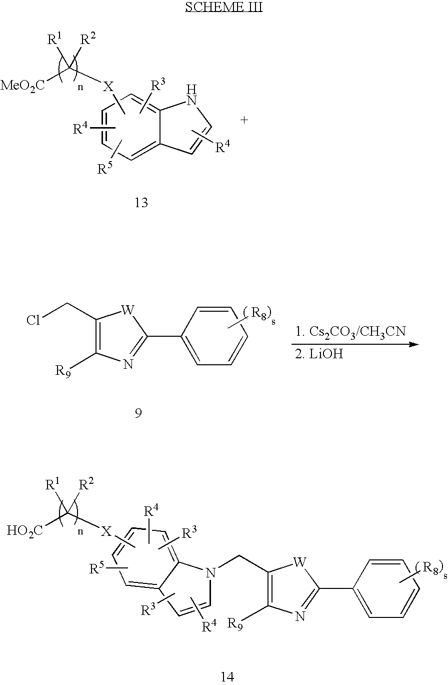

Scheme III depicts the convergent synthesis of certain embodiments of the invention when the [B] ring system has Formula (III), and X¹ is NH ( e.g., [B]=indole). Oxazole and thiazole intermediates were prepared as previously described in Scheme II.

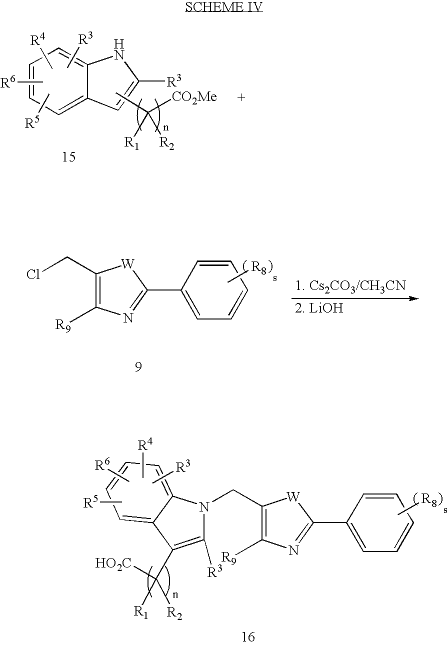

Scheme IV depicts the convergent synthesis of certain embodiments of the invention when the [B] ring system has Formula (VI), and X¹ is NH (e.g., [B]=indole). Oxazole and thiazole intermediates were prepared as previously described in Scheme II.

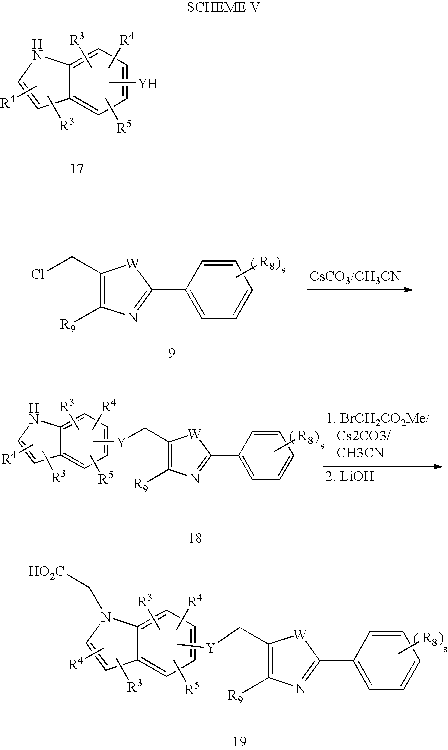

Scheme V depicts the convergent synthesis of certain embodiments of the invention when the [B] ring system has Formula (V), and X¹ is NH (e.g., [B]=indole). Oxazole and thiazole intermediates were prepared as previously described in Scheme II.

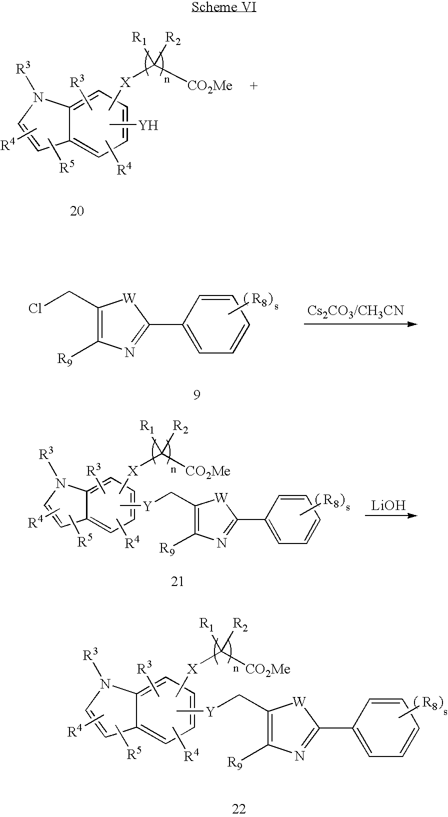

Scheme VI depicts the convergent synthesis of certain embodiments of the invention when the [B] ring system has Formula (IV), and X¹ is NH (e.g., [B]=indole). Oxazole and thiazole intermediates were prepared as previously described in Scheme II.

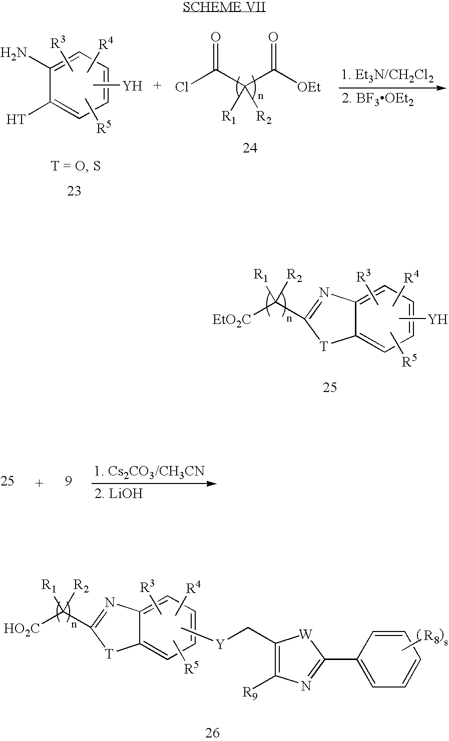

Scheme VII depicts the convergent synthesis of certain embodiments of the invention when the [B] ring system has Formula (V), X¹ is N, X³ is O or S (e.g., [B]=benzoxazole or benzothiaphene). Oxazole and thiazole intermediates corresponding to intermediate 9 were prepared as previously described in Scheme II.

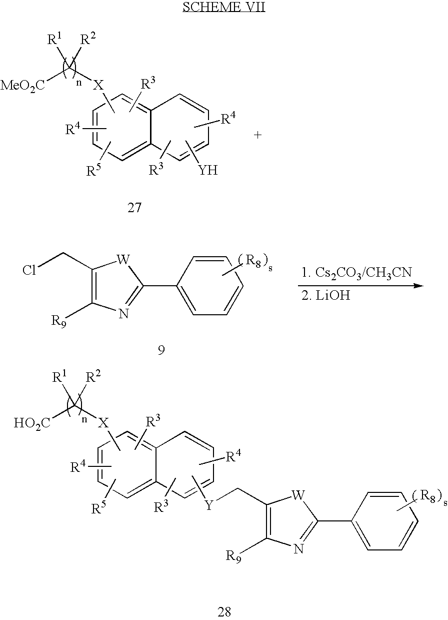

Scheme VIII depicts the convergent synthesis of certain embodiments of the invention when the [B] ring system has Formula (IX), E¹-E⁸ are C, and Z is N ( e.g., [B]=naphthalene). Oxazole and thiazole intermediates corresponding to intermediate 9 were prepared as previously described in Scheme II.

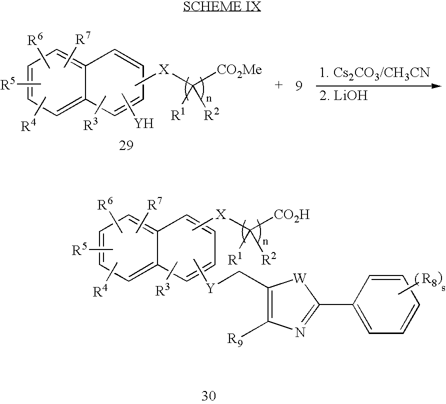

Scheme IX depicts the convergent synthesis of certain embodiments of the invention when the [B] ring system has Formula (X), E¹-E⁸ are C, and Z is N (e.g., [B]=naphthalene). Oxazole and thiazole intermediates corresponding to intermediate 9 were prepared as previously described in Scheme II.

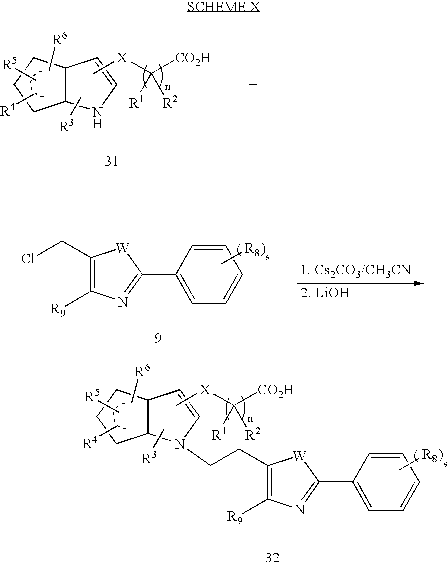

Scheme X depicts the convergent synthesis of certain embodiments of the invention when the [B] ring system has Formula (VIII), E¹ is N, X²-X⁶ are C, and Z is N (e.g., [B]=pyrrolothiophene or imidazolothiophene). Oxazole and thiazole intermediates corresponding to intermediate 9 were prepared as previously described in Scheme II.

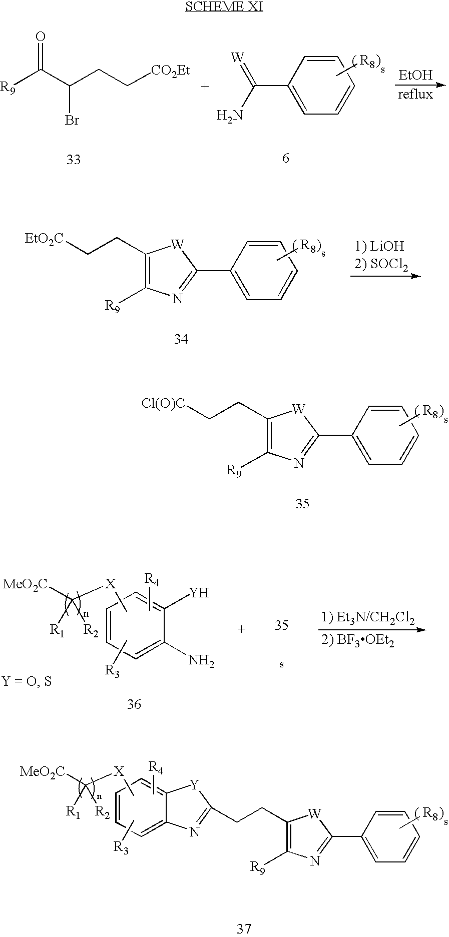

Scheme XI depicts the convergent synthesis of certain embodiments of the invention when the [B] ring system has Formula (III), X¹ is N, X³ (Y above) is S or N, and Z is N ( e.g., [B]=benzimidazole or benzothiaphene). Oxazole and thiazole intermediates corresponding to intermediate 9 were prepared as previously described in Scheme II.

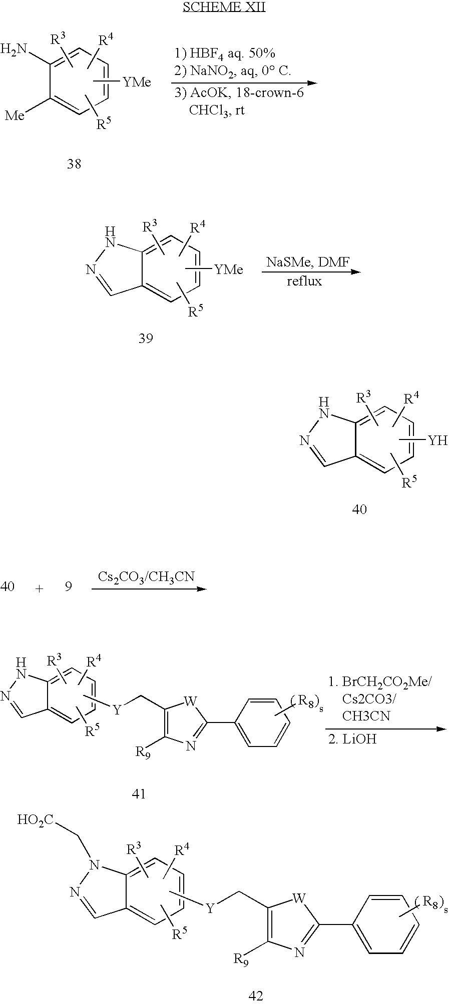

Scheme XII depicts the convergent synthesis of certain embodiments of the invention when the [B] ring system has Formula (IV), X¹ is NH, X² is N, and Z is N (e.g., [B]=indazole). Oxazole and thiazole intermediates corresponding to intermediate 9 were prepared as previously described in Scheme II.

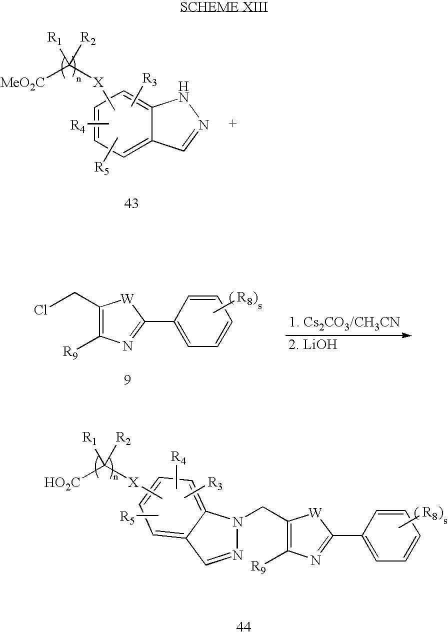

Scheme XIII depicts the convergent synthesis of certain embodiments of the invention when the [B] ring system has Formula (III), X¹ is NH, X² is N, and Z is N (e.g., [B]=indazole). Oxazole and thiazole intermediates corresponding to intermediate 9 were prepared as previously described in Scheme II.

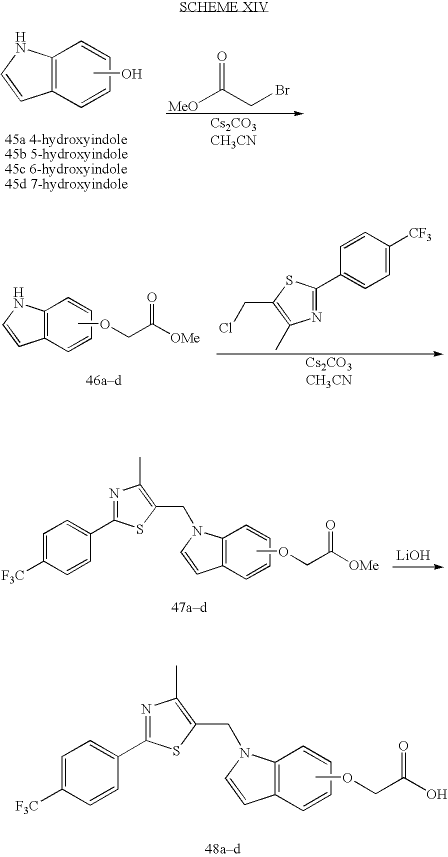

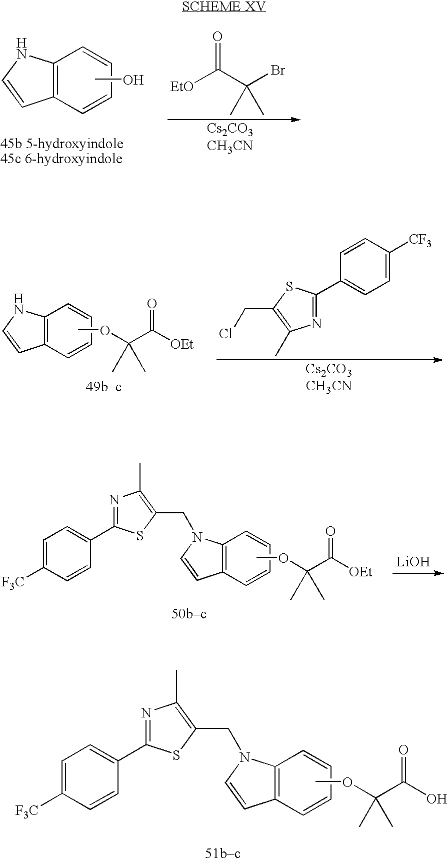

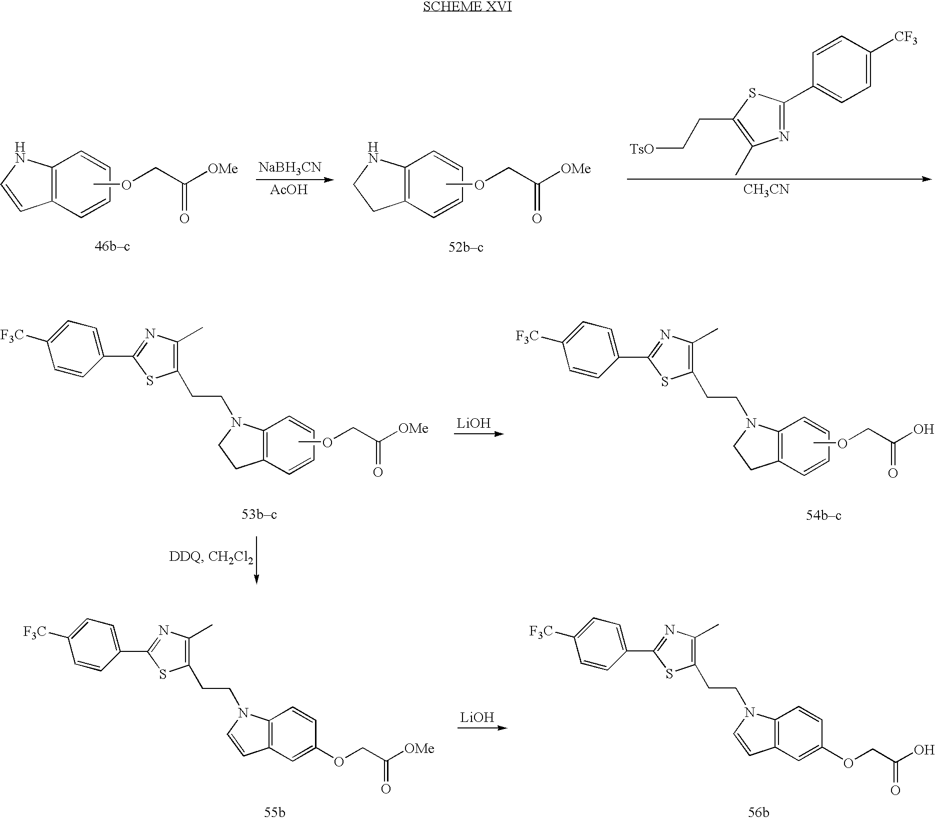

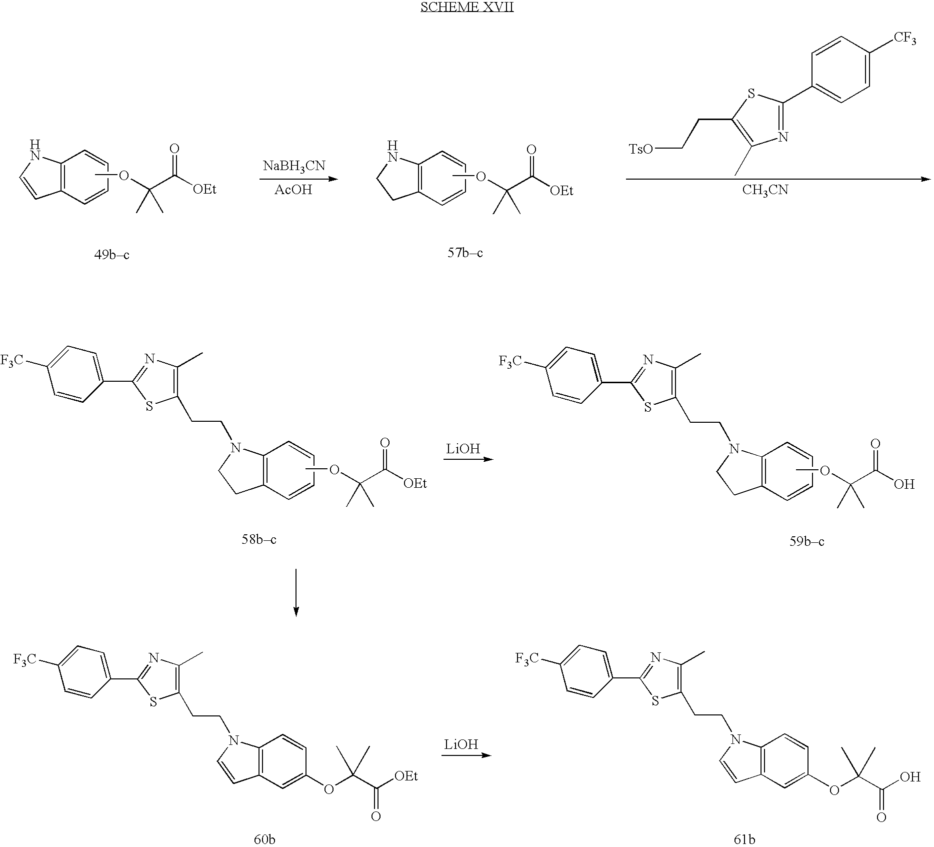

### SYNTHESES OF OTHER EXAMPLES

Several prophetic examples of the present invention have heterocyclic elements [B] which not generically described above. Such heterocycles may be synthesized de novo or often, purchased. The following synthetic methods may used to prepare heterocyclic elements [B] not described above. These descriptions are organized alphabetically. Many of these classic ring-forming reactions tolerate the presence of alkyl substituents as disclosed herein. The skilled artisan recognizes that these methods may be extended to countless variants.

Chromenes

Chromenes may be from phenols and 1,3-carbonyldielectrophiles: Simonis et al. Aust. J. Chem. 1972, 25, 1367): 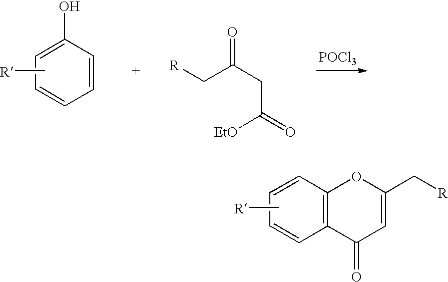
Cinnolines

Cinnoline derivatives may be formed by diazotization of o-aminoarylpropiolic acids (available from Pd-catalyzed addition of propionic acid to aniline) followed by hydration and cyclization. The method is applicable for any o-amino arylacetylenes (V. von Richter, Ber. 1883, 16, 677): 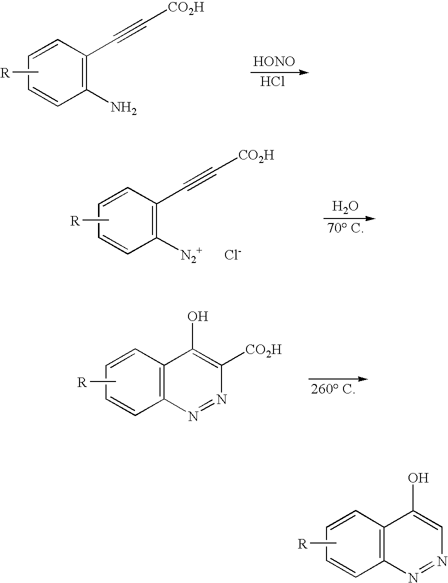
Coumarins

Coumarins are available by condensing phenols with β-keto esters or equivalent 1,3 dielectrophiles in the presence of Lewis acid catalysts (H. v. Pechmann, C. Duisberg, Ber. 1883, 16, 2119): 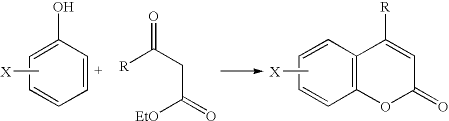
Dioxindoles

Dioxindoles are available by condensing N-substituted anilines with alpha-ketomalonates (A. Guyot, J. Martinet, Compt. Rend. 1913, 156, 1625): 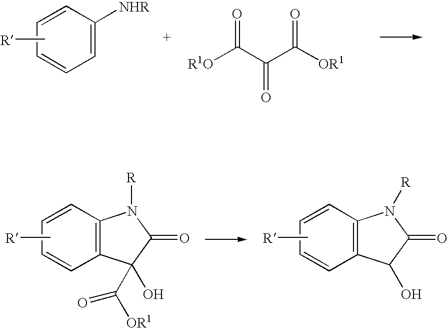
Indoles

Substituted indoles may be prepared from the aryl hydrazones of aldehydes (generally available from aldehydes and substituted arylhydrazines) according to the method of Fischer: (Ber. 1883, 16, 2241; Accts. Chem. Research 1981, 14, 275):

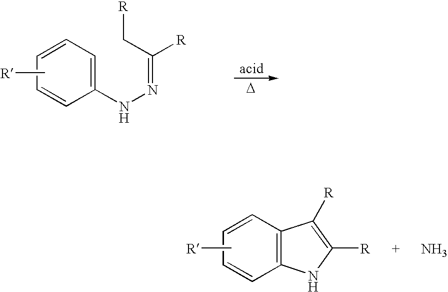

Substituted indoles may also be prepared via the method of Bischler-Moehlau (A. Bischler et al., Ber. 1892, 25, 2860; Heterocyclic Compounds 1952, 3, 22.)

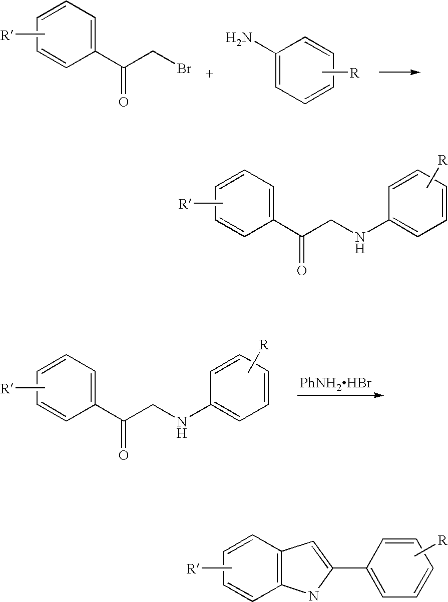

Indoles may be prepared according to the method of von Baeyer (v. Baeyer A., Emmerling A., Ber., 1869, 2, 679):

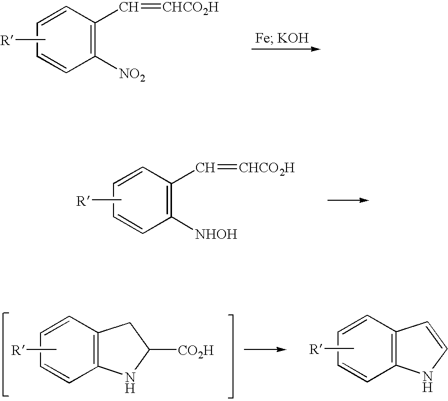

Indoles may be by intramolecular cyclization of N-(2-alkylphenyl)alkanamides in the presence of strong base (W. Madelung, Ber. 1912, 45, 1128):

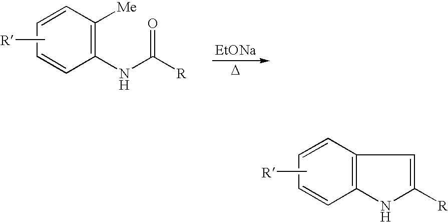

5-hydroxyindoles may be synthesized by condensing p-benzoquinone with β-aminocrotonic esters (C. D. Nenitzescu, Bull. Soc. Chim. Romania 1929, 11, 37; review R. K. Brown in The Chemistry of Heterocyclic Compounds, W. J. Houlihan, Ed. Wiley, New York, 1972, p. 413).

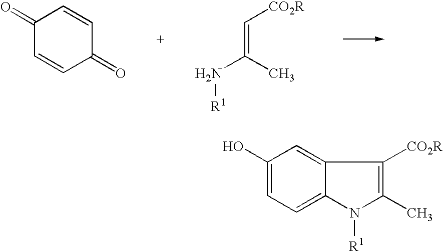

Indoles may be prepared from condensation of an o-nitrotoluene with oxalic ester, reduction to amine, and cyclization to indole (A. Reissert, Ber. 1897, 30, 1030): 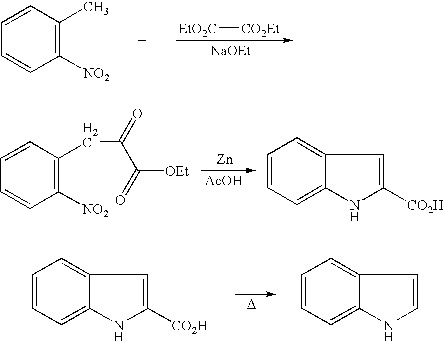
Indolines

Indoline derivatives may be formed by the reaction of arylamines with α-haloacid chlorides or oxalyl chloride, followed by cyclization of the resulting amides with aluminum chloride: (R. Stollé, Ber. 1913, 46, 3915; ibid 1914, 47, 2120; see also J. Prakt. Chem. 1923, 105, 137; 128, 1 (1930): 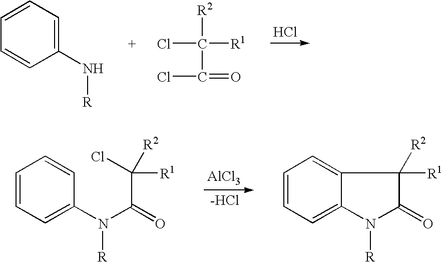
Isoguinolines

Isoquinolines are available from cyclization of acylated aminomethyl phenyl carbinols or their ethers with phosphorus pentoxide in toluene or xylene. (A. Pictet and A. Gams, Ber. 1910, 43, 2384.)

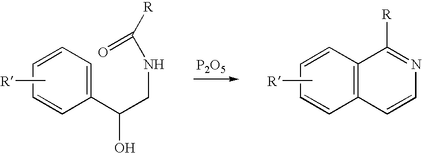

Isoquinolines are also available by cyclization of acylated aminomethyl phenyl carbinols or their ethers with phosphorus pentoxide in toluene or xylene (Heterocycles 1994, 39, 903):

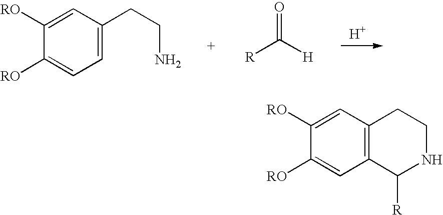

Cyclodehydration of β-phenethylamides to 3,4-dihydroisoquinoline derivatives by means of condensing agents such as phosphorous pentoxide or zinc chloride A. Bischler, B. Napieralski, Ber. 26, 1903 (1893): 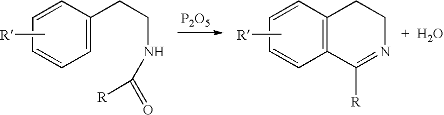
Oxindoles

Oxindoles may be synthesized from secondary aryl amines and the acid addition compound of glyoxal; primary aryl amines give glycine or glycinamide derivatives (O. Hinsberg Ber. 1888, 21, 110): 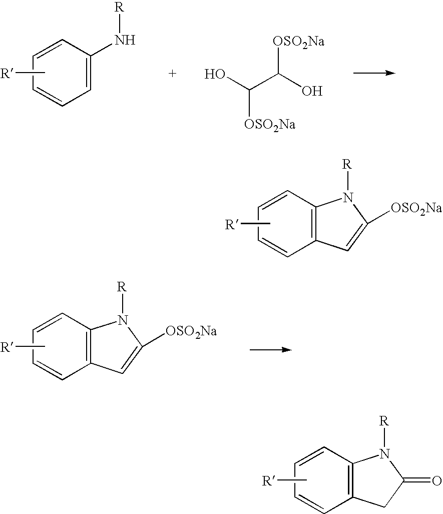
Oxazoles

Oxazoles may be prepared using the method of Fischer (Tetrahedron Lett. 1971, 4391): 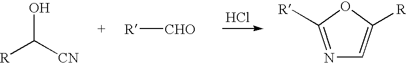
Purines

Preparation of 4,5-diaminopyrimidines by introduction of the amino group into the 5-position of 4-amino-6-hydroxy- or 4,6-diaminopyrimidines by nitrosation and ammonium sulfide reduction, followed by ring closure with formic acid or chlorocarbonic ester (W. Traube, Ber. 1900, 33, 1371):

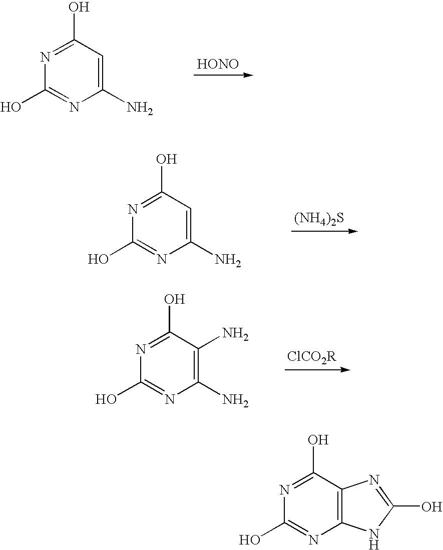

For reviews of pyrimidine chemistry, including their syntheses, see J. H. Davidson, The Nucleic Acids I (New York, 1955) p 131; A. R. Katritzky, Quart. Rev. 1956, 10, 397; idem, Rev. Pure Appl. Chem. 1961, 11, 178; J. H. Lister, Purines (Wiley, New York, 1971) pp 31-90.

Quinazolines

4-oxo-3,4-dihydroquinazolines may be formed by cyclization of anthranilic acid and amides (S. v. Niementowski, J. Prakt. Chem. 1895, 51, 564): 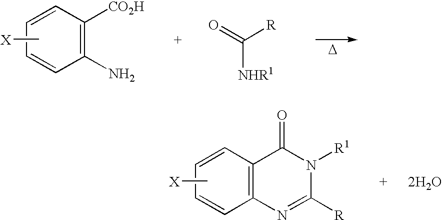
Quinolines

Substituted quinolines are available from aniline and 1,3 diketones: (Combes et al. J. Org. Chem. 1972, 37, 3952)

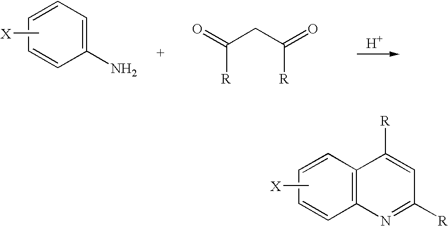

Quinolines may be prepared according to the method of Knorr (Knorr et al. J. Org. Chem. 1969, 34, 1709):

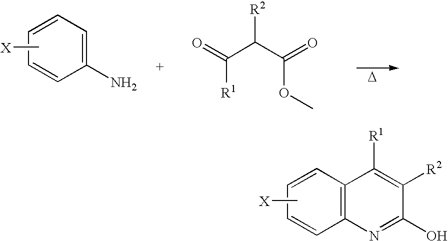

Quinolines may be prepared according to the method of Riehm (Heterocyclic Compounds 1952, 4, 16.)

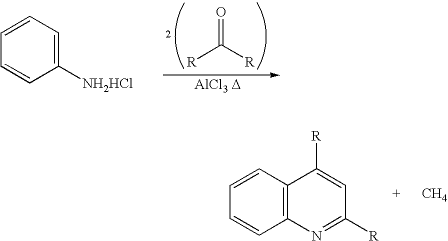

Hydroxyquinolines may be prepared from o-acylaminoacetophenones in alcoholic sodium hydroxide. Two isomers are produced; the relative proportions are mainly determined by the residue on the amino nitrogen (Camps, Ber. 1899, 22, 3228):

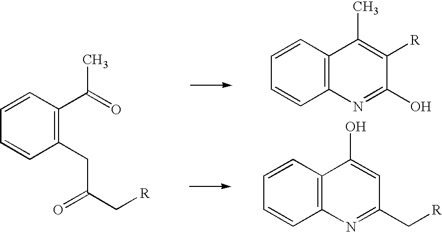

Quinolines may be prepared from the thermal condensation of arylamines with β-ketoesters followed by cyclization of the intermediate Schiff bases to 4-hydroxyquinolines (M. Conrad, L. Limpach, Ber. 1887, 20, 944.; ibid, 1891, 24, 2990):

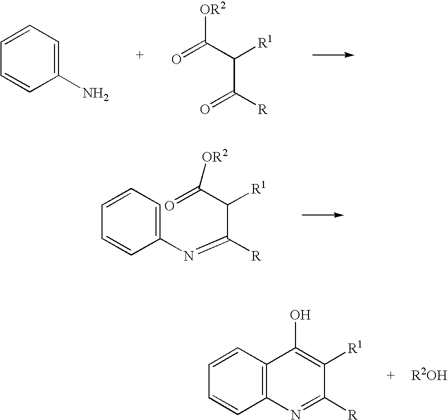

Substituted quinolines are available from aniline and 1,3-diketones: (Combes et al. J. Org. Chem. 1972, 37, 3952):

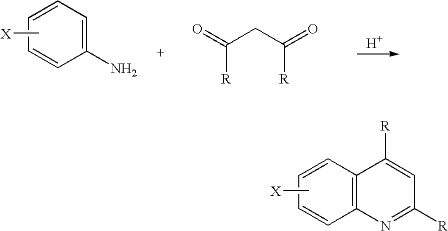

Quinolines may be prepared from anilines and β-ketoesters (Knorr et al. J. Org. Chem. 1969,34, 1709):

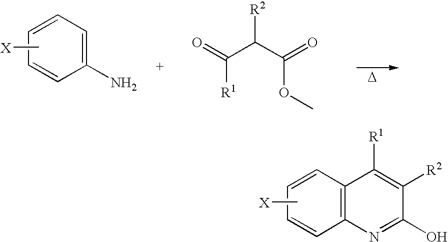

Quinolines may also be prepared from anilines and two equivalents of ketone (Riehm Heterocyclic Compounds 1952, 4, 16):

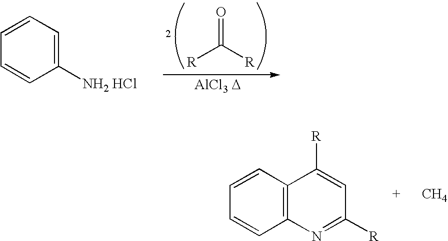

Quinolines may be prepared from primary aromatic amines and α,β-unsaturated carbonyl compounds under acid conditions. When the latter are prepared in situ from two molecules of aldehyde or an aldehyde and methyl ketone, the reaction is known as the Beyer method for quinolines (O. Doebner, W. v. Miller, Ber. 1883, 16, 2464):

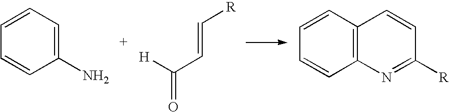

Quinolines may be prepared from base-catalyzed condensation of 2-aminobenzaldehydes with ketones to form quinoline derivatives: (P. Friedlaender, Ber. 1882, 15, 2572):

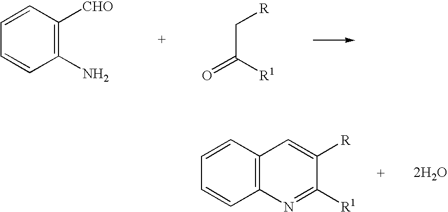

γ-Hydroxyquinolines derivatives may be prepared from anthranilic acids and carbonyl compounds (S. v. Niementowski, Ber. 1894, 27, 1394; ibid, 1895, 28, 2809; ibid, 1905, 38, 2044; ibid-1907; 40, 4285:

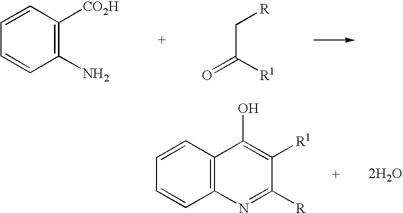

Formation of quinoline-4-carboxylic acids by condensation of isatic acids from isatin with α-methylene carbonyl compounds; subsequent decarboxylation yields quinolines (W. Pfitzinger, J. Prakt. Chem. 1886, 33, 100):

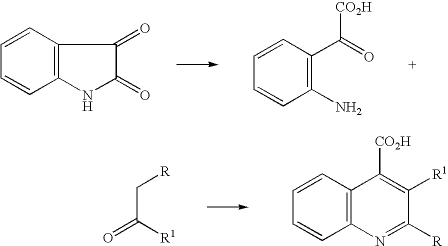

Quinolines may be prepared from aromatic amines, glycerol, an oxidizing agent and sulfuric acid (Z. H. Skraup, Ber. 1880, 13, 2086): 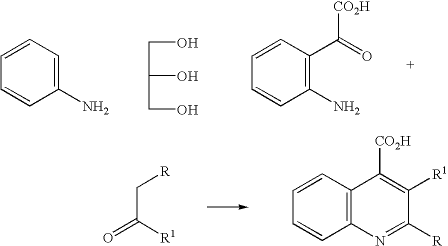
Quinoxalines

Quinoxaline may be synthesized from o-phenylenediamines and 1,2 dielectrophiles:

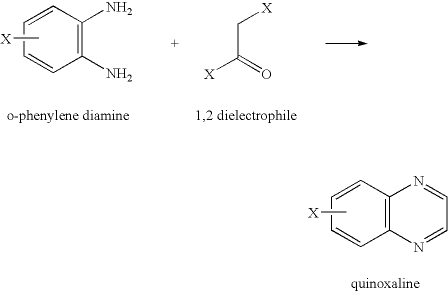

Specific examples include condensation of o-phenylenediamines with various ketoacid derivatives [Cheeseman et al. in The Chemistry of Heterocyclic Compounds Vol 35 Weissberger, A.; Taylor, E. C. Eds; John Wiley & Sons: New York, 1979; pp. 78-111]. Various tetrahydro quinoxalines are available from reduction of unsaturated precursors.

Thiazoles

4-carboxylate thiazoles may be prepared from alkyl isocyanoacetate and thionoesters. The process is suitable for making thiazoles with other electron withdrawing groups in the 4-position (Hartman G. D.; Weinstock, L. M. Org Synth Collective Vol. 6, 620):

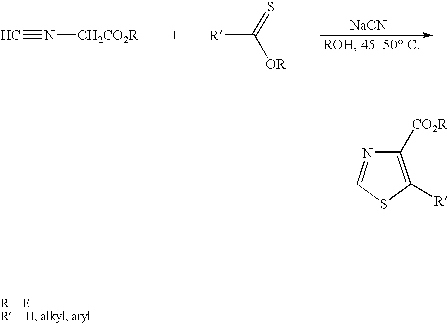

Substituted thiazoles have been prepared from thioamides and chloracetates (Bioorg. Med. Chem Lett. 2003, 13, 3491):

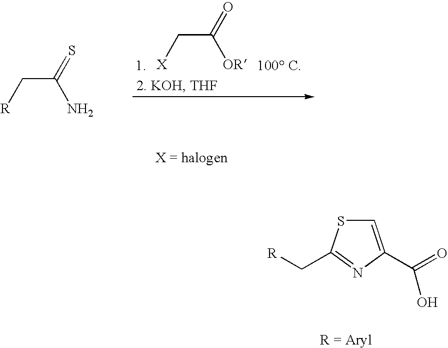

### EXAMPLES

The following examples describe embodiments of the invention. Other embodiments within the scope of the claims herein will be apparent to one skilled in the art from consideration of the specification or practice of the invention as disclosed herein. It is intended that the specification, together with the examples, be considered to be exemplary only, with the scope and spirit of the invention being indicated by the claims which follow the examples. In the examples, all percentages are given on a weight basis unless otherwise indicated.

### SYNTHESIS OF EXAMPLE 1

### {3-[4-methyl-2-(4-trifluoromethyl-phenyl)-thiazol-5-ylmethoxy]-phenyl}-acetic acid

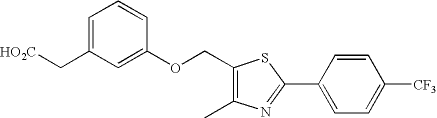

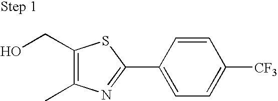

To a solution of lithium aluminum hydride (10 mL of 1.0 M solution in THF, 10.0 mmol) in THF (20 mL) at 0° C., was added a solution of 4-methyl-2-(4-trifluoromethyl-phenyl)-thiazole-5-carboxylic acid ethyl ester (3.0 g, 9.5 mmol) in dry THF (30 mL). After stirring at 0° C. for 10 min, the reaction mixture was warmed up to room temperature and continued to stir for 1.5 h. The reaction was quenched by slow addition of water (3 mL), 1N NaOH (40 mL). The resulting mixture was filtered through Celite and the filtrate was extracted with ethyl acetate (50 mL×2). The combined organic solution was washed with brine and dried over Na₂SO₄. After removal of solvent, 2.51 g (97% yield) of the desired product was obtained as a bright yellow solid. ¹H NMR (400 MHz, CDCl₃), δ (ppm): 8.01 (d, 2H), 7.67 (d, 2H), 4.85 (s, 2H), 2.47 (s, 3H).

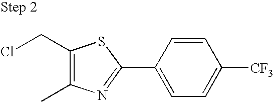

To a cold (0° C.) stirred solution of the product from Step 1 (2.51 g, 9.19 mmol) and Et₃N (2.56 mL, 18.37 mmol) in dry CH₂Cl₂ (150 mL), was slowly added MsCl (1.07 mL, 13.78 mmol). The reaction mixture was stirred at 0° C. The reaction mixture was diluted with 200 mL of CH₂Cl₂ washed with saturated NaHCO₃, water, brine, and dried over Na₂SO₄. Removal of solvent affords 2.65 g (99% yield) of the desired product as brown solid. ¹H NMR (400 MHz, CDCl₃), δ (ppm): 8.01 (d, 2H), 7.68 (d, 2H), 4.80 (s, 2H), 2.51 (s, 3H).

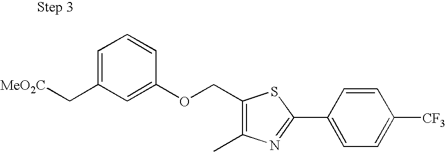

To a solution of methyl 3-hydroxyphenylacetate (199.4 mg, 1.2 mmol) and the product from Step 2 (286 mg, 0.98 mmol) in CH₃CN (10 mL) was added Cs₂CO₃ (489 mg, 1.5 mmol). The resulting suspension was stirred at room temperature for 20 h. The reaction mixture was concentrated in vacuo and the residue was diluted with ethyl acetate (20 mL), washed with water, brine, and dried over Na₂SO₄. After removal of solvent, the crude product was purified by chromatography to afford 264 mg (64% yield) of the desired product. ¹H NMR (400 MHz, CDCl₃), δ (ppm): 8.03 (d, 2H), 7.68 (d, 2H), 7.28 (m, 1H), 6.93 (m, 3H), 5.21 (s, 2H), 3.71 (s, 3H), 3.63 (s, 2H), 2.53 (s, 3H).

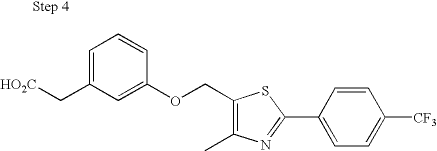

To a solution of the product from Step 3 (264 mg, 0.63 mmol) in THF/MeOH (3:1) (5 mL) was added 1N LiOH (1.5 mL, 1.5 mmol). The reaction mixture was kept at room temperature for 20 h. The reaction mixture was concentrated under nitrogen and the residue was diluted with water (5 mL). The aqueous mixture was partitioned with diethyl ether (2 mL). After separation, the aqueous solution was neutralized with 1N HCl (1.5 mL) and then extracted with ethyl acetate (10 mL). The organic layer was washed with water, brine, dried over Na₂SO₄. After removal of solvent, the crude product was purified by chromatography to afford the desired product. ¹H NMR (400 MHz, CDCl₃), δ (ppm): 8.01 (d, 2H), 7.67 (d, 2H), 7.28 (m, 1H), 6.93 (m, 3H), 5.19 (s, 2H), 3.64 (s, 2H), 2.51 (s, 3H).

### SYNTHESIS OF EXAMPLE 2

### {3-[4-methyl-2-(4-trifluoromethyl-phenyl)-thiazol-5-ylmethylsulfanyl]-phenyl}-acetic acid

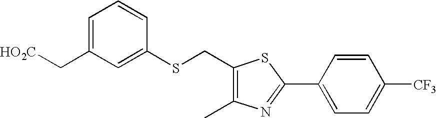

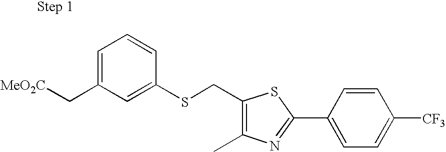

Using the procedure of Example 1, Step 3 and substituting methyl 3-mercaptophenylacetate for methyl 3-hydroxyphenylacetate, the desired product was obtained in 53% yield. ¹H NMR (400 MHz, CDCl₃), δ (ppm): 8.00 (d, 2H), 7.67 (d, 2H), 7.28 (m, 3H), 7.19 (m, 1H), 4.24 (s, 2H), 3.69 (s, 3H), 3.60 (s, 2H), 2.30 (s, 3H).

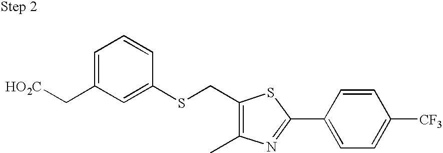

The compound from Step 1 was hydrolyzed using the procedure from Example 1, Step 4 to give the desired product in satisfactory yield. ¹H NMR (400 MHz, CDCl₃), δ (ppm): 7.93 (d, 2H), 7.63 (d, 2H), 7.28 (m, 3H), 7.19 (m, 1H), 4.21 (s, 2H), 3.59 (s, 2H), 2.23 (s, 3H).

### SYNTHESIS OF EXAMPLE 3

### {5-[4-methyl-2-(4-trifluoromethyl-phenyl)-thiazol-5-ylmethoxy]-indol-1-yl}-acetic acid

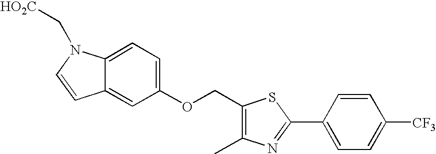

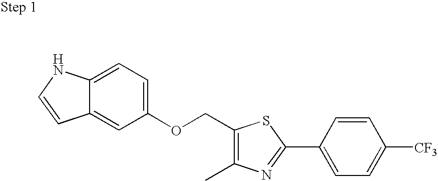

To a solution of the product from Example 1, Step 2 (1.0 mmol) in CH₃CN (3 mL) was added 1H-indole-5-ol (1.2 mmol) and Cs₂CO₃ (1.5 mmol). The resulting reaction mixture was diluted with CH₃CN (8 mL) and stirred for 21 h. The reaction mixture was concentrated under nitrogen. The residue was diluted with EtOAc (15 ML) and washed with water and brine then dried (Na₂SO₄) and concentrated in vacuo. The residue was purified by chromatography to give the desired product in 28% yield. ¹H NMR (400 MHz, CDCl₃), δ (ppm): 8.12 (b, 1H), 8.03 (d, 2H), 7.68 (d, 2H), 7.29 (d, 1H), 7.22(s, 2H), 6.91 (d, 1H), 6.51 (s, 1H), 5.25 (s, 2H), 2.52 (s, 3H).

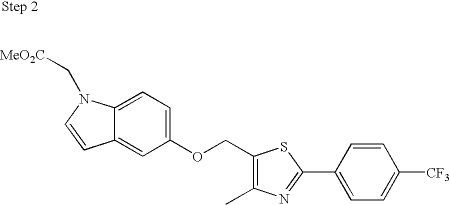

To a solution of the product from Step 1 (0.27 mmol) in CH₃CN (5 mL) was added methyl bromoacetate (0.54 mmol) and Cs₂CO₃ (1.5 mmol). The reaction mixture was heated at 75° C. for 24 h. The reaction mixture was cooled and solids removed by filtration. The filtrate was concentrated under nitrogen and the residue purified by chromatography to give the desired product in 63% yield. ¹H NMR (400 MHz, CDCl₃), δ (ppm): 8.04 (d, 2H), 7.69 (d, 2H), 7.23 (s, 1H), 7.21 (d, 1H), 7.11 (s, 1H), 6.96 (d, 1H), 6.53 (s, 1H), 5.26 (s, 2H), 4.86 (s, 1H), 3.77 (s, 3H), 2.54 (s, 3H).

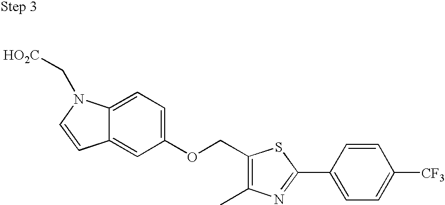

To a solution of the product from Step 2 (0.17 mmol) in THF/MeOH (3:1) (3 mL) was added 1N LiOH (0.85 mmol). The reaction mixture was stifred at room temperature for 48 h. The reaction mixture was concentrated under nitrogen and the residue was diluted with water (5 mL). The aqueous mixture was extracted with ether (2 mL). The aqueous solution was acidified with 1N HCl (1.5 mL) and then extracted with ethyl acetate (5 mL). The organic solution was washed with water, brine, dried over Na₂SO₄. After removal of solvent, the crude product was purified by chromatography to afford the desired product in 87% yield. ¹H NMR (400 MHz, CDCl₃), δ (ppm): 8.00 (d, 2H), 7.66 (d, 2H), 7.21 (s, 1H), 7.16 (d, 1H), 7.07 (s, 1H), 6.96 (d, 1H), 6.51 (s, 1H), 5.23 (s, 2H), 4.87 (s, 1H), 2.51 (s, 3H).

### SYNTHESIS OF EXAMPLE 4

### {4-[4-methyl-2-(4-trifluoromethyl-phenyl)-thiazol-5-ylmethoxy]-indol-1-yl}-acetic acid

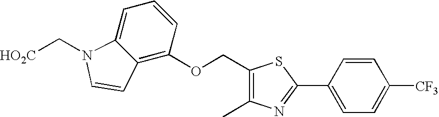

The compound of Prepared using the procedure as in Example 3, Steps 1-3, but substituting 1H-indole-4-ol for 1H-indole-5-ol in Step 1. ¹H NMR (400 MHz, CDCl₃), δ (ppm): 8.01 (d, 2H), 7.66 (d, 2H), 7.17 (t, 1H), 7.00 (s, 1H), 6.93 (d, 1H), 6.68 (s, 1H), 6.65 (d, 2H), 5.34 (s, 2H), 4.86 (s, 1H), 2.53 (s, 3H).

### SYNTHESIS OF EXAMPLE 5

### {6-[4-methyl-2-(4-trifluoromethyl-phenyl)-thiazol-5-ylmethoxy]-indol-1-yl}-acetic acid

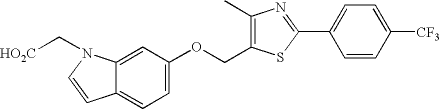

The compound of Example 5 was prepared using the procedure as in Example 3, Steps 1-3, but substituting 1H-indole-6-ol for 1H-indole-5-ol in Step 1. ¹H NMR (400 MHz, CDCl₃), δ (ppm): 8.07 (d, 2H), 7.71 (d, 2H), 7.51 (d, 1H), 7.08 (s, 1H), 6.92 (s, 1H), 6.84 (d, 1H), 6.47 (s, 1H), 5.29 (s, 2H), 4.84 (s, 1H), 2.55 (s, 3H).

### SYNTHESIS OF EXAMPLE 6

### {7-[4-methyl-2-(4-trifluoromethyl-phenyl)-thiazol-5-ylmethoxy]-indol-1-yl}-acetic acid

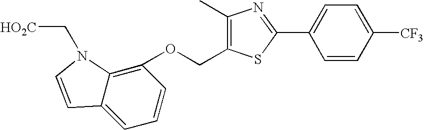

The compound of Example 6 was prepared using the procedure as in Example 3, Steps 1-3, but substituting 1H-indole-7-ol for 1H-indole-5-ol in Step 1. ¹H NMR (400 MHz, CDCl₃), δ (ppm): 7.87 (d, 2H), 7.52 (d, 2H), 7.26 (d, 1H), 6.96 (t, 1H), 6.93 (s, 1H), 6.67 (d, 1H), 6.51 (s, 1H), 5.22 (s, 2H), 5.07 (s, 1H), 2.47 (s, 3H).

### SYNTHESES OF EXAMPLE 7

### {5-[4-Methyl-2-(4-trifluoromethyl-phenyl)-thiazol-5-ylmethoxy]-1H-indol-3-yl}-acetic acid

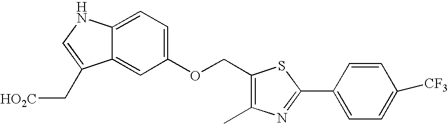

The compound of Example 7 was prepared using the procedure as in Example 1, Steps 3-4, but substituting methyl (5-hydroxy-1H-indol-3-yl)acetate for methyl 3-hydroxyphenylacetate in Step 1. ¹H NMR (400 MHz, CDCl₃), δ (ppm): 8.02 (d, 2H), 7.67 (d, 2H), 7.27 (d, 1H), 7.19 (s, 1H), 7.17 (s, 1H), 6.92 (d, 1H), 5.24 (s, 2H), 3.74 (s, 2H), 2.50 (s, 3H).

### SYNTHESES OF EXAMPLE 8

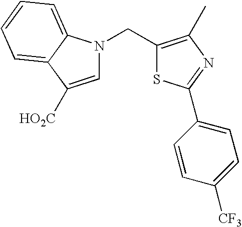

The compound of Example 8 was prepared using the procedure in Example 1, Steps 3-4, but substituting 1H-indole-3-carboxylic acid methyl ester for methyl 3-hydroxyphenylacetate in Step 1. ¹H NMR (400 MHz, CDCl₃), δ (ppm): 8.25 (m, 1H), 7.97 (d, 2H), 7.94 (s, 1H), 7.66 (d, 2H), 7.54 (m, 1H), 7.44 (m, 2H), 5.51 (s, 2H), 2.61 (s, 3H).

### SYNTHESES OF EXAMPLE 9

### 1-[4-methyl-2-(4-trifluoromethyl-phenyl)-thiazol-5-ylmethyl]-1H-indole-4-carboxylic acid

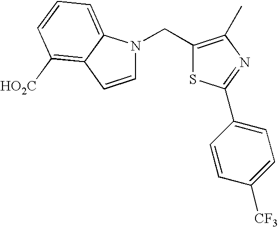

The compound of Example 9 was prepared using the procedure in Example 1, Steps 3-4, but substituting 1H-indole-3-carboxylic acid methyl ester for methyl 4-hydroxyphenylacetate in Step 1. ¹H NMR (400 MHz, CDCl₃), δ (ppm): 8.01 (m, 1H), 7.93 (d, 2H), 7.63 (m, 3H), 7.29 (m, 3H), 5.51 (s, 2H), 2.59 (s, 3H).

### SYNTHESES OF EXAMPLE 10

### 1-[4-methyl-2-(4-trifluoromethyl-phenyl)-thiazol-5-ylmethyl]-1H-indole-5-carboxylic acid

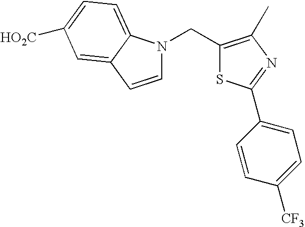

The compound of Example 10 was prepared using the procedure in Example 1, Steps 3-4, but substituting 1H-indole-3-carboxylic acid methyl ester for methyl 5-hydroxyphenylacetate in Step 1. 1H NMR (400 MHz, MeOD), δ (ppm): 8.56 (s, 1H), 8.31 (s, 1H), 8.01 (d, 2H), 7.91 (d, 2H), 7.77 (d, 2H), 7.48 (d, 1H), 6.64 (m, 1H), 5.65 (s, 2H), 2.60 (s, 3H).

### SYNTHESES OF EXAMPLES 11-24

The syntheses of Examples 11-24 are described herein with reference to Schemes XIV-XVII.

### SYNTHESIS OF EXAMPLE 11

### {1-[4-Methyl-2-(4-trifluoromethyl-phenyl)-thiazol-5-ylmethyl]-1H-indol-4-yloxy}acetic acid

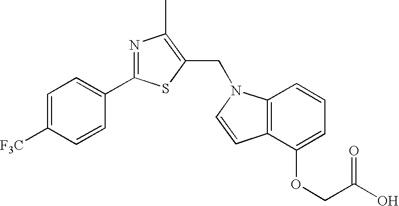

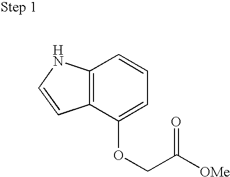

(1H-Indol-4-yloxy)-acetic acid methyl ester (46a): Cesium carbonate (1.89 g, 5.8 mmol, 1.5 equiv) was added to a suspension of 4-hydroxyindole (45a) (514 mg, 3.86 mmol) in 20 mL of dry acetonitrile at rt. The solution was stirred for 5 min and then methylbromoacetate (390 μl, 4.2 mmol, 1.1 equiv) was added and stirred for an additional 0.5 h. The resulting solution was diluted with EtOAc (200mL) and subsequently washed with water (2×100 mL) and brine (100 mL). The organic layer was dried (Na₂SO₄), filtered and concentrated. The crude product was purified by silica gel flash column chromatography (40% EtOAc in Hexanes) to afford the desired product 46a (630 mg, 78%) as a white solid. MS: 206.02 (M+1).
Step 2

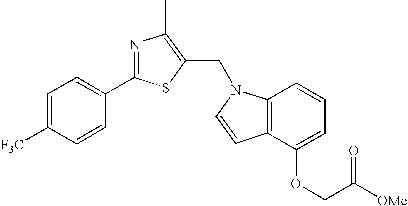

{1-[4-Methyl-2-(4-trifluoromethyl-phenyl)-thiazol-5-ylmethyl]-1H-indol-4-yloxy}-acetic acid methyl ester (47a): Cesium carbonate (170 mg, 0.52 mmol, 2 equiv) was added to a solution of 2-chloromethyl-4-methyl-5-(4-trifluoromethyl-phenyl)-thiazole (100 mg, 0.34 mmol, 1.3 equiv) and 46a (56 mg, 0.27 mmol) in 2 mL of dry acetonitrile and stirred overnight at 70° C. The mixture was diluted with EtOAc (40 mL), washed with water (30 mL) and brine (30 mL). The organic layer was dried (Na₂SO₄), concentrated and purified by silica gel flash column chromatography (25% EtOAc in Hexanes) to afford the desired product 47a (65 mg, 51%) as a light yellow solid. MS: 460.83 (M+1).
Step 3

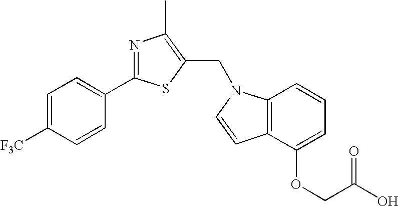

{1-[4-Methyl-2-(4-trifluoromethyl-phenyl)-thiazol-5-ylmethyl]-1H-indol-4-yloxy} acetic acid (48a): Lithium Hydroxide (1M in H₂O, 560 μl, 0.56 mmol, 4 equiv) was added to a stirring solution of 47a (65 mg, 0.14 mmol) in a 2 mL of THF/MeOH (3:1 (v/v)) at rt. After the starting material was consumed (tlc) the reaction was neutralized with 1N HCl, diluted with EtOAc (40 mL) and subsequently washed with water (30 mL) and brine (30 mL). The organic layer was dried (Na2SO4), filtered and concentrated. The crude product was further purified by silica gel flash column chromatography (dichloromethane/MeOH/AcOH 92:7.5:0.5) to provide the desired product 48a (35 mg, 56%) as a white solid. MS: 446.87 (M+1).

### SYNTHESIS OF EXAMPLE 12

### {1-[4-Methyl-2-(4-trifluoromethyl-phenyl)-thiazol-5-ylmethyl]-1H-indol-5-yloxy}-acetic acid

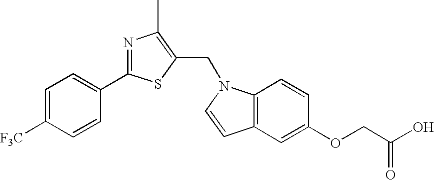

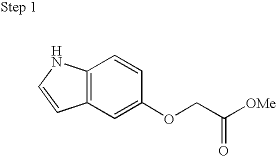

(1H-Indol-5-yloxy)-acetic acid methyl ester (46b): Compound 46b was prepared according to the method for 46a utilizing 5-hydroxyindole. Compound 46b was prepared in 78% yield. MS: 206.02 (M+1).

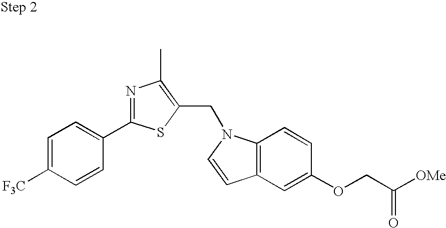

{1-[4-Methyl-2-(4-trifluoromethyl-phenyl)-thiazol-5-ylmethyl]-1H-indol-5-yloxy}-acetic acid methyl ester (47b): Compound 47b was prepared according to the method for 47a utilizing compound 46b as the starting material. Compound 47b was prepared in 29% yield. MS: 460.90 (M+1).

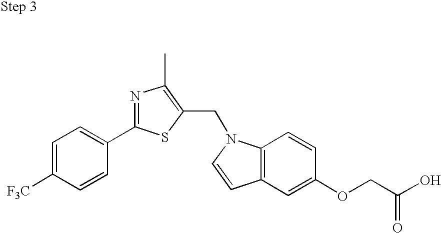

{1-[4-Methyl-2-(4-trifluoromethyl-phenyl)-thiazol-5-ylmethyl]-1H-indol-5-yloxy}-acetic acid (48b): Compound 48b was prepared according to the method for compound 48a utilizing compound 47b as the starting material. Compound 48b was prepared in 79% yield. MS: 446.79 (M+1).

### SYNTHESIS OF EXAMPLE 13

### {1-[4-Methyl-2-(4-trifluoromethyl-phenyl)-thiazol-5-ylmethyl]-1H-indol-6-yloxy}-acetic acid

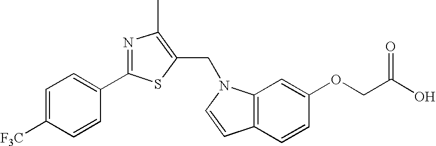

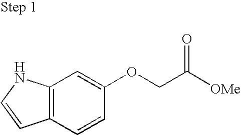

(1H-Indol-6-yloxy)-acetic acid methyl ester (46c): Compound 46c was prepared according to the method for 46a utilizing 6-hydroxyindole. Compound 46c was prepared in 82% yield.

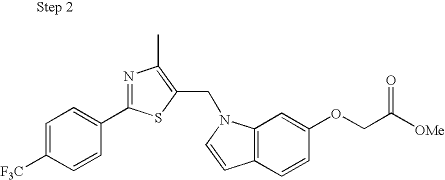

{1-[4-Methyl-2-(4-trifluoromethyl-phenyl)-thiazol-5-ylmethyl]-1H-indol-6-yloxy}-acetic acid methyl ester (47c): Compound 47c was prepared according to the method for 47a utilizing compound 46c as the starting material. Compound 47c was prepared in 44% yield. MS: 460.90 (M+1).

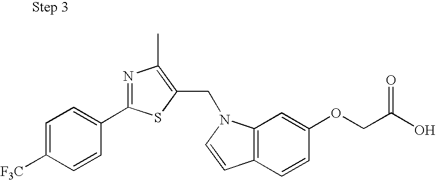

{1-[4-Methyl-2-(4-trifluoromethyl-phenyl)-thiazol-5-ylmethyl]-1H-indol-6-yloxy}-acetic acid (48c): Compound 48c was prepared according to the method for compound 48a utilizing compound 47c as the starting material. Compound 48c was prepared in 64% yield. MS: 446.87 (M+1).

### SYNTHESIS OF EXAMPLE 14

### {1-[4-Methyl-2-(4-trifluoromethyl-phenyl)-thiazol-5-ylmethyl]-1H-indol-7-yloxy}-acetic acid

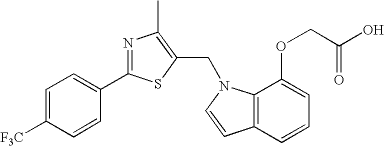

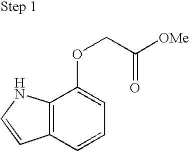

(1H-Indol-7-yloxy)-acetic acid methyl ester (46d): Compound 46d was prepared according to the method for 46a utilizing 7-hydroxyindole. Compound 46d was prepared in 53% yield.

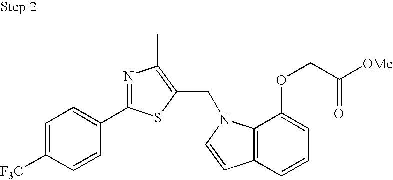

{1-[4-Methyl-2-(4-trifluoromethyl-phenyl)-thiazol-5-ylmethyl]-1H-indol-7-yloxy}-acetic acid methyl ester (47d): Compound 47d was prepared according to the method for 47a utilizing compound 46d as the starting material. Compound 47d was prepared in 18% yield. MS: 460.87 (M+1).

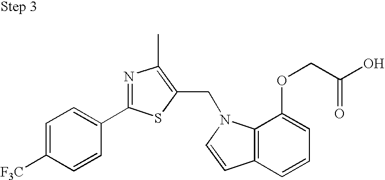

{1-[4-Methyl-2-(4-trifluoromethyl-phenyl)-thiazol-5-ylmethyl]-1H-indol-7-yloxy}-acetic acid (48d): Compound 48d was prepared according to the method for compound 48a utilizing compound 47d as the starting material. Compound 48d was prepared in 75% yield. MS: 446.87 (M+1).

### SYNTHESIS OF EXAMPLE 15

### 2-Methyl-2-{1-[4-methyl-2-(4-trifluoromethyl-phenyl)-thiazol-5-ylmethyl]-1H-indol-5-yloxy}-propionic acid

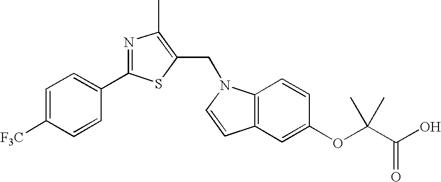

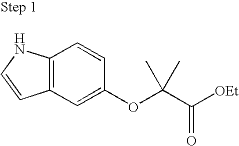

2-(1H-Indol-5-yloxy)-2-methyl-propionic acid ethyl ester (49b): Cesium carbonate (1.45 g, 4.5 mmol, 1.5 equiv) was added to a suspension of 5-hydroxyindole (45b) (395 mg, 2.97 mmol) in 15 mL of dry acetonitrile at rt. Ethyl 2-bromoisobutyrate (480 μl, 3.3 mmol, 1.1 equiv) was added and the solution was stirred overnight at 70° C. The resulting solution was diluted with EtOAc (600 mL) and subsequently washed with water (2×50 mL) and brine (50 mL). The organic layer was dried (Na₂SO₄), filtered and concentrated. The crude product was then purified by silica gel flash column chromatography (20% EtOAc in Hexanes) to afford the desired product 49b (586 mg, 80%) as a colorless liquid. MS: 248.04 (M+1).

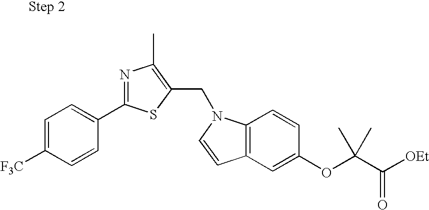

2-Methyl-2-{1-[4-methyl-2-(4-trifluoromethyl-phenyl)-thiazol-5-ylmethyl]-1H-indol-5-yloxy}-propionic acid ethyl ester (50b): Cesium carbonate (461 mg, 1.41 mmol, 2 equiv) was added to a solution of 2-chloromethyl-4-methyl-5-(4-trifluoromethyl-phenyl)-thiazole (271 mg, 0.93 mmol, 1.3 equiv) and 49b (184 mg, 0.74 mmol) in 3 mL of dry acetonitrile and stirred overnight at 70° C. The mixture was diluted with EtOAc (60 mL), washed with water (60 mL) and brine (60 mL). The organic layer was dried (Na₂SO₄), concentrated and purified by silica gel flash column chromatography (15% EtOAc in Hexanes) to afford the desired product 50b (252 mg, 67%) as a light yellow oil. MS: 503.51 (M+1).

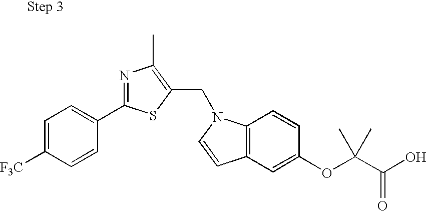

2-Methyl-2-{1-[4-methyl-2-(4-trifluoromethyl-phenyl)-thiazol-5-ylmethyl]-1H-indol-5-yloxy}-propionic acid (51b): Lithium Hydroxide (IM in H₂O, 2 mL, 2.0 mmol, 4 equiv) was added to a stirring solution of 50b (252 mg, 0.50 mmol) in a 4 mL of THF/MeOH (3:1 (v/v)) at rt. After the starting material was consumed (tlc) the reaction was neutralized with 1N HCl, diluted with EtOAc (50 mL) and subsequently washed with water (50 mL) and brine (SOmL). The organic layer was dried (NaSO₄), filtered and concentrated. The crude product was further purified by silica gel flash column chromatography (dichloromethane/MeOH/AcOH 97:3:0.5) to provide the desired product 51b (175 mg, 74%) as a yellow oil. MS: 474.88 (M+1).

### SYNTHESIS OF EXAMPLE 16

### 2-Methyl-2-{1-[4-methyl-2-(4-trifluoromethyl-phenyl)-thiazol-5-ylmethyl]-1H-indol-6-yloxy}-propionic acid

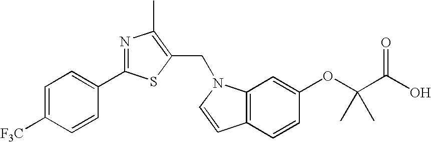

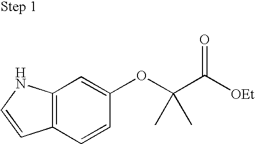

2-(1H-Indol-6-yloxy)-2-methyl-propionic acid ethyl ester (49c): Compound 49c was prepared according to the method for compound 49b utilizing 6-hydroxyindole (45c) as the starting material. Compound 49c was prepared in 54% yield. MS: 247.99 (M+1).

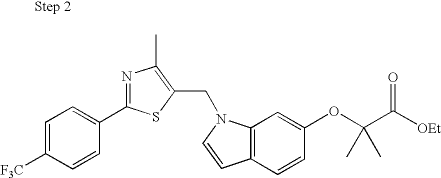

2-Methyl-2-{1-[4-methyl-2-(4-trifluoromethyl-phenyl)-thiazol-5-ylmethyl]-1H-indol-6-yloxy}-propionic acid ethyl ester (50c): Compound 50c was prepared according to the method for compound 50b utilizing 49c as the starting material. Compound 50c was prepared in 67% yield. ¹H NMR (400 MHz, CDCl₃) 7.93 (d, 2H), 7.63 (d, 2H), 7.48 (d, 1H), 7.06 (d, 1H), 6.88 (d, 1H), 6.76 (dd, 1H), 6.49 (dd, 1H), 5.25 (s, 2H), 4.19 (q, 2H), 2.56 (s, 3H), 1.58 (s, 6H), 1.21 (t, 3H); MS: 502.87 (M+1).

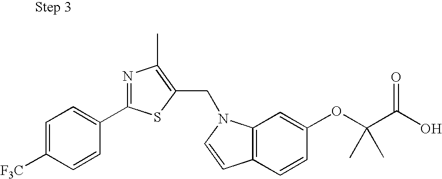

2-Methyl-2-{1-[4-methyl-2-(4-trifluoromethyl-phenyl)-thiazol-5-ylmethyl]-1H-indol-6-yloxy}-propionic acid (51c): Compound 51c was prepared according to the method for compound 51b utilizing compound 50c as the starting material. Compound 51c was prepared in 78% yield. ¹H NMR (400 MHz, CDCl₃) 7.88 (d, 2H), 7.59 (d, 2H), 7.52 (d, 1H), 7.10 (d, 1H), 6.93 (br d, 1H), 6.81 (dd, 1H), 6.52 (dd, 1H), 5.35 (s, 2H), 2.56 (s, 3H), 1.57 (s, 6H); MS: 474.88 (M+1).

### SYNTHESIS OF EXAMPLE 17

### (1-{2-[4-Methyl-2-(4-trifluoromethyl-phenyl)-thiazol-5-yl]-ethyl}-2,3-dihydro-1H-indol-5-yloxy)-acetic acid

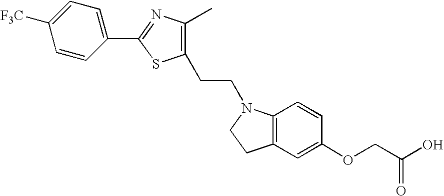

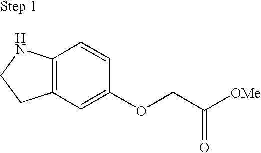

(2,3-Dihydro-1H-indol-5-yloxy)-acetic acid methyl ester (52b): Sodium cyanoborohydride (230 mg, 3.65 mmol, 3 equiv) was added to a stirring solution of 46b in AcOH (10 mL). The reaction was stirred at rt for 0.5 h until no starting material remained. The reaction was concentrated, diluted with EtOAc (100 mL, washed with saturated sodium bicarbonate (100 mL) and brine (100 mL). The organic layer was dried (Na₂SO₄), filtered and concentrated to afford the desired product 52b (214 mg, 85%) as a colorless oil. ¹H NMR (400 MHz, CDCl₃) 6.76 (m, 1H), 6.60-6.53 (m, 2H), 4.53 (s, 2H), 3.78 (s, 3H), 3.55 (br s, 1H), 3.51 (t, 2H), 2.97 (t, 2H).

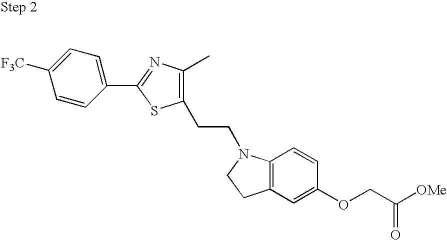

(1-{2-[4-Methyl-2-(4-trifluoromethyl-phenyl)-thiazol-5-yI]-ethyl}-2,3-dihydro-1H-indol-5-yloxy)-acetic acid methyl ester (53b): 2-(4-toluene-sulfonylethyl)-4-methyl-5-(4-trifluoromethyl-phenyl)-thiazole (229 mg, 0.52 mmol) was added to a stirring solution of 52b (214 mg, 1.03 mmol, 2 equiv) in acetonitrile (3 mL). The reaction mixture was irradiated in a microwave reactor for 10 min at 180° C. The reaction was then diluted with EtOAc (50 mL) and then washed with saturated sodium bicarbonate (50 mL) and brine (50 mL). The organic layer was then dried (Na₂SO₄), filtered, concentrated and purified by silica gel flash column chromatography (25% EtOAc in Hexanes) to afford 53b (172 mg, 70%) as a yellow solid. ¹H NMR (400 MHz, CDCl₃) 7.98 (d, 2H), 7.65 (d, 2H), 6.80 (s, 1H), 6.64 (dd, 1H), 6.38 (d, 1H), 4.56 (s, 2H), 3.80 (s, 3H), 3.38 (t, 2H), 3.28 (t, 2H), 3.06 (t, 2H), 2.97 (t, 2H), 2.45 (s, 3H); MS: 477.01 (M+1).

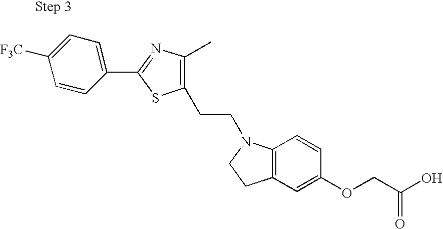

(1-{2-[4-Methyl-2-(4-trifluoromethyl-phenyl)-thiazol-5-yl]-ethyl}-2,3-dihydro-1H-indol-5-yloxy)-acetic acid (54b): 1N LiOH (210 μl, 0.210 mmol, 4equiv) was added to a stirring solution of 53b in 2 mL of a 3:1 (v/v) mixture of THF/MeOH. The resulting solution was stirred for 3 h at rt until no starting material remained. The reaction was then quenched with Dowex 50-WX4-50 resin until neutral, filtered and concentrated. The residue was further purified by silica gel flash column chromatography (95:4:1 dichloromethane/MeOH/AcOH) to afford 54b (5 mg, 21%) as a yellow oil. ¹H NMR (400 MHz, CDCl₃) 7.97 (d, 2H), 7.65 (d, 2H), 6.80 (s, 1H), 6.65 (br d, 1H), 6.39 (br d, 1H), 4.18 (s, 2H), 3.39 (br t, 2H), 3.28 (m, 2H), 3.06 (br t, 2H), 2.98 (br t, 2H), 2.45 (s, 3H); MS: 462.91 (M+1).

### SYNTHESIS OF EXAMPLE 18

### (1-{2-[4-Methyl-2-(4-trifluoromethyl-phenyl)-thiazol-5-yl]-ethyl}-1H-indol-5-yloxy)-acetic acid

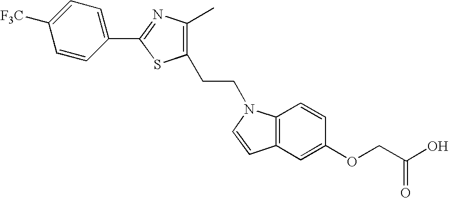

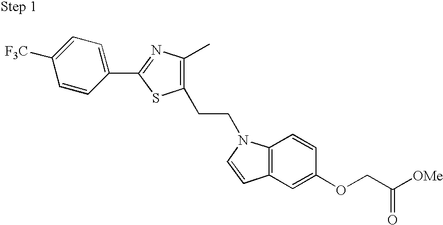

(1-{2-[4-Methyl-2-(4-trifluoromethyl-phenyl)-thiazol-5-yl]-ethyl}-1H-indol-5-yloxy)-acetic acid methyl ester (55b): DDQ (85 mg, 0.372 mmol, 1.3 equiv) was added to a stirring solution of 53b (142 mg, 0.298 mmol) in dichloromethane (5 mL). The reaction mixture was stirred at rt for 1 h. The resulting solution was filtered through a short pug of silica gel (50% EtOAc in hexanes) to afford 55b (79 mg, 56%) as a yellow solid. ¹H NMR (400 MHz, CDCl₃) 7.95 (d, 2H), 7.66 (d, 2H), 7.20 (d, 1H), 7.07 (d, 1H), 6.94 (dd, 1H), 6.90 (d, 1H), 6.38 (d, 1H), 4.68 (s, 2H), 4.34 (t, 2H), 3.82 (s, 3H), 3.26 (t, 2H), 2.09 (s, 3H); MS: 474.88 (M+1).

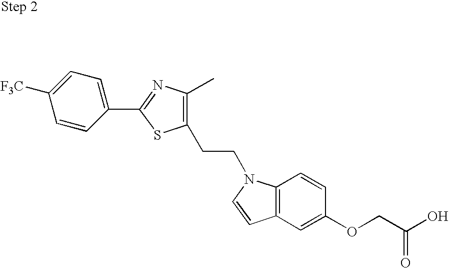

(1-{2-[4-Methyl-2-(4-trifluoromethyl-phenyl)-thiazol-5-yl]-ethyl}-1H-indol-5-yloxy)-acetic acid (56b): 1N LiOH (830 μl, 0.832 mmol, 5 equiv) was added to a stirring solution of 55b in 5 mL of a 4:1 (v/v) mixture of THF/H₂O. The resulting solution was stirred for 1 h at rt until no starting material remained. The reaction was then quenched with Dowex 50-WX4-50 resin until neutral, filtered and concentrated. The residue was then further purified by silica gel flash column chromatography (92:8:0.5 dichloromethane/MeOH/AcOH) to afford 56b (60 mg, 78%) as a white solid. ¹H NMR (400 MHz, CDCl₃) 7.95 (d, 2H), 7.66 (d, 2H), 7.24-6.92 (m, 3H), 6.90 (d, 1H), 6.38 (d, 1H), 4.70 (s, 2H), 4.35 (t, 2H), 3.26 (t, 2H), 2.07 (s, 3H); MS: 460.88 (M+1).

### SYNTHESIS OF EXAMPLE 19

### 2-Methyl-2-(1-{2-[4-methyl-2-(4-trifluoromethyl-phenyl)-thiazol-5-yl]-ethyl}-2,3-dihydro-1H-indol-5-yloxy)-propionic acid

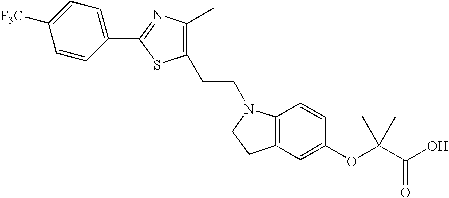

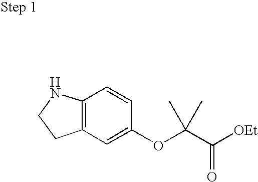

2-(2,3-Dihydro-1H-indol-5-yloxy)-2-methyl-propionic acid ethyl ester (57b): Compound 57b was prepared according to the method for compound 52b utilizing compound 49b as the starting material. Compound 57b was prepared in 92% yield. ¹H NMR (400 MHz, CDCl₃) 6.71 (m, 1H), 6.56 (m, 1H), 6.49 (m, 1H), 4.22 (q, 2H), 3.64 (s, 1H), 3.51 (t, 2H), 2.95 (t, 2H), 1.48 (s, 6H), 1.29 (t, 3H).

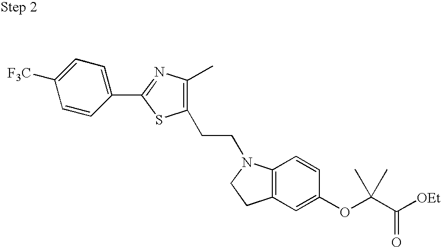

2-Methyl-2-(1-{2-[4-methyl-2-(4-trifluoromethyl-phenyl)-thiazol-5-yl]-ethyl)-2,3-dihydro-1H-indol-5-yloxy)-propionic acid ethyl ester (58b): Compound 58b was prepared according to the method for compound 53b utilizing compound 57b as the starting material. Compound 58b was prepared in 43% yield. ¹H NMR (400 MHz, CDCl₃) 7.98 (d, 2H), 7.64 (d, 2H), 6.74 (s, 1H), 6.64 (d, 1H), 6.32 (d, 1H), 4.24 (q, 2H), 3.37 (t, 2H), 3.28 (t, 2H), 3.05 (t, 2H), 2.94 (t, 2H), 2.45 (s, 3H), 1.52 (s, 6H), 1.29 (t, 3H).

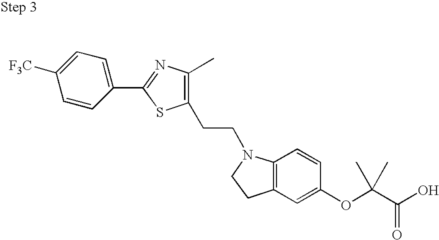

2-Methyl-2-(1-{2-[4-methyl-2-(4-trifluoromethyl-phenyl)-thiazol-5-yl]-ethyl})-2,3-dihydro-1H-indol-5-yloxy)-propionic acid (59b): Compound 59b was prepared according to the method for compound 51b utilizing compound 58b as the starting material. Compound 59b was prepared in 70% yield. ¹H NMR (400 MHz, CDCl₃) 7.98 (d, 2H), 7.65 (d, 2H), 6.77 (s, 1H), 6.70 (d, 1H), 6.34 (d, 1H), 3.40 (t, 2H), 3.31 (t, 2H), 3.07 (t, 2H), 2.97 (t, 2H), 2.45 (s, 3H), 1.52 (s, 6H).

### SYNTHESIS OF EXAMPLE 20

### 2-Methyl-2-(1-{2-[4-methyl-2-(4-trifluoromethyl-phenyl)-thiazol-5-yl]-ethyl}-2,3-dihydro-1H-indol-6-yloxy)-propionic acid

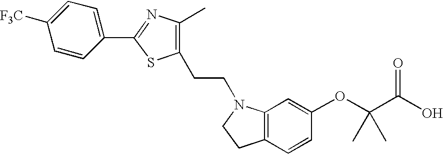

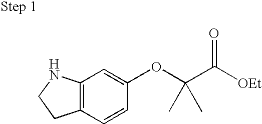

2-(2,3-Dihydro-1H-indol-6-yloxy)-2-methyl-propionic acid ethyl ester (57c): Compound 57c was prepared according to the method for compound 52b utilizing compound 49c as the starting material. Compound 57c was prepared in 92% yield. ¹H NMR (400 MHz, CDCl₃) 6.90 (m, 1H), 6.16-6.14 (m, 2H), 4.21 (q, 2H), 3.68 (s, 1H), 3.51 (t, 2H), 2.91 (t, 2H), 1.52 (s, 6H), 1.25 (t, 3H).

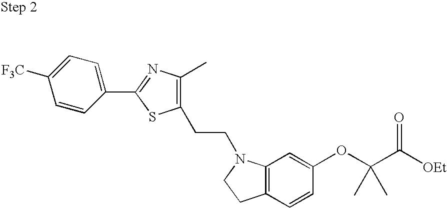

2-Methyl-2-(1-{2-[4-methyl-2-(4-trifluoromethyl-phenyl)-thiazol-5-yl]-ethyl}-2,3-dihydro-1H-indol-6-yloxy)-propionic acid ethyl ester (58c): Compound 58c was prepared according to the method for compound 53b utilizing compound 57c as the starting material. Compound 58b was prepared in 41% yield. ¹H NMR (400 MHz, CDCl₃) 7.98 (d, 2H), 7.65 (d, 2H), 6.91 (d, 1H), 6.11 (dd, 1H), 6.06 (s, 1H), 4.21 (q, 2H), 3.41 (t, 2H), 3.29 (t, 2H), 3.05 (t, 2H), 2.93 (t, 2H), 2.45 (s, 3H), 1.56 (s, 6H), 1.24 (t, 3H); MS: 518.91 (M+1).

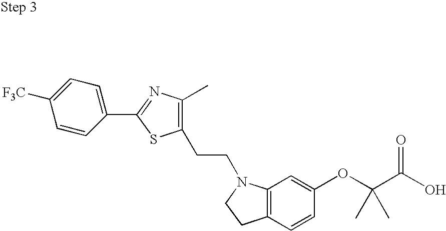

2-Methyl-2-(1-{2-[4-methyl-2-(4-trifluoromethyl-phenyl)-thiazol-5-yl]-ethyl}-2,3-dihydro-1H-indol-6-yloxy)-propionic acid (59c): Compound 59c was prepared according to the method for compound 51b utilizing compound 58c as the starting material. Compound 59c was prepared in 7% yield. ¹H NMR (400 MHz, CDCl₃) 7.98 (d, 2H), 7.65 (d, 2H), 6.94 (m, IH), 6.22 (m, 1H), 6.02 (s, 1H), 3.46 (t, 2H), 3.31 (t, 2H), 3.06 (t, 2H), 2.96 (t, 2H), 2.44 (s, 3H), 1.56 (s, 6H).

### SYNTHESIS OF EXAMPLE 21

### 2-Methyl-2-(1-{2-[4-methyl-2-(4-trifluoromethyl-phenyl)-thiazol-5-yl]-ethyl}-1H-indol-5-yloxy)-propionic acid

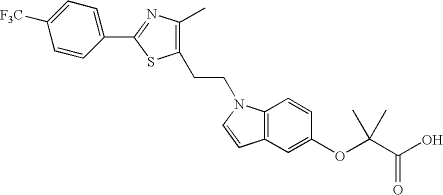

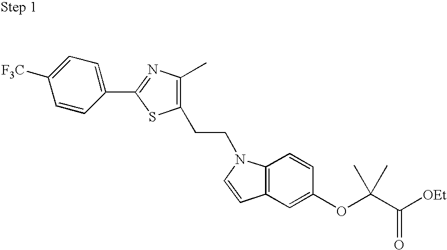

2-Methyl-2-(1-{2-[4-methyl-2-(4-trifluoromethyl-phenyl)-thiazol-5-yl]-ethyl)-1H-indol-5-yloxy)-propionic acid ethyl ester (60b): Tetrachloro-1,4-benzoquinone (59 mg, 0.241 mmol) was added to a stirring solution of 58b (125 mg, 0.241 mmol) at rt. The resulting solution was stirred for 2 h upon which more tetrachloro-1,4-benzoquinone (59 mg, 0.241 mmol) was added. The reaction was stirred an additional 2 h, concentrated and directly purified by silica gel flash column chromatography (20% EtOAc in hexanes) to afford 60b (77 mg, 62%). ¹H NMR (400 MHz, CDCl₃) 7.94 (d, 2H), 7.64 (d, 2H), 7.13 (m, 2H), 6.87 (m, 2H), 6.35 (d, 1H), 4.33-4.24 (m, 4H), 3.25 (t, 2H), 2.06 (s, 3H), 1.56 (s, 6H), 1.29 (t, 3H).

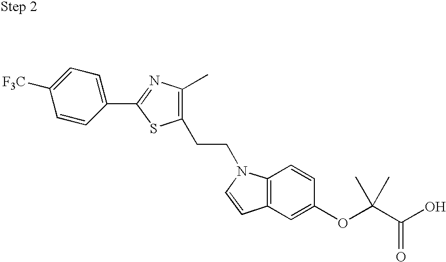

2-Methyl-2-(1-{2-[4-methyl-2-(4-trifluoromethyl-phenyl)-thiazol-5-yl]-ethyl}-1H-indol-5-yloxy)-propionic acid (61b): Compound 61b was prepared according to the method for compound 51b utilizing compound 60b as the starting material. Compound 61b was prepared in 98% yield. ¹H NMR (400 MHz, CDCl₃) 7.92 (d, 2H), 7.62 (d, 2H), 7.25-7.13 (m, 2H), 6.92-6.88 (m, 2H), 6.38 (d, 1H), 4.32 (t, 2H), 3.24 (t, 2H), 2.07 (s, 3H), 1.56 (s, 6H); MS: 488.98 (M+1).

### SYNTHESIS OF EXAMPLE 22

### (1-{2-[4-Methyl-2-(4-trifluoromethyl-phenyl)-thiazol-5-yl]-ethyl}-1H-indol-6-yloxy)-acetic acid

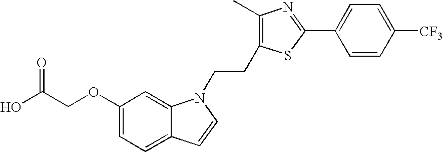

Example 22 was prepared according to a method analogous to that used in Example 18 utilizing compound 46c as the starting material. ¹H NMR (400 MHz, CDCl₃) 7.88 (d, 2H), 7.62 (d, 2H), 7.51 (d, 1H), 6.85 (d, 1H), 6.80 (dd, 1H), 6.74 (s, 1H), 6.43 (d, 1H), 4.65 (s, 2H), 4.26 (t, 2H), 3.21 (t, 2H), 2.09 (s, 3H); MS: 460.89 (M+1).

### SYNTHESIS OF EXAMPLE 23

### 2-Methyl-2-(1-{2-[4-methyl-2-(4-trifluoromethyl-phenyl)-thiazol-5-yl]-ethyl}-1H-indol-6-yloxy)-propionic acid

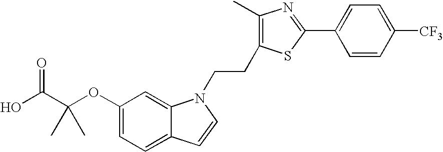

Example 23 was prepared according to a method analogous to that used in Example 21 utilizing compound 49c as the starting material. ¹H NMR (400 MHz, CDCl₃) 7.90 (d, 2H), 7.61 (d, 2H), 7.47 (d, 1H), 6.88 (d, 1H), 6.86 (s, 1H), 6.78 (dd, 1H), 6.42 (d, 1H), 4.26 (t, 2H), 3.21 (t, 2H), 2.06 (s, 3H), 1.55 (s, 6H); MS: 488.99 (M+1).

### SYNTHESIS OF EXAMPLE 24

### {1-[4-Methyl-2-(4-trifluoromethyl-phenyl)-thiazol-5-ylmethyl]-1H-indol-3-yl}acetic acid

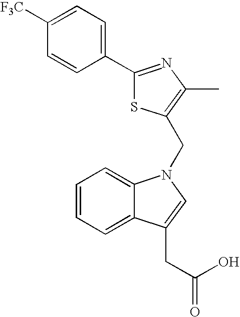

Example 24 was prepared according to a method analogous to that used in Example 8 utilizing 1H-indol-3-yl-acetic acid methyl ester instead of 1H-indol-3-carboxylic acid methyl ester as the starting material. Example 24 was prepared in 42% yield (two steps). ¹H NMR (400 MHz, CDCl₃), δ (ppm): 7.95 (d, 2H), 7.65 (m, 3H), 7.40 (t, 1H), 7.20 (m, 3H), 5.40 (s, 2H), 3.85 (s, 2H), 2.60 (s, 3H).

### SYNTHESIS OF EXAMPLE 25

### (1-2-[4-Methyl-2-(4-trifluoromethyl-phenyl)-thiazol-5-yl]-ethyl}-2,3-dihydro-1H-indol-6-yloxy)-acetic acid

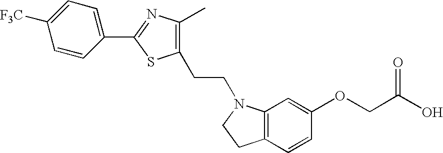

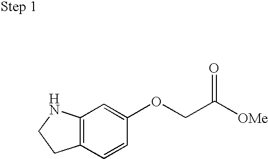

(2,3-Dihydro-1H-indol-6-yloxy)-acetic acid methyl ester (52c): Compound 52c was prepared according to the method for compound 52b utilizing compound 46c as the starting material. Compound 52c was prepared in >99% yield. ¹H NMR (400 MHz, CDCl₃) 6.97 (d, 1H), 6.25 (d, 1H), 6.21 (dd, 1H), 4.58 (s, 2H), 3.80 (s, 3H), 3.56 (t, 2H), 2.95 (t, 2H). 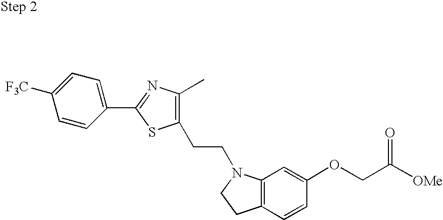
ethyl}-2,3-was prepared according to the method for compound 53b utilizing compound 52c as the starting material. Compound 53c (137 mg) was used semi-crude (mixture of product and starting material) in the next reaction. MS: 476.84 (M+1).

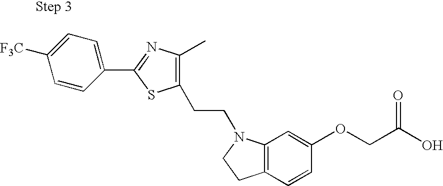

(1-{2-[4-Methyl-2-(4-trifluoromethyl-phenyl)-thiazol-5-yl]-ethyl}-2,3-dihydro-1H-indol-6-yloxy)-acetic acid (54c): Compound 54c was prepared according to the method for compound 54b utilizing compound 53c as the starting material. Compound 54c was prepared in 35% yield. ¹H NMR (400 MHz, CDCl₃) 7.96 (d, 2H), 7.65 (d, 2H), 6.96 (d, 1H), 6.14 (d, 1H), 6.07 (s, 1H), 4.59 (s, 2H), 3.44 (t, 2H), 3.32 (t, 2H), 3.05 (t, 2H), 2.94 (t, 2H), 2.44 (s, 3H); MS: 462.90 (M+1).

### SYNTHESES OF INTERMEDIATES

The heterocyclic and coupled phenyl ring components corresponding to element [C] as claimed herein may be prepared in the following schemes. By varying the R and Z groups in Scheme XVIII, a variety of substituted oxazole-phenyl compounds may be at once envisioned: 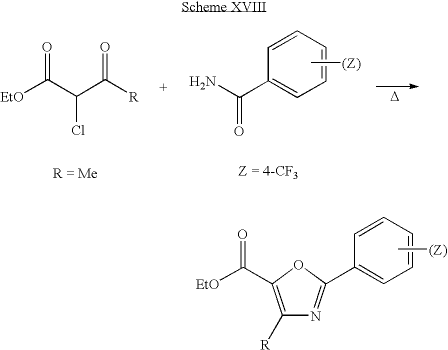
Synthesis of Intermediate (I-4), 1H-Indol-7-ol

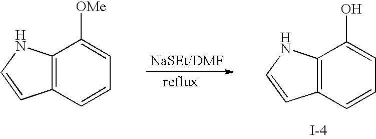

1H-Indol-7-ol (I-4). To a solution of 7-Methoxy-1H-indole (2.0 g, 13.58 mmol, 1.0 equiv.) in DMF (20 mL) was added NaSEt (2.8 g, 34.0 mmol, 2.5 equiv.). The resulting mixture was heated to 155° C. under N₂ with stirring. After stirred at same temperature for 15 h. the reaction mixture was cooled to room temperature, neutralized with 1N HCl (34 mL). The resulting mixture was partitioned with ethyl acetate (300 mL). After separation, the organic layer was washed with water, brine, dried over Na₂SO₄. After removal of solvent, the crude product was purified by chromatography to afford 1.337 g (74% yield) of intermediate I-4 as black solid. ¹H NMR (400 MHz, CDCl₃), δ (ppm): 8.45 (b, 1H), 7.27 (d, 1H), 7.23 (s, 1H), 6.98 (t, 1H), 6.61 (d, 1H), 6.57 (s, 1H), 3.90 (b, 1H).
Synthesis of Intermediate (I-5), 1H-Indol-6-ol

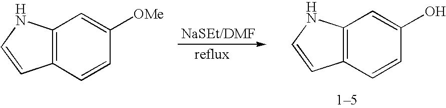

The intermediate I-5 was a bright brown solid. Which was prepared followed the procedure described for intermediate I-4 with 27% yield. ¹H NMR (400 MHz, CDCl₃), δ (ppm): 8.00 (b, 1H), 7.11 (s, 1H), 6.87 (s, 1H), 6.71 (d, 1H), 6.61 (d, 1H), 6.50 (s, 1H).

### GENERAL METHOD FOR PREPARING SUBSTITUTED INDOLE ISOMERS

Several embodiments of the invention were prepared according the Scheme XIX

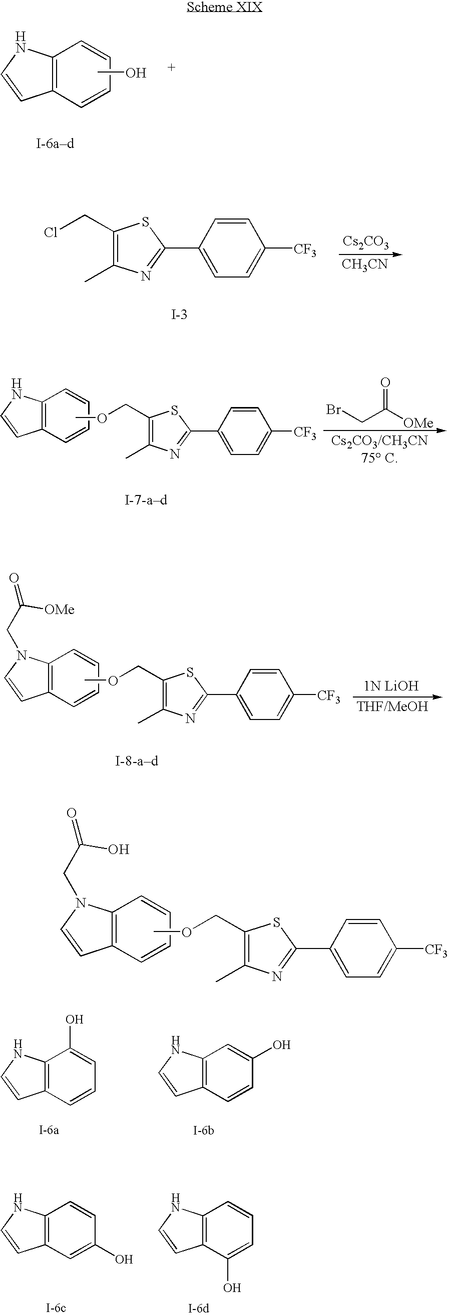

Scheme XIX depicts the parallel synthesis of intermediates I-7(a-d). Intermediates I-6(a-d) (1.2 mmol, 1.2 equiv) were charged in 4 reaction vials, respectively. To each of these vials was-added 2 mL of solution of intermediate I-3 in CH₃CN (1.0 mmol, 1.0 equiv) (prepared by dissolving 1.71 g (6.0 mmol) of I-3 in 12 mL of CH₃CN) followed by Cs₂CO₃ (490 mg, 1.5 mmol, 1.5 equiv.). The resulting suspensions were further diluted by addition of 8 mL of CH₃CN and then stirred at room temperature for 21 h. The reaction mixtures were concentrated under an N₂ stream and the residues were diluted with ethyl acetate (15 mL), washed with water, brine, dried over Na₂SO₄. After removal of solvent, the crude products were purified by chromatography. Their ¹H NMR data were described as below.

SYNTHESIS OF INTERMEDIATE (I-7a) 7-[4-Methyl-2-(4-trifluoromethyl-phenyl)-thiazol-5-ylmethoxy]-1H-indole. ¹H NMR (400 MHz, CDCl₃), δ (ppm): 8.45 (b, 1H), 8.04 (d, 2H), 7.69 (d, 2H), 7.33 (d, 1H), 7.20 (s, 1H), 7.06 (t, 1H), 6.76 (d, 1H), 6.56 (s, 1H), 5.37 (s, 2H), 2.55 (s, 3H).

SYNTHESIS OF INTERMEDIATE (I-7b) 6-[4-Methyl-2-(4-trifluoromethyl-phenyl)-thiazol-5-ylmethoxy]-1H-indole. ¹H NMR (400 MHz, CDCl₃), δ (ppm): 8.10 (b, 1H), 8.02 (d, 2H), 7.68 (d, 2H), 7.53 (d, 1H), 7.10 (s, 1H), 6.99 (s, 1H), 6.87 (d, 1H), 6.49 (s, 1H), 5.24 (s, 2H), 2.53 (s, 3H).

SYNTHESIS OF INTERMEDIATE (I-7c) 5-[4-Methyl-2-(4-trifluoromethyl-phenyl)-thiazol-5-ylmethoxy]-1H-indole. ¹H NMR (400 MHz, CDCl₃), δ (ppm): 8.12 (b, 1H), 8.03 (d, 2H), 7.68 (d, 2H), 7.29 (d, 1H), 7.22(s, 2H), 6.91 (d, 1H), 6.51 (s, 1H), 5.25 (s, 2H), 2.52 (s, 3H).

SYNTHESIS OF INTERMEDIATE (I-7d) 4-[4-Methyl-2-(4-trifluoromethyl-phenyl)-thiazol-5-ylmethoxy]-1H-indole. ¹H NMR (400 MHz, CDCl₃), δ (ppm): 8.25 (b, 1H), 8.04 (d, 2H), 7.68 (d, 2H), 7.13 (m, 3H), 6.69 (s, 1H), 2H), 6.64 (d, 1H), 5.37 (s, 2H), 2.55 (s, 3H).

### PARALLEL SYNTHESES OF INTERMEDIATES I-8(a-d)

Solutions of intermediates I-7(a-d) (0.27 mmol, 1.0 equiv) in 5 mL of CH₃CN were charged in 4 reaction vials, respectively. To each of these vials was added methyl bromoacetate (50 μL, 0.54 mmol, 2.0 equiv) followed by Cs₂CO₃ (133 mg, 0.40 mmol, 1.5 equiv.). After the vials were capped, the resulting suspensions were heated to 75° C. then stirred at the same temperature for 24 h. The reaction mixtures were cooled to room temperature and then filtered. The organic solution was concentrated under N₂ blow and the residues were purified by chromatography. Their ¹H NMR data were described as below.

{7-[4-methyl-2-(4-trifluoromethyl-phenyl)-thiazol-5-ylmethoxy]-indol-1-yl}-acetic acid methyl ester (I-8a). ¹H NMR (400 MHz, CDCl₃), δ (ppm): 8.06 (d, 2H), 7.72 (d, 2H), 7.29 (d, 1H), 7.05 (t, 1H), 6.96 (s, 1H), 6.75 (d, 1H), 6.53 (s, 1H), 5.29 (s, 2H), 5.09 (s, 1H), 3.59 (s, 3H), 2.55 (s, 3H).

{6-[4-methyl-2-(4-trifluoromethyl-phenyl)-thiazol-5-ylmethoxy]-indol-1-yl}-acetic acid methyl ester (I-8b). ¹H NMR (400 MHz, CDCl₃), δ (ppm): 8.04 (d, 2H), 7.69 (d, 2H), 7.54 (d, 1H), 7.04 (s, 1H), 6.92 (d, 1H), 6.83 (s, 1H), 6.54 (s, 1H), 5.27 (s, 2H), 4.83 (s, 1H), 3.77 (s, 3H), 2.54 (s, 3H).

{5-[4-methyl-2-(4-trifluoromethyl-phenyl)-thiazol-5-ylmethoxy]-indol-1-yl}-acetic acid methyl ester (I-8c). ¹H NMR (400 MHz, CDCl₃), δ (ppm): 8.04 (d, 2H), 7.69 (d, 2H), 7.23 (s, 1H), 7.21 (d, 1H), 7.11 (s, 1H), 6.96 (d, 1H), 6.53 (s, 1H), 5.26 (s, 2H), 4.86 (s, 1H), 3.77 (s, 3H), 2.54 (s, 3H).

{4-[4-methyl-2-(4-trifluoromethyl-phenyl)-thiazol-5-ylmethoxy]-indol-1-yl}-acetic acid methyl ester (I-8d). ¹H NMR (400 MHz, CDCl₃), δ (ppm): 8.05 (d, 2H), 7.69 (d, 2H), 7.17 (t, 1H), 7.04 (s, 1H), 6.95 (d, 1H), 6.70 (m, 2H), 5.38 (s, 2H), 4.87 (s, 1H), 3.77 (s, 3H), 2.56 (s, 3H).

### PARALLEL SYNTHESIS OF COMPOUNDS I-9(a-d)

To 4 reaction vials charged with intermediates I-8(a-d) (1.0 equiv.), respectively, were added THF/MeOH (3:1) (3 mL) followed by 1N LiOH (5.0 equiv.). The resulting mixtures were stirred at room temperature for 48 h. The reaction mixtures were concentrated under N₂ blow and residues were diluted with water (2 mL). The aqueous layers were partitioned with ether (2 mL). After removal of organic layers, the aqueous layers were neutralized by 1N HCl and then extracted with ethyl acetate (5 mL). The organic layers were washed with water, brine, dried over Na₂SO₄. Removal of solvent affords compounds 9a-d. Their ¹H NMR data were described as below.

{7-[4-methyl-2-(4-trifluoromethyl-phenyl)-thiazol-5-ylmethoxy]-indol-1-yl}-acetic acid (9a). ¹H NMR (400 MHz, CDCl₃), δ (ppm): 7.87 (d, 2H), 7.52 (d, 2H), 7.26 (d, 1H), 6.96 (t, 1H), 6.93 (s, 1H), 6.67 (d, 1H), 6.51 (s, 1H), 5.22 (s, 2H), 5.07 (s, 1H), 2.47 (s, 3H).

{6-[4-methyl-2-(4-trifluoromethyl-phenyl)-thiazol-5-ylmethoxy]-indol-1-yl}-acetic acid (9b). ¹H NMR (400 MHz, CDCl₃), δ (ppm): 8.07 (d, 2H), 7.71 (d, 2H), 7.51 (d, 1H), 7.08 (s, 1H), 6.92 (s, 1H), 6.84 (d, 1H), 6.47 (s, 1H), 5.29 (s, 2H), 4.84 (s, 1H), 2.55 (s, 3H).

{5-[4-methyl-2-(4-trifluoromethyl-phenyl)-thiazol-5-ylmethoxy]-indol-1-yl}-acetic acid (9c). ¹H NMR (400 MHz, CDCl₃), δ (ppm): 8.00 (d, 2H), 7.66 (d, 2H), 7.21 (s, 1H), 7.16 (d, 1H), 7.07 (s, 1H), 6.96 (d, 1H), 6.51 (s, 1H), 5.23 (s, 2H), 4.87 (s, 1H), 2.51 (s, 3H).

{4-[4-methyl-2-(4-trifluoromethyl-phenyl)-thiazol-5-ylmethoxy]-indol-1-yl}-acetic acid (9d). ¹H NMR (400 MHz, CDCl₃), δ (ppm): 8.01 (d, 2H), 7.66 (d, 2H), 7.17 (t, 1H), 7.00 (s, 1H), 6.93 (d, 1H), 6.68 (s, 1H), 6.65 (d, 2H), 5.34 (s, 2H), 4.86 (s, 1H), 2.53 (s, 3H).
Synthesis of (3-Hydroxy-phenyl)-acetic acid methyl ester (I-4)

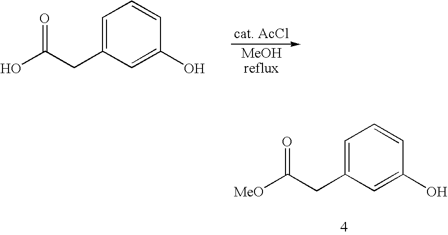

This intermediate is used in the preparation of Example 1

To a solution of (3-Hydroxy-phenyl)-acetic acid (75.87 g, 499 mmol) in MeOH (300 mL) was added acetyl chloride (0.5 mL). The resulting mixture was heated to reflux under N₂ with stirring. After refluxing for 5h, the reaction mixture was concentrated under reduced pressure. The residue was purified by flash chromatography to afford 82.28 g (99% yield) of intermediate I-4 as colorless oil. ¹H NMR (400 MHz, CDCl₃), δ (ppm): 7.18 (t, 1H), 6.82 (d, 1H), 6.70 (m, 2H), 5.33 (b, 1H), 3.70 (s, 3H), 3.55 (s, 2H).
Synthesis of (3-Mercapto-phenyl)-acetic acid methyl ester (I-5)

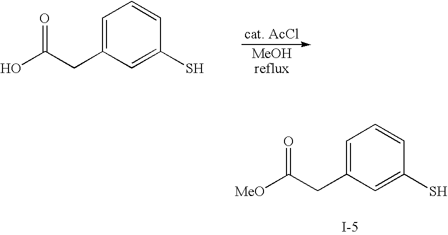

This intermediate is used in the preparation of Example 2 Intermediate I-5 was prepared according to the method described above for intermediate 4 and was isolated as a colorless oil. ¹H NMR (400 MHz, CDCl₃), δ (ppm): 7.21 (m, 3H), 7.11 (m, 1H), 3.72 (s, 3H), 3.59 (s, 2H).

### SYNTHESES OF RING ALKYLATED INTERMEDIATES FOR USE IN EMBODIMENTS HAVING [B] WITH SUBSTITUENTS

Synthesis of (3-hydroxy-5-trifluoromethanesulfonyloxy-phenyl)-acetic acid methyl ester (I-6).

Intermediate I-6, used to prepare ring methylated embodiments, was prepared according to Scheme XX:

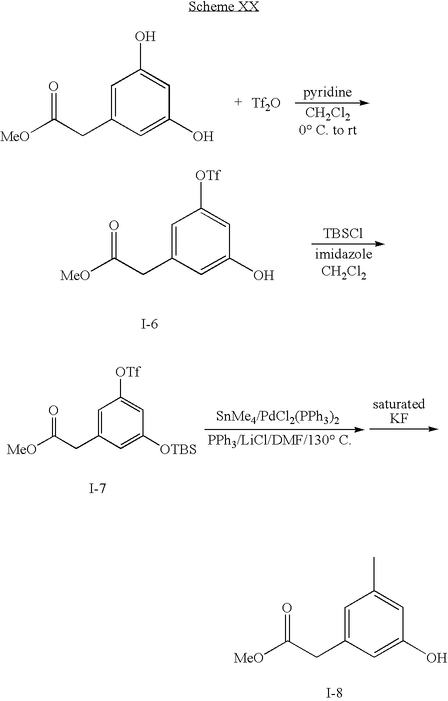

A suspension of (3,5-dihydroxy-phenyl)-acetic acid methyl ester (5.0 g, 27.45 mmol, 1.0 equiv.) and pyridine (2.44 mL, 30.19 mmol, 1.1 equiv.) in CH₂Cl₂ (100 mL) was cooled to 0° C. To the resulting cold mixture was added Tf₂O (4.62 mL, 27.45 mmol, 1.0 equiv.) drop wise with stirring over 30 minutes. After stirring at 0° C. for 15 minutes, the reaction solution was removed from the cooling bath and stirred for an additional 16 h. The reaction mixture was then diluted with CH₂Cl₂ (150 mL) and sequentially washed with water, brine, dried over Na₂SO₄. After removal of solvent, the crude product was purified by chromatography to afford 3.2 g (37% yield) of intermediate I-6. ¹H NMR (400 MHz, CDCl₃), δ (ppm): 6.78 (d, 2H), 6.68 (s, 1H), 5.71 (b, 1H), 3.73 (s, 3H), 3.61 (s, 2H).
Synthesis of [3-(tert-Butyl-dimethyl-silanyloxy)-5-trifluoromethanesulfonyloxy-phenyl)-acetic acid methyl ester (I-7).

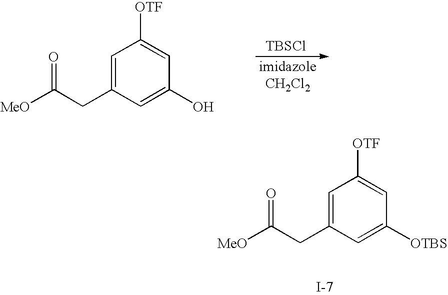

To a solution of intermediate I-6 (7.96 g, 28.5 mmol, 1.0 equiv.) in CH₂Cl₂ (200 mL) was added TBSCl (5.16 g, 34.2 mmol, 1.2 equiv.) followed by addition of imidazole (2.33 g, 34.2 mmol, 1.2 equiv.). The reaction mixture was stirred at room temperature for 3 h. TLC showed complete reaction. The reaction mixture was diluted with CH₂Cl₂ (200 mL), washed with water, brine, dried over Na₂SO₄. After removal of solvent, the crude product was purified by chromatography to afford 8.0 g (66% yield) of intermediate 7 as colorless oil. ¹H NMR (400 MHz, CDCl₃), δ (ppm): 6.84 (s, 1H), 6.81 (s, 1H), 6.67 (s, 1H), 3.73 (s, 3H), 3.44 (s, 2H), 0.99 (s, 9H), 0.38 (s, 6H).
Synthesis of (3-Hydroxy-5-methyl-phenyl)-acetic acid methyl ester (I-8).

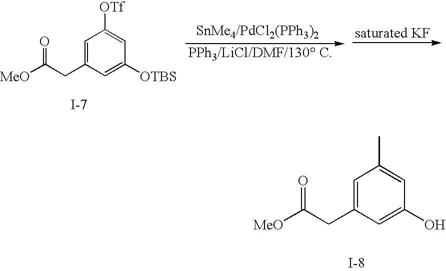

To a high pressure reaction flask was added intermediate I-7 (475.8 mg, 1.11 mmol, 1.0 equiv.), DMF (10 mL), PdCl₂(PPh₃)₂ (117 mg, 0.17 mmol, 0.15 equiv.), PPh₃ (58 mg, 0.22 mmol, 0.2 equiv.), LiCI (377 mg, 8.88 mmol, 8.0 equiv.) and SnMe₄. After the reaction flask was sealed, the reaction mixture was heated to 130° C. with stirring and then stirred at same temperature for 6 h. The reaction mixture was cooled to room temperature and then saturated KF aqueous solution (2 mL) was added. After stirring for 20 min, the mixture was diluted with ethyl acetate (50 mL), sequentially washed with saturated KF, water, brine, and dried over Na₂SO₄. After removal of solvent, the crude product was purified by chromatography to afford 45 mg (23% yield) of intermediate I-8 as colorless oil.: ¹H NMR (400 MHz, CDCl₃), δ (ppm): 6.66 (s, 1H), 6.57 (s, 2H), 4.94 (b, 1H), 3.70 (s, 3H), 3.54 (s, 2H), 2.28 (s, 3H).

Intermediates I-9, I-10, and I-11 were prepared from intermediate I-8 according to Scheme XXI:

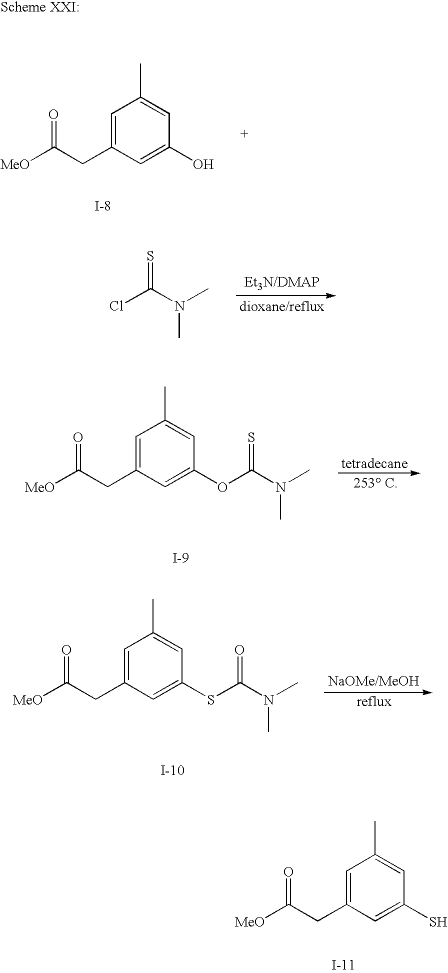

Synthesis of (3-Dimethylthiocarbamoyloxy-5-methyl-phenyl)-acetic acid methyl ester (I-9). To a solution of intermediate I-8 (4.67 g, 25.9 mmol, 1.0 equiv.) in dioxane (150 mL), was added dimethylthiocarbamoyl chloride (3.84 g, 31.1 mmol, 1.2 equiv.), Et₃N (7.22 mL, 51.8 mmol, 2.0 equiv.) and DMAP (316 mg, 2.59 mmol, 0.1 equiv.). The resulting mixture was heated to reflux and then stirred for 14 h. The reaction mixture was concentrated under reduced pressure and the residue was diluted with ethyl acetate (250 mL). The organic mixture was sequentially washed with water, brine, and dried over Na₂SO₄. After removal of solvent, the crude product was purified by chromatography to afford 2.06 g of intermediate I-9 as green oil. ¹H NMR (400 MHz, CDCl₃), δ (ppm): 7.00 (s, 1H), 6.84 (s, 1H), 6.83 (s, 1H), 3.72 (s, 3H), 3.63 (s, 2H), 3.50 (s, 3H), 3.34 (s, 3H), 2.37 (s, 3H).

Synthesis of (3-Dimethylcarbamoylsulfanyl-5-methyl-phenyl)-acetic acid methyl ester (I-10). A high pressure reaction flask was charged with intermediate I-9 (2.06 g, 7.69 mmol) and tetradecane (15 mL). After the flask was sealed, the suspension was heated to 255° C. with stirring. The reaction mixture was stirred at the same temperature for 16 h. and then was cooled to room temperature. After tetradecane was decanted, the residue was washed with hexane (2×15 mL). The crude product was used. without further purification. ¹H NMR (400 MHz, CDCl₃), δ (ppm): 7.25 (s, 1H), 7.24 (s, 1H), 7.14 (s, 1H), 3.71 (s, 3H), 3.63 (s, 2H), 3.06 (b, 6H), 2.37 (s, 3H).

Synthesis of (3-Mercapto-5-methyl-phenyl)-acetic acid methyl ester (I-11). To a solution of crude product I-10 (7.96 mmol, 1.0 equiv.) in dry MeOH (10 mL) was added 0.5N NaOMe solution in MeOH (17 mL, 8.5 mmol, 1.1 equiv.). The resulting solution was sealed in a high pressure reaction flask and heated to 60° C. with stirring, After stirred at the same temperature for 15 h, the reaction mixture was cooled to room temperature and then neutralized with 1N HCl (8.5 mL). The resulting mixture was concentrated under reduced pressure. The residue was diluted with ethyl acetate (50 mL) and washed with water, brine, dried over Na₂SO₄. After removal of solvent, the crude product was purified by chromatography to afford intermediate I-11 as colorless oil. ¹H NMR (400 MHz, CDCl₃), δ (ppm): 6.66 (s, 1H), 6.57 (s, 2H), 3.68 (s, 3H), 3.55 (s, 2H), 2.31 (s, 3H).
Synthesis of (5-Chlorosulfonyl-2-methyl-phenyl)-Acetic Acid Methyl Ester (I-12)

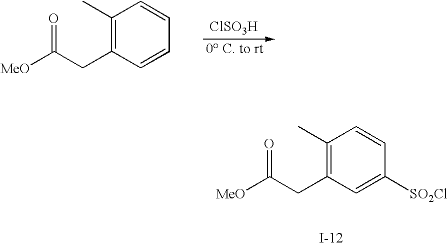

To vigorously stirred, cold (0° C.) chlorosulfonic acid (16.0 g, 150 mol, 3.0 equiv.) was added o-Tolyl-acetic acid methyl ester (8.2 g, 50 mmol, 1.0 equiv.) over the course of 30 min. After completion of addition, the reaction mixture was warmed to room temperature and continually stirred for an additional 5 h. The reaction mixture was then slowly poured into ice-water, and extracted with CHCl₃ (30 mL×2). The combined organic layer was sequentially washed with water, brine, and dried over Na₂SO₄. After removal of solvent, the crude product was purified by chromatography to afford 6.2 g (47.3% yield) of intermediate I-12 as white solid. ¹H NMR (400 MHz, CDCl₃), δ (ppm): 7.84 (d, 1H), 7.83 (s, 1H), 7.42 (d, 1H), 3.75 (s, 2H), 3.73 (s, 3H), 2.43 (s, 3H).
Synthesis of (3-Chlorosulfonyl-4-methyl-phenyl)-Acetic Acid Methyl Ester (I-13)

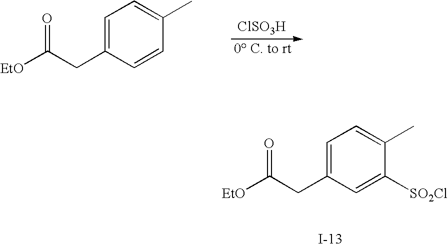

Intermediate I-13 was prepared using the method used to prepare intermediate I-12. Intermediate I-13 was isolated as a colorless oil. ¹H NMR (400 MHz, CDCl₃), δ (ppm): 8.00 (s, 1H), 7.56 (d, 2H), 7.40 (d, 1H), 4.19 (q, 2H), 3.70 (s, 2H), 2.78 (s, 3H), 1.29 (t, 3H).

Compounds of Examples 1-10 were assayed to measure their biological activity with respect to their EC50 for modulating PPAR-alpha, PPAR-gamma, and PPAR-delta as set forth in Table 2. Compounds of Examples 11-25 were assayed to measure their biological activity with respect to their EC50 for modulating PPAR-alpha, PPAR-gamma, and PPAR-delta as set forth in Table 3.

TABLE 2
BIOLOGICAL ACTIVITY OF EXAMPLES 1-10

| | PPAR α EC50 μM | PPAR γ EC50 μM | PPAR δ EC50 μM |
|---|---|---|---|
| Ex- | A = >10 μM | A = >10 μM | A = >10 μM |
| ample | B = <10-1 μM | B = <10-1 μM | B = <10-1 μM |
| Number | C = <1 μM | C = <1 μM | C = <1 μM |
| 1 | C | B | C |
| 2 | C | B | C |
| 3 | A | A | A |
| 4 | A | B | C |
| 5 | A | B | C |
| 6 | A | A | A |
| 7 | A | C | C |
| 8 | A | A | A |
| 9 | A | A | A |
| 10 | A | A | A |

TABLE 3
BIOLOGICAL ACTIVITY OF EXAMPLES 11-25

| | PPAR alpha | PPAR delta | PPAR gamma |
|---|---|---|---|
| | A > 100 μM | A > 100 μM | A > 100 μM |
| | B = 5-100 μM | B = 5-100 μM | B = 5-100 μM |
| Example # | C = <5 μM | C = <5 μM | C = <5 μM |
| 11 | A | A | A |
| 12 | B | B | B |
| 13 | B | C | B |
| 14 | A | B | B |
| 15 | C | C | B |
| 16 | B | C | B |
| 17 | B | B | B |
| 18 | B | B | B |
| 19 | C | C | B |
| 20 | C | B | B |
| 21 | B | B | B |
| 22 | B | B | B |
| 23 | C | B | B |
| 24 | B | C | B |
| 25 | B | C | B |

It should be understood by a person of ordinary skill in the art that the foregoing examples illustrate embodiments of the invention but that the invention is not to be limited by the examples.

1. A compound having a structure of Formula (I) or a pharmaceutically acceptable salt, ester, thioester, amide, pro-drug or solvate thereof
[A]-[B]-[C] (I)
wherein
(a) [A] is [H]-[L];
wherein [H] represents a COOH (or a hydrolyzable ester thereof) or tetrazole group
[L] is: 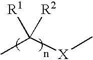
wherein:
each R¹ and each R² are independently H or C₁₋₃ alkyl, or R¹ and R² which are bonded to the same carbon atom may together with the carbon atom to which they are bonded, form a 3-6 membered cycloalkyl ring
n=0, 1 or 2
X=O, S or null
(b) [B] is a ring system selected from the group consisting of: 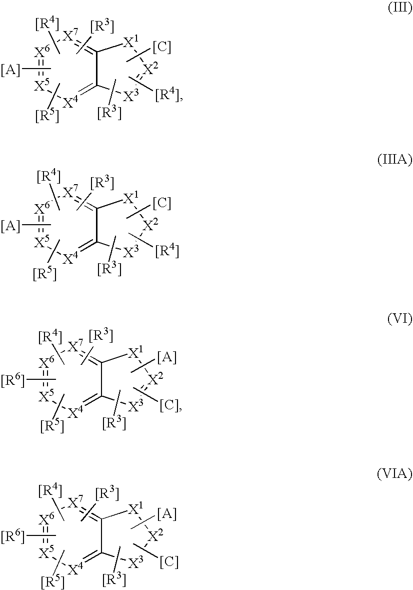
wherein X¹ is NH, O, or S; except when any of [C], [A], or R³-R⁴ is attached to X¹, X¹ is N;
X2-X7 are each independently CH, N, or C when [C], [A], R³, R⁴, R⁵, or R⁶ is attached or when [B] is IIIA or VIA, X₂ and X₃ are each independently CH₂, NH, or, when [C], [A], R³, or R⁴ is attached, CH, C, or N;
Each R³, each R⁴, each R⁵, and each R⁶ are each independently hydrogen, perhaloaryloxy, alkanoylalkyl, alkanoylalkoxy, alkanoyloxy, N-aryl-N-alkylamino, heterocyclylalkoxy, heterocyclylthio, hydroxyalkoxy, carboxamidoalkoxy, alkoxycarbonylalkoxy, alkoxycarbonylalkenyloxy, aralkanoylalkoxy, aralkenoyl, N-alkylcarboxamido, N-haloalkylcarboxamido, N-cycloalkylcarboxamido, N-arylcarboxamidoalkoxy, cycloalkylcarbonyl, cyanoalkoxy, heterocyclylcarbonyl, carboxy, heteroaralkylthio, heteroaralkoxy, cycloalkylamino, acylalkyl, acylalkoxy, aroylalkoxy, heterocyclyloxy, aralkylaryl, aralkyl, aralkenyl, aralkynyl, heterocyclyl, haloalkylthio, alkanoyloxy, alkoxy, alkoxyalkyl, cycloalkoxy, cycloalkylalkoxy, hydroxy, amino, thio, nitro, alkylamino, alkylthio, arylamino, aralkylamino, arylthio, arylthioalkyl, alkylsulfonyl, alkylsulfonamido, monoarylamidosulfonyl, arylsulfonyl, heteroarylthio, heteroarylsulfonyl, heterocyclylsulfonyl, heterocyclylthio, alkanoyl, alkenoyl, aroyl, heteroaroyl, aralkanoyl, heteroaralkanoyl, haloalkanoyl, alkyl, alkenyl, alkynyl, alkenyloxy, alkylenedioxy, haloalkylenedioxy, cycloalkyl, cycloalkylalkanoyl, halo, haloalkyl, haloalkoxy, hydroxyhaloalkyl, hydroxyhaloalkoxy, hydroxyalkyl, aryl, aryloxy, aralkoxy, saturated heterocyclyl, heteroaryl, heteroaryloxy, heteroaryloxyalkyl, heteroaralkyl, arylalkenyl, carboalkoxy, alkoxycarboxamido, alkylamidocarbonylamido, arylamidocarbonylamido, carboalkoxyalkyl, carboalkoxyalkenyl, carboxamido, carboxamidoalkyl, and cyanocycloalkylalkyl, cycloalkenyl, alkoxycarbonyl, aralkylthio, alkylthio, alkylsulfinyl, arylsulfinyl, dialkylamino, aminoalkyl, dialkylaminoalkyl, aminoaryl, alkylaminoaryl, acylamino; aminocarbonylalkoxy, aminocarbonylamino, aminocarbonylaminoalkyl, aminothiocarbonylamino, aminothiocarbonylaminoalkyl and may be attached to any X¹-X⁷;
d) [C] is 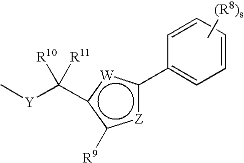
wherein Y is O, S, or (CR¹²R¹³)ᵣ where r is 0-2;
each R¹² and each R¹³ are each independently H, fluorine or C₁₋₆ alkyl;
one of W and Z is N, the other is S or O;
R¹⁰ and R¹¹ are each independently H, phenyl, benzyl, fluorine, C₁₋₆ alkyl, or allyl;
R⁹ is H, CH₃, or CF₃;
Each R⁸ is independently CF₃, C₁₋₆ alkyl, OCH₃ or halogen;
s is 0, 1, 2, 3, 4 or 5.

2. The Compound of claim 1 wherein [B] is selected from the group consisting of VI and VIA.

3. The Compound of claim 2 wherein X¹ is N or NH.

4. The Compound of claim 3 wherein one of X²-X⁷ is N or NH.

5. The compound of claim 3 wherein none of X²-X⁷ are heteroatoms.

6. The compound of claim 5 wherein X¹ is N and [C] is attached to X¹.

7. The compound of claim 5 wherein [B]=VI

8. A compound according to claim 7 wherein [B] has the structure selected from the group consisting of:

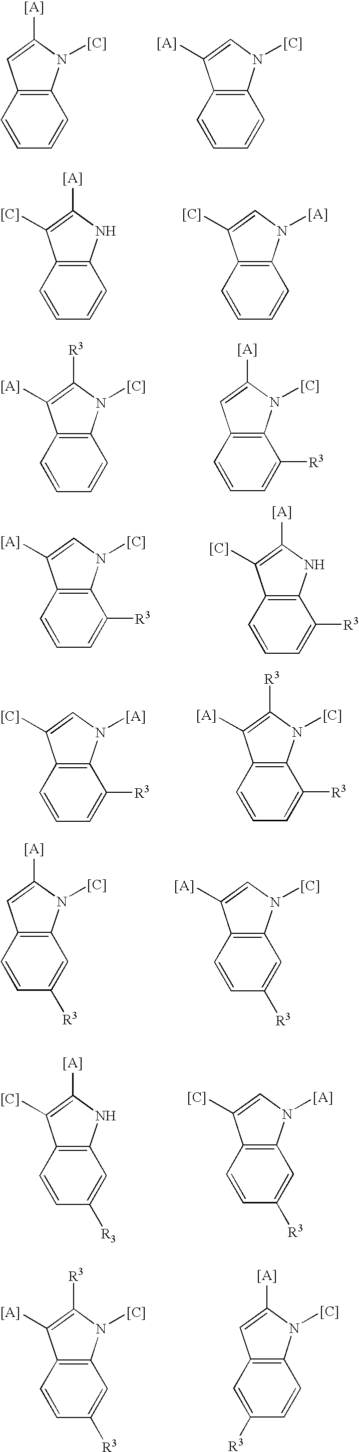

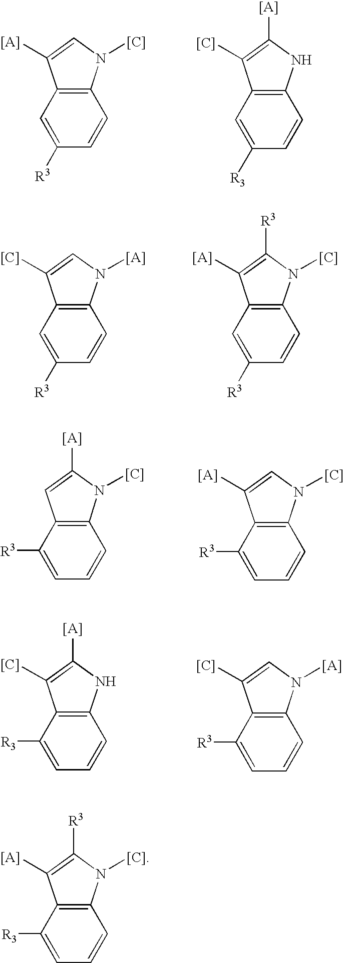

9. The compound of claim 8 wherein X¹ is N and [C] is attached to X¹.

10. The compound of claim 9 wherein X=O or null.

11. The compound of claim 10 wherein n=1.

12. The compound of claim 11 wherein R═R═H.

13. The compound of claim 11 wherein R═R methyl.

14. The compound of claim 11 wherein Y═CR¹²R¹³ and r=0 or 1.

15. The compound of claim 14 wherein W═S and Z=N.

16. The compound of claim 2 wherein X¹ is O or S and X² or X³ is N.

17. The compound of claim 15 wherein the compound has the following structure or a pharmaceutically acceptable salt, ester, thioester, amide, pro-drug or solvate thereof:

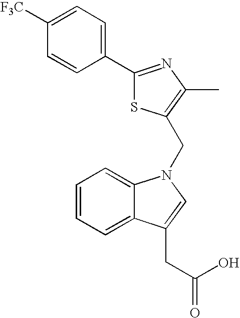

18. The compound of claim 1 wherein [B] is selected from the group consisting of III and IIIA

19. The Compound of claim 18 wherein X¹ is N or NH.

20. The Compound of claim 19 wherein one of X²-X⁷ is N or NH.

21. A compound according to claim 20 wherein [B] has the structure selected from the group consisting of: 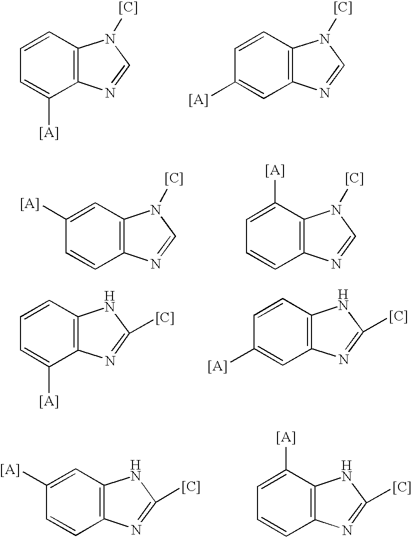
wherein [B] is optionally singly or doubly substituted with R³.

22. A compound according to claim 20 wherein [B] has the structure selected from the group consisting of: 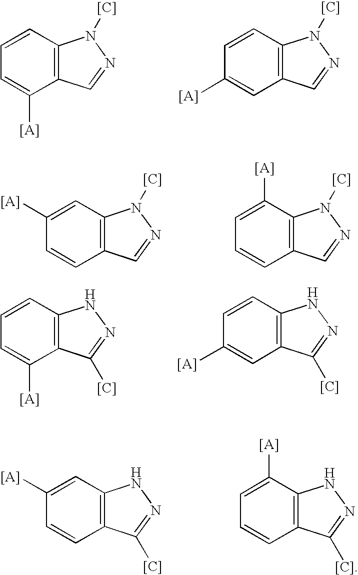
wherein [B] is optionally singly or doubly substituted with R³.

23. The compound of claim 19 wherein none of X²-X7 are heteroatoms.

24. The compound of claim 23 wherein [B]=III

25. The compound according to claim 24 wherein [B] has the structure selected from the group consisting of: 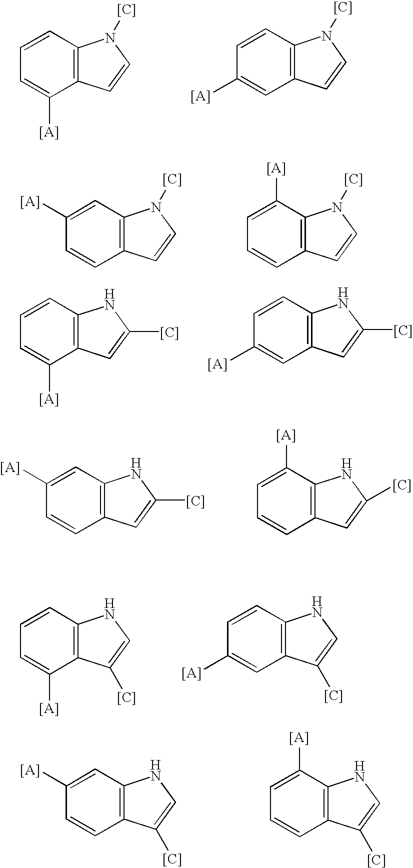
wherein [B] is optionally singly or doubly substituted with R³.

26. The compound of claim 25 wherein X¹ is N and [C] is attached to X¹.

27. The compound of claim 26 wherein X=O or null.

28. The compound of claim 27 wherein n=1.

29. The compound of claim 28 wherein R¹═R²═H.

30. The compound of claim 28 wherein R¹═R²=methyl.

31. The compound of claim 28 wherein Y═CR¹²R¹³ and r=0 or 1.

32. The compound of claim 31 wherein W═S and Z=N.

33. The compound according to claim 32 wherein R⁹=methyl.

34. The compound according to claim 32 wherein the R⁸ substitution pattern is selected from the group consisting of: 4-perhaloalkyl; 4-halogen; 3,4, dihalo; 3-halo, 4-perfluoroalkyl.

35. The compound according to claim 34 wherein R⁹=methyl.

36. The compound according to claim 34 wherein [A] is attached to X5 or X6.

37. The compound according to claim 26 wherein each R³, each R⁴, each R⁵, and each R are each independently H, C₁₋₃alkyl, OCH₃, CF₃, or halogen and may be attached to any X¹-X⁷.

38. The compound according to claim 26 wherein [A] is attached to X⁵ or X⁶.

39. The compound of claim 23 wherein [B]=IIIA

40. The compound of claim 39 wherein X¹ is N and [C] is attached to X¹.

41. The compound of claim 40 wherein X=O or null.

42. The compound of claim 41 wherein n=1.

43. The compound of claim 42 wherein R¹═R²═H.

44. The compound of claim 42 wherein R¹═R²═methyl.

45. The compound of claim 42 wherein Y=C R¹²R¹³ and r=0 or 1.

46. The compound of claim 45 wherein W═S and Z=N.

47. The compound according to claim 46 wherein R⁹=methyl.

48. The compound according to claim 46 wherein the R⁸ substitution pattern is selected from the group consisting of: 4-perhaloalkyl; 4-halogen; 3,4, dihalo; 3-halo, 4-perfluoroalkyl.

49. The compound according to claim 48 wherein R⁹=methyl.

50. The compound according to claim 48 wherein [A] is attached to X⁵ or X⁶.

51. The compound according to claim 40 wherein each R³, each R⁴, each R⁵, and each R are each independently H, C₁₋₃alkyl, OCH₃, CF₃, or halogen and may be attached to any X¹-X⁷.

52. The compound according to claim 40 wherein [A] is attached to X⁵ or X⁶.

53. The compound according to claim 18 wherein X¹ is O or S, wherein one of X² or X³ is N and the other of X² and X³ is attached to [C].

54. The compound of claim 53 wherein X=O or null.

55. The compound of claim 54 wherein n=1.

56. The compound of claim 55 wherein R¹═R²═H.

57. The compound of claim 55 wherein R¹═R²═methyl.

58. The compound of claim 55 wherein Y=C R¹²R¹³ and r=0 or 1.

59. The compound of claim 58 wherein W═S and Z=N.

60. The compound according to claim 59 wherein R⁹=methyl.

61. The compound according to claim 59 wherein the R⁸ substitution pattern is selected from the group consisting of: 4-perhaloalkyl; 4-halogen; 3,4, dihalo; 3-halo, 4-perfluoroalkyl.

62. The compound according to claim 61 wherein R⁹=methyl.

63. The compound according to claim 61 wherein [A] is attached to X⁵ or X⁶.

64. The compound according to claim 53 wherein each R³, each R⁴, each R⁵, and each R⁶ are each independently H, C₁₋₃alkyl, OCH₃, CF₃, or halogen and may be attached to any X¹-X⁷.

65. The compound according to claim 53 wherein [A] is attached to X⁵ or X⁶.

66. The compound according to claim 26 wherein the compound is selected from the group consisting of:

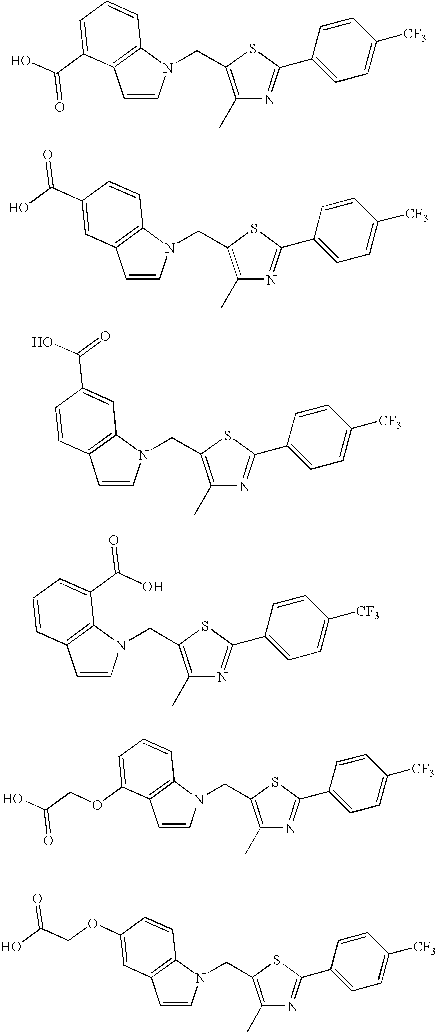

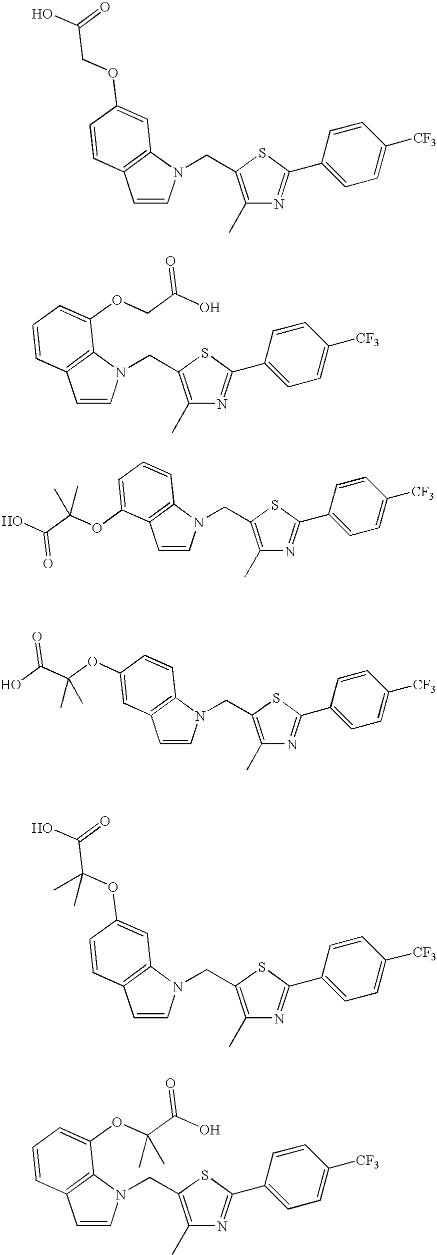

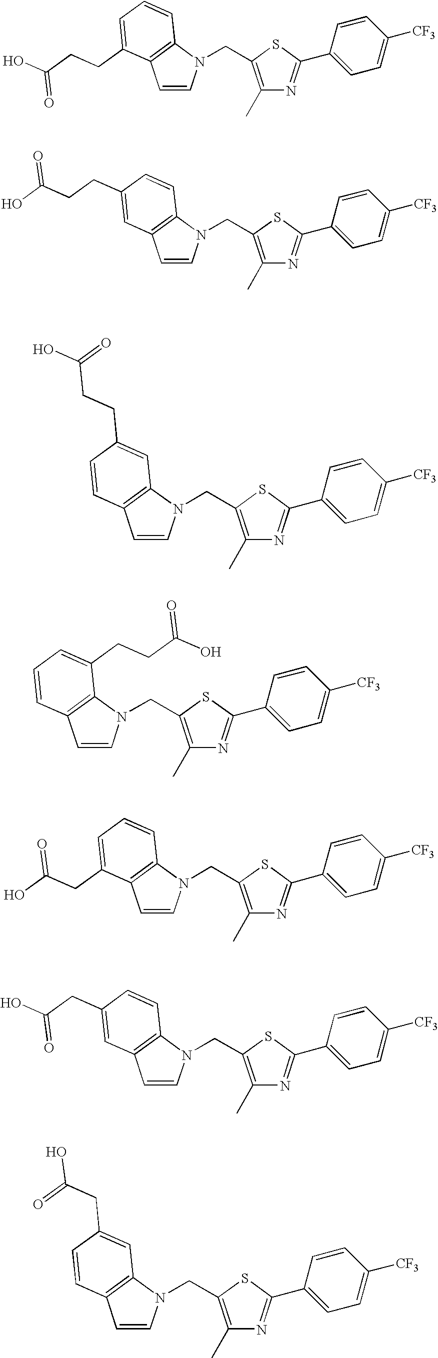

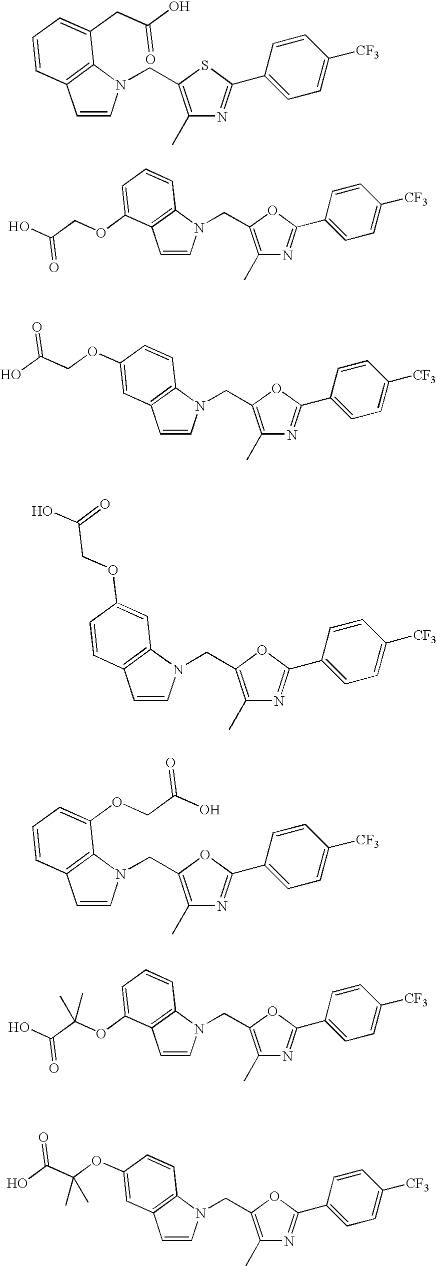

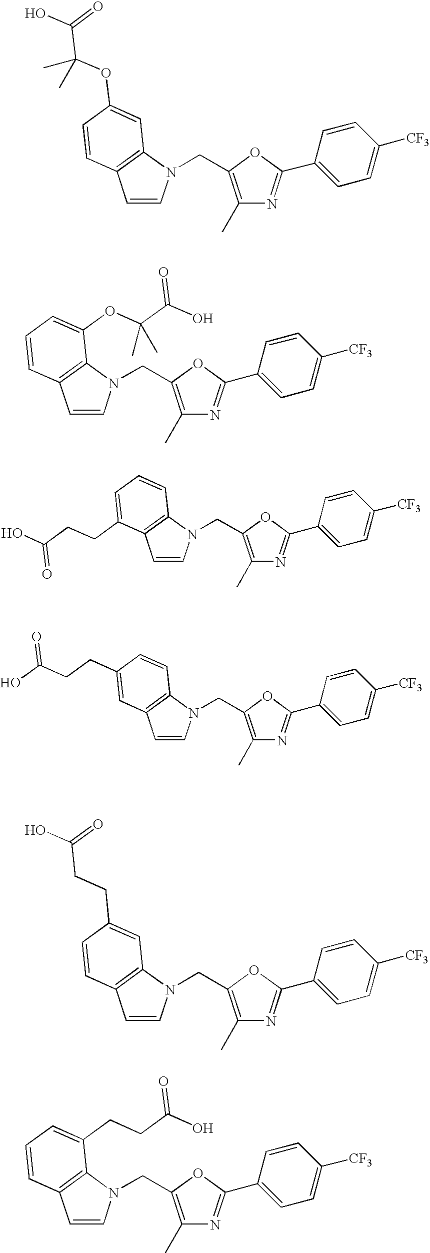

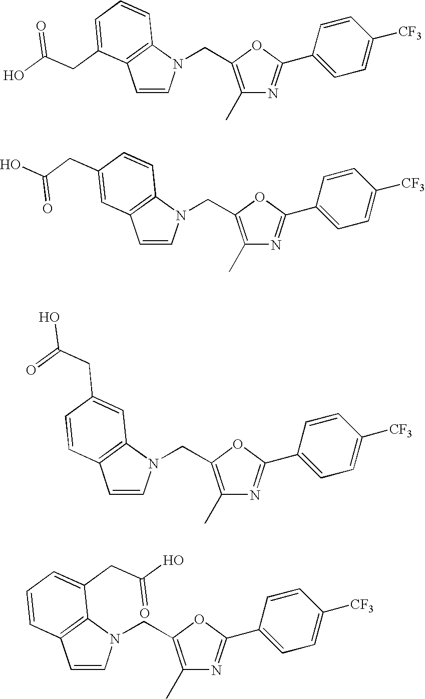

67. The compound according to claim 20 wherein the compound is selected from the group consisting of:

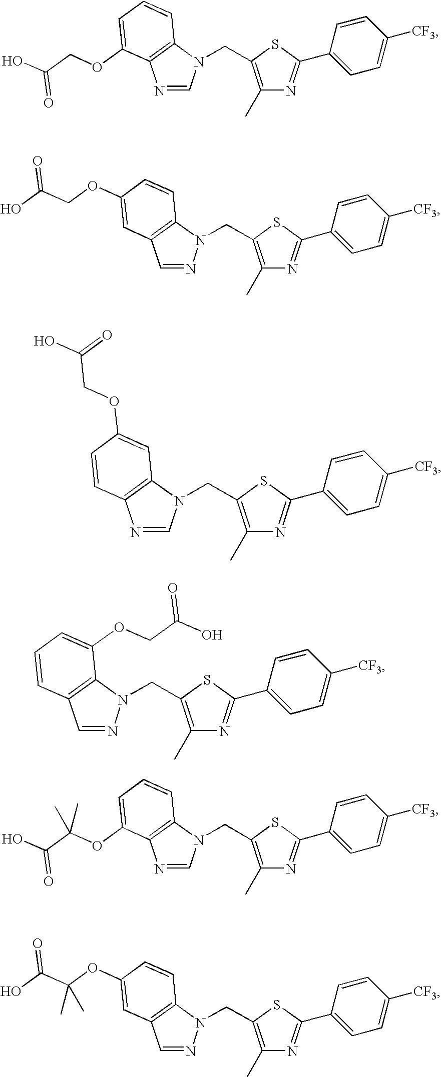

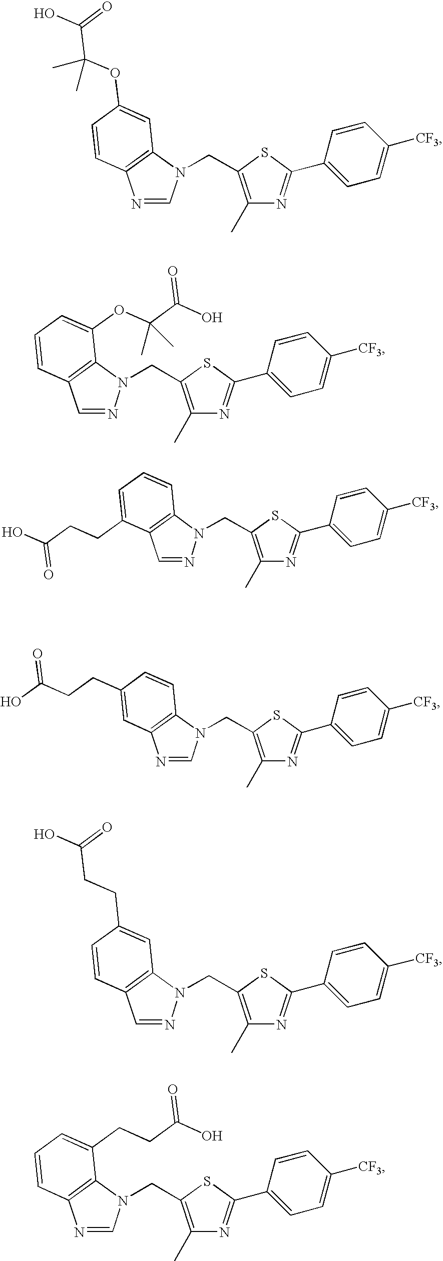

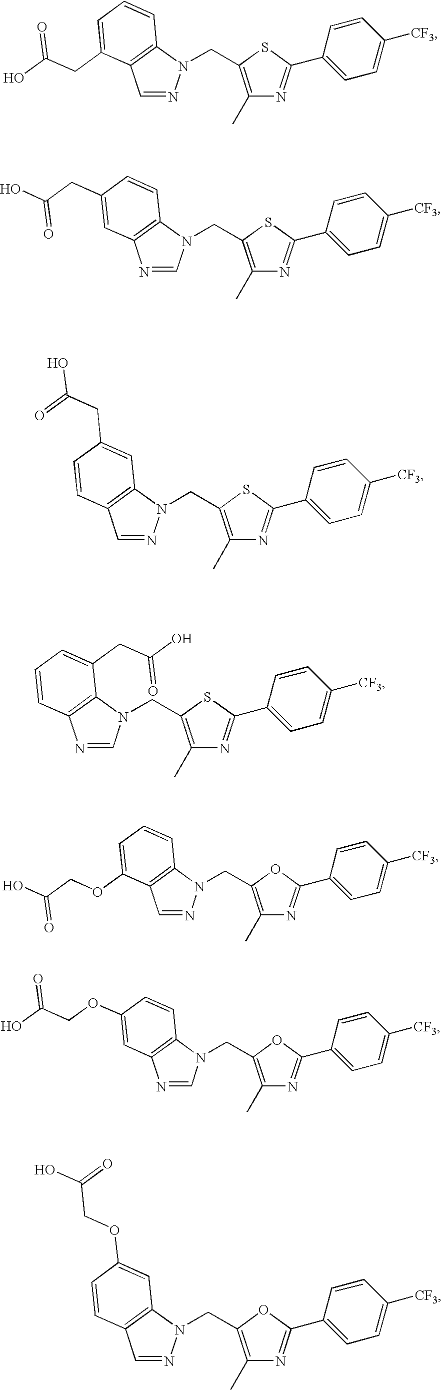

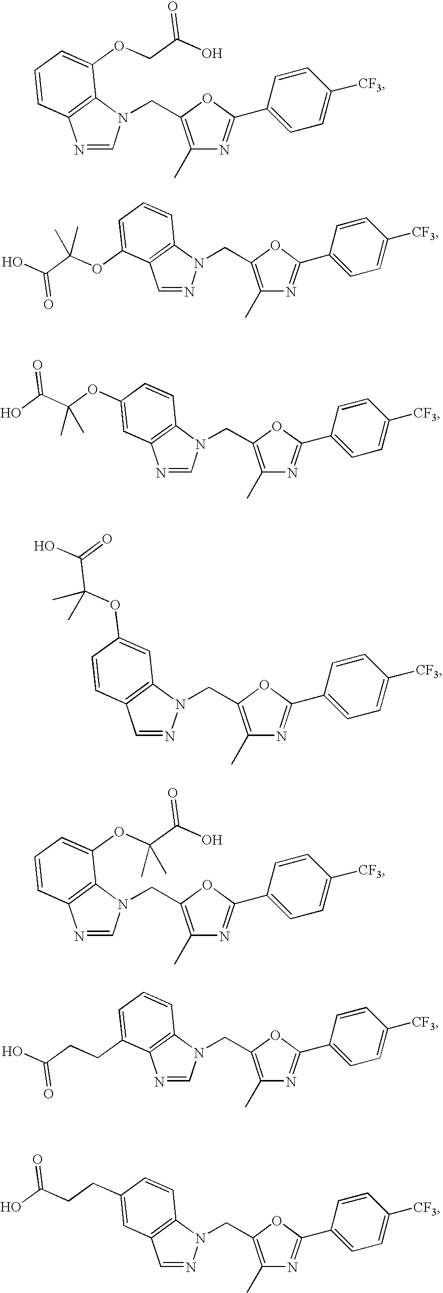

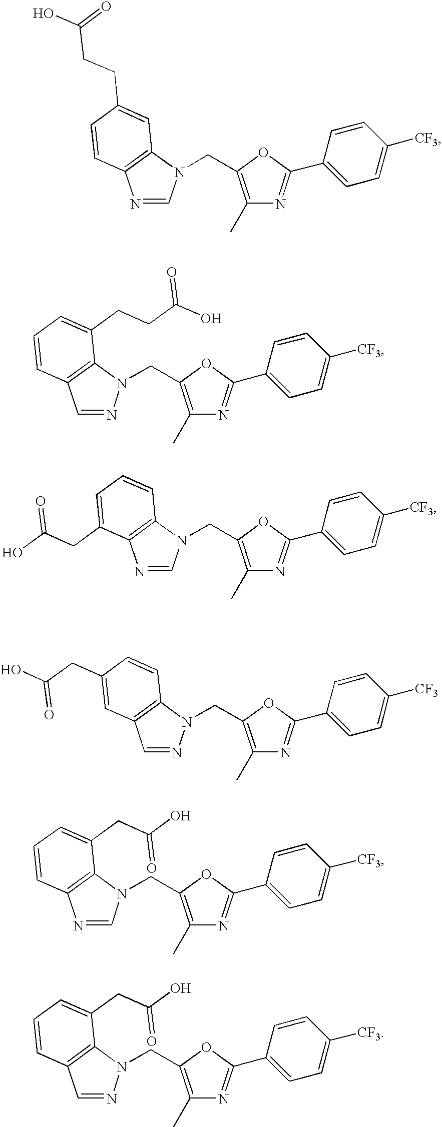

68. The compound according to claim 62 wherein the compound is selected from the group consisting of:

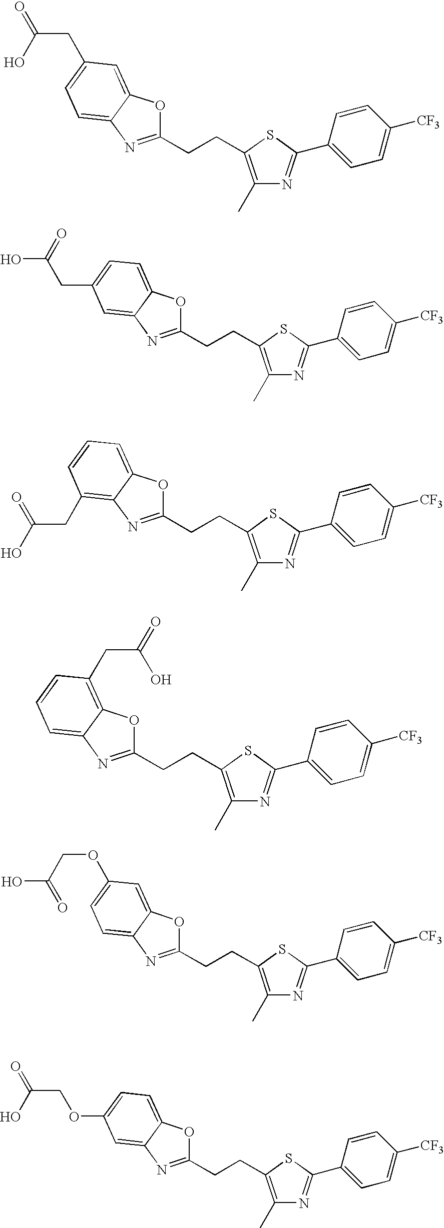

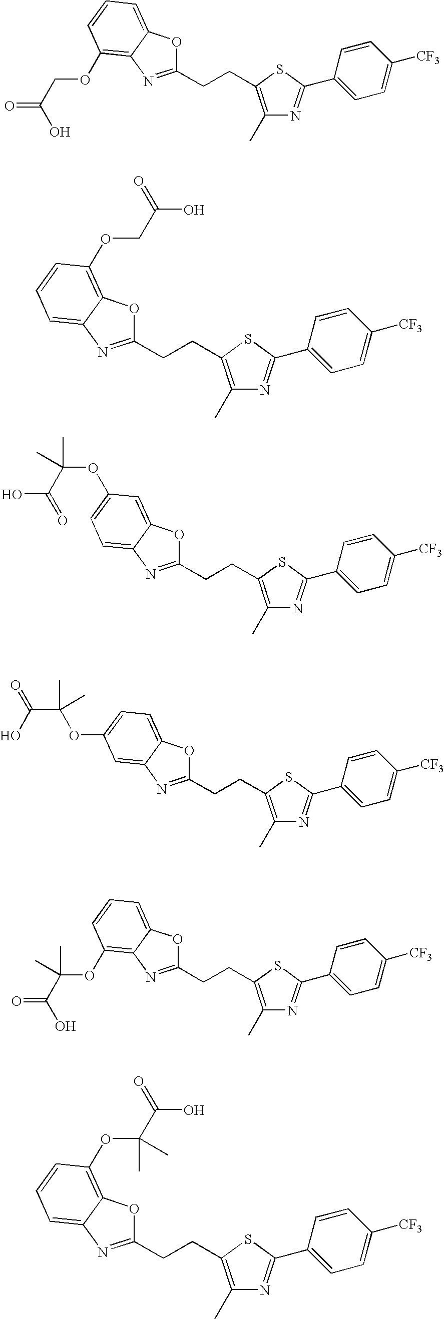

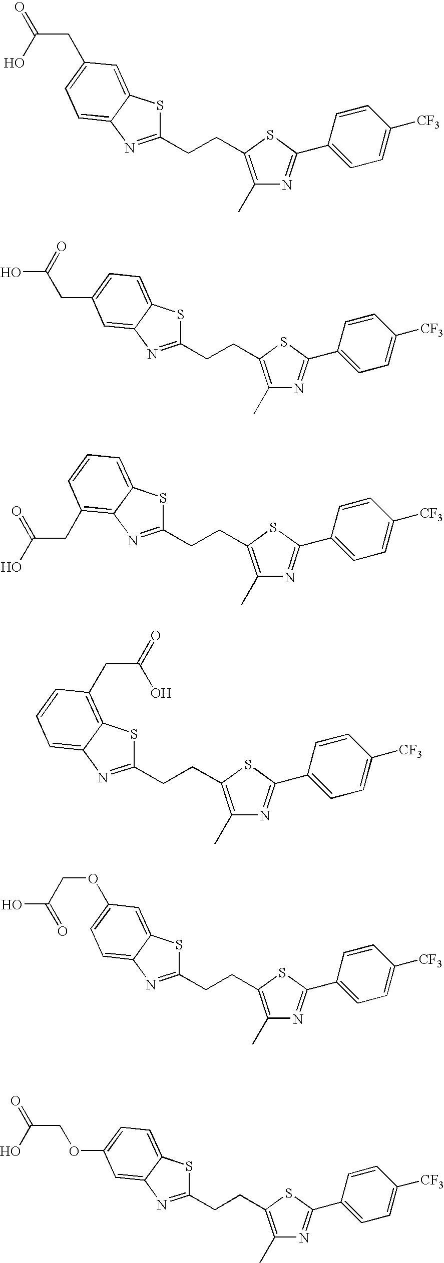

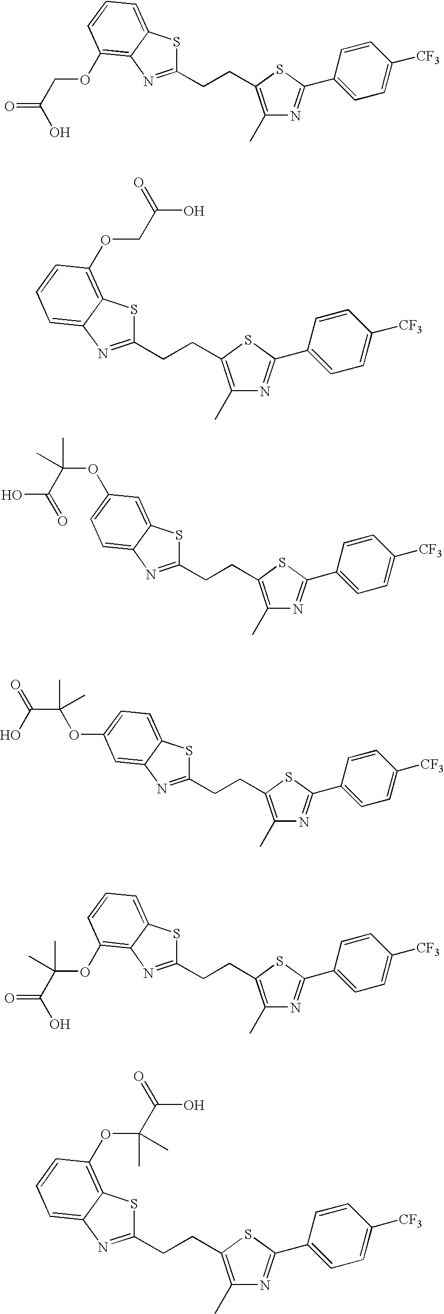

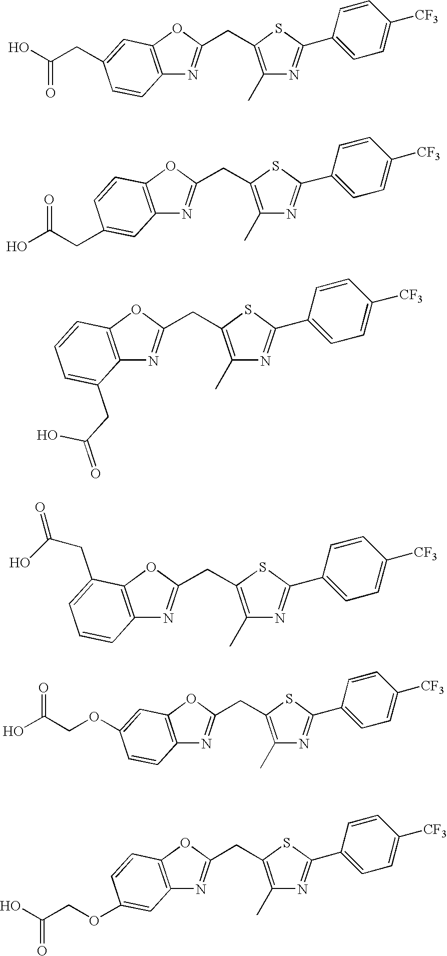

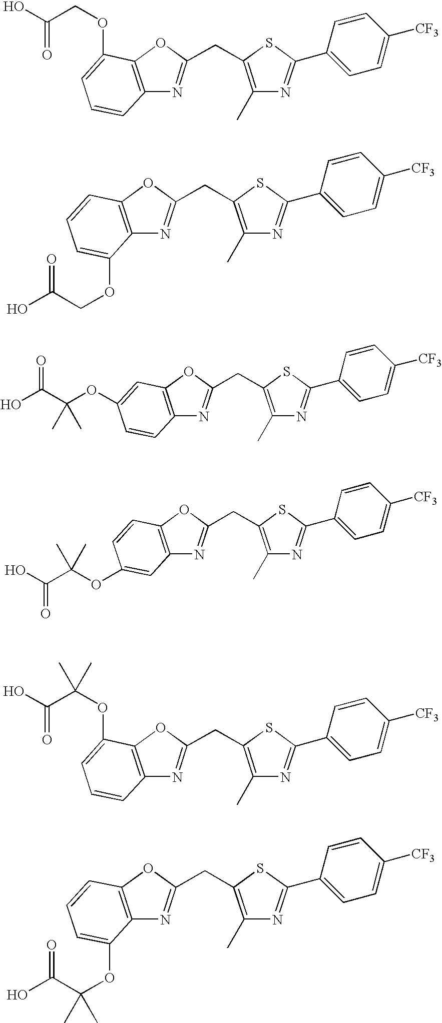

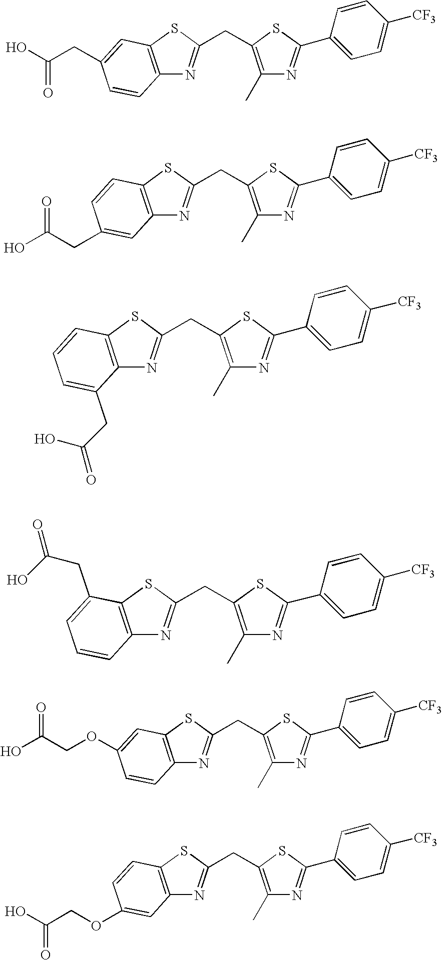

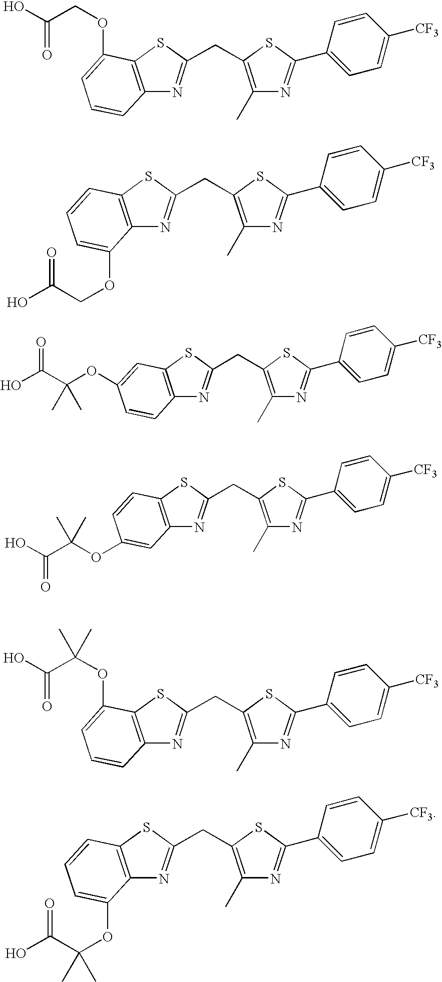

69. The compound according to claim 6 wherein the compound is selected from the group consisting of:

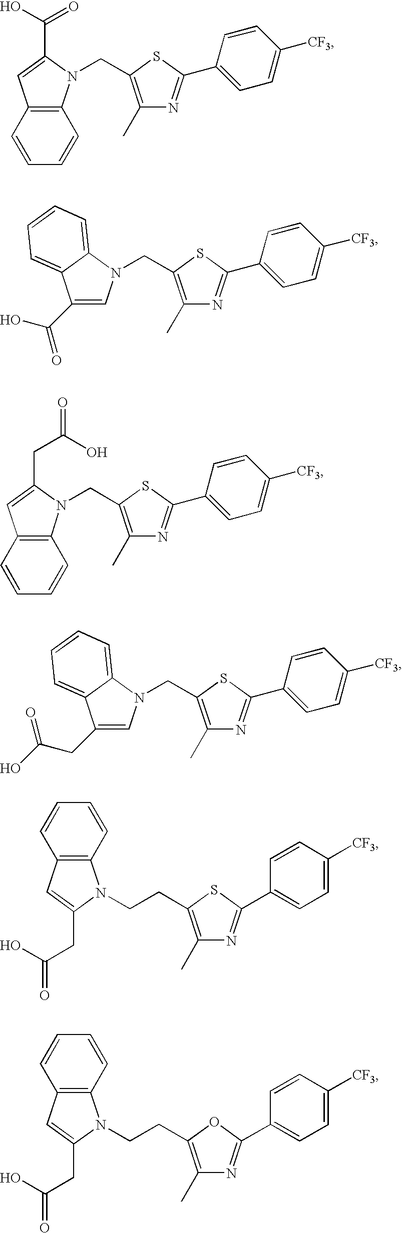

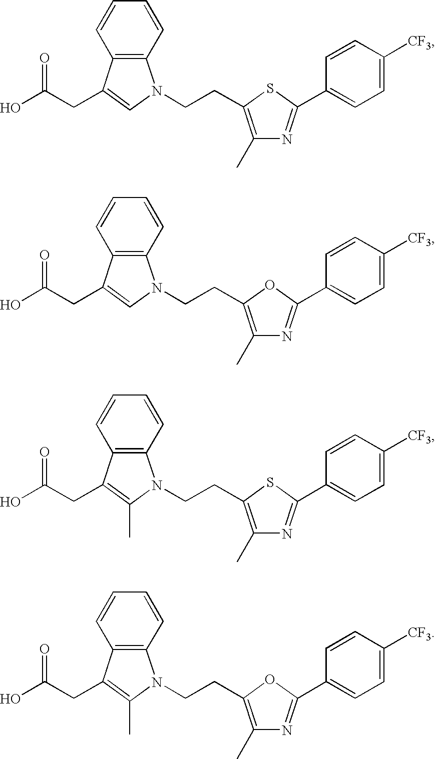

70. The compound according to claim 37 wherein the compound is selected from the group consisting of:

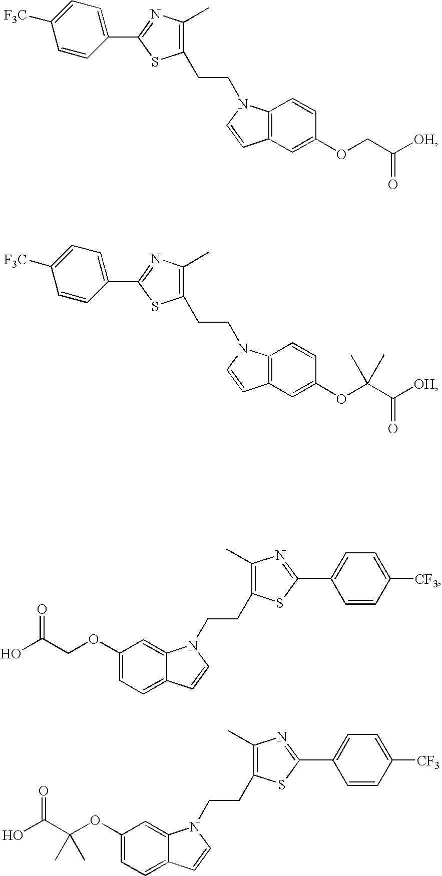

71. The compound according to claim 50 wherein the compound is selected from the group consisting of:

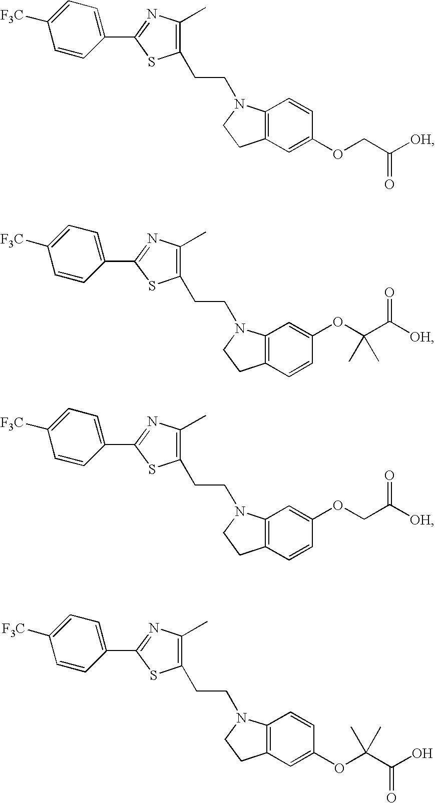

72. A compound having a structure selected from the following or a pharmaceutically acceptable salt, ester, thioester, amide, pro-drug or solvate thereof:

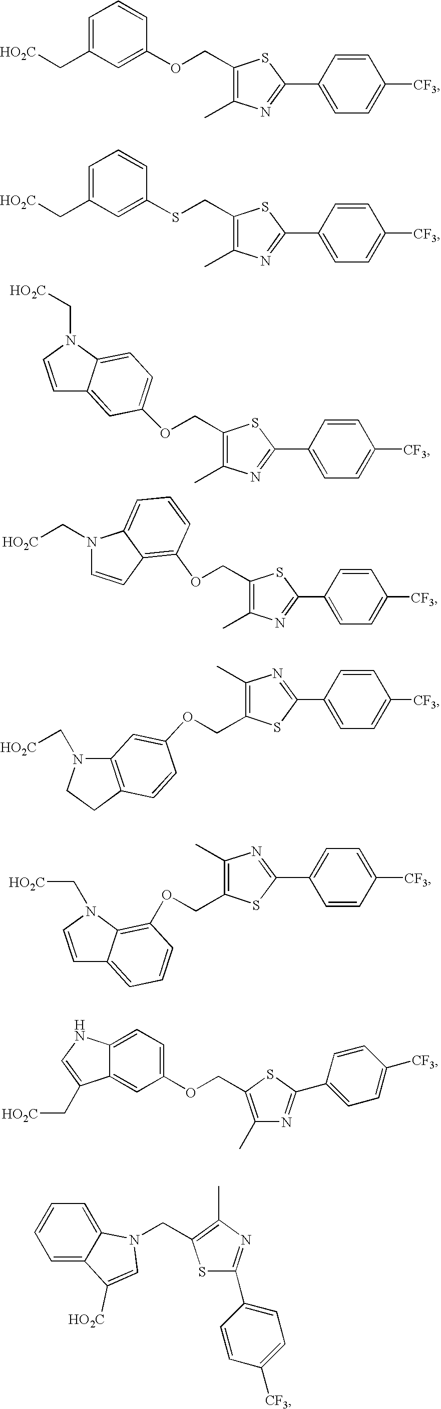

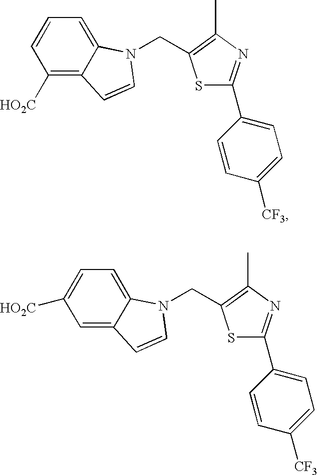

73. A compound having a structure of Formula (I) or a pharmaceutically acceptable salt, ester, thioester, amide, pro-drug or solvate thereof
[A]-[B]-[C] (I)
wherein
(c) [A] is [H]-[L];
wherein [H] represents a COOH (or a hydrolyzable ester thereof) or tetrazole group
[L] is: 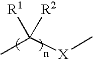
wherein:
each R¹ and each R² are independently H or C₁₋₃ alkyl, or R¹ and R² which are bonded to the same carbon atom may together with the carbon atom to which they are bonded, form a 3-6 membered cycloalkyl ring
n=0, 1 or 2
X=O, S or null
(d) [B] is a ring system selected from the group consisting of: 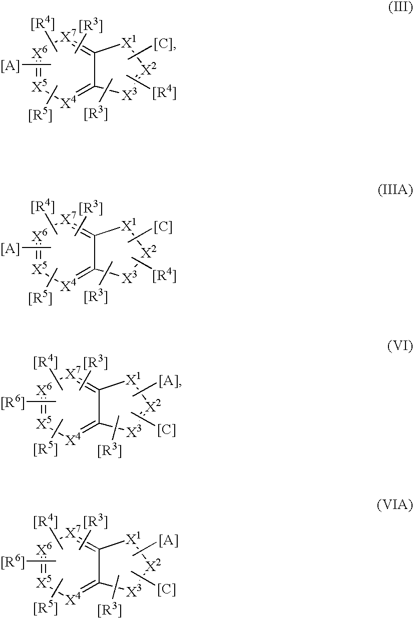
wherein X¹ is NH, O, or S; except when any of [C], [A], or R³-R⁴ is attached to X¹, X¹ is N;
X²-X⁷ are each independently CH, N, or C when [C], [A], R³,R⁴,R⁵, or R⁶ is attached or when [B] is IIIA or VIA, X₂ and X₃ are each independently CH₂, NH, or, when [C], [A], R³, or R⁴ is attached, CH, C, or N;
Each R³, each R⁴, each R⁵, and each R⁶ are each independently hydrogen, perhaloaryloxy, alkanoylalkyl, alkanoylalkoxy, alkanoyloxy, N-aryl-N-alkylamino, heterocyclylalkoxy, heterocyclylthio, hydroxyalkoxy, carboxamidoalkoxy, alkoxycarbonylalkoxy, alkoxycarbonylalkenyloxy, aralkanoylalkoxy, aralkenoyl, N-alkylcarboxamido, N-haloalkylcarboxamido, N-cycloalkylcarboxamido, N-arylcarboxamidoalkoxy, cycloalkylcarbonyl, cyanoalkoxy, heterocyclylcarbonyl, carboxy, heteroaralkylthio, heteroaralkoxy, cycloalkylamino, acylalkyl, acylalkoxy, aroylalkoxy, heterocyclyloxy, aralkylaryl, aralkyl, aralkenyl, aralkynyl, heterocyclyl, haloalkylthio, alkanoyloxy, alkoxy, alkoxyalkyl, cycloalkoxy, cycloalkylalkoxy, hydroxy, amino, thio, nitro, alkylamino, alkylthio, arylamino, aralkylamino, arylthio, arylthioalkyl, alkylsulfonyl, alkylsulfonamido, monoarylamidosulfonyl, arylsulfonyl, heteroarylthio, heteroarylsulfonyl, heterocyclylsulfonyl, heterocyclylthio, alkanoyl, alkenoyl, aroyl, heteroaroyl, aralkanoyl, heteroaralkanoyl, haloalkanoyl, alkyl, alkenyl, alkynyl, alkenyloxy, alkylenedioxy, haloalkylenedioxy, cycloalkyl, cycloalkylalkanoyl, halo, haloalkyl, haloalkoxy, hydroxyhaloalkyl, hydroxyhaloalkoxy, hydroxyalkyl, aryl, aryloxy, aralkoxy, saturated heterocyclyl, heteroaryl, heteroaryloxy, heteroaryloxyalkyl, heteroaralkyl, arylalkenyl, carboalkoxy, alkoxycarboxamido, alkylamidocarbonylamido, arylamidocarbonylamido, carboalkoxyalkyl, carboalkoxyalkenyl, carboxamido, carboxamidoalkyl, and cyanocycloalkylalkyl, cycloalkenyl, alkoxycarbonyl, aralkylthio, alkylthio, alkylsulfinyl, arylsulfinyl, dialkylamino, aminoalkyl, dialkylaminoalkyl, aminoaryl, alkylaminoaryl, acylamino; aminocarbonylalkoxy, aminocarbonylamino, aminocarbonylaminoalkyl, aminothiocarbonylamino, aminothiocarbonylaminoalkyl and may be attached to any X¹-X⁷;
e) [C] is 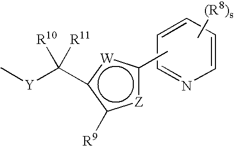
wherein Y is O, S, or (CR¹²R¹³)ᵣ where r is 0-2;
each R¹² and each R¹³ are each independently H, fluorine or C₁₋₆ alkyl;
one of W and Z is N, the other is S or O;
R¹⁰ and R¹¹ are each independently H, phenyl, benzyl, fluorine, C₁₋₆ alkyl, or allyl;
R⁹ is H, CH₃, or CF₃;
Each R⁸ is independently CF₃, C₁₋₆ alkyl, OCH₃ or halogen; and
s is 0, 1, 2, 3, 4 or 5.

74. The compound according to claim 1 wherein the compound is an hPPAR-delta modulator.

75. The compound according to claim 74 wherein the compound is a selective hPPAR-delta modulator.

76. A pharmaceutical composition comprising a compound according to claim 74.

77. The pharmaceutical composition according to claim 76 further comprising a pharmaceutical acceptable diluent or carrier.

78. The pharmaceutical composition according to claim 76 for use in the treatment of an hPPAR-delta mediated disease or condition.

79. The pharmaceutical composition according to claim 78 wherein said hPPAR-delta mediated disease or condition is dyslipidemia, syndrome X, heart failure, hypercholesteremia, cardiovascular disease, type II diabetes mellitus, type 1 diabetes, insulin resistance hyperlipidemia, obesity, anorexia bulimia, inflammation and anorexia nervosa.

80. A compound according to claim 74 for use in the manufacture of a medicament for the prevention or treatment of a hPPAR-delta-mediated disease or condition.

81. A compound, pharmaceutically acceptable prodrug, pharmaceutically active metabolite, or pharmaceutically acceptable salt comprising a compound according to claim 74 having an EC50 value less than 1 μM as measured by a functional cell assay.

82. A method for raising HDL in a subject comprising the administration of a therapeutic amount of a hPPAR-delta modulator compound according to claim 74.

83. Use of a hPPAR-delta modulator compound according to claim 74 for the manufacture of a medicament for the raising of HDL in a patient in need thereof.

84. A method for treating Type 2 diabetes, decreasing insulin resistance or lowering blood pressure in a subject comprising the administration of a therapeutic amount of a hPPAR-delta modulator compound according to claim 74.

85. Use of a hPPAR-delta modulator compound according to claim 74 for the manufacture of a medicament for the treatment of Type 2 diabetes, decreasing insulin resistance or lowering blood pressure in a patient in need thereof.

86. A method for decreasing LDLc in a subject comprising the administration of a therapeutic amount of a hPPAR delta modulator compound according to claim 74.

87. Use of a hPPAR-delta modulator compound according to claim 74 for the manufacture of a medicament for decreasing LDLc in a patient in need thereof.

88. A method for shifting LDL particle size from small dense to normal dense LDL in a subject comprising the administration of a therapeutic amount of a hPPAR-delta modulator compound according to claim 74.

89. Use of a hPPAR-delta modulator compound according to claim 74 for the manufacture of a medicament for shifting LDL particle size from small dense to normal LDL in a patient in need thereof.

90. A method for treating atherosclerotic diseases including vascular disease, coronary heart disease, cerebrovascular disease and peripheral vessel disease in a subject comprising the administration of a therapeutic amount of a hPPAR-delta modulator compound according to claim 74.

91. Use of a hPPAR-delta modulator compound according to claim 74 for the manufacture of a medicament for the treatment of atherosclerotic diseases including vascular disease, coronary heart disease, cerebrovascular disease and peripheral vessel disease in a patient in need thereof.

92. A method for treating inflammatory diseases, including rheumatoid arthritis, asthma, osteoarthritis and autoimmune disease in a subject comprising the administration of a therapeutic amount of a hPPAR-delta modulator compound according to claim 74.

93. Use of a hPPAR-delta modulator compound according to claim 74 for the manufacture of a medicament for the treatment of inflammatory diseases, including rheumatoid arthritis, asthma, osteoarthritis and autoimmune disease in a patient in need thereof.

94. A method of treatment of a hPPAR-delta mediated disease or condition comprising administering a therapeutically effective amount of a compound according to claim 74 or a pharmaceutically acceptable salt, ester, amide, or prodrug thereof.

95. A method of modulating a peroxisome proliferator-activated receptor (PPAR) function comprising contacting said PPAR with a compound of claim 74 and monitoring a change in cell phenotype, cell proliferation, activity of said PPAR, or binding of said PPAR with a natural binding partner.

96. The method of claim 95, wherein said PPAR is selected from the group consisting of PPAR-alpha, PPAR-delta, and PPAR-gamma.

97. A method of treating a disease comprising identifying a patient in need thereof, and administering a therapeutically effective amount of a compound of claim 74 to said patient wherein said disease is selected from the group consisting of obesity, diabetes, hyperinsulinemia, metabolic syndrome X, polycystic ovary syndrome, climacteric disorders associated with oxidative stress, inflammatory response to tissue injury, pathogenesis of emphysema, ischemia-associated organ injury, doxorubicin-induced cardiac injury, drug-induced hepatotoxicity, atherosclerosis, and hypertoxic lung injury.
